(19) United States
(12) Patent Application Publication
(10) Pub. No.: US 2026/0114112 A1
(43) Pub. Date: Apr. 23, 2026

(54) ORGANIC COMPOUND, ELECTRONIC COMPONENT AND ELECTRONIC APPARATUS

(71) Applicant: SHAANXI LIGHTE OPTOELECTRONICS MATERIAL CO., LTD., Xi'an, Shaanxi (CN)

(72) Inventors: Xianbin XU, Xi 'an, Shaanxi (CN); Lei YANG, Xi 'an, Shaanxi (CN)

(21) Appl. No.: 19/161,537

(22) PCT Filed: Apr. 12, 2024
(86) PCT No.: PCT/CN2024/087615

§ 371 Date: Sep. 2, 2025

(30) Foreign Application Priority Data
Aug. 3, 2023 (CN) 202310973289.4

(51) Int. Cl. H10K 50/11; C07B 59/00; C07D 413/10; H10K 85/30; H10K 85/60

(52) U.S. Cl. CPC H10K 50/11; C07B 59/004; C07D 413/10; H10K 85/342; H10K 85/626; H10K 85/633; H10K 85/636; H10K 85/654; H10K 85/656; H10K 85/6572; H10K 85/6574; H10K 85/6576; C07B 2200/05

(57) ABSTRACT

The present application relates to the technical field of organic electroluminescent materials. Provided are an organic compound, an organic electroluminescent device and an electronic device. The organic compound provided by the present application has a structure as represented by Formula 1, and can significantly improve the performance of a device.

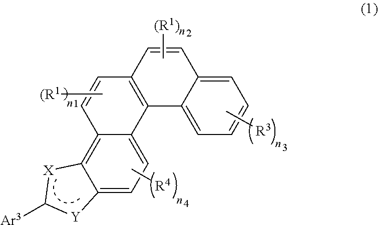

### CROSS-REFERENCE TO RELATED APPLICATIONS

The present application claims priority to Chinese Patent Application No. 202310973289.4 filed on Aug. 3, 2023, and the entire contents of the Chinese Patent Application are hereby incorporated in the present application.

### TECHNICAL FIELD

The present application relates to the technical field of organic electroluminescent materials and in particular to an organic compound, electronic component and electronic apparatus.

### BACKGROUND

Organic electroluminescent devices, such as organic light-emitting diodes (OLEDs), typically include a cathode and an anode disposed opposite each other, and a functional layer disposed between the cathode and the anode. The functional layer is composed of multiple organic or inorganic film layers, and generally includes an organic light-emitting layer, a hole transport layer, an electron transport layer, etc. When a voltage is applied to the cathode and the anode, an electric field is generated between the two electrodes, under the action of the electric field, electrons on the cathode side move towards the electroluminescent layer, and holes on the anode side also move towards the light-emitting layer, electrons and holes combine in the electroluminescent layer to form excitons, which are in an excited state and release energy outward, thereby causing the electroluminescent layer to emit light externally.

In the existing organic electroluminescent devices, the most primary problems are reflected in the lifetime and efficiency. With the large area display trend, the driving voltage is also correspondingly increased, and the luminescence efficiency and current efficiency need to be improved, so it is necessary to continue researching and developing novel materials in order to further improve the performance of the organic electroluminescent devices.

### SUMMARY

Against the above problems in the prior art, an objective of the present application is to provide an organic compound, and an organic electroluminescent device and an electronic apparatus comprising the same. The organic compound, when used in an organic electroluminescent device, can improve the performance of the device.

A first aspect of the present application provides an organic compound having a structure shown in Formula 1:

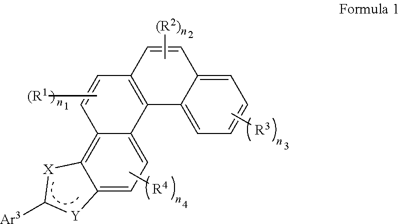

wherein, one of X and Y is —O— or —S— and the other is —N═;
each R¹, R², R³ and R⁴ are the same or different, and are each independently selected from the group consisting of hydrogen, deuterium, cyano, halogen, alkyl having 1 to 10 carbon atoms, haloalkyl having 1 to 10 carbon atoms, deuterated alkyl having 1 to 10 carbon atoms, aryl having 6 to 20 carbon atoms, deuterated aryl having 6 to 20 carbon atoms, heteroaryl having 3 to 20 carbon atoms, cycloalkyl having 3 to 10 carbon atoms, and a group shown in Formula 2, and one and only one of R¹, R², R³ and R⁴ is the group shown in Formula 2;

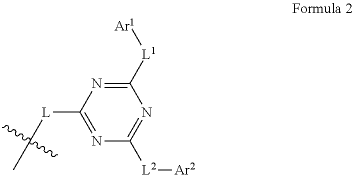

n₁ is the number of R¹ and is selected from the group consisting of 0, 1, and 2; when n₁ is greater than 1, any two R¹s are the same or different;
n₂ is the number of R² and is selected from the group consisting of 0, 1, and 2; when n₂ is greater than 1, any two R²s are the same or different;
n₃ is the number of R³ and is selected from the group consisting of 0, 1, 2, 3, and 4; when n₃ is greater than 1, any two R³s are the same or different;
n⁴ is the number of R⁴ and is selected from the group consisting of 0, 1, and 2; when n₄ is greater than 1, any two R⁴s are the same or different;
L, L¹, and L² are the same or different, and are each independently selected from the group consisting of a single bond, substituted or unsubstituted arylene having 6 to 30 carbon atoms, and substituted or unsubstituted heteroarylene having 3 to 30 carbon atoms;
Ar¹, Ar², and Ar³ are the same or different, and are each independently selected from the group consisting of substituted or unsubstituted aryl having 6 to 30 carbon atoms, and substituted or unsubstituted heteroaryl having 3 to 30 carbon atoms;
the substituent(s) in L, L¹, L², Ar¹, Ar², and Ar³ are the same or different, and are each independently selected from the group consisting of deuterium, cyano, halogen, alkyl having 1 to 10 carbon atoms, haloalkyl having 1 to 10 carbon atoms, deuterated alkyl having 1 to 10 carbon atoms, aryl having 6 to 20 carbon atoms, deuterated aryl having 6 to 20 carbon atoms, heteroaryl having 3 to 20 carbon atoms, cycloalkyl having 3 to 10 carbon atoms, and trialkylsilyl having 3 to 12 carbon atoms;
optionally, any two adjacent substituents in Ar¹, Ar², and Ar³ form a saturated or unsaturated 5- to 15-membered ring.

A second aspect of the present application provides an organic electroluminescent device, comprising an anode and a cathode disposed opposite each other, and a functional layer disposed between the anode and the cathode; wherein the functional layer comprises the organic compounds described above.

A third aspect of the present application provides an electronic apparatus, comprising the organic electroluminescent device described in the second aspect.

The compounds of the present application contain a phenanthrobenzoxazole/thiazole core structure in their structures, which is connected to a triazine compound, and the compounds respectively serve as electron-transport red-light host materials. Among them, the phenanthrobenzoxazole/thiazole has a large conjugation system, which can enhance the intermolecular interactions and improve the carrier mobility of the compounds after connecting with triazine. When the compounds of the present application are used as the electron transport materials in the mixed host materials, they can improve the carrier balance in the light-emitting layer, broaden the carrier recombination zone, enhance the efficiency of exciton generation and utilization, and improve the luminescence efficiency and lifetime of the device.

### BRIEF DESCRIPTION OF THE DRAWINGS

The drawings are used for a further understanding of the present application and constitute a part of the specification and are used to explain the present application together with the following specific embodiments, but do not constitute a limitation of the present application.

FIG. 1 is a schematic structural diagram of an organic electroluminescent device according to one embodiment of the present application.

FIG. 2 is a schematic structural diagram of an electronic apparatus according to one embodiment of the present application.

Reference Signs

| | | | |
|---|---|---|---|
| 100, Anode; | 200, Cathode; | 300, Functional layer; | 310, Hole injection |
| | | | layer; |
| 321, Hole transport | 322, Hole adjustment | 330, Organic light- | 340, Electron transport |
| layer; | layer; | emitting layer; | layer; |
| 350, Electron injection | 400, Electronic | | |
| layer; | apparatus | | |

### DETAILED DESCRIPTION OF EMBODIMENTS

Exemplary embodiments will now be described more comprehensively with reference to the accompanying drawings. The exemplary embodiments, however, can be implemented in a variety of forms and should not be interpreted as being limited to the examples set forth herein; On the contrary, these examples are provided to make the present application more comprehensive and complete, and to convey the concepts of these exemplary embodiments fully to those skill in the art. Features, structures, or characteristics described herein can be combined in one or more embodiment(s) in any suitable manner. In the following description, many specific details are provided to give a full understanding of the examples of the present application.

In a first aspect, the present application provides an organic compound having a structure shown in Formula 1 below:

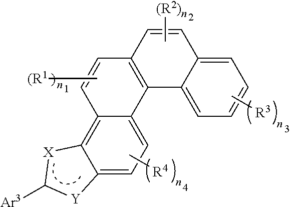

wherein one of X and Y is —O— or —S— and the other is —N═;
each R¹, R², R³ and R⁴ are the same or different, and are each independently selected from the group consisting of hydrogen, deuterium, cyano, halogen, alkyl having 1 to 10 carbon atoms, haloalkyl having 1 to 10 carbon atoms, deuterated alkyl having 1 to 10 carbon atoms, aryl having 6 to 20 carbon atoms, deuterated aryl having 6 to 20 carbon atoms, heteroaryl having 3 to 20 carbon atoms, cycloalkyl having 3 to 10 carbon atoms, and a group shown in Formula 2, and one and only one of R¹, R², R³ and R⁴ is the group shown in Formula 2;

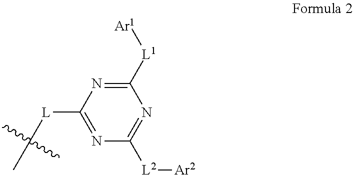

n₁ is the number of R¹ and is selected from the group consisting of 0, 1, and 2; when n₁ is greater than 1, any two R¹s are the same or different;
n₂ is the number of R² and is selected from the group consisting of 0, 1, and 2; when n₂ is greater than 1, any two R²s are the same or different;
n₃ is the number of R³ and is selected from the group consisting of 0, 1, 2, 3, and 4; when n₃ is greater than 1, any two R³s are the same or different;
n⁴ is the number of R⁴ and is selected from the group consisting of 0, 1, and 2; when n₄ is greater than 1, any two R⁴s are the same or different;
L, L¹, and L² are the same or different, and are each independently selected from the group consisting of a single bond, substituted or unsubstituted arylene having 6 to 30 carbon atoms, and substituted or unsubstituted heteroarylene having 3 to 30 carbon atoms;
Ar¹, Ar², and Ar³ are the same or different, and are each independently selected from the group consisting of substituted or unsubstituted aryl having 6 to 30 carbon atoms, and substituted or unsubstituted heteroaryl having 3 to 30 carbon atoms;
the substituent(s) in L, L¹, L², Ar¹, Ar², and Ar³ are the same or different, and are each independently selected from the group consisting of deuterium, cyano, halogen, alkyl having 1 to 10 carbon atoms, haloalkyl having 1 to 10 carbon atoms, deuterated alkyl having 1 to 10 carbon atoms, aryl having 6 to 20 carbon atoms, deuterated aryl having 6 to 20 carbon atoms, heteroaryl having 3 to 20 carbon atoms, cycloalkyl having 3 to 10 carbon atoms, and trialkylsilyl having 3 to 12 carbon atoms;
optionally, any two adjacent substituents in Ar¹, Ar², and Ar³ form a saturated or unsaturated 5- to 15-membered ring.

The

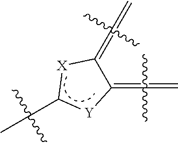

moiety of Formula 1 is a heteroaryl, e.g. when X is O or S, Y is —N═, this structure is denoted as

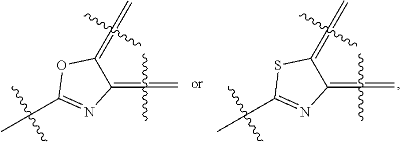 [SMILES: *c1nc(=*)c(=*)o1 ; *c1nc(=*)c(=*)s1]

respectively; or, when Y is O or S, X is —N═, and this structure is denoted aa

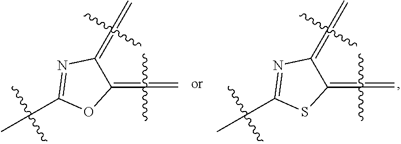 [SMILES: *c1nc(=*)c(=*)o1 ; *c1nc(=*)c(=*)s1]

respectively.

In the present application, the terms “optional” and “optionally” mean that the event or circumstance described later may or may not occur. For example, “optionally, any two adjacent substituents form a ring” means that these two substituents may or may not form a ring, including scenarios both where two adjacent substituents form a ring and where two adjacent substituents do not form a ring. For another example, “optionally, any two adjacent substituents form a ring” refers to that any two adjacent substituents are interconnected to form a ring, or any two adjacent substituents may exist independently of each other. “Any two adjacent” may include that two substituents are present on the same atom, and may also include that one substituent is present on each of adjacent atoms; among them, where two substituents are present on the same atom, the two substituents may form a saturated or unsaturated spiro-ring with the atom to which they are jointly attached; where a substituent is present on each of two adjacent atoms, these two substituents may be fused into a ring.

In the present application, a saturated or unsaturated 5- to 15-membered ring refers to a carbocyclic or heterocyclic ring comprising 5 to 15 ring atoms; Examples include, but are not limited to, cyclopentane, cyclohexane, benzene ring, fluorene ring, pyran ring, tetrahydropyran ring, piperidine ring, tetrahydropiperidine ring, and the like.

In the present application, cycloalkyl having 5 to 10 carbon atoms refers to cycloalkyl formed of 5 to 10 carbon atoms, such as, but not limited to, cyclopentyl, cyclohexyl, adamantyl, and the like.

In the present application, an unsaturated 6- to 15-membered ring refers to an unsaturated ring formed of 6 to 15 ring atoms, such as, but not limited to, a benzene ring, a fluorene ring, a furan ring, a pyran ring, and the like.

In the present application, the descriptive expressions “each . . . . . . be independently”, “each . . . . . . be respectively independently”, and “ . . . . . . be each independently” may be interchanged and all these expressions should be interpreted in a broad sense. They may both refer to specific options expressed by the same symbol in separate groups are mutually non-influential, and to specific options expressed by the same symbols within the same group are mutually non-influential. For example,

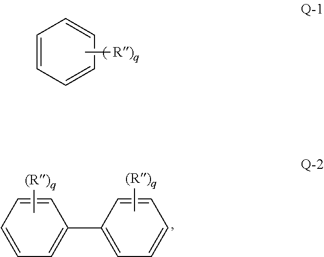

wherein each q is independently 0, 1, 2, or 3, and each R″ is independently selected from hydrogen, deuterium, fluorine, or chlorine″, means that Formula Q−1 represents that there are q substituents R″ on the benzene ring, and each R″ may be the same or different, with mutual non-influence between the options for each R″; Formula Q−2 represents that there are q substituents R″ on each benzene ring of biphenyl, and the number q of R″ substituents on the two benzene rings may be the same or different, and each R″ may be the same or different, with mutual non-influence between the options for each R″.

In the present application, the term “substituted or unsubstituted” means that the functional group following this term may or may not have a substituent (hereinafter referred to as Rc for ease of description). For example, “substituted or unsubstituted aryl” refers to an aryl having a substituent Rc or an unsubstituted aryl. Among them, the above substituent, i.e., Rc, may be, for example, deuterium, halogen, cyano, heteroaryl, aryl, alkyl, haloalkyl, deuterated alkyl, deuterated aryl, haloaryl, a cycloalkyl, etc. The number of substitutions may be one or more.

In the present application, “more” refers to two or more, for example, 2, 3, 4, 5, 6, etc.

The hydrogen atoms in the structure of the compounds of the present application include various isotopic atoms of hydrogen element, such as hydrogen (H), deuterium (D), or tritium (T).

In the present application, the number of carbon atoms of a substituted or unsubstituted functional group refers to the total number of carbon atoms. For example, if L is substituted arylene having 12 carbon atoms, the total number of carbon atoms in the arylene and its substituents is 12.

In the present application, aryl refers to an optional functional group or a substituent derived from an aromatic carbon ring. An aryl may be a monocyclic aryl (e.g., phenyl) or a polycyclic aryl. In other words, an aryl may be a monocyclic aryl, a fused-ring aryl, two or more monocyclic aryls linked by conjugated carbon-carbon single bond, a monocyclic aryl and a fused-ring aryl linked by conjugated carbon-carbon single bond, or two or more fused-ring aryls linked by conjugated carbon-carbon single bond. That is, unless otherwise specified, two or more aromatic groups linked by conjugated carbon-carbon single bond may also be regarded as an aryl of the present application. Among them, a fused-ring aryl may include, for example, a bicyclic fused aryl (e.g., naphthyl), a tricyclic fused aryl (e.g., phenanthryl, fluorenyl, and anthryl), etc. The aryl does not contain heteroatoms such as B, N, O, S, P, Se and Si. Examples of aryl include, but are not limited to, phenyl, naphthyl, fluorenyl, phenyl-naphthyl, spirobifluorenyl, anthryl, phenanthryl, biphenyl, terphenyl, triphenylene, perylenyl, benzo[9,10]phenanthryl, pyrenyl, benzofluoranthryl, chrysenyl, and the like.

In the present application, an arylene involved refers to a divalent or multivalent group formed by further removing one or more hydrogen atom(s) from an aryl.

In the present application, terphenyl includes

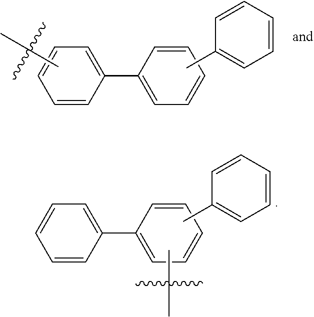

In the present application, the number of carbon atoms in substituted aryl refers to the total number of carbon atoms of aryl and the substituent(s) on the aryl. For example, substituted aryl having 18 carbon atoms, refers to the total number of carbon atoms of the aryl and the substituent(s) thereon is 18. In the present application, the number of carbon atoms of a substituted or unsubstituted aryl (arylene) may be 6, 8, 9, 10, 11, 12, 13, 14, 15, 16, 17, 18, 19, 20, 21, 22, 23, 24, 25, 26, 27, 28, 29, or 30, etc. In some embodiments, the substituted or unsubstituted aryl is a substituted or unsubstituted aryl having 6 to 30 carbon atoms, in other embodiments, the substituted or unsubstituted aryl is a substituted or unsubstituted aryl having 6 to 20 carbon atoms, in other embodiments, the substituted or unsubstituted aryl is a substituted or unsubstituted aryl having 6 to 25 carbon atoms, in other embodiments, the substituted or unsubstituted aryl is a substituted or unsubstituted aryl having 6 to 18 carbon atoms, and in other embodiments, the substituted or unsubstituted aryl is a substituted or unsubstituted aryl having 6 to 15 carbon atoms.

In the present application, fluorenyl may be substituted by one or more substituents. In the case that the above-mentioned fluorenyl is substituted, the substituted fluorenyl may be:

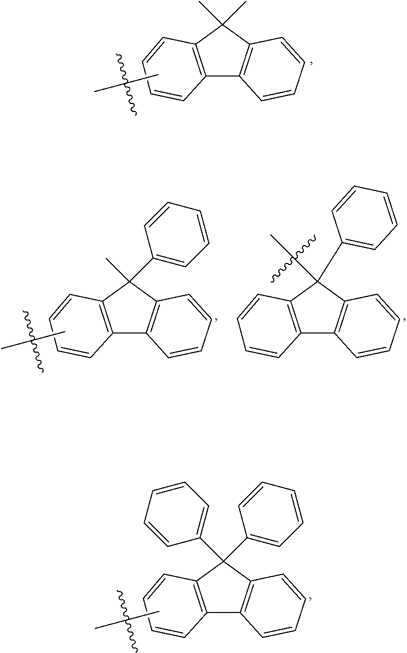

etc, but are not limited thereto.

In the present application, aryl as the substituent(s) in L, L¹, L², Ar¹, Ar², and Ar³ is, for example, but is not limited to, phenyl, naphthyl, phenanthryl, biphenyl, fluorenyl, etc.

In the present application, heteroaryl refers to a monovalent aromatic ring containing 1, 2, 3, 4, 5, or 6 heteroatoms or a derivative thereof. The heteroatoms may be one or more of B, O, N, P, Si, Se, and S. Heteroaryl may be monocyclic heteroaryl or polycyclic heteroaryl. In other words, heteroaryl may be a single aromatic ring system, or multiple aromatic ring systems linked by conjugated carbon-carbon single bond, with each of the aromatic ring systems being an aromatic monocyclic ring or an aromatic fused ring. Exemplarily, heteroaryl may include thienyl, furyl, pyrrolyl, imidazolyl, thiazolyl, oxazolyl, oxadiazolyl, triazolyl, pyridyl, dipyridyl, pyrimidinyl, triazinyl, acridinyl, pyridazinyl, pyrazinyl, quinolinyl, quinazolinyl, quinoxalinyl, phenoxazinyl, phthalazinyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl, and the like, but not limited thereto.

In the present application, heteroarylene involved refers to a divalent or multivalent group formed by further removing one or more hydrogen atoms from heteroaryl.

In the present application, the number of carbon atoms of substituted or unsubstituted heteroaryl (heteroarylene) may be selected from 3, 4, 5, 6, 7, 8, 9, 10, 11, 12, 13, 14, 15, 16, 17, 18, 19, 20, 21, 22, 23, 24, 25, 26, 27, 28, 29, or 30, etc. In some embodiments, the substituted or unsubstituted heteroaryl is a substituted or unsubstituted heteroaryl having 3 to 18 carbon atoms, in other embodiments, the substituted or unsubstituted heteroaryl is a substituted or unsubstituted heteroaryl having 3 to 12 carbon atoms, and in other embodiments, the substituted or unsubstituted heteroaryl is a substituted or unsubstituted heteroaryl having 5 to 12 carbon atoms. In the present application, heteroaryl as substituent(s) in L, L¹, L², Ar¹, Ar², and Ar³ is, for example, but not limited to, pyridyl, carbazolyl, quinolinyl, isoquinolinyl, phenanthrolinyl, benzoxazolyl, benzothiazolyl, benzimidazolyl, dibenzothienyl, and dibenzofuranyl.

In the present application, the substituted heteroaryl may mean that one or more than two hydrogen atoms in the heteroaryl are replaced by a group such as deuterium atom, halogen, —CN, aryl, heteroaryl, trialkylsilyl, alkyl, cycloalkyl, or haloalkyl. It should be understood that the number of carbon atoms in the substituted heteroaryl refers to the total number of carbon atoms in the heteroaryl and the substituent(s) thereon.

In the present application, alkyl having 1 to 10 carbon atoms may include straight-chain alkyl having 1 to 10 carbon atoms, and branched alkyl having 3 to 10 carbon atoms. The number of carbon atoms of an alkyl is for example 1, 2, 3, 4, 5, 6, 7, 8, 9, or 10, and the specific examples of the alkyl include but are not limited to, methyl, ethyl, n-propyl, isopropyl, n-butyl, isobutyl, tert-butyl, n-pentyl, isopentyl, neopentyl, n-hexyl, etc.

In the present application, halogen is for example, fluorine, chlorine, bromine, or iodine.

In the present application, the specific examples of trialkylsilyl include, but are not limited to, trimethylsilyl, triethylsilyl, etc.

In the present application, the specific examples of haloalkyl include, but are not limited to, trifluoromethyl.

In the present application, the specific examples of deuterated alkyl include, but are not limited to, trideuteromethyl.

In the present application, deuterated aryl refers to aryl containing deuterium substitution, such as, but not limited to, pentadeuterophenyl, heptadeuteronaphthyl, deuterobiphenyl, and the like.

In the present application, haloaryl refers to aryl having halogen substituent, such as, but not limited to, fluorophenyl, fluoronaphthyl, fluorobiphenyl, and the like.

In the present application, the number of carbon atoms of cycloalkyl having 3 to 10 carbon atoms is, for example, 3, 4, 5, 6, 7, 8, or 10. Specific examples of cycloalkyl include, but are not limited to, cyclopentyl, cyclohexyl, and adamantyl.

In the present application, a non-positional connection bond refers to a single bond

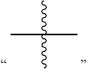

extending from a ring system, which represents that one end of the connection bond may connect to any position in the ring system through which the bond passes, and the other end connects to the rest of the compound molecule. For example, as shown in Formula (f) below, a naphthyl represented by Formula (f) is connected to other positions of the molecule through two non-positional connection bonds passing through the bicyclic structure, which indicates any of possible connecting mode shown in Formula (f-1) to Formula (f-10):

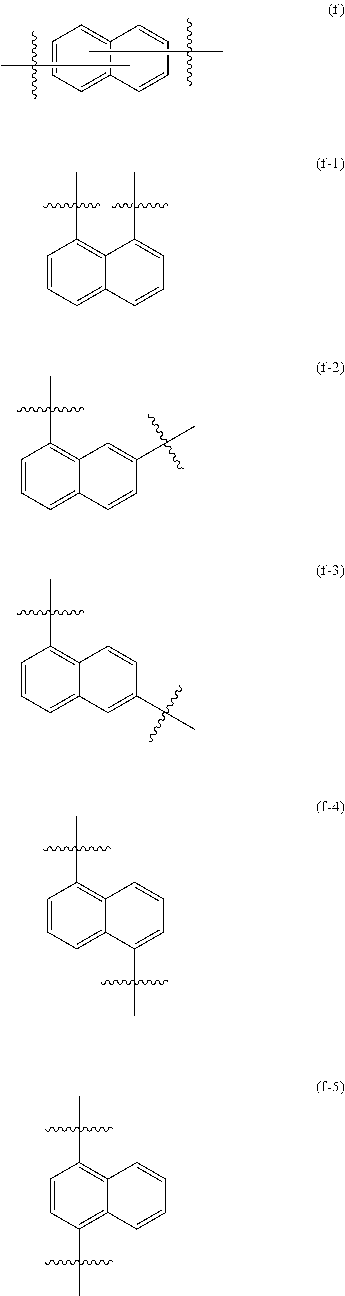

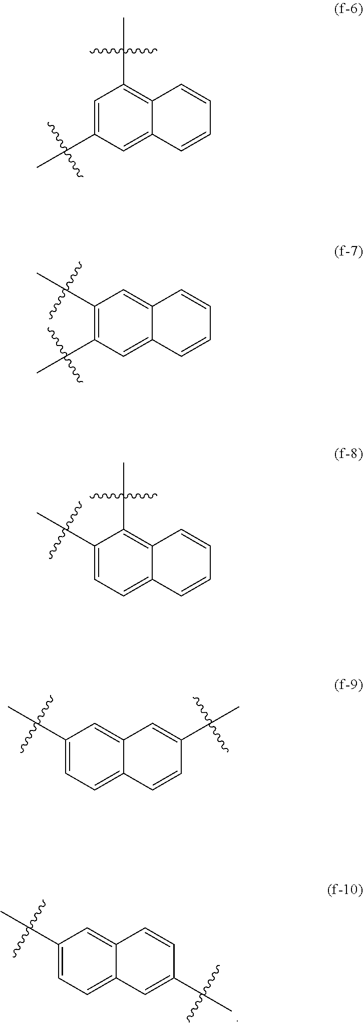 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, a dibenzofuranyl represented by Formula (X′) is connected to other positions of the molecule through a non-positional connection bond extending from the center of benzene ring at one side, which indicates any of possible connecting mode shown in Formula (X′-1) to Formula (X′-4):

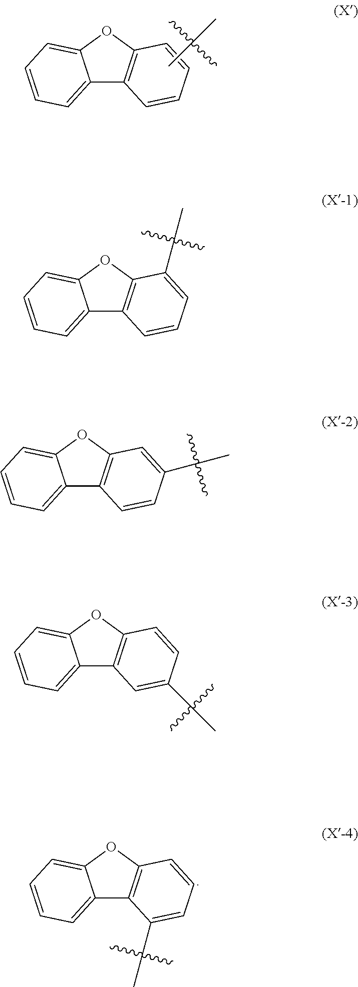

The non-positioned substituent in the present application refers to a substituent connected by a single bond extending from the center of the ring system, indicating that the substituent may be connected to any possible position in the ring system. For example, as shown in Formula (Y) below, the substituent R′ represented by Formula (Y) is linked to a quinoline ring through a non-positional connection bond, which indicates any of possible connecting mode shown in Formula (Y-1) to Formula (Y-7):

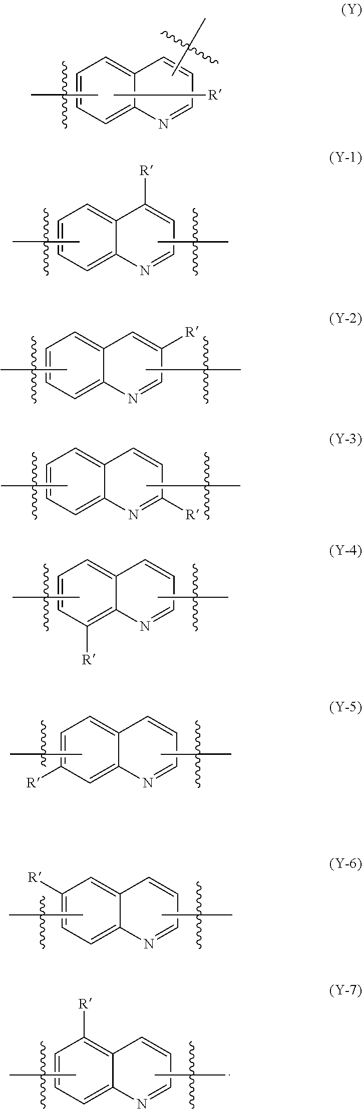

In some embodiments, Formula 1 is selected from the group consisting of the structures shown in Formula (1-1) or Formula (1-2) below:

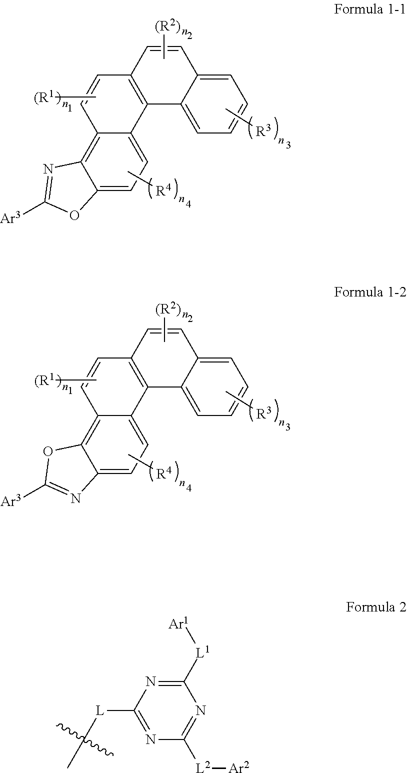

In some embodiments, one of R¹ has the structure shown in Formula 2, when n₁ is greater than 1, the rest of R¹ and each R², R³ and R⁴ are each independently selected from the group consisting of hydrogen, deuterium, cyano, halogen, alkyl having 1 to 4 carbon atoms, deuterated alkyl having 1 to 4 carbon atoms, haloalkyl having 1 to 4 carbon atoms, aryl having 6 to 12 carbon atoms, deuterated aryl having 6 to 12 carbon atoms, and heteroaryl having 5 to 12 carbon atoms.

In some embodiments, one of R² has the structure shown in Formula 2, when n₂ is greater than 1, the rest of R² and each R¹, R³ and R⁴ are each independently selected from the group consisting of hydrogen, deuterium, cyano, halogen, alkyl having 1 to 4 carbon atoms, deuterated alkyl having 1 to 4 carbon atoms, haloalkyl having 1 to 4 carbon atoms, aryl having 6 to 12 carbon atoms, deuterated aryl having 6 to 12 carbon atoms, and heteroaryl having 5 to 12 carbon atoms.

In some embodiments, one of R³ has the structure shown in Formula 2, when n₃ is greater than 1, the rest of R³ and each R¹, R² and R⁴ are each independently selected from the group consisting of hydrogen, deuterium, cyano, halogen, alkyl having 1 to 4 carbon atoms, deuterated alkyl having 1 to 4 carbon atoms, haloalkyl having 1 to 4 carbon atoms, aryl having 6 to 12 carbon atoms, deuterated aryl having 6 to 12 carbon atoms, and heteroaryl having 5 to 12 carbon atoms.

In some embodiments, one of R⁴ has the structure shown in Formula 2, when n₄ is greater than 1, the rest of R⁴ and each R¹, R² and R³ are each independently selected from the group consisting of hydrogen, deuterium, cyano, halogen, alkyl having 1 to 4 carbon atoms, deuterated alkyl having 1 to 4 carbon atoms, haloalkyl having 1 to 4 carbon atoms, aryl having 6 to 12 carbon atoms, deuterated aryl having 6 to 12 carbon atoms, and heteroaryl having 5 to 12 carbon atoms.

In some embodiments, each R¹, R², R³, and R⁴ are the same or different, and one and only one of R¹, R², R³, and R⁴ is a group shown in Formula 2, and the rest are each independently selected from the group consisting of hydrogen, deuterium, cyano, fluorine, methyl, ethyl, isopropyl, tert-butyl, and phenyl.

In some embodiments, Ar¹, Ar², and Ar³ are the same or different, and are each independently selected from the group consisting of substituted or unsubstituted aryl having 6, 7, 8, 9, 10, 11, 12, 13, 14, 15, 16, 17, 18, 19, 20, 21, 22, 23, 24, or 25 carbon atoms, and substituted or unsubstituted heteroaryl having 5, 6, 7, 8, 9, 10, 12, 13, 14, 15, 16, 17, or 18 carbon atoms.

In some embodiments, Ar¹, Ar², and Ar³ are the same or different, and are each independently selected from the group consisting of substituted or unsubstituted aryl having 6 to 25 carbon atoms, and substituted or unsubstituted heteroaryl having 5 to 18 carbon atoms.

Alternatively, the substituent(s) in Ar¹, Ar², and Ar³ are the same or different, and are each independently selected from the group consisting of deuterium, halogen, cyano, alkyl having 1 to 4 carbon atoms, deuterated alkyl having 1 to 4 carbon atoms, trialkylsilyl having 3 to 7 carbon atoms, naphthyl, phenyl, and pentadeuterophenyl;

optionally, any two adjacent substituents in Ar¹, Ar², and Ar³ form a fluorene ring.

Further alternatively, Ar³ is selected from the group consisting of substituted or unsubstituted aryl having 6 to 15 carbon atoms and substituted or unsubstituted heteroaryl having 12 to 18 carbon atoms.

In some embodiments, Ar¹, Ar², and Ar³ are the same or different, and are each independently selected from the group consisting of substituted or unsubstituted phenyl, substituted or unsubstituted biphenyl, substituted or unsubstituted terphenyl, substituted or unsubstituted naphthyl, substituted or unsubstituted phenanthryl, substituted or unsubstituted fluorenyl, substituted or unsubstituted spirobifluorenyl, substituted or unsubstituted triphenylene, substituted or unsubstituted pyridyl, substituted or unsubstituted dibenzothienyl, substituted or unsubstituted dibenzofuranyl, and substituted or unsubstituted carbazolyl.

Alternatively, the substituent(s) in Ar¹, Ar², and Ar³ are the same or different, and are each independently selected from the group consisting of deuterium, fluorine, cyano, methyl, ethyl, isopropyl, tert-butyl, trimethylsilyl, phenyl, naphthyl, and pentadeuterophenyl.

Further alternatively, Ar³ is selected from the group consisting of substituted or unsubstituted phenyl, substituted or unsubstituted biphenyl, substituted or unsubstituted naphthyl, substituted or unsubstituted phenanthryl, substituted or unsubstituted dibenzofuranyl, and substituted or unsubstituted dibenzothienyl.

In some embodiments, Ar¹ and Ar² are each independently selected from substituted or unsubstituted group W; wherein the unsubstituted group W is selected from the group consisting of the following groups:

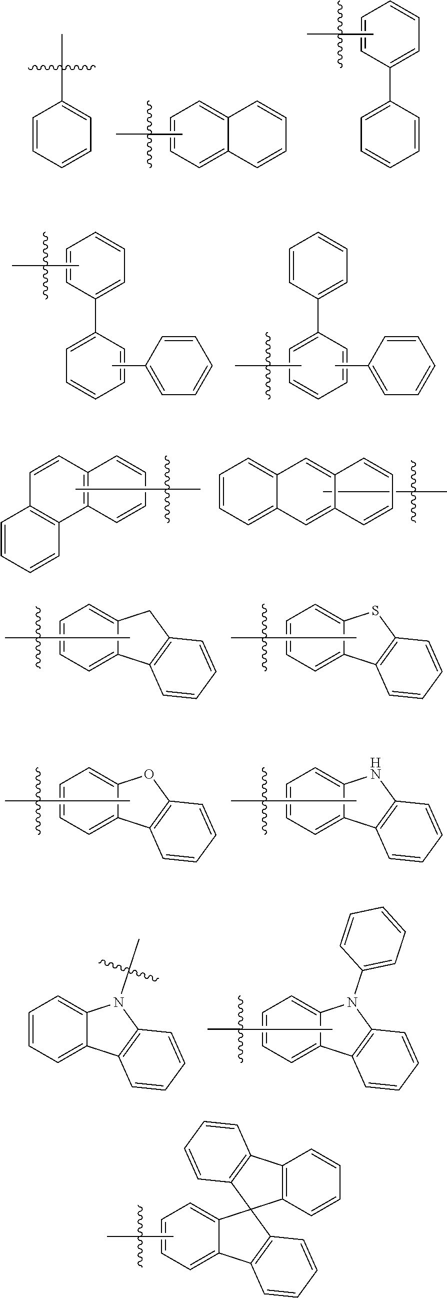

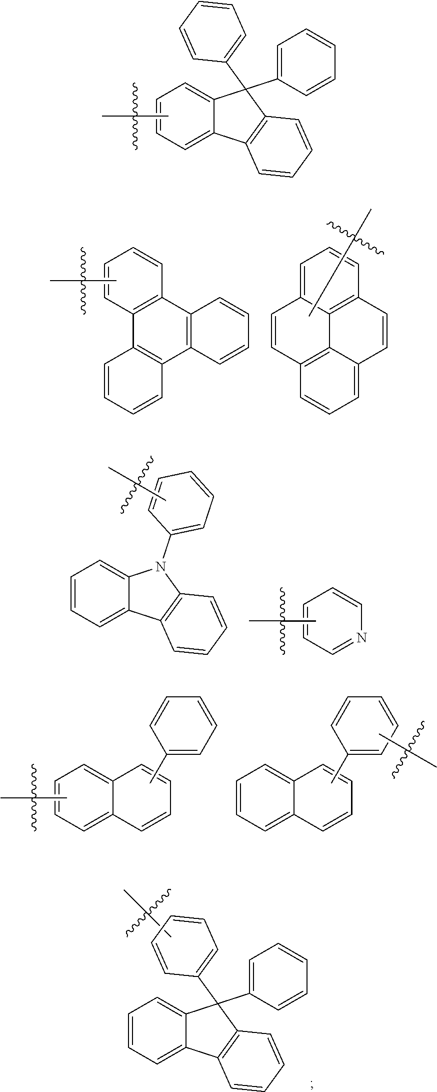

The substituted group W has one or more than two substituents, and the substituents on the substituted group W are each independently selected from the group consisting of deuterium, fluorine, cyano, methyl, ethyl, isopropyl, tert-butyl, and phenyl, and when the number of substituents on the group W is greater than 1, the substituents are each the same or different.

In some embodiments, Ar¹ and Ar² are the same or different, and are each independently selected from the group consisting of the following groups:

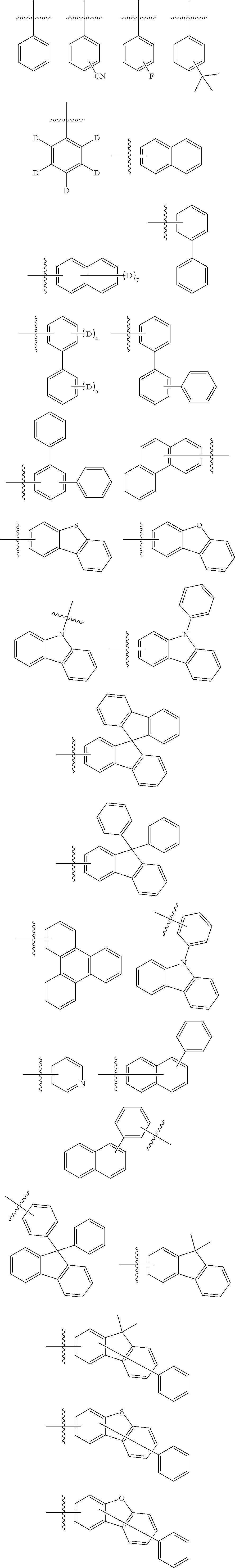

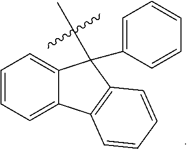 [SMILES: *C1(c2ccccc2)c2ccccc2-c2ccccc21]

In some embodiments, Ar¹ and Ar² are the same or different, and are each independently selected from the group consisting of the following groups:

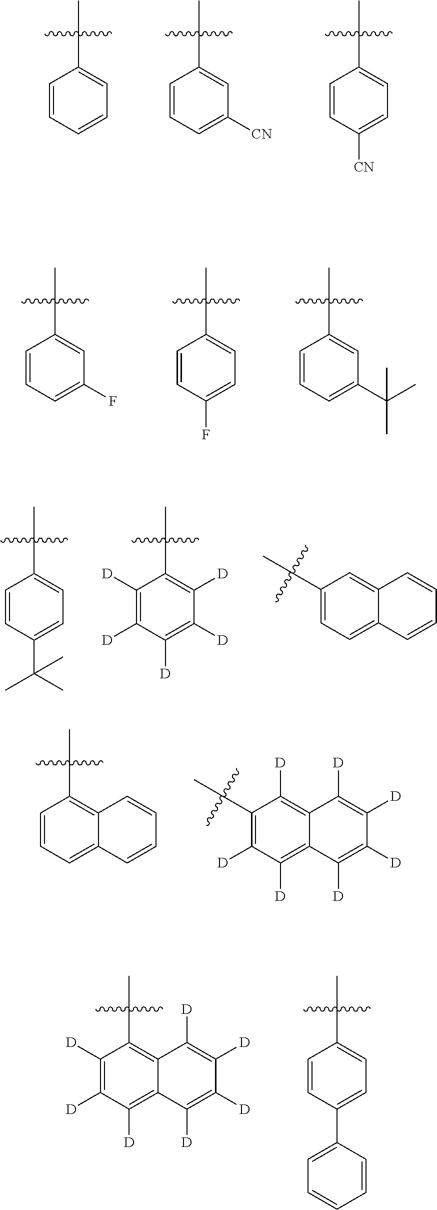

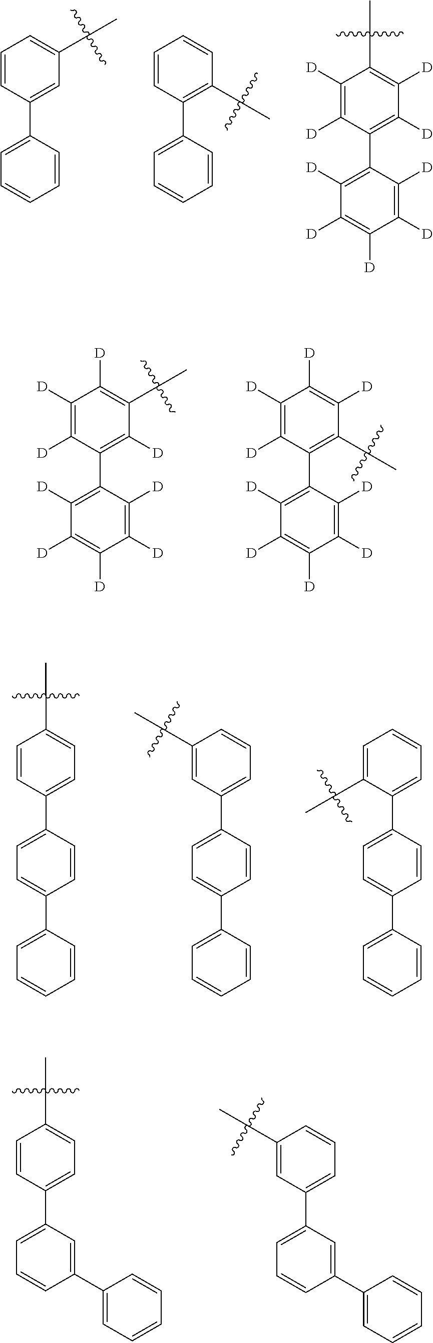

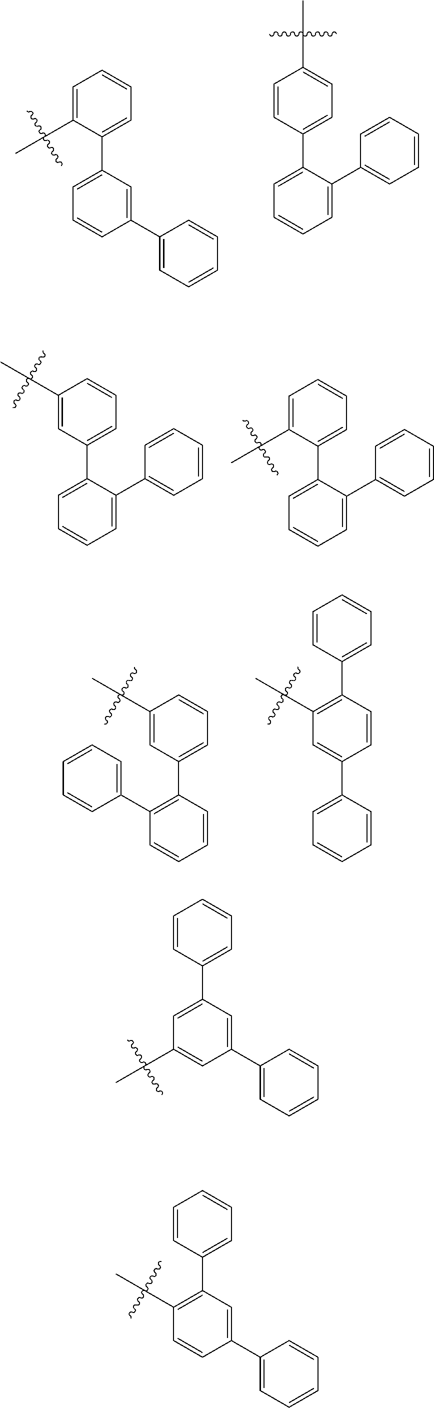

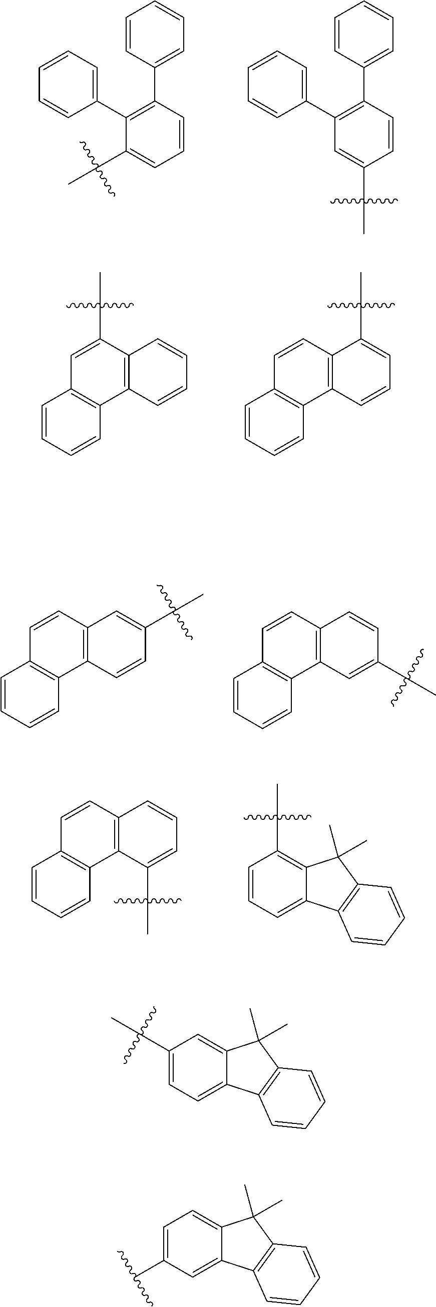

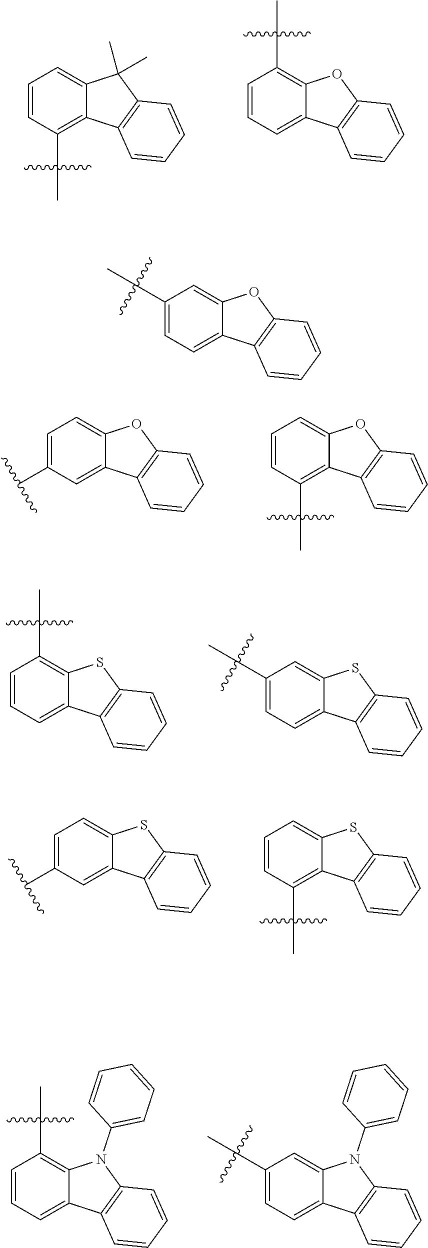

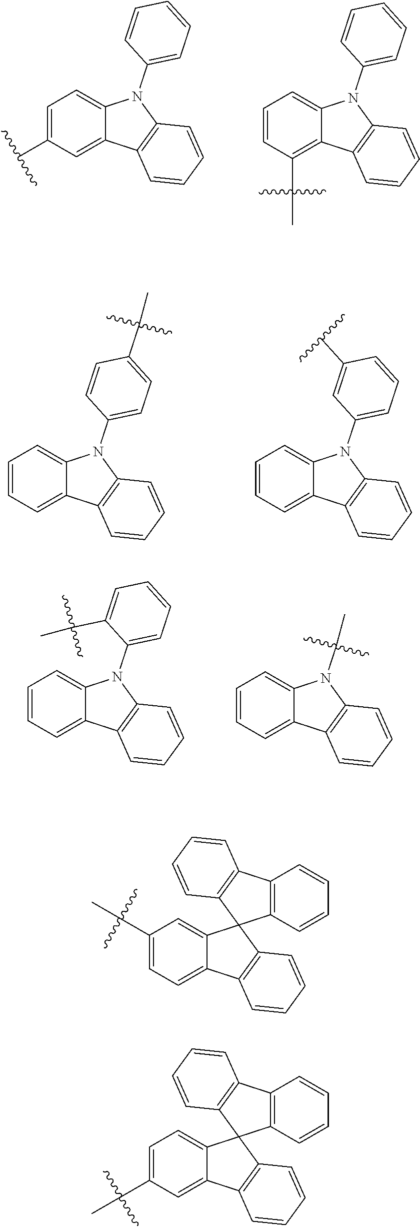

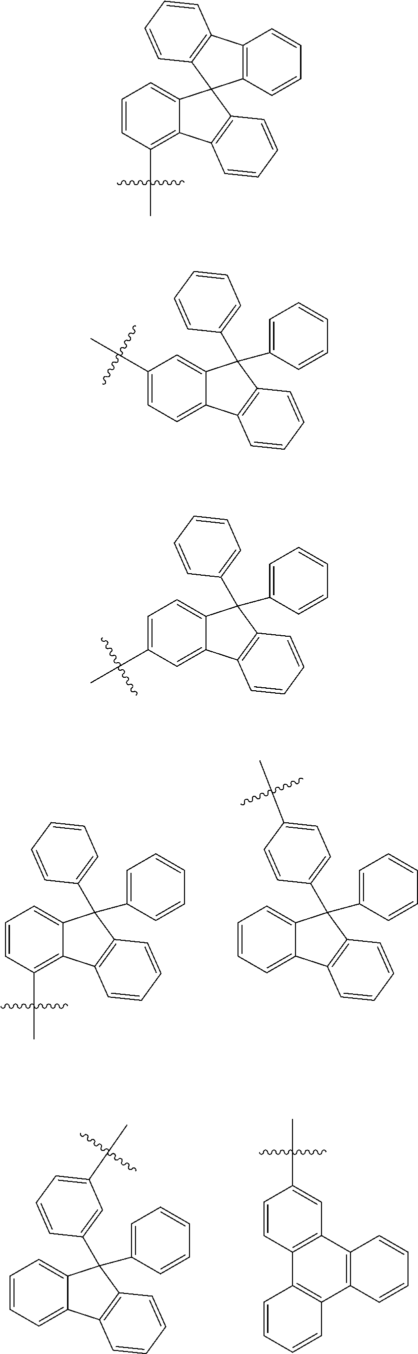

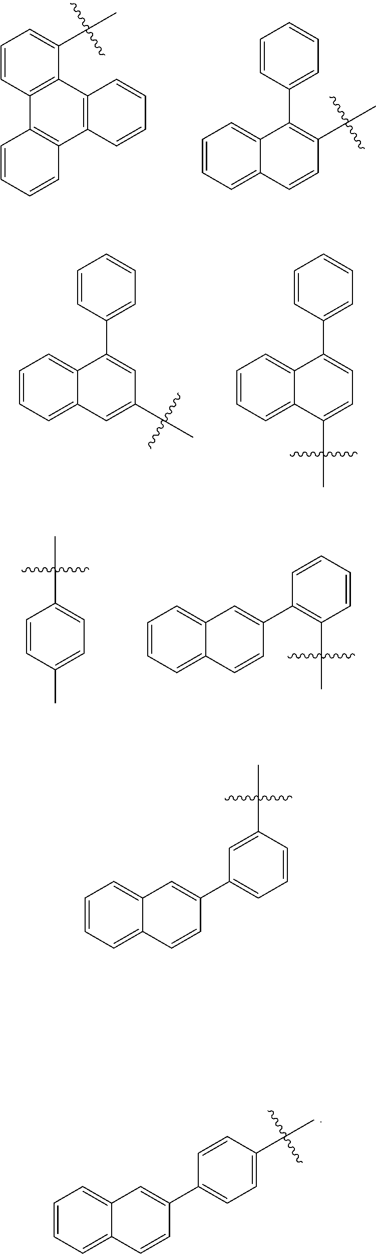

In some embodiments, Ar³ is selected from substituted or unsubstituted group Q; wherein the unsubstituted group Q is selected from the group consisting of the following groups:

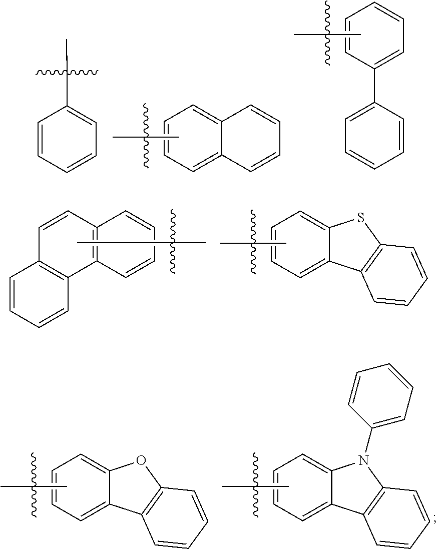

The substituted group Q has one or more than two substituents, and the substituents on the substituted group Q are each independently selected from the group consisting of deuterium, fluorine, cyano, methyl, ethyl, isopropyl, tert-butyl, and phenyl, and when the number of substituents on the group Q is greater than 1, the substituents are each the same or different.

In some embodiments, Ar³ is selected from the group consisting of the following groups:

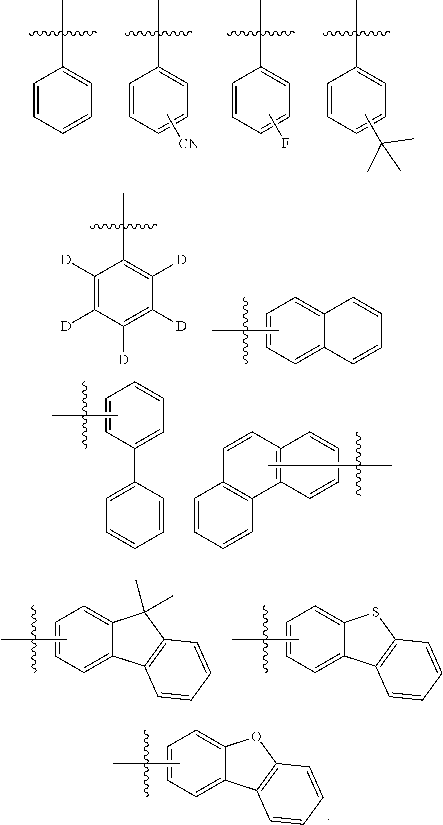

In some embodiments Ar³ is selected from the group consisting of the following groups:

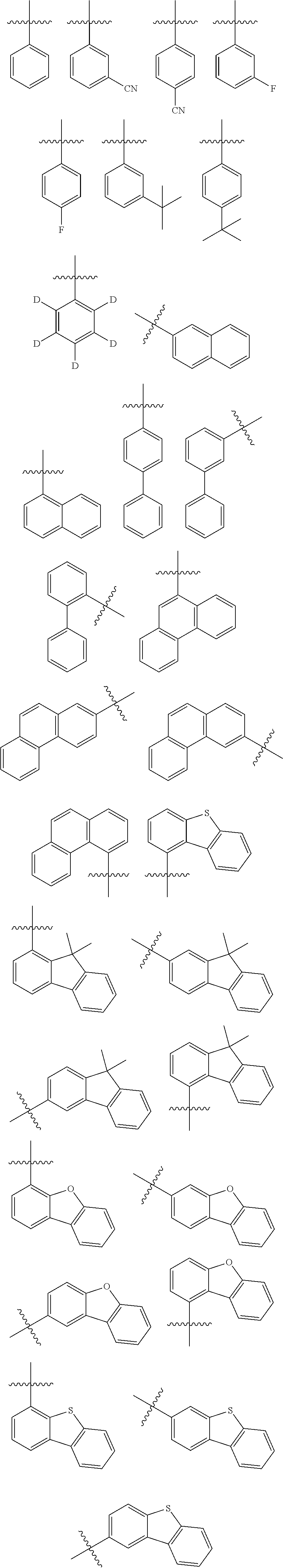

In some embodiments, L¹, L² and L are the same or different, and are each independently selected from the group consisting of a single bond, substituted or unsubstituted arylene having 6 to 15 carbon atoms, and substituted or unsubstituted heteroarylene having 12 to 18 carbon atoms.

Alternatively, L¹, L² and L are the same or different, and are each independently selected from the group consisting of a single bond, and substituted or unsubstituted arylene having 6 to 12 carbon atoms.

In some embodiments, L¹, L² and L are the same or different, and are each independently selected from the group consisting of a single bond, substituted or unsubstituted arylene having 6, 7, 8, 9, 10, 11, 12, 13, 14, or 15 carbon atoms, and substituted or unsubstituted heteroarylene having 12, 13, 14, 15, 16, 17, or 18 carbon atoms.

In some embodiments, the substituent(s) in L¹, L² and L are each independently selected from the group consisting of deuterium, fluorine, cyano, alkyl having 1 to 5 carbon atoms, trialkylsilyl having 3 to 7 carbon atoms, and phenyl.

In some embodiments, L¹, L² and L are the same or different, and are each independently selected from the group consisting of a single bond, substituted or unsubstituted phenylene, substituted or unsubstituted naphthylene, substituted or unsubstituted biphenylene, substituted or unsubstituted fluorenylene, substituted or unsubstituted phenanthrylene, substituted or unsubstituted dibenzothienylene, substituted or unsubstituted dibenzofuranylene, and substituted or unsubstituted carbazolylene.

In some embodiments, the substituent(s) in L¹, L², and L are the same or different, and are each independently selected from the group consisting of deuterium, fluorine, cyano, methyl, ethyl, isopropyl, tert-butyl, trimethylsilyl, and phenyl.

Alternatively, L is selected from the group consisting of a single bond, substituted or unsubstituted phenylene, and substituted or unsubstituted naphthylene.

In some embodiments, L is selected from the group consisting of a single bond and the following groups:

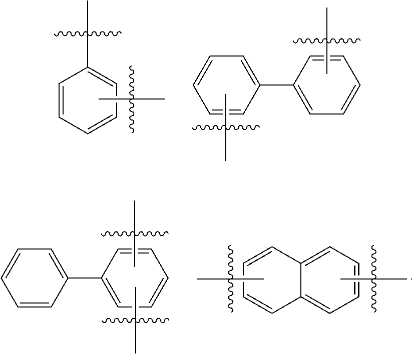

In some embodiments, L¹ and L² are each independently selected from the group consisting of a single bond and the following groups:

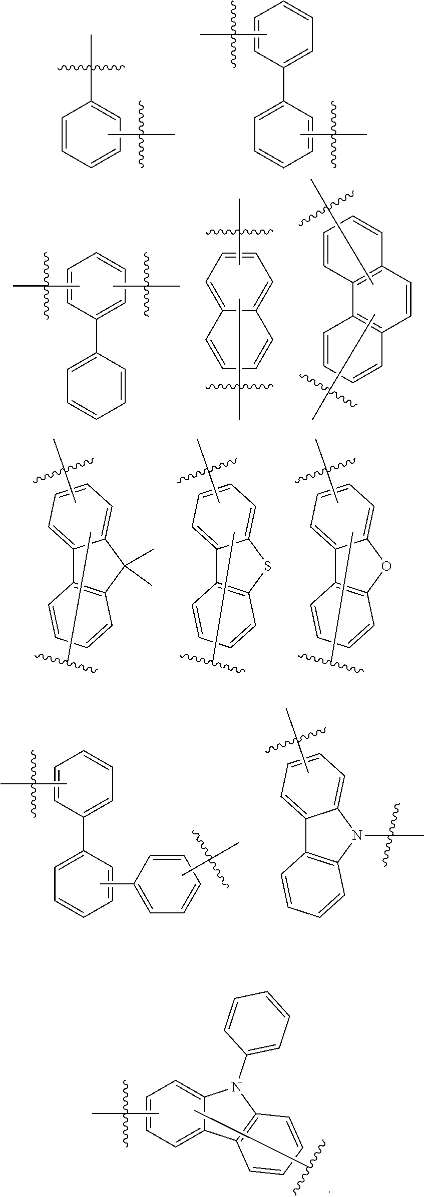

In some specific embodiments, L¹ and L² are each independently selected from the group consisting of a single bond and the following groups:

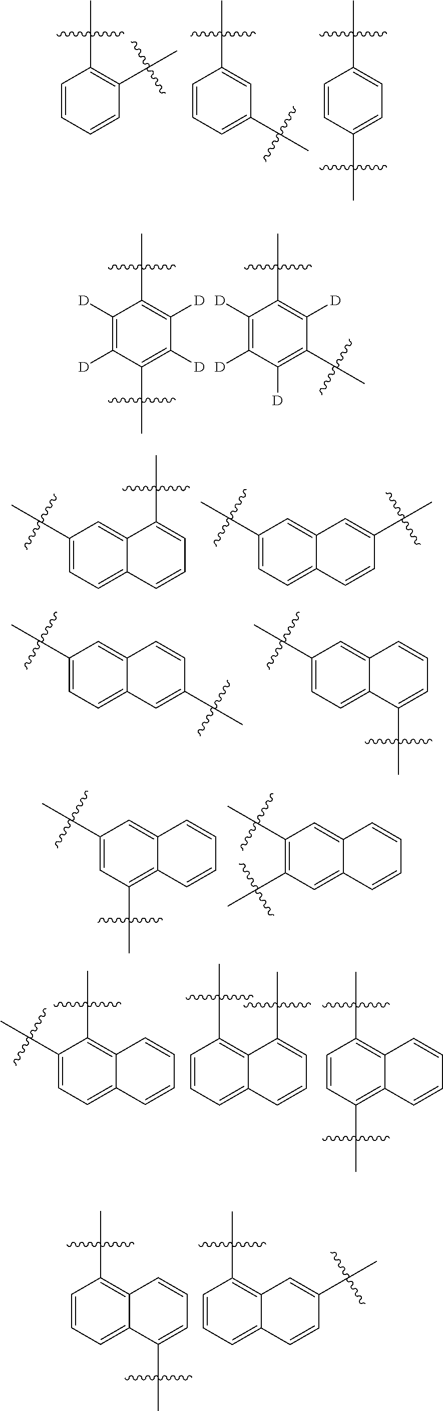

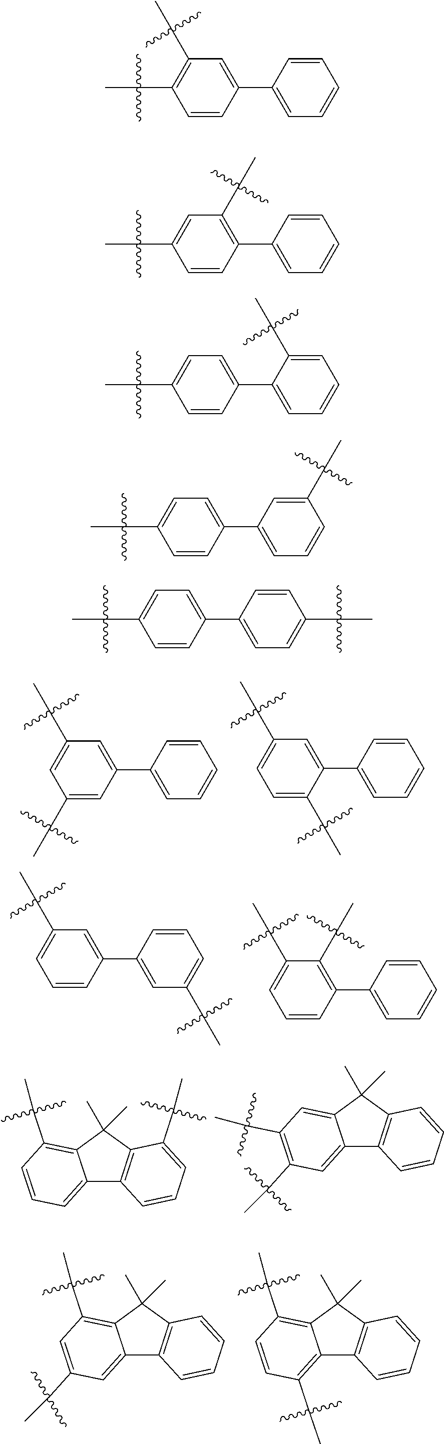

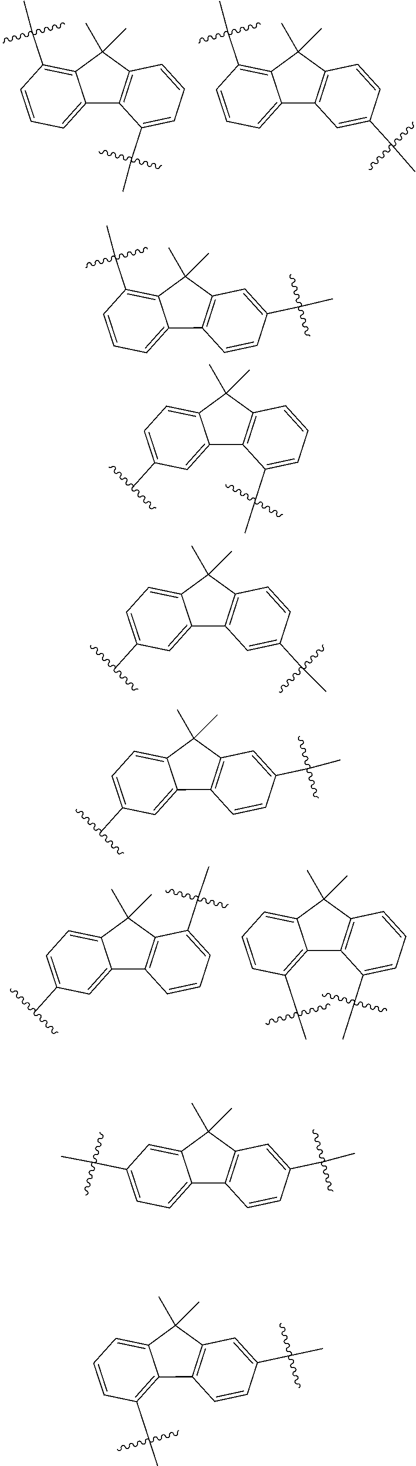

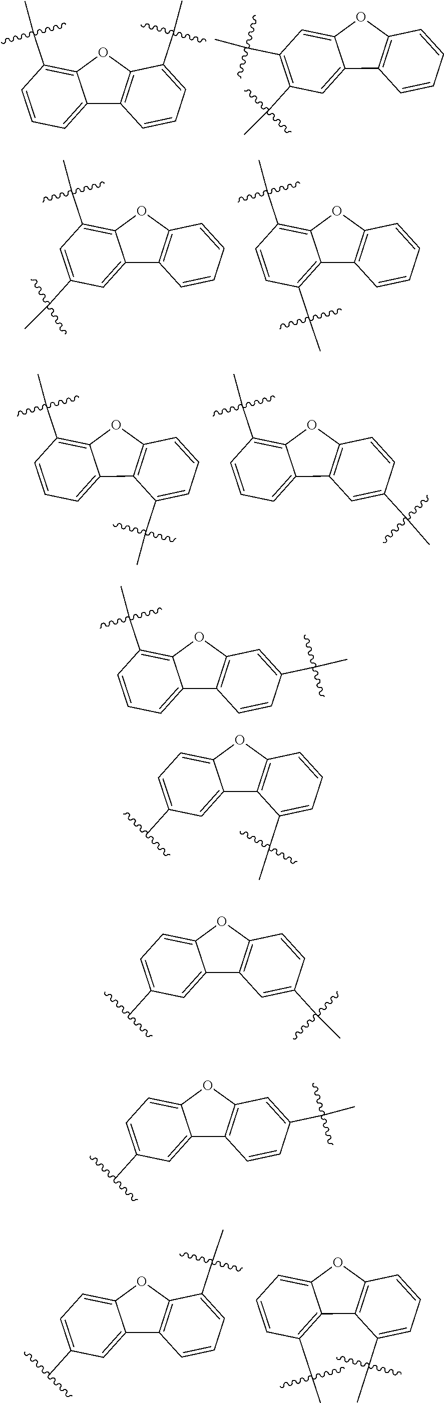

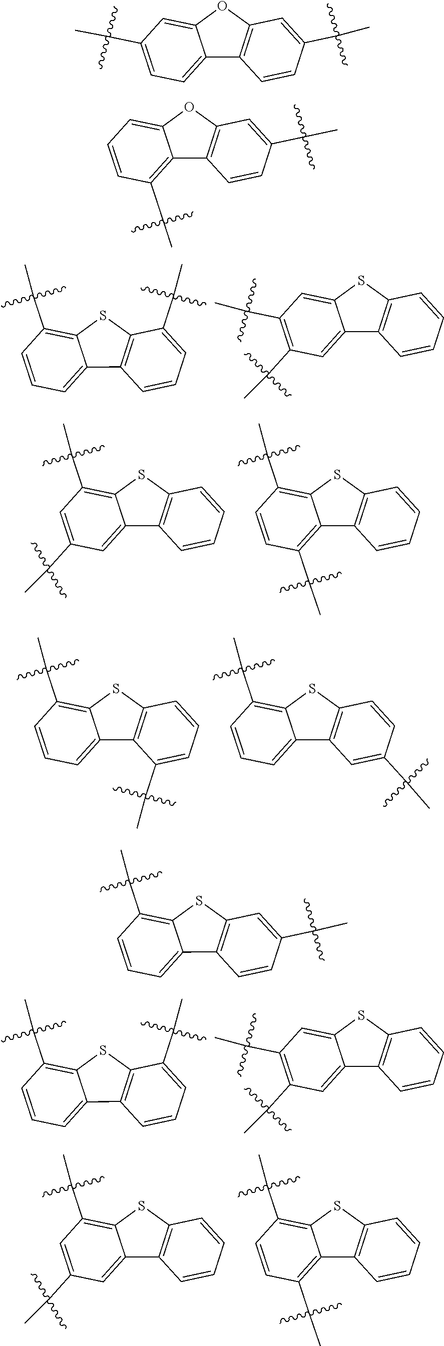

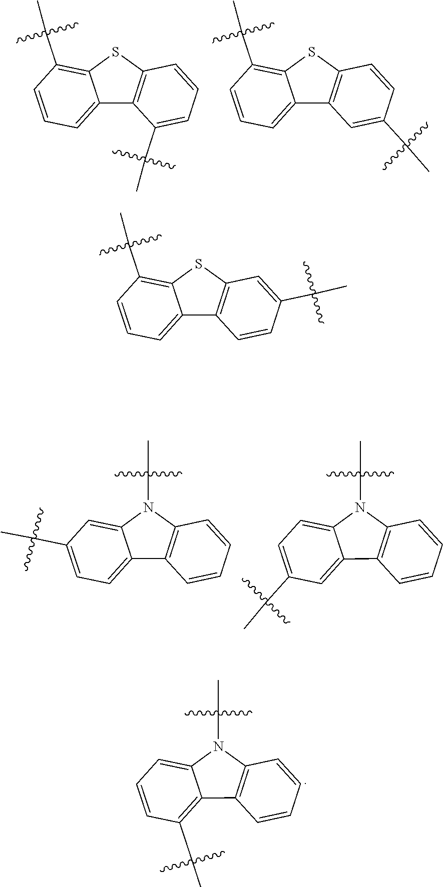

In some specific embodiments, L is selected from the group consisting of a single bond and the following groups:

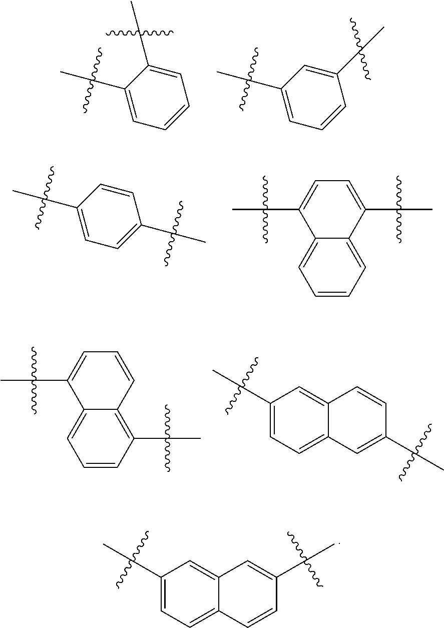

In some embodiments,

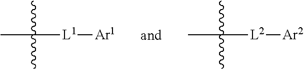

are the same or different, and are each independently selected from the group consisting of the following groups:

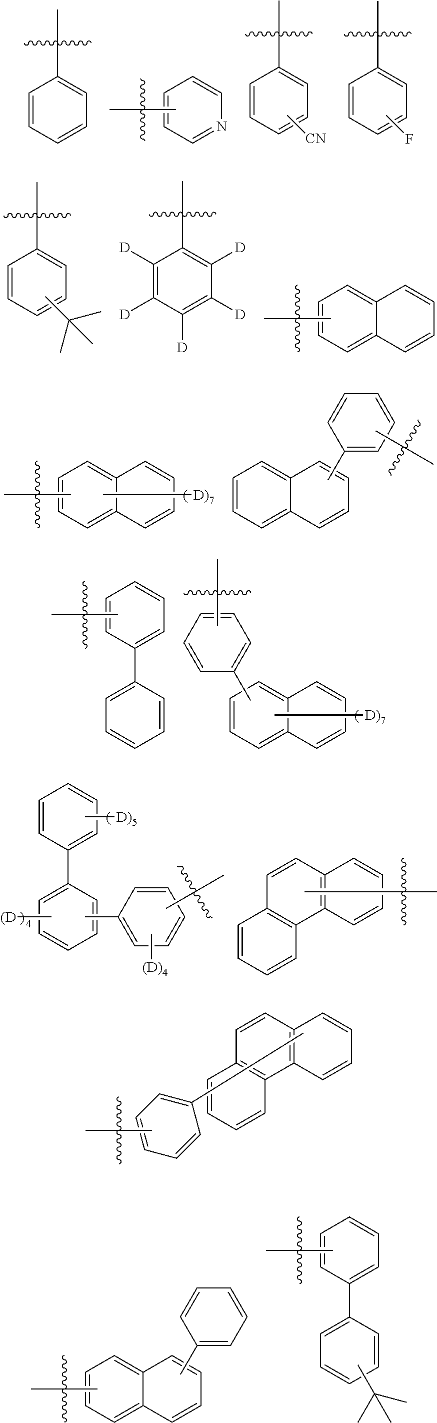

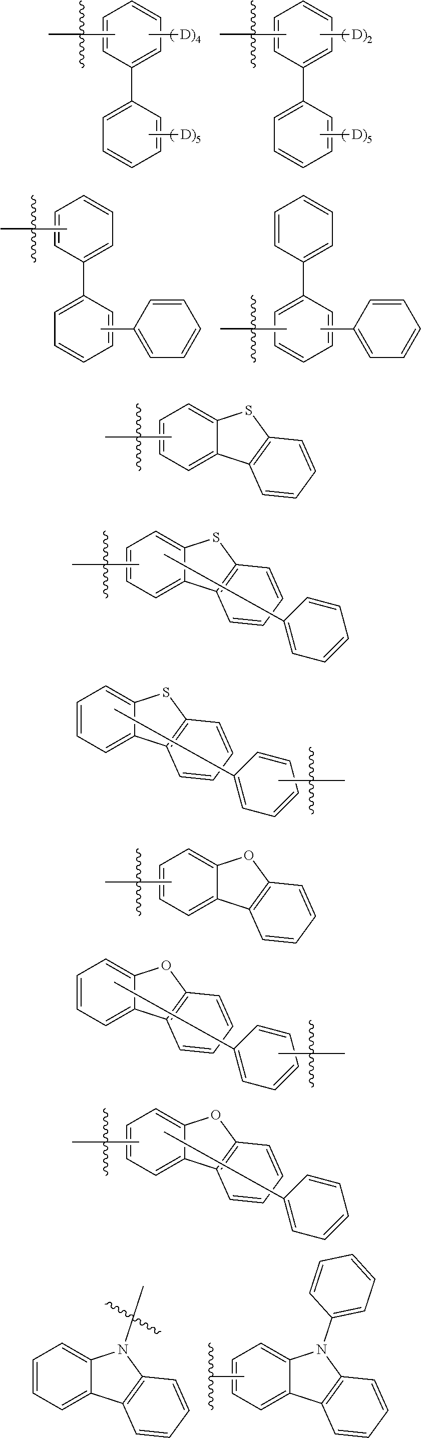

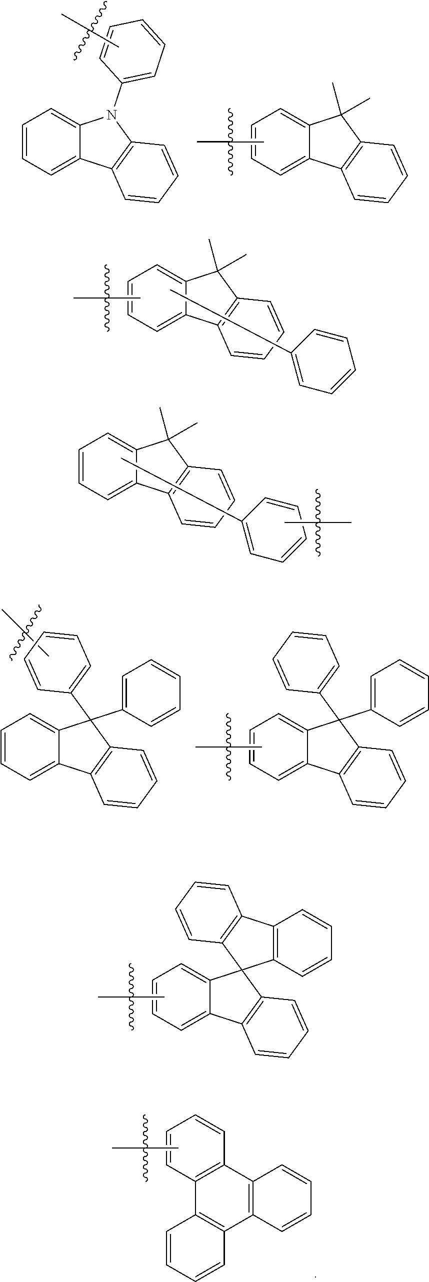

Specifically,

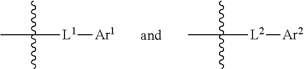

are the same or different, and are each independently selected from the group consisting of the following groups:

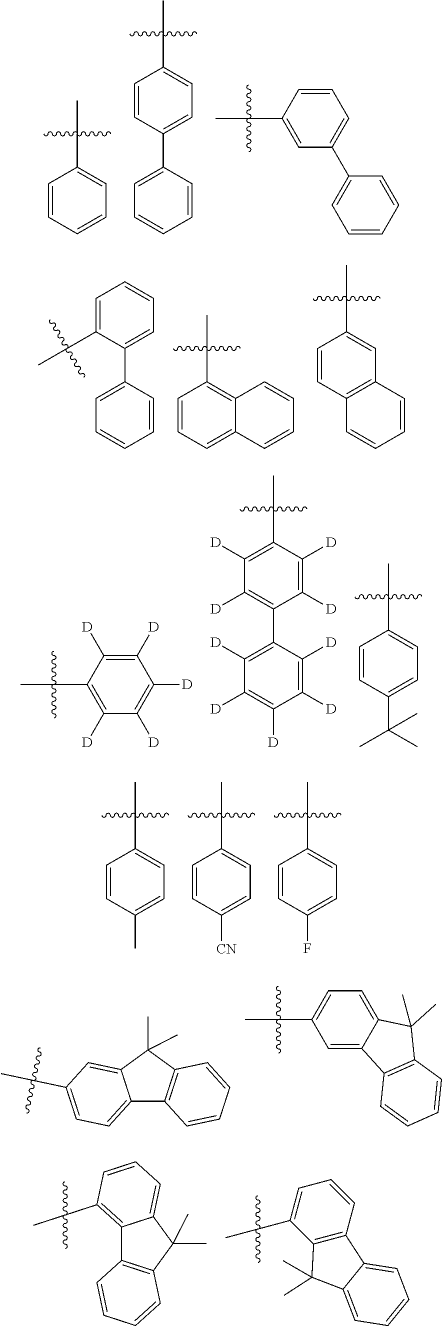

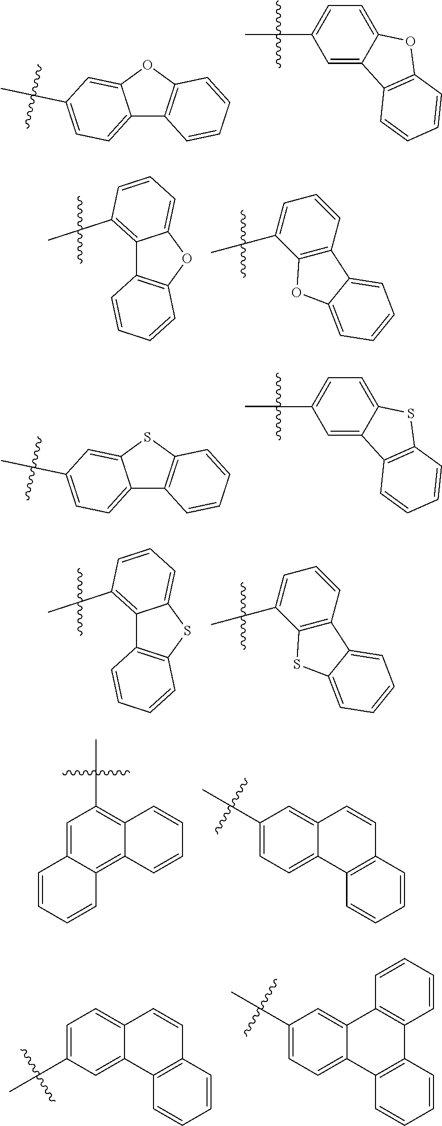

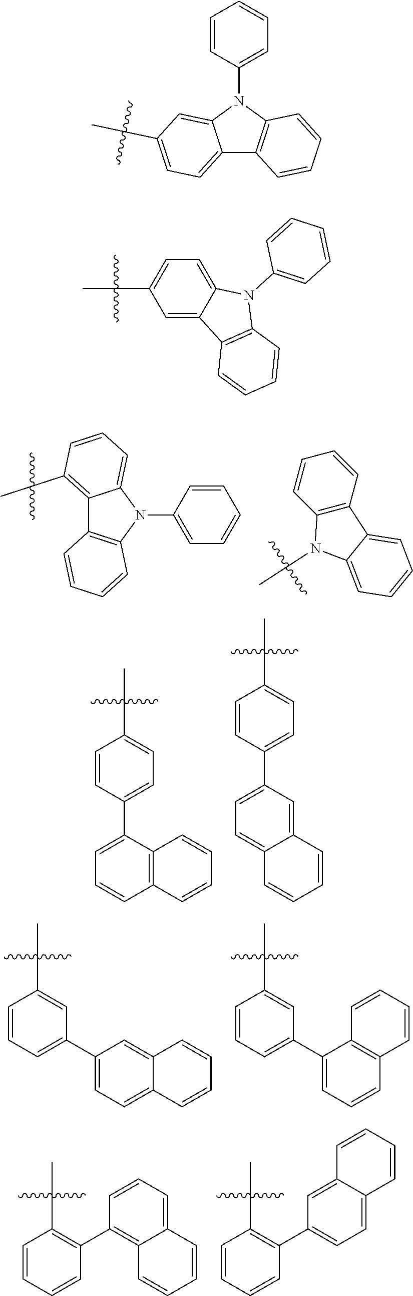

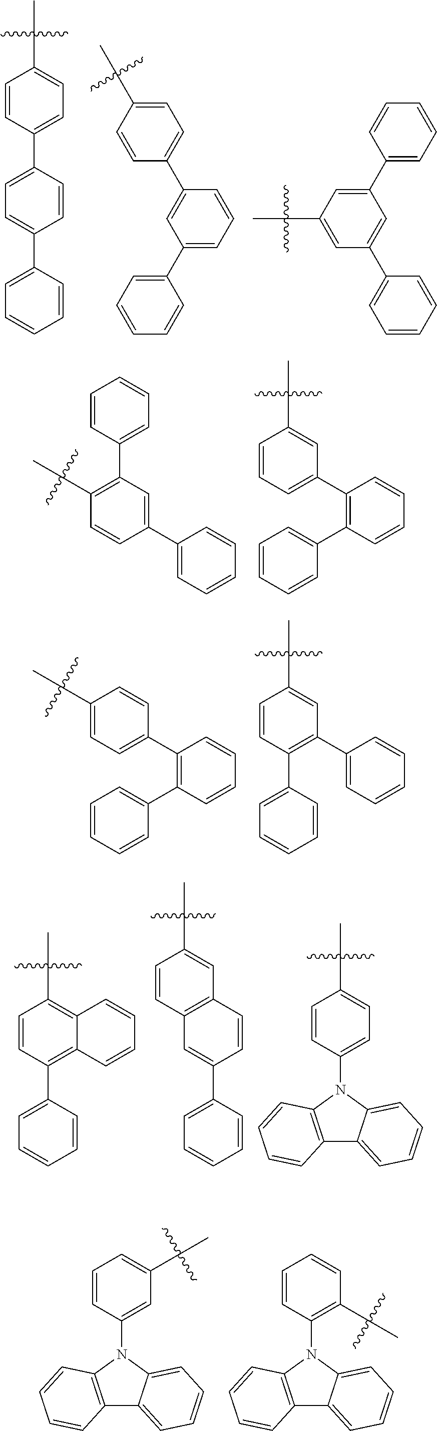

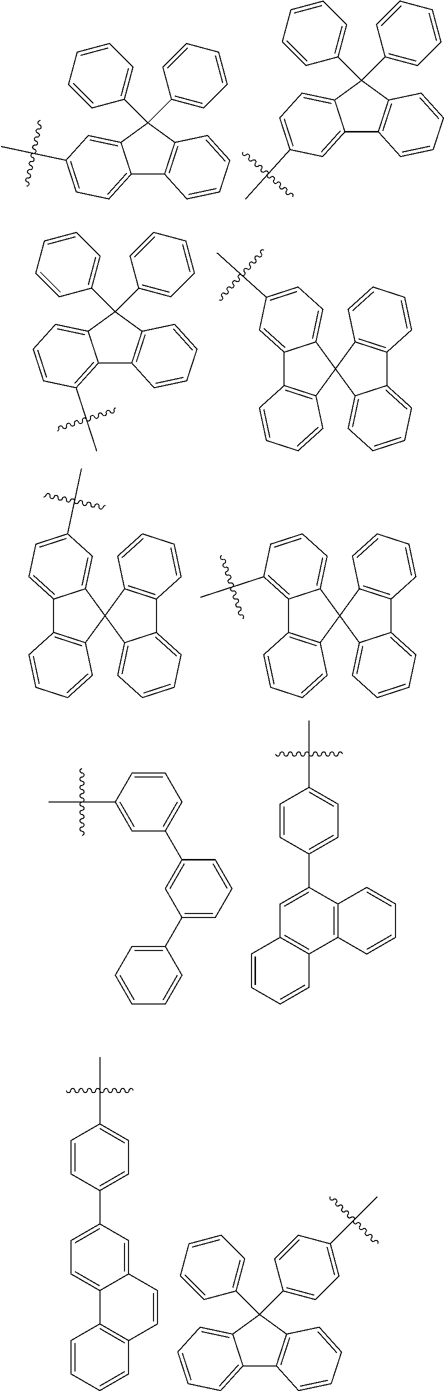

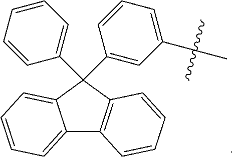 [SMILES: *c1cccc(C2(c3ccccc3)c3ccccc3-c3ccccc32)c1]

In some embodiments, the structure

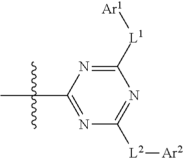

shown in Formula 2 is selected from the group consisting of the following groups:

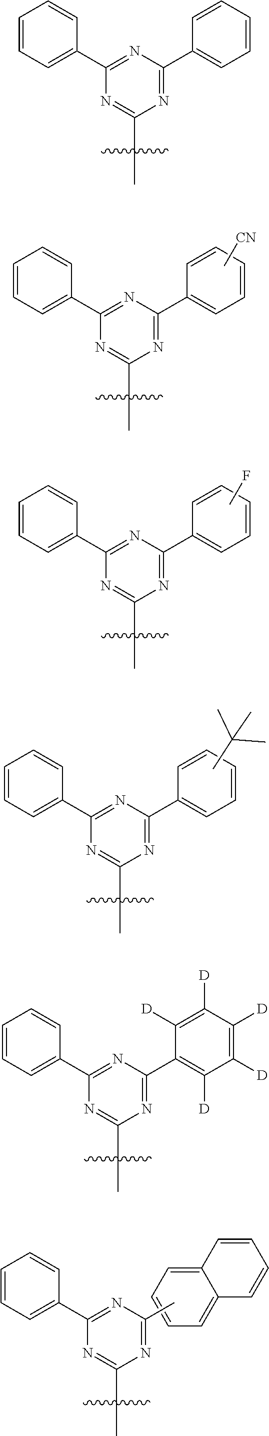

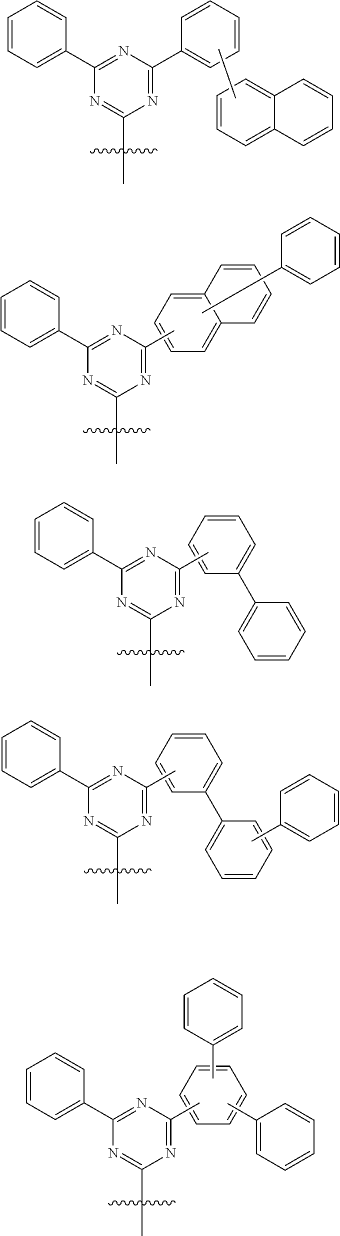

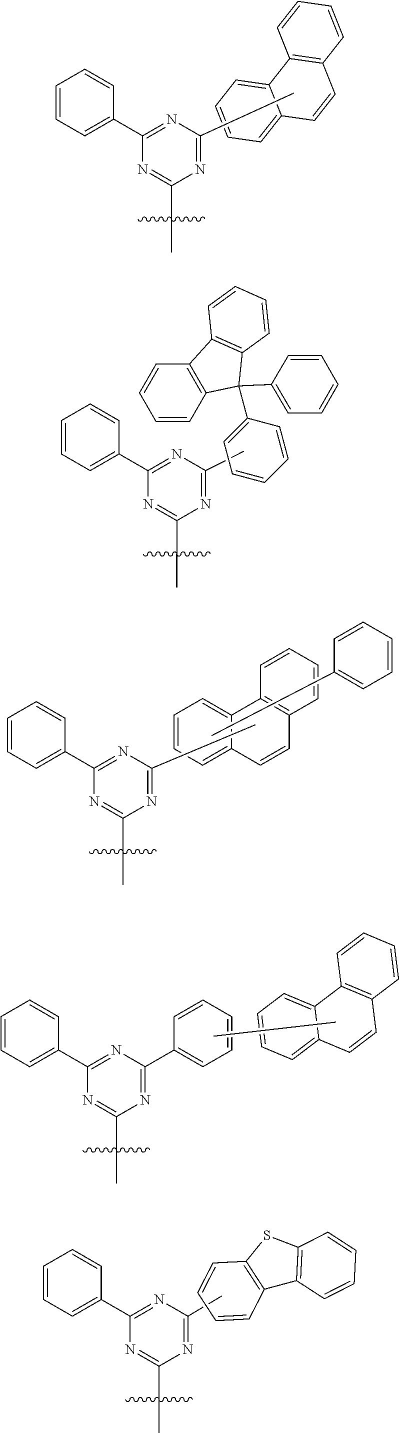

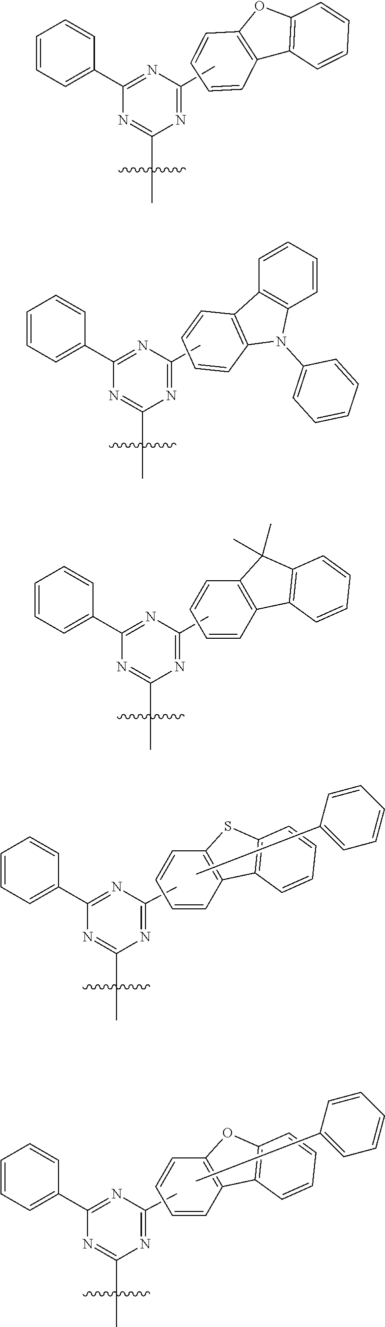

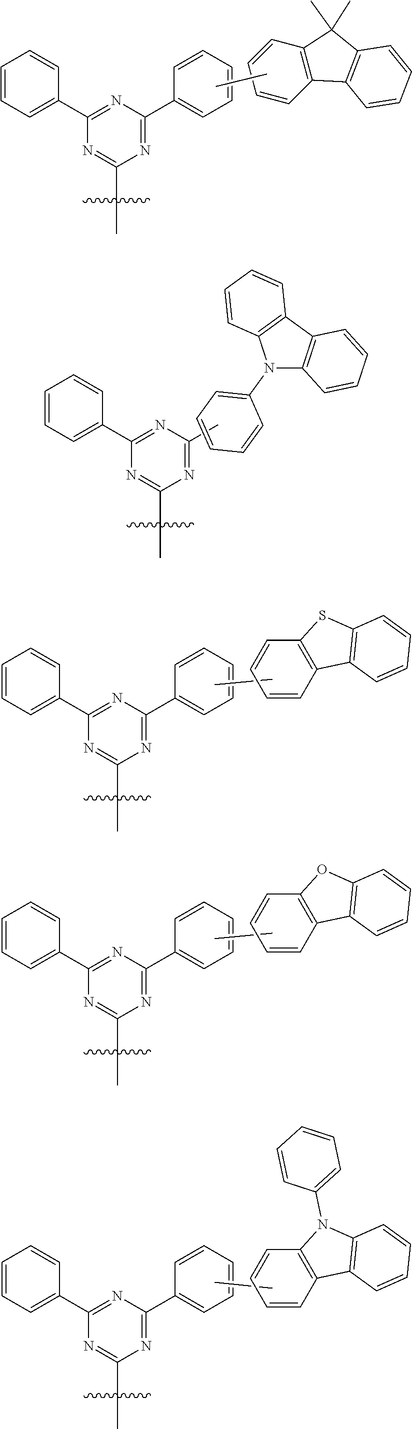

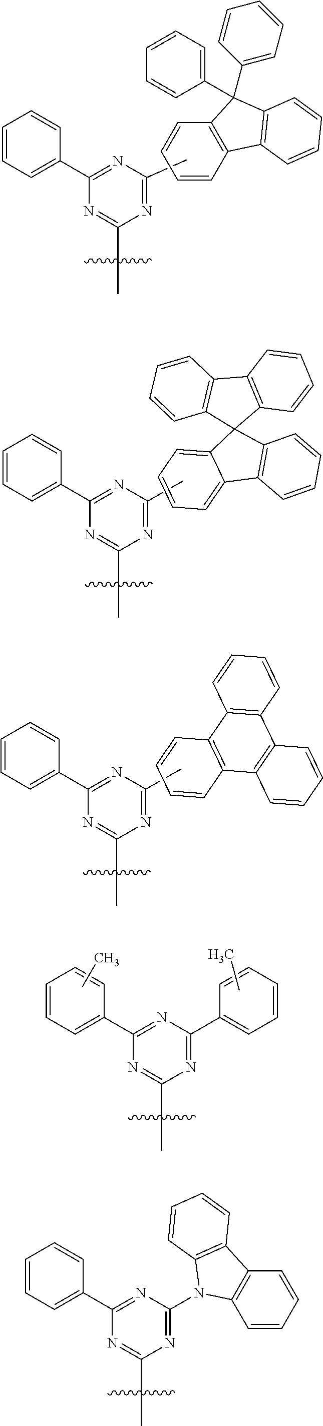

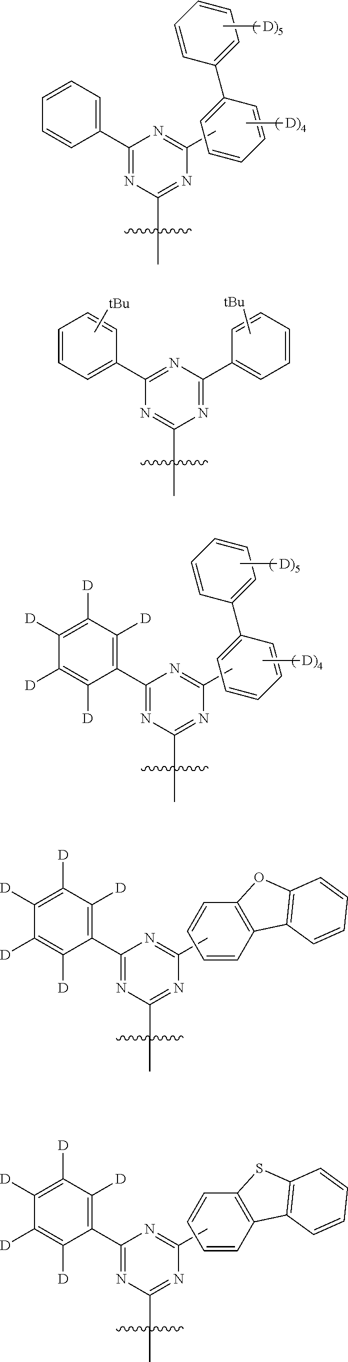

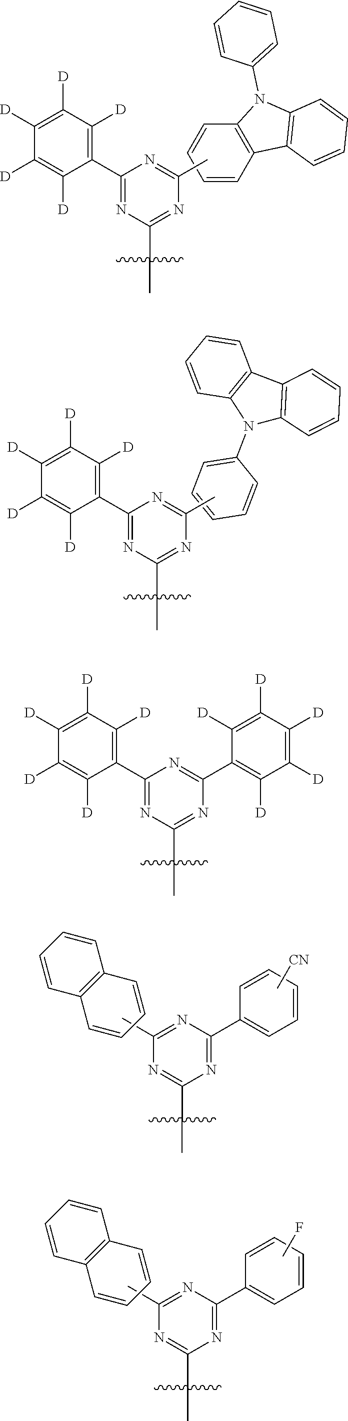

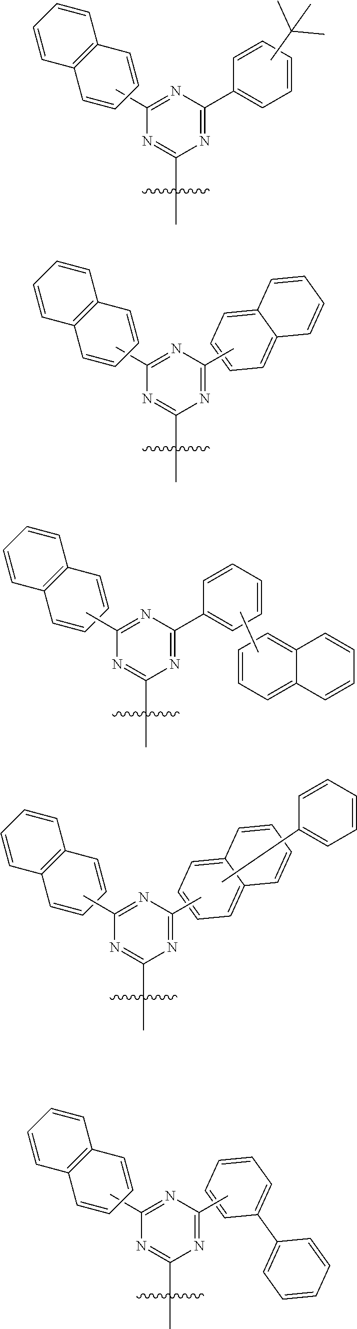

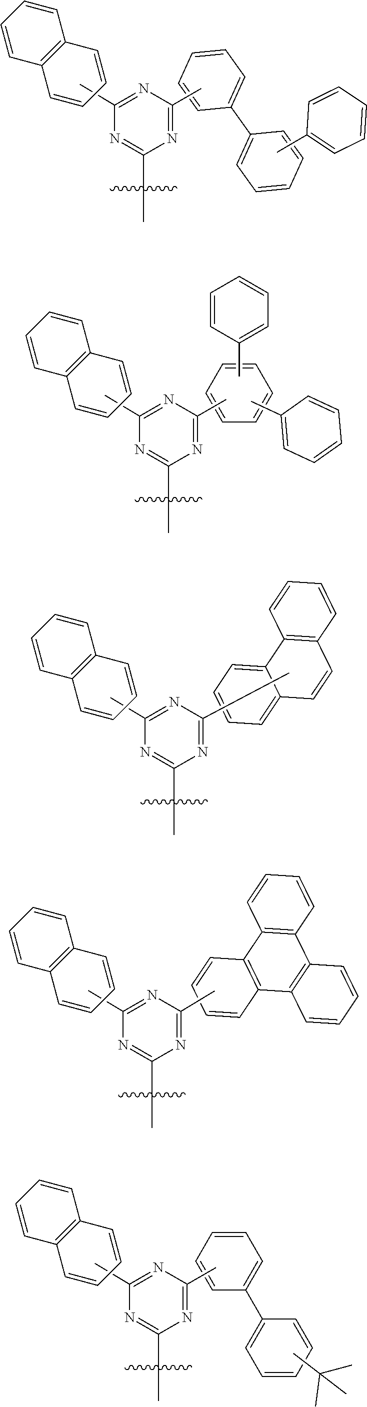

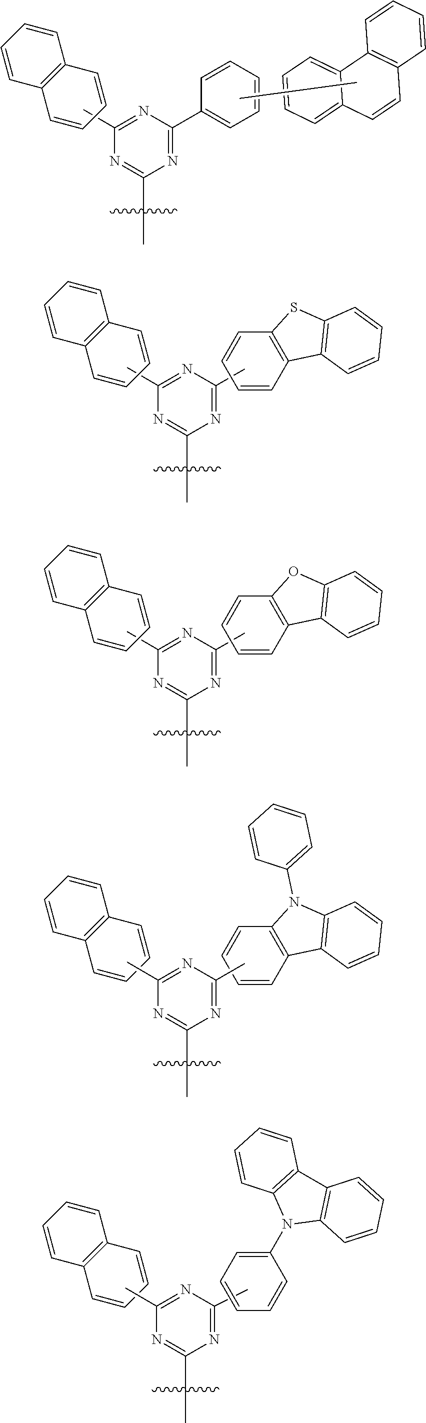

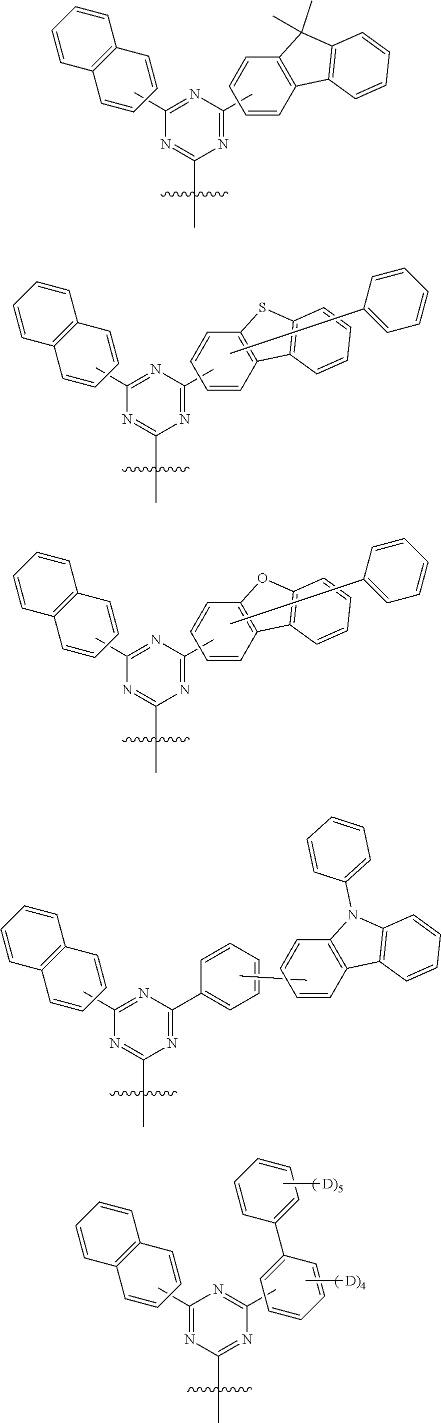

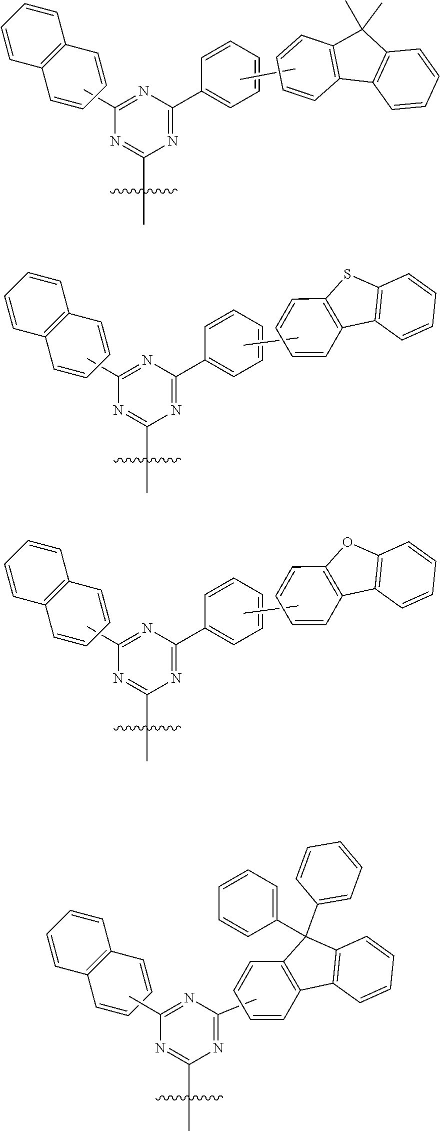

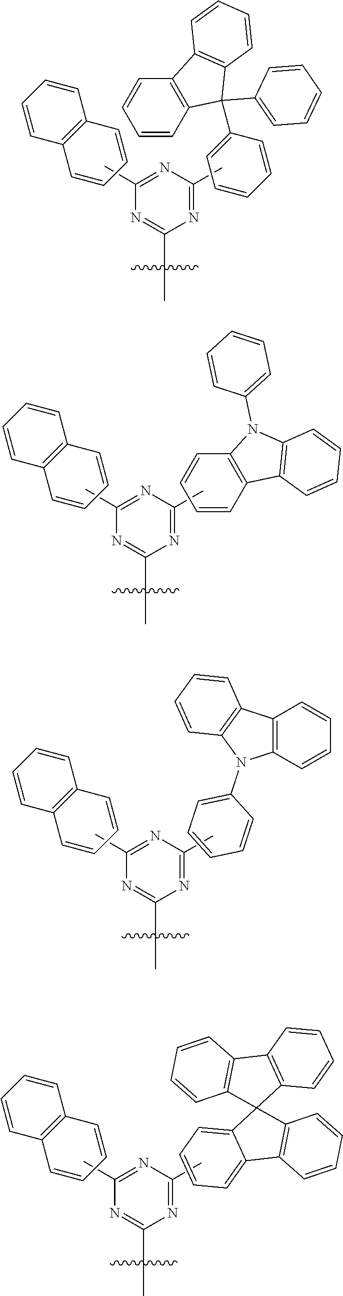

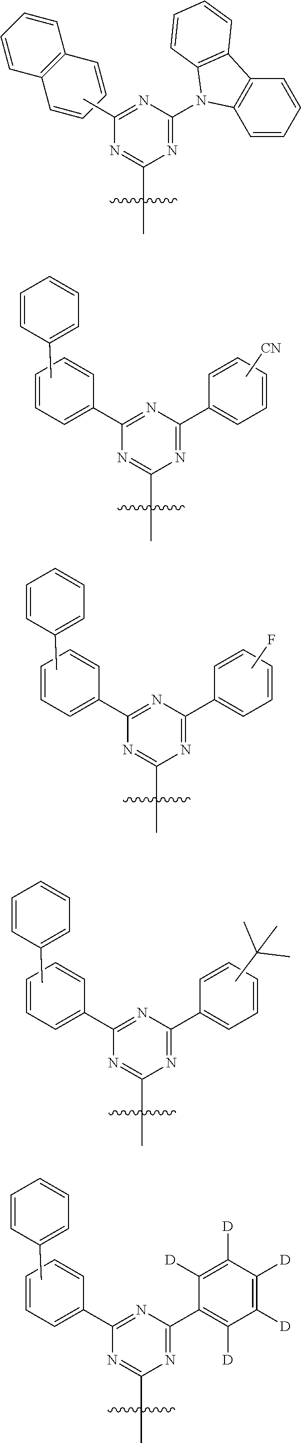

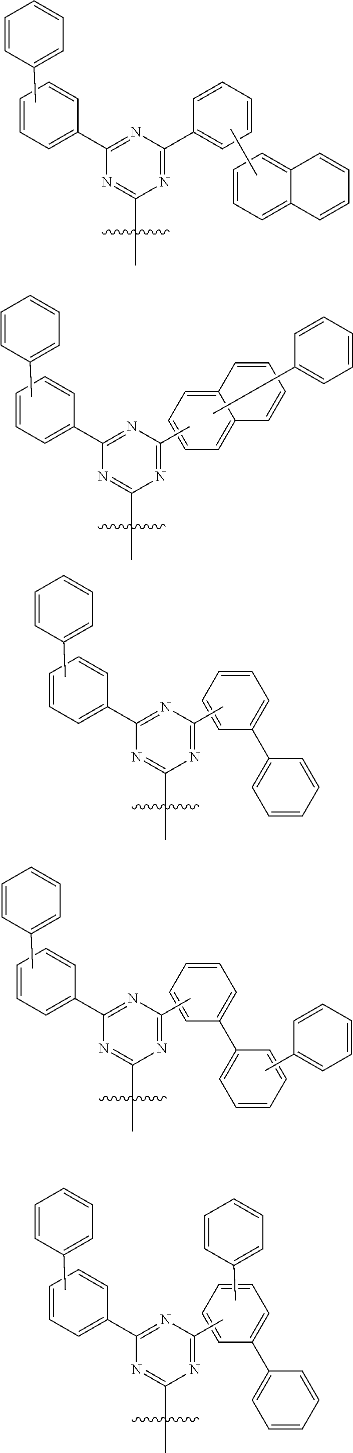

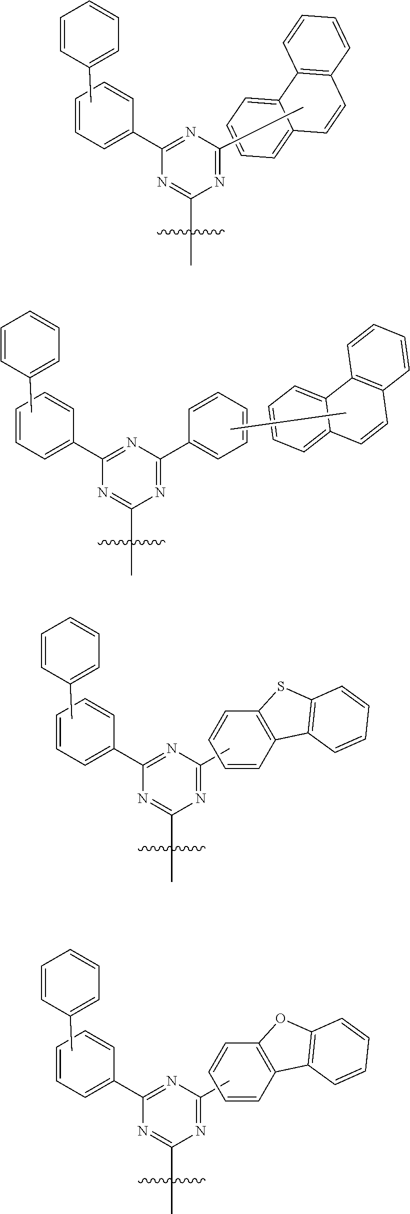

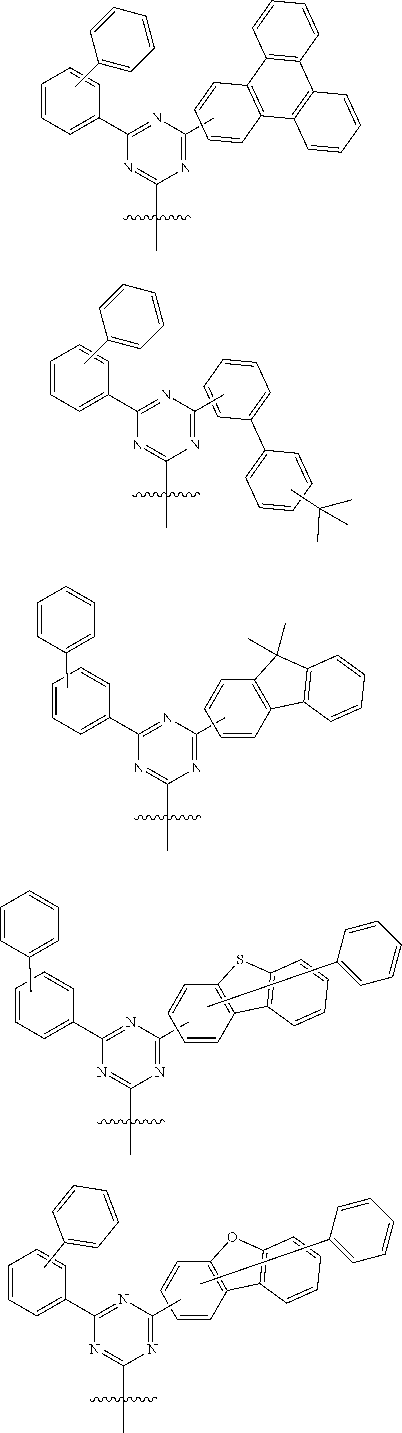

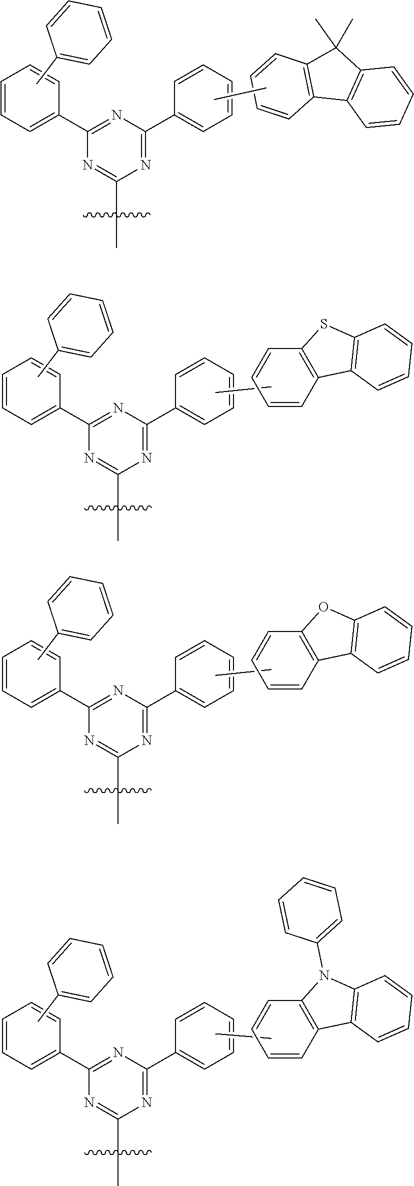

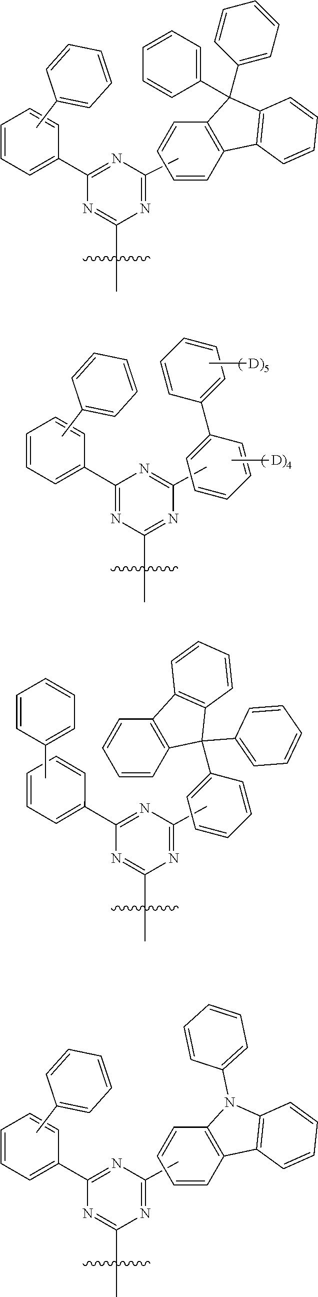

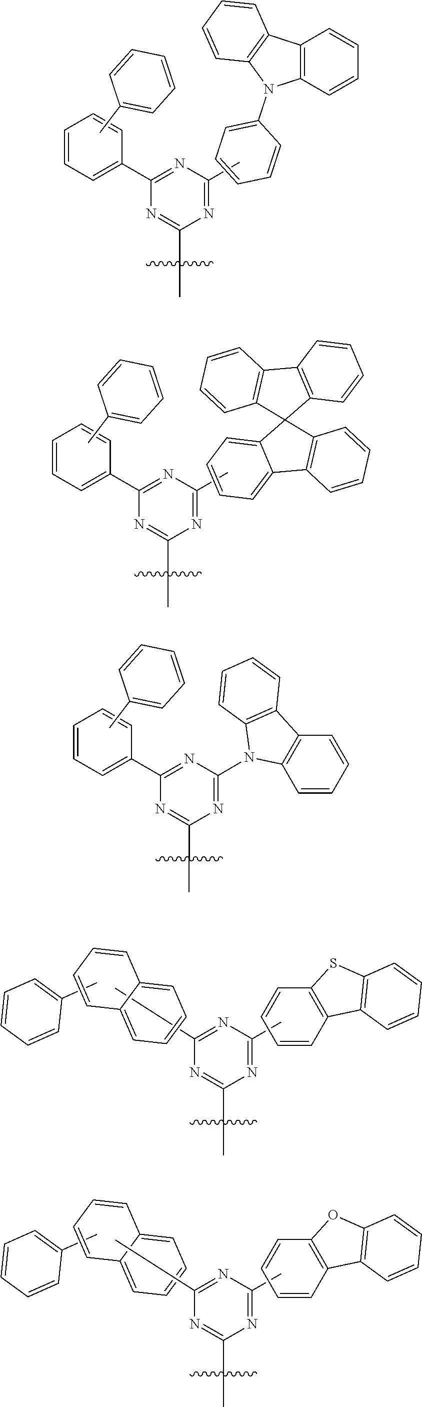

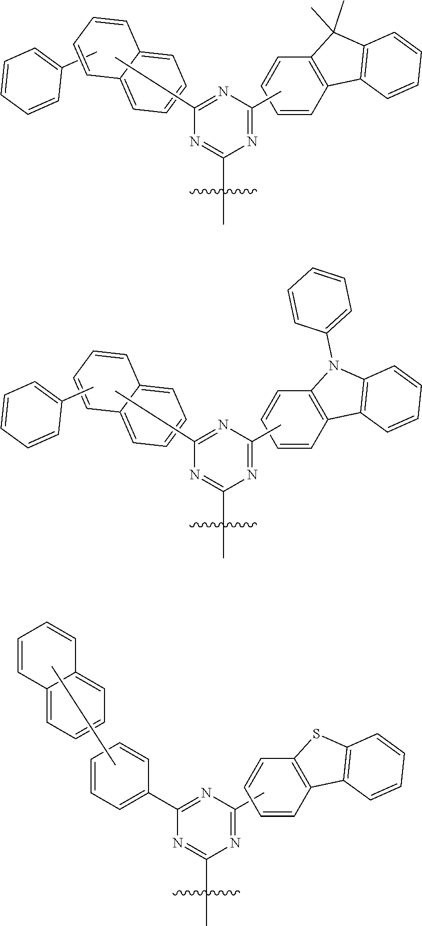

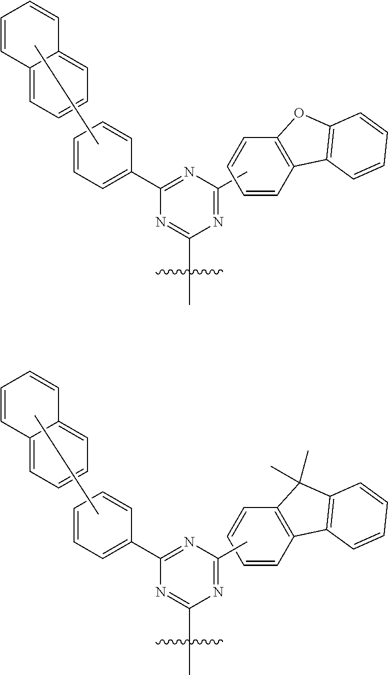

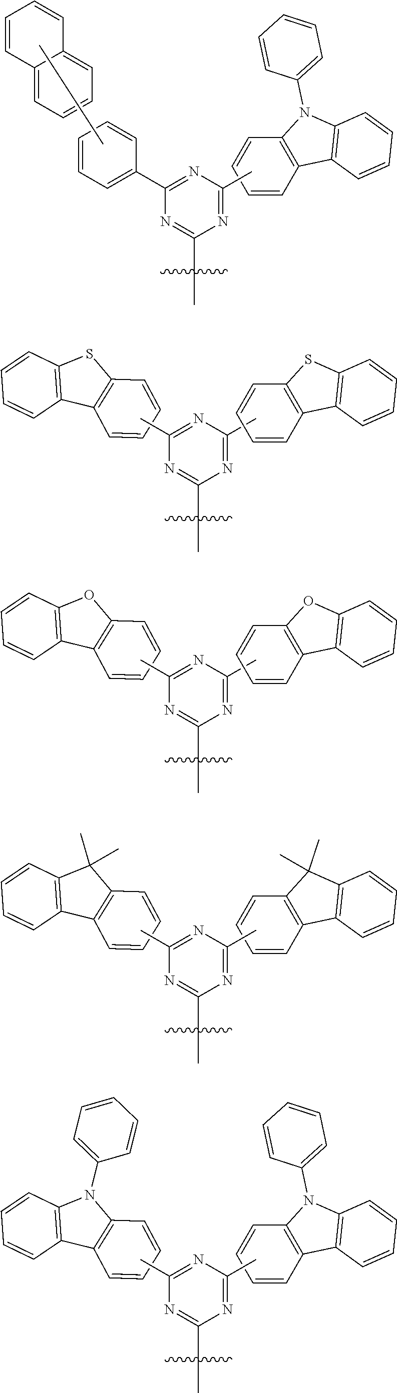

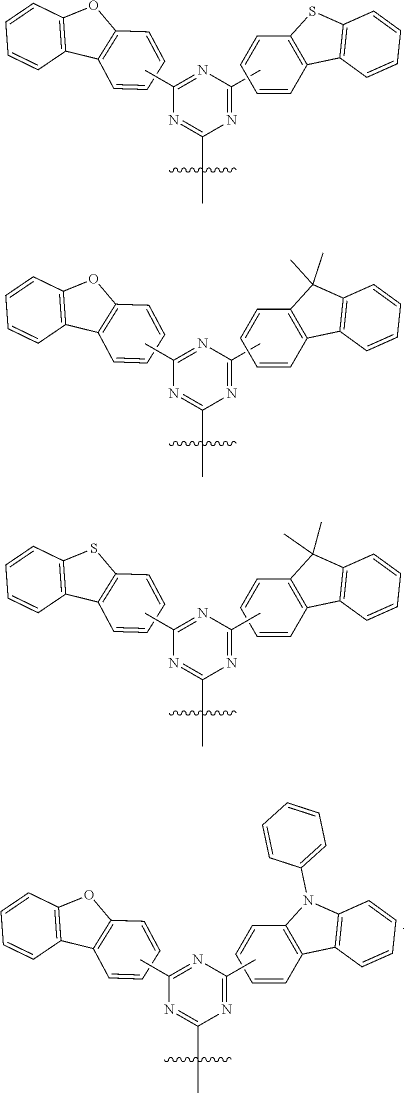

Specifically, the structure

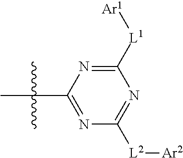

shown in Formula 2 is selected from the group consisting of the following group:

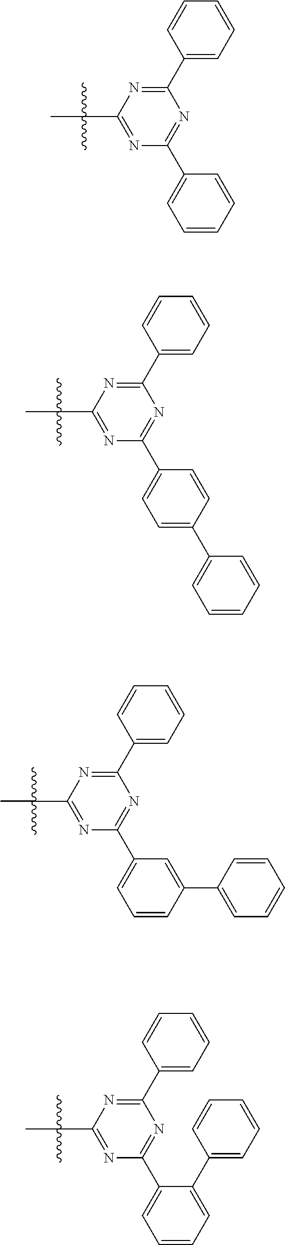 [SMILES: *c1nc(-c2ccccc2)nc(-c2ccccc2)n1 ; *c1nc(-c2ccccc2)nc(-c2ccc(-c3ccccc3)cc2)n1 ; *c1nc(-c2ccccc2)nc(-c2cccc(-c3ccccc3)c2)n1 ; *c1nc(-c2ccccc2)nc(-c2ccccc2-c2ccccc2)n1]

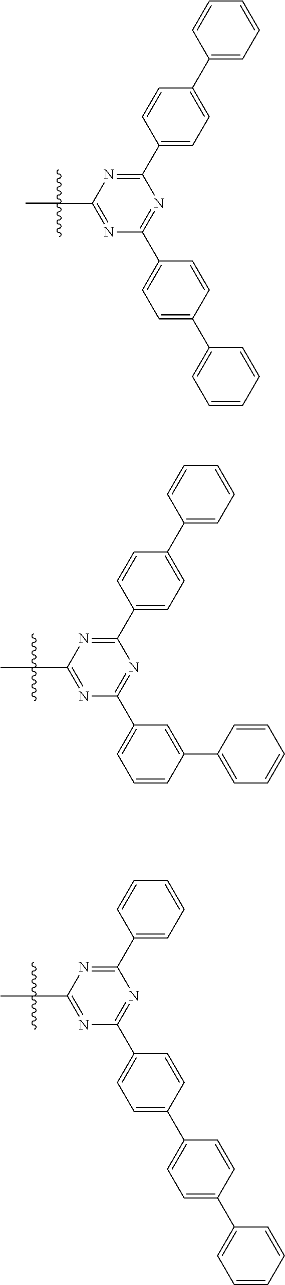 [SMILES: *c1nc(-c2ccc(-c3ccccc3)cc2)nc(-c2ccc(-c3ccccc3)cc2)n1 ; *c1nc(-c2ccc(-c3ccccc3)cc2)nc(-c2cccc(-c3ccccc3)c2)n1 ; *c1nc(-c2ccccc2)nc(-c2ccc(-c3ccc(-c4ccccc4)cc3)cc2)n1]

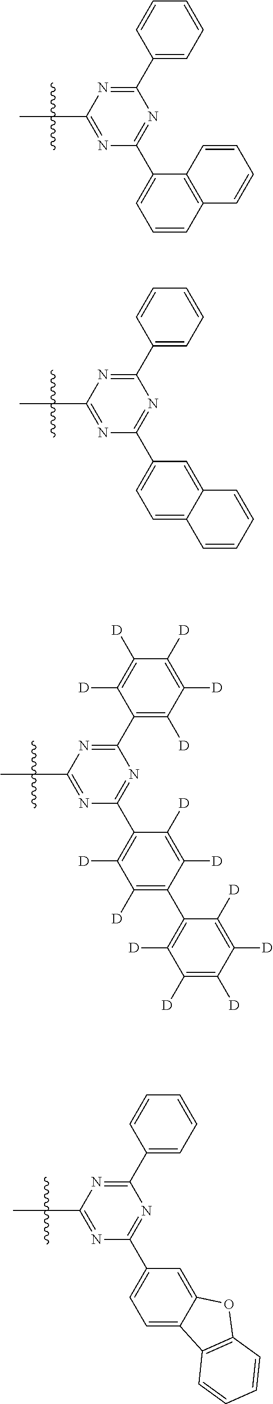 [SMILES: *c1nc(-c2ccccc2)nc(-c2cccc3ccccc23)n1 ; *c1nc(-c2ccccc2)nc(-c2ccc3ccccc3c2)n1 ; *c1nc(-c2c([2H])c([2H])c([2H])c([2H])c2[2H])nc(-c2c([2H])c([2H])c(-c3c([2H])c([2H])c([2H])c([2H])c3[2H])c([2H])c2[2H])n1 ; *c1nc(-c2ccccc2)nc(-c2ccc3c(c2)oc2ccccc23)n1]

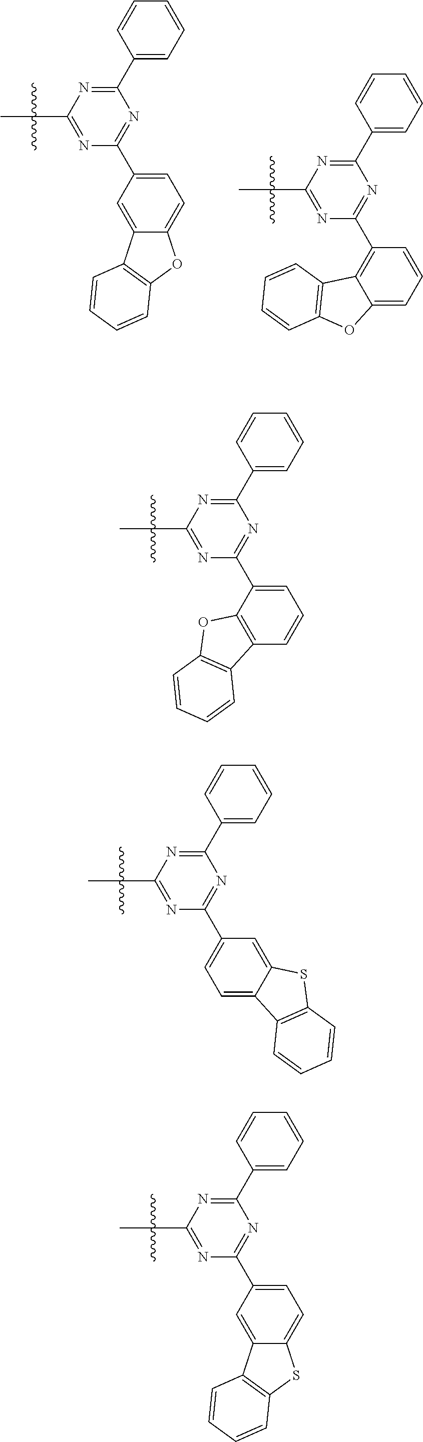

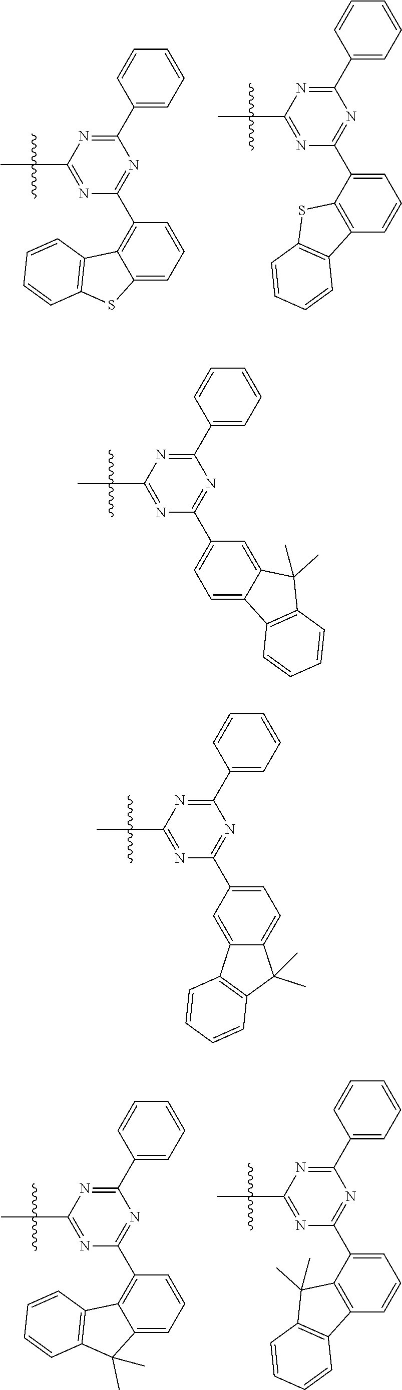

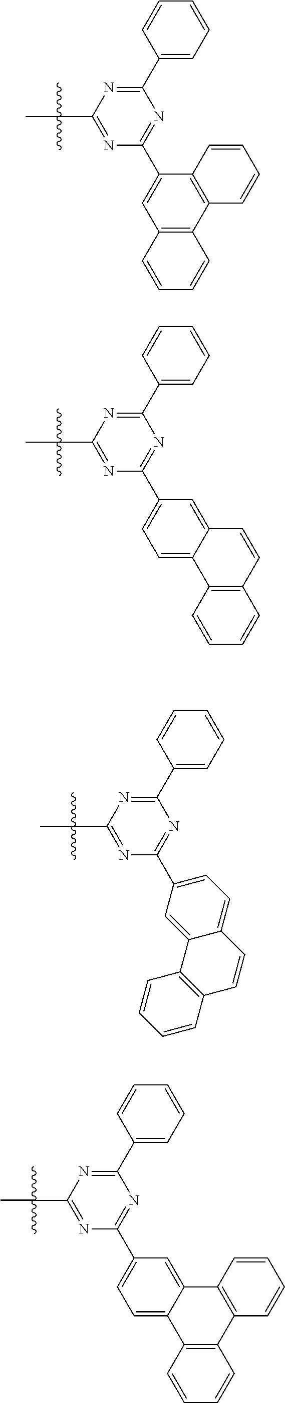

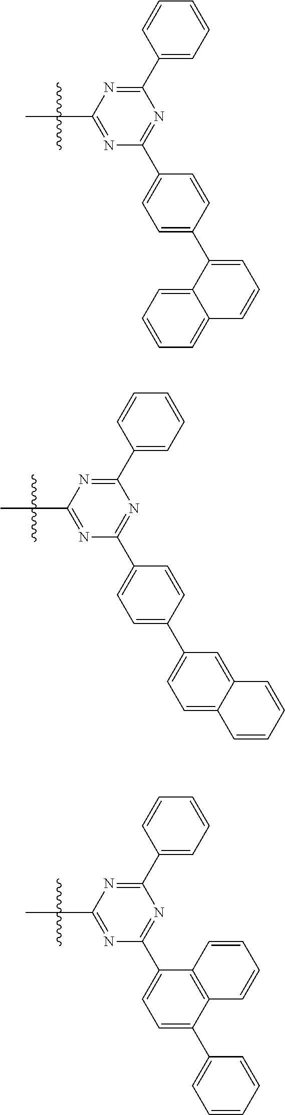

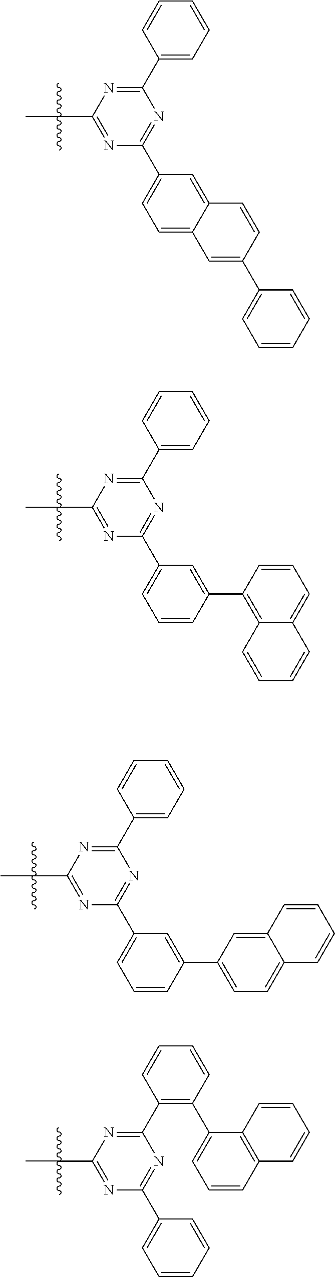 [SMILES: *c1nc(-c2ccccc2)nc(-c2ccc3cc(-c4ccccc4)ccc3c2)n1 ; *c1nc(-c2ccccc2)nc(-c2cccc(-c3cccc4ccccc34)c2)n1 ; *c1nc(-c2ccccc2)nc(-c2cccc(-c3ccc4ccccc4c3)c2)n1 ; *c1nc(-c2ccccc2)nc(-c2ccccc2-c2cccc3ccccc23)n1]

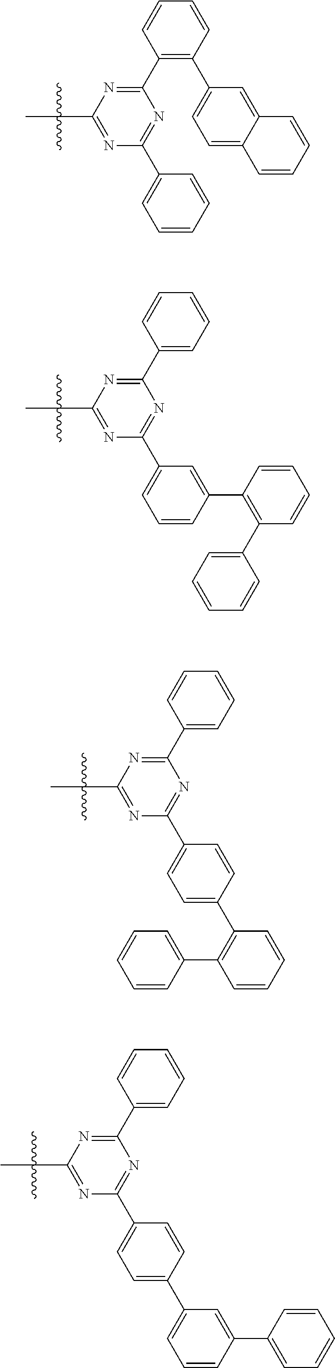 [SMILES: *c1nc(-c2ccccc2)nc(-c2ccccc2-c2ccc3ccccc3c2)n1 ; *c1nc(-c2ccccc2)nc(-c2cccc(-c3ccccc3-c3ccccc3)c2)n1 ; *c1nc(-c2ccccc2)nc(-c2ccc(-c3ccccc3-c3ccccc3)cc2)n1 ; *c1nc(-c2ccccc2)nc(-c2ccc(-c3cccc(-c4ccccc4)c3)cc2)n1]

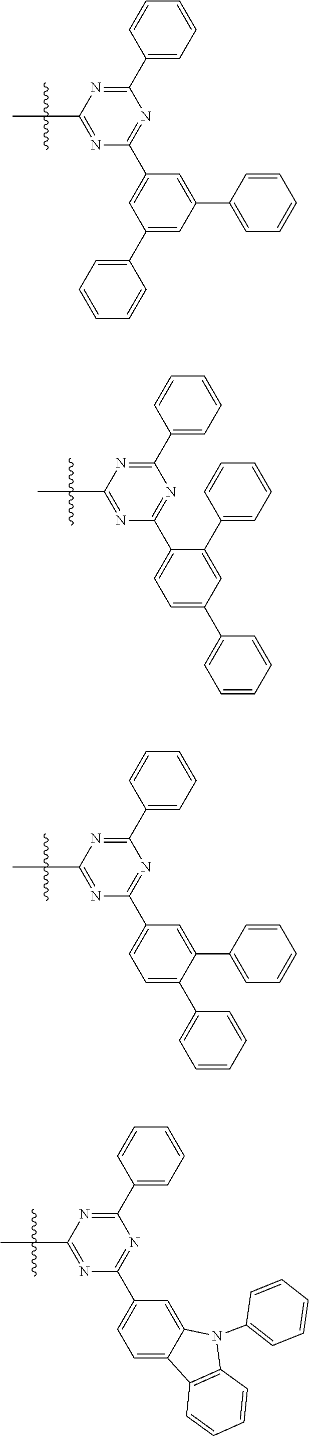 [SMILES: *c1nc(-c2ccccc2)nc(-c2cc(-c3ccccc3)cc(-c3ccccc3)c2)n1 ; *c1nc(-c2ccccc2)nc(-c2ccc(-c3ccccc3)cc2-c2ccccc2)n1 ; *c1nc(-c2ccccc2)nc(-c2ccc(-c3ccccc3)c(-c3ccccc3)c2)n1 ; *c1nc(-c2ccccc2)nc(-c2ccc3c4ccccc4n(-c4ccccc4)c3c2)n1]

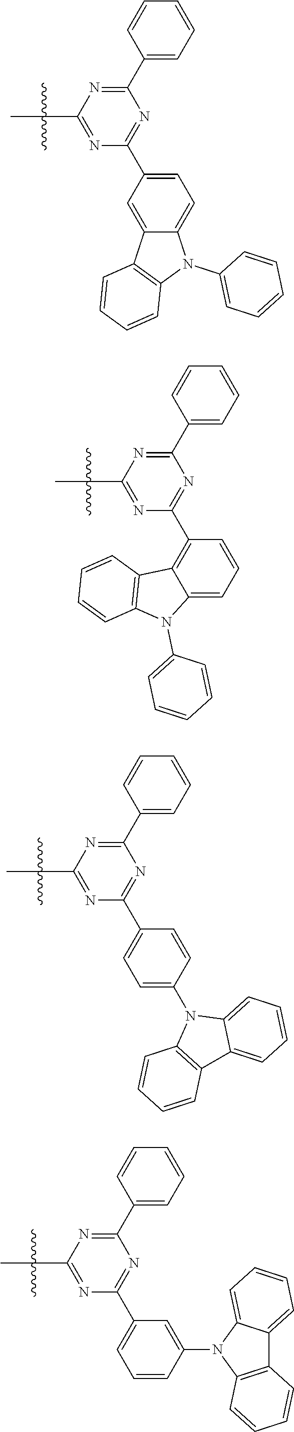

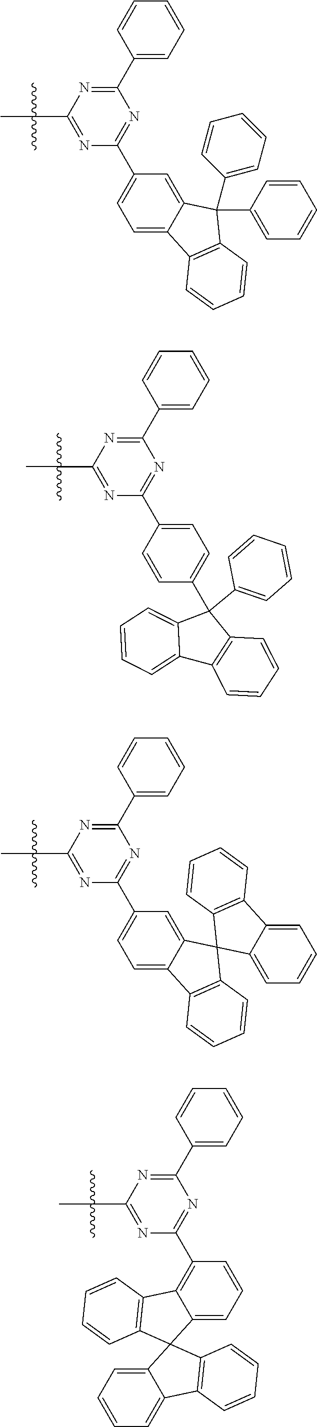

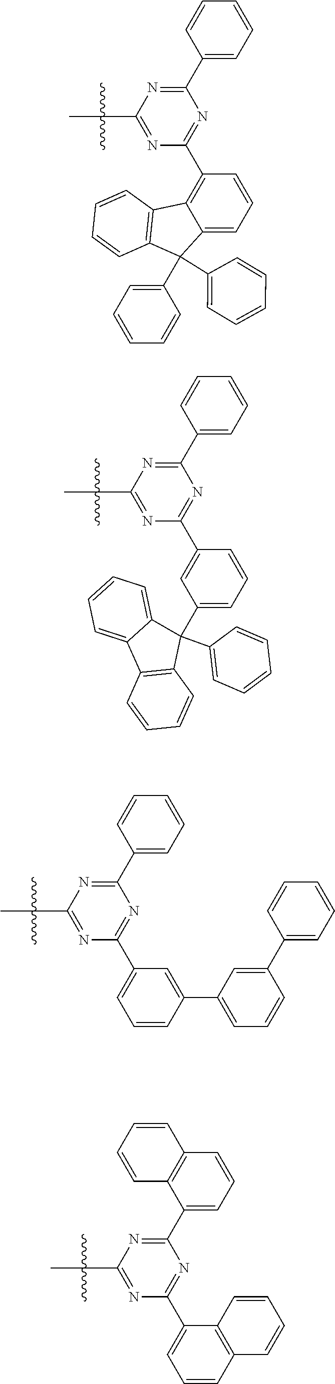 [SMILES: *c1nc(-c2ccccc2)nc(-c2cccc3c2-c2ccccc2C3(c2ccccc2)c2ccccc2)n1 ; *c1nc(-c2ccccc2)nc(-c2cccc(C3(c4ccccc4)c4ccccc4-c4ccccc43)c2)n1 ; *c1nc(-c2ccccc2)nc(-c2cccc(-c3cccc(-c4ccccc4)c3)c2)n1 ; *c1nc(-c2cccc3ccccc23)nc(-c2cccc3ccccc23)n1]

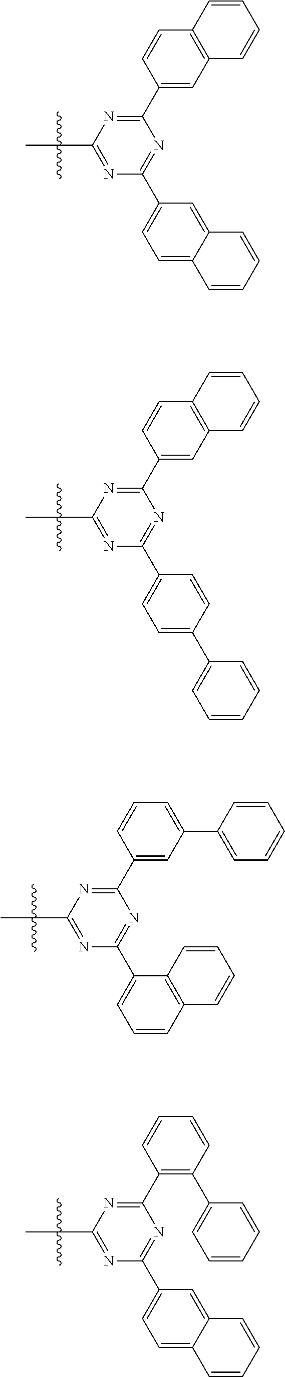 [SMILES: *c1nc(-c2ccc3ccccc3c2)nc(-c2ccc3ccccc3c2)n1 ; *c1nc(-c2ccc(-c3ccccc3)cc2)nc(-c2ccc3ccccc3c2)n1 ; *c1nc(-c2cccc(-c3ccccc3)c2)nc(-c2cccc3ccccc23)n1 ; *c1nc(-c2ccc3ccccc3c2)nc(-c2ccccc2-c2ccccc2)n1]

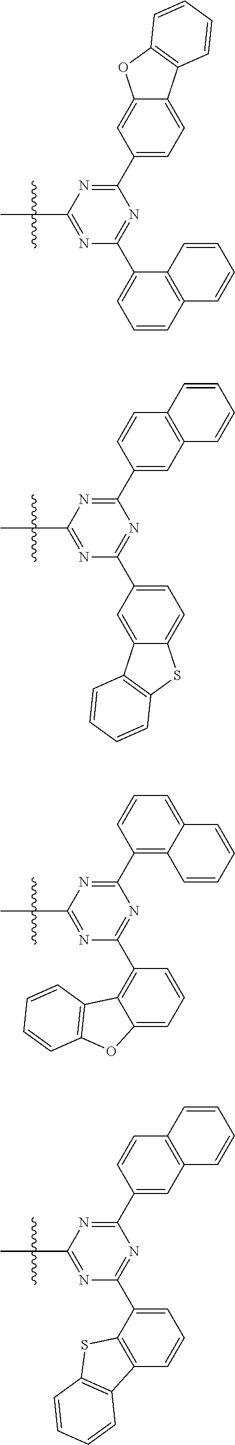 [SMILES: *c1nc(-c2ccc3c(c2)oc2ccccc23)nc(-c2cccc3ccccc23)n1 ; *c1nc(-c2ccc3ccccc3c2)nc(-c2ccc3sc4ccccc4c3c2)n1 ; *c1nc(-c2cccc3ccccc23)nc(-c2cccc3oc4ccccc4c23)n1 ; *c1nc(-c2ccc3ccccc3c2)nc(-c2cccc3c2sc2ccccc23)n1]

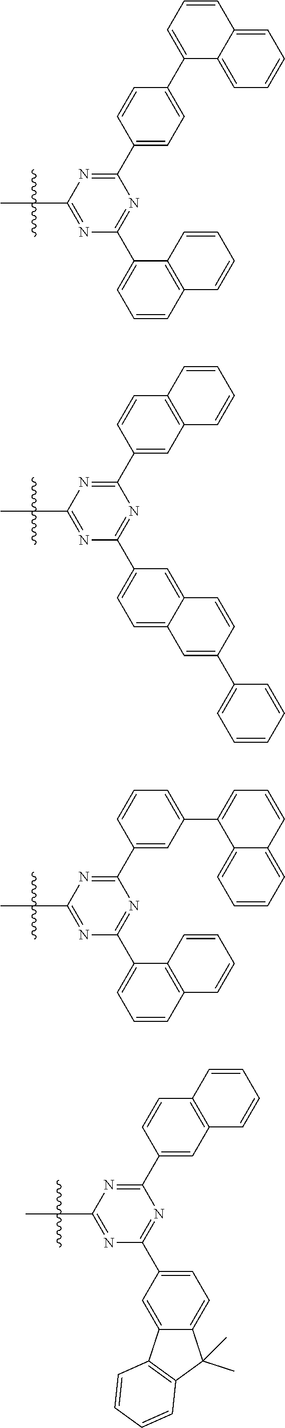 [SMILES: *c1nc(-c2ccc(-c3cccc4ccccc34)cc2)nc(-c2cccc3ccccc23)n1 ; *c1nc(-c2ccc3ccccc3c2)nc(-c2ccc3cc(-c4ccccc4)ccc3c2)n1 ; *c1nc(-c2cccc(-c3cccc4ccccc34)c2)nc(-c2cccc3ccccc23)n1 ; *c1nc(-c2ccc3c(c2)-c2ccccc2C3(C)C)nc(-c2ccc3ccccc3c2)n1]

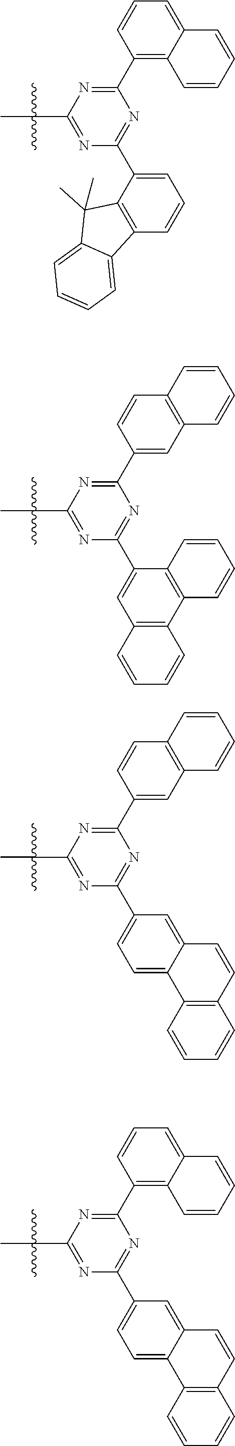

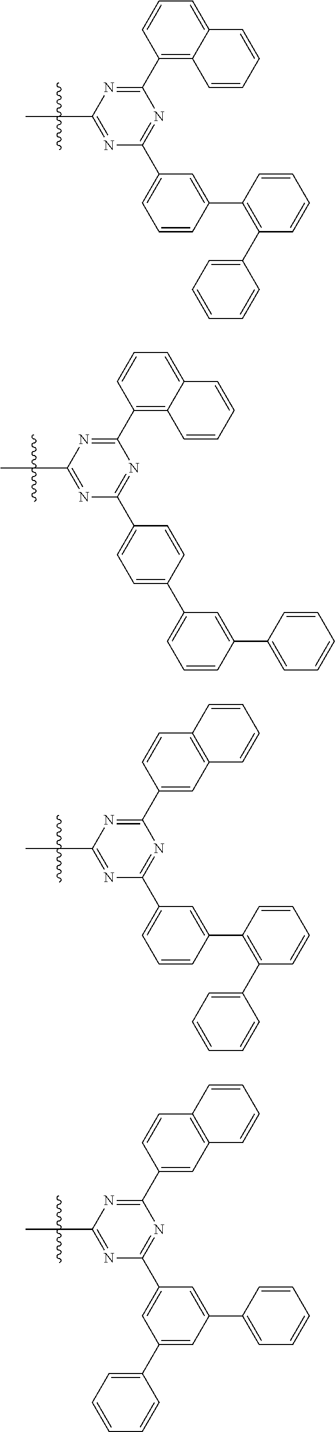

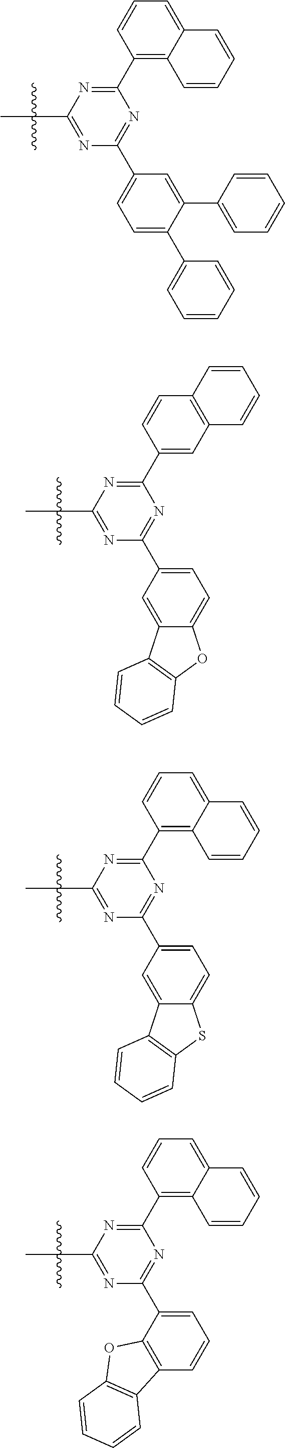

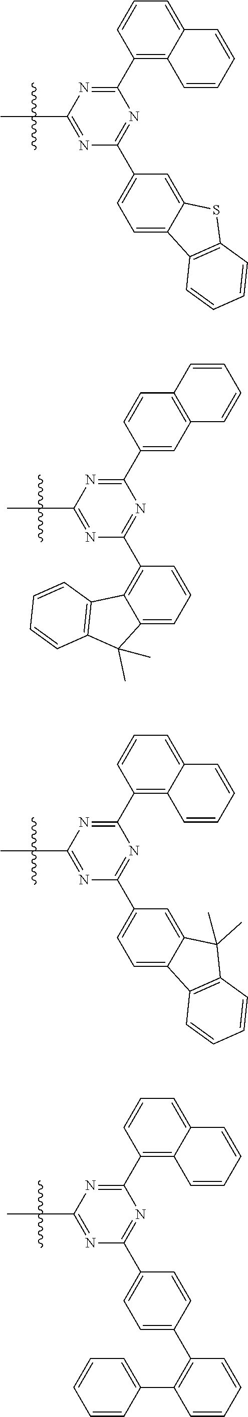 [SMILES: *c1nc(-c2ccc3c(c2)sc2ccccc23)nc(-c2cccc3ccccc23)n1 ; *c1nc(-c2ccc3ccccc3c2)nc(-c2cccc3c2-c2ccccc2C3(C)C)n1 ; *c1nc(-c2ccc3c(c2)C(C)(C)c2ccccc2-3)nc(-c2cccc3ccccc23)n1 ; *c1nc(-c2ccc(-c3ccccc3-c3ccccc3)cc2)nc(-c2cccc3ccccc23)n1]

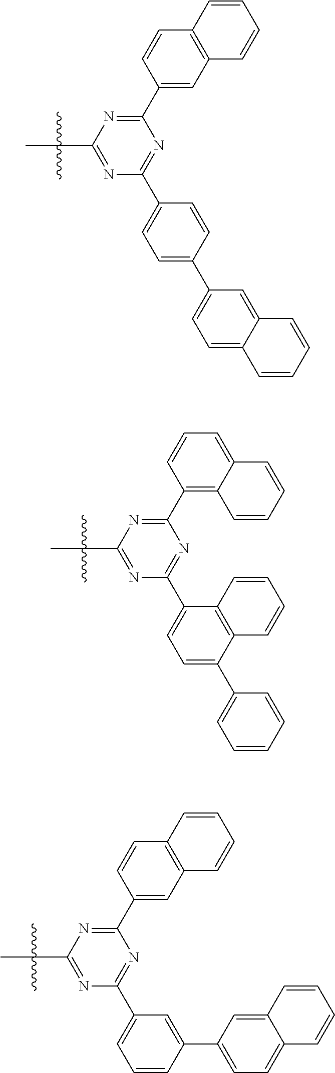 [SMILES: *c1nc(-c2ccc(-c3ccc4ccccc4c3)cc2)nc(-c2ccc3ccccc3c2)n1 ; *c1nc(-c2cccc3ccccc23)nc(-c2ccc(-c3ccccc3)c3ccccc23)n1 ; *c1nc(-c2cccc(-c3ccc4ccccc4c3)c2)nc(-c2ccc3ccccc3c2)n1]

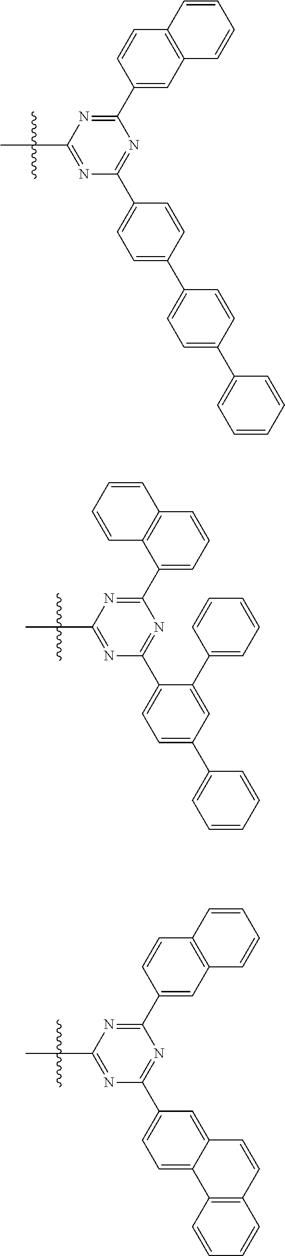 [SMILES: *c1nc(-c2ccc(-c3ccc(-c4ccccc4)cc3)cc2)nc(-c2ccc3ccccc3c2)n1 ; *c1nc(-c2ccc(-c3ccccc3)cc2-c2ccccc2)nc(-c2cccc3ccccc23)n1 ; *c1nc(-c2ccc3ccccc3c2)nc(-c2ccc3c(ccc4ccccc43)c2)n1]

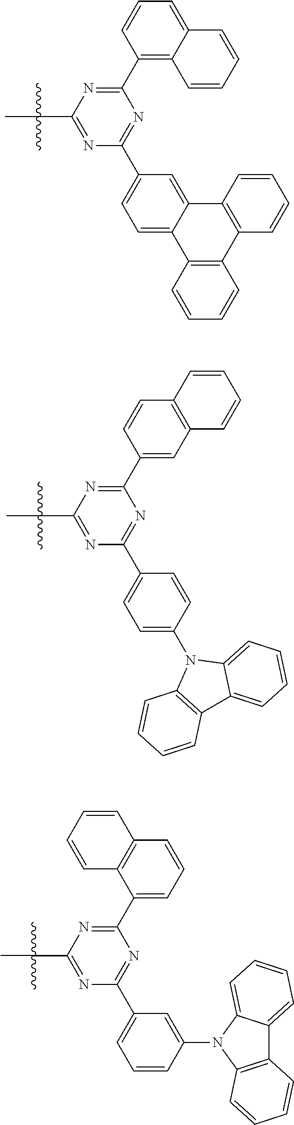 [SMILES: *c1nc(-c2ccc3c4ccccc4c4ccccc4c3c2)nc(-c2cccc3ccccc23)n1 ; *c1nc(-c2ccc(-n3c4ccccc4c4ccccc43)cc2)nc(-c2ccc3ccccc3c2)n1 ; *c1nc(-c2cccc(-n3c4ccccc4c4ccccc43)c2)nc(-c2cccc3ccccc23)n1]

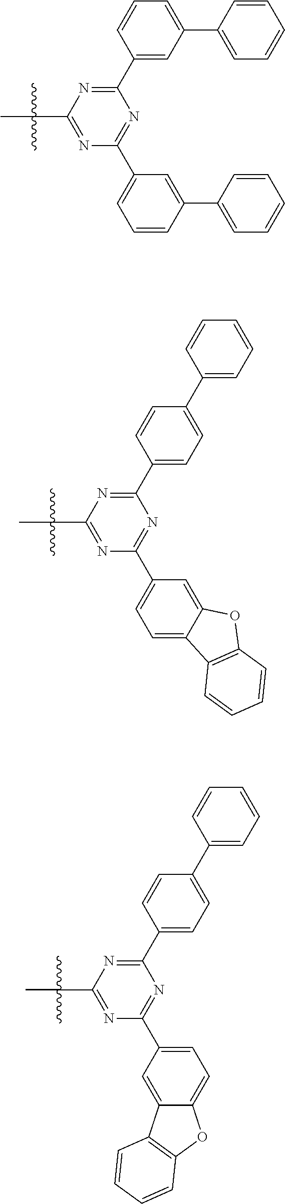 [SMILES: *c1nc(-c2cccc(-c3ccccc3)c2)nc(-c2cccc(-c3ccccc3)c2)n1 ; *c1nc(-c2ccc(-c3ccccc3)cc2)nc(-c2ccc3c(c2)oc2ccccc23)n1 ; *c1nc(-c2ccc(-c3ccccc3)cc2)nc(-c2ccc3oc4ccccc4c3c2)n1]

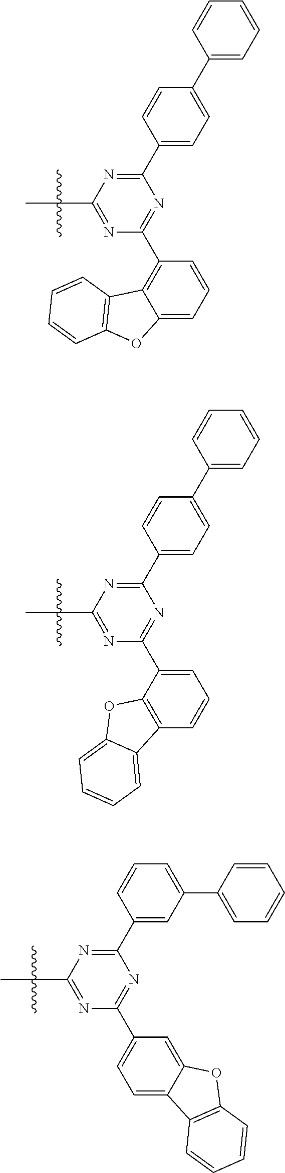 [SMILES: *c1nc(-c2ccc(-c3ccccc3)cc2)nc(-c2cccc3oc4ccccc4c23)n1 ; *c1nc(-c2ccc(-c3ccccc3)cc2)nc(-c2cccc3c2oc2ccccc23)n1 ; *c1nc(-c2cccc(-c3ccccc3)c2)nc(-c2ccc3c(c2)oc2ccccc23)n1]

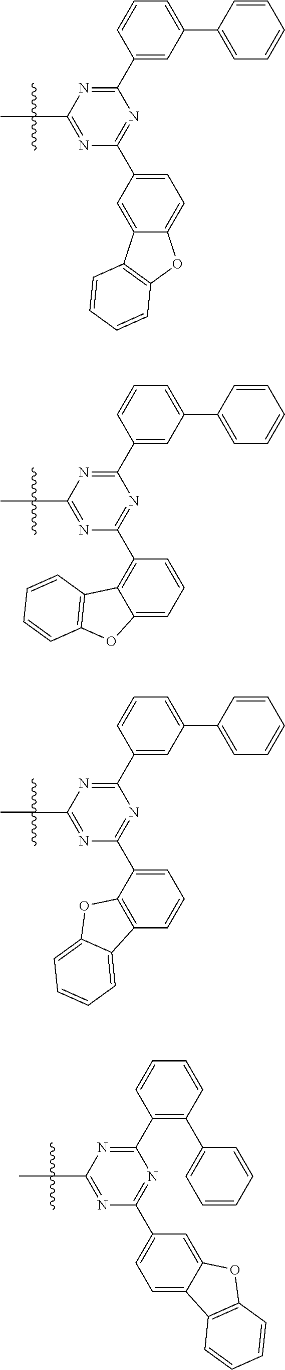 [SMILES: *c1nc(-c2cccc(-c3ccccc3)c2)nc(-c2ccc3oc4ccccc4c3c2)n1 ; *c1nc(-c2cccc(-c3ccccc3)c2)nc(-c2cccc3oc4ccccc4c23)n1 ; *c1nc(-c2cccc(-c3ccccc3)c2)nc(-c2cccc3c2oc2ccccc23)n1 ; *c1nc(-c2ccc3c(c2)oc2ccccc23)nc(-c2ccccc2-c2ccccc2)n1]

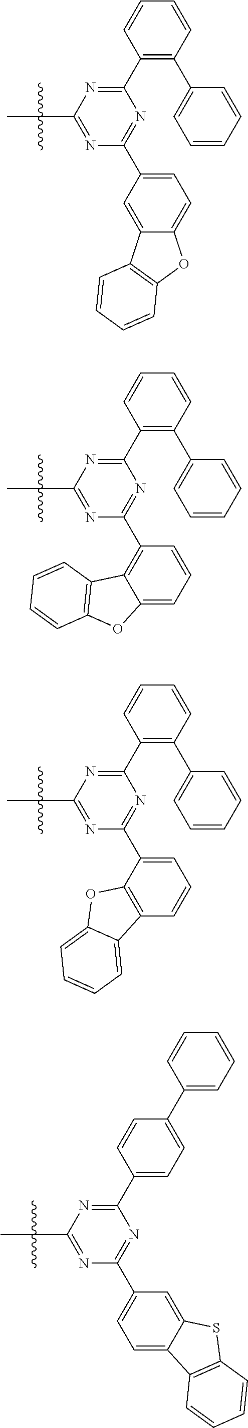

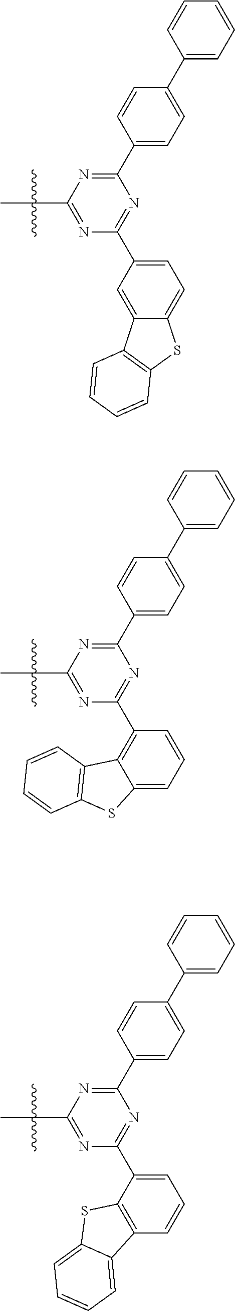 [SMILES: *c1nc(-c2ccc(-c3ccccc3)cc2)nc(-c2ccc3sc4ccccc4c3c2)n1 ; *c1nc(-c2ccc(-c3ccccc3)cc2)nc(-c2cccc3sc4ccccc4c23)n1 ; *c1nc(-c2ccc(-c3ccccc3)cc2)nc(-c2cccc3c2sc2ccccc23)n1]

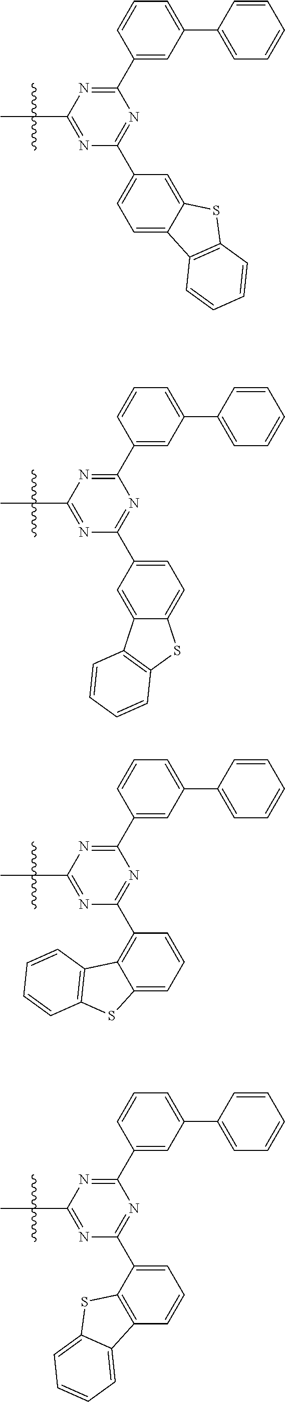

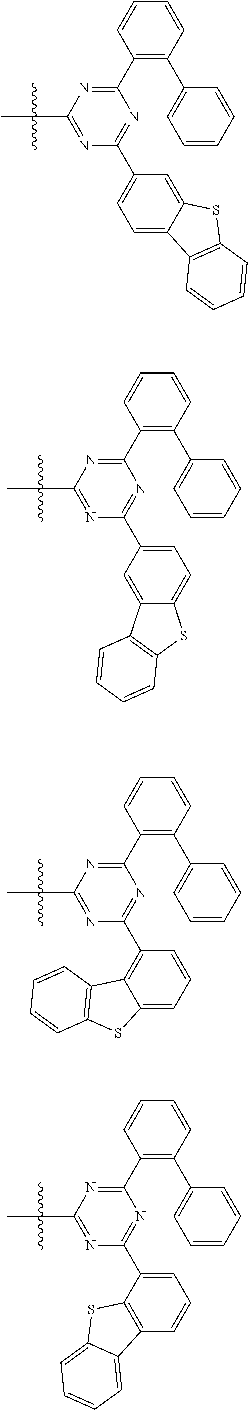 [SMILES: *c1nc(-c2ccc3c(c2)sc2ccccc23)nc(-c2ccccc2-c2ccccc2)n1 ; *c1nc(-c2ccc3sc4ccccc4c3c2)nc(-c2ccccc2-c2ccccc2)n1 ; *c1nc(-c2ccccc2-c2ccccc2)nc(-c2cccc3sc4ccccc4c23)n1 ; *c1nc(-c2ccccc2-c2ccccc2)nc(-c2cccc3c2sc2ccccc23)n1]

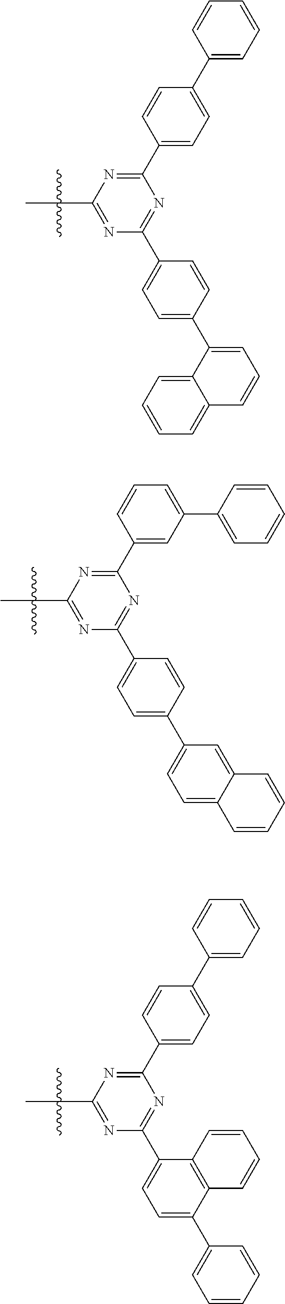 [SMILES: *c1nc(-c2ccc(-c3ccccc3)cc2)nc(-c2ccc(-c3cccc4ccccc34)cc2)n1 ; *c1nc(-c2ccc(-c3ccc4ccccc4c3)cc2)nc(-c2cccc(-c3ccccc3)c2)n1 ; *c1nc(-c2ccc(-c3ccccc3)cc2)nc(-c2ccc(-c3ccccc3)c3ccccc23)n1]

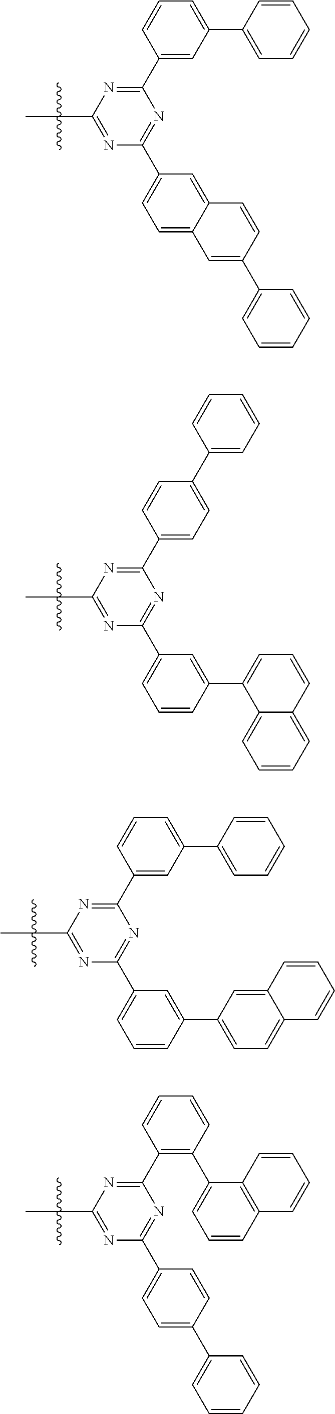 [SMILES: *c1nc(-c2cccc(-c3ccccc3)c2)nc(-c2ccc3cc(-c4ccccc4)ccc3c2)n1 ; *c1nc(-c2ccc(-c3ccccc3)cc2)nc(-c2cccc(-c3cccc4ccccc34)c2)n1 ; *c1nc(-c2cccc(-c3ccccc3)c2)nc(-c2cccc(-c3ccc4ccccc4c3)c2)n1 ; *c1nc(-c2ccc(-c3ccccc3)cc2)nc(-c2ccccc2-c2cccc3ccccc23)n1]

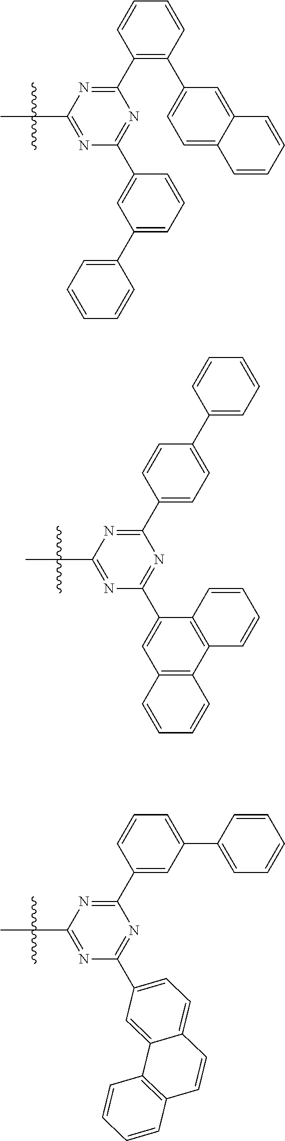 [SMILES: *c1nc(-c2cccc(-c3ccccc3)c2)nc(-c2ccccc2-c2ccc3ccccc3c2)n1 ; *c1nc(-c2ccc(-c3ccccc3)cc2)nc(-c2cc3ccccc3c3ccccc23)n1 ; *c1nc(-c2cccc(-c3ccccc3)c2)nc(-c2ccc3ccc4ccccc4c3c2)n1]

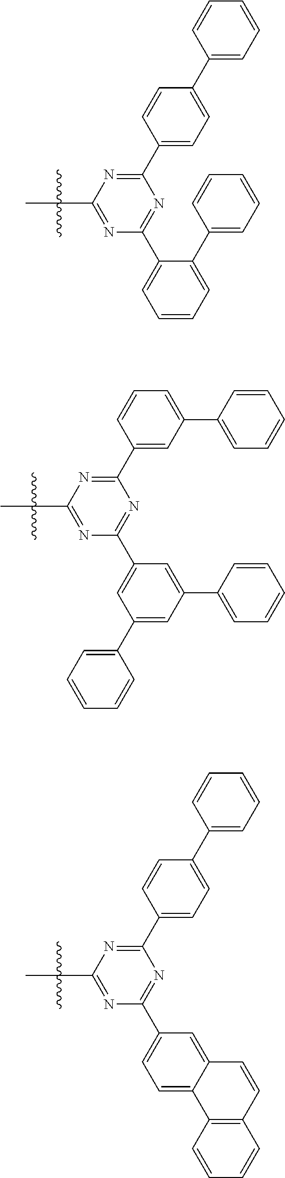 [SMILES: *c1nc(-c2ccc(-c3ccccc3)cc2)nc(-c2ccccc2-c2ccccc2)n1 ; *c1nc(-c2cccc(-c3ccccc3)c2)nc(-c2cc(-c3ccccc3)cc(-c3ccccc3)c2)n1 ; *c1nc(-c2ccc(-c3ccccc3)cc2)nc(-c2ccc3c(ccc4ccccc43)c2)n1]

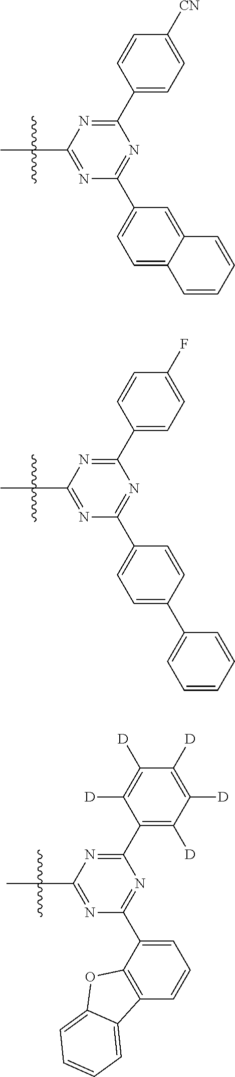 [SMILES: *c1nc(-c2ccc(C#N)cc2)nc(-c2ccc3ccccc3c2)n1 ; *c1nc(-c2ccc(F)cc2)nc(-c2ccc(-c3ccccc3)cc2)n1 ; *c1nc(-c2c([2H])c([2H])c([2H])c([2H])c2[2H])nc(-c2cccc3c2oc2ccccc23)n1]

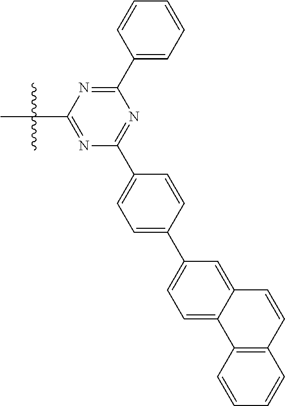 [SMILES: *c1nc(-c2ccccc2)nc(-c2ccc(-c3ccc4c(ccc5ccccc54)c3)cc2)n1]

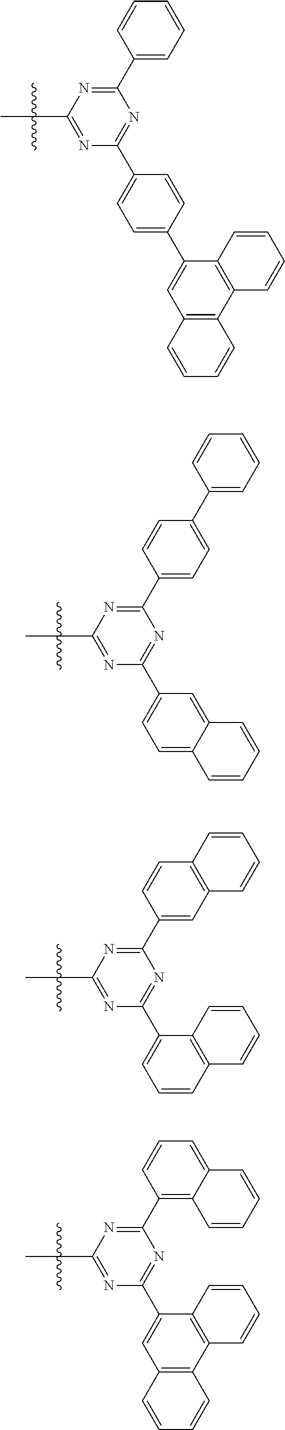 [SMILES: *c1nc(-c2ccccc2)nc(-c2ccc(-c3cc4ccccc4c4ccccc34)cc2)n1 ; *c1nc(-c2ccc(-c3ccccc3)cc2)nc(-c2ccc3ccccc3c2)n1 ; *c1nc(-c2ccc3ccccc3c2)nc(-c2cccc3ccccc23)n1 ; *c1nc(-c2cccc3ccccc23)nc(-c2cc3ccccc3c3ccccc23)n1]

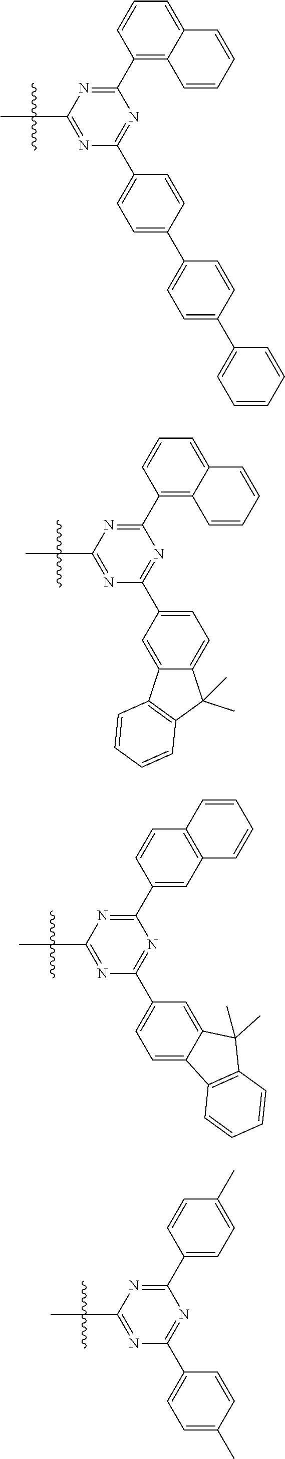

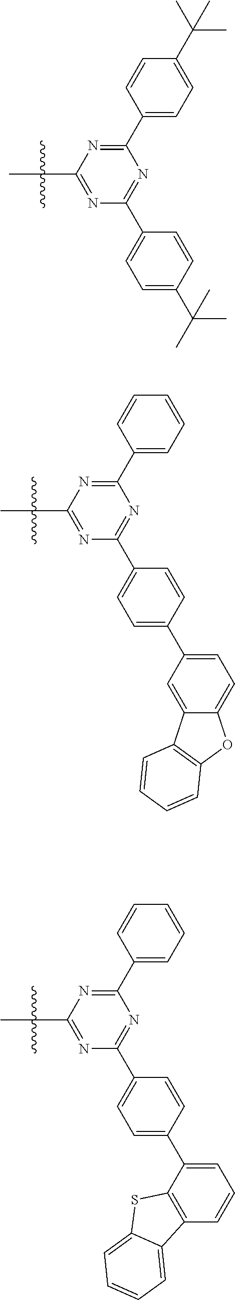 [SMILES: *c1nc(-c2ccc(C(C)(C)C)cc2)nc(-c2ccc(C(C)(C)C)cc2)n1 ; *c1nc(-c2ccccc2)nc(-c2ccc(-c3ccc4oc5ccccc5c4c3)cc2)n1 ; *c1nc(-c2ccccc2)nc(-c2ccc(-c3cccc4c3sc3ccccc34)cc2)n1]

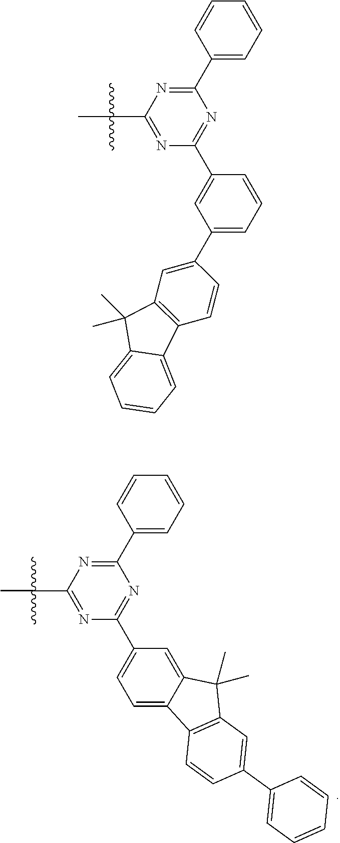 [SMILES: *c1nc(-c2ccccc2)nc(-c2cccc(-c3ccc4c(c3)C(C)(C)c3ccccc3-4)c2)n1 ; *c1nc(-c2ccccc2)nc(-c2ccc3c(c2)C(C)(C)c2cc(-c4ccccc4)ccc2-3)n1]

In some embodiments, the compound has the structure shown in Formula II-1 to Formula II-16:

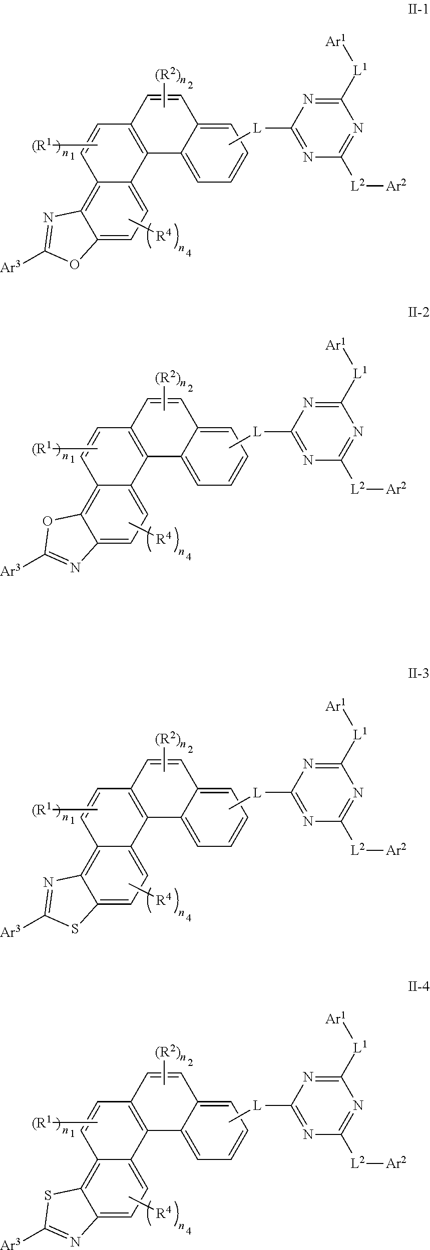

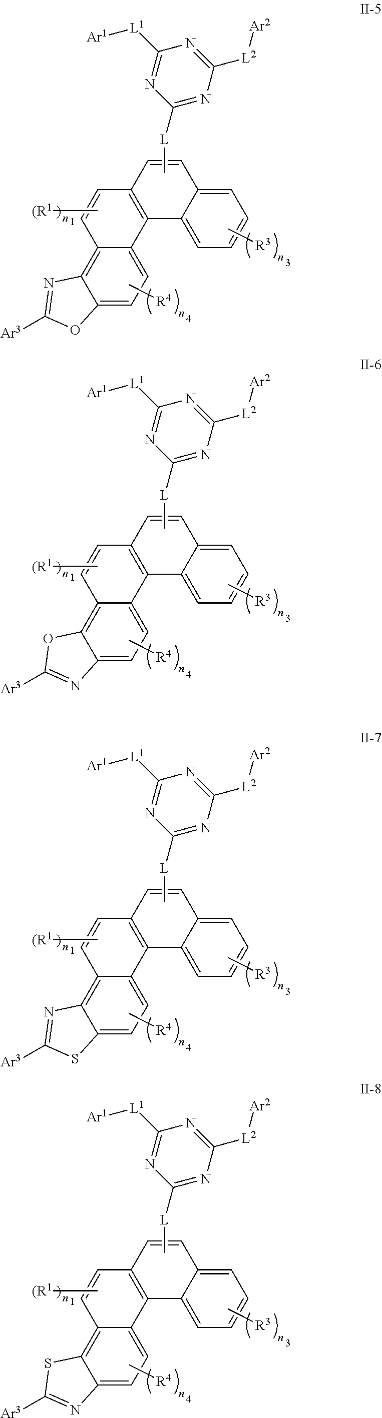

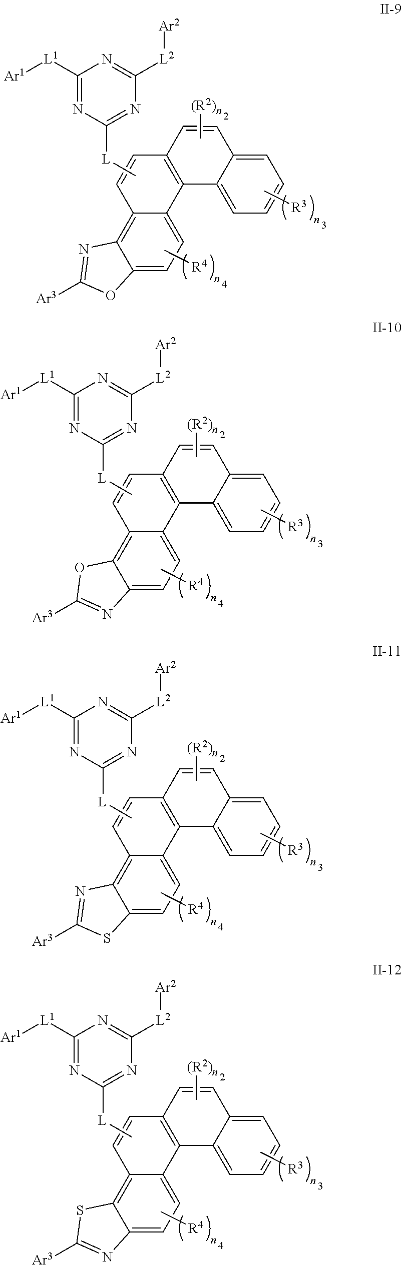

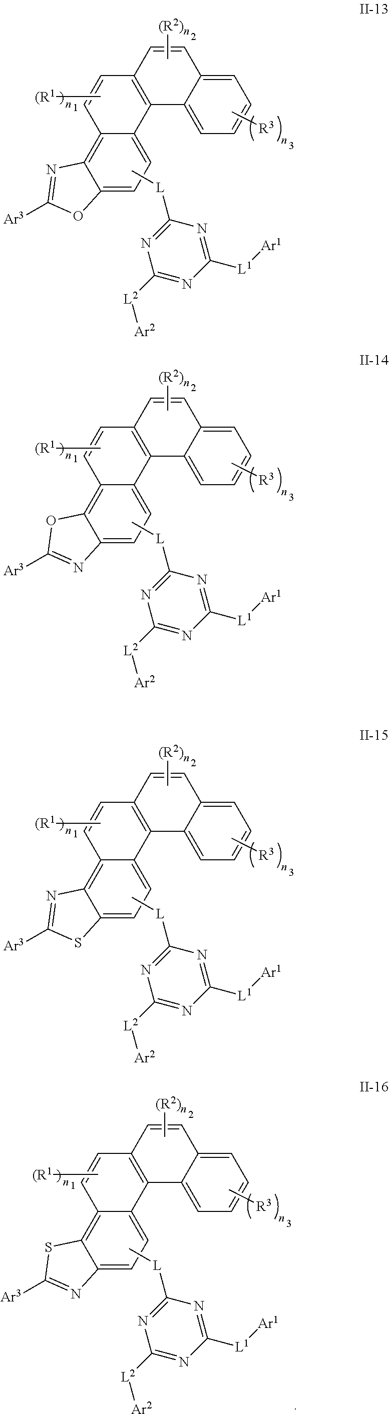

In some embodiments, the compound is selected from the group consisting of the following compounds:

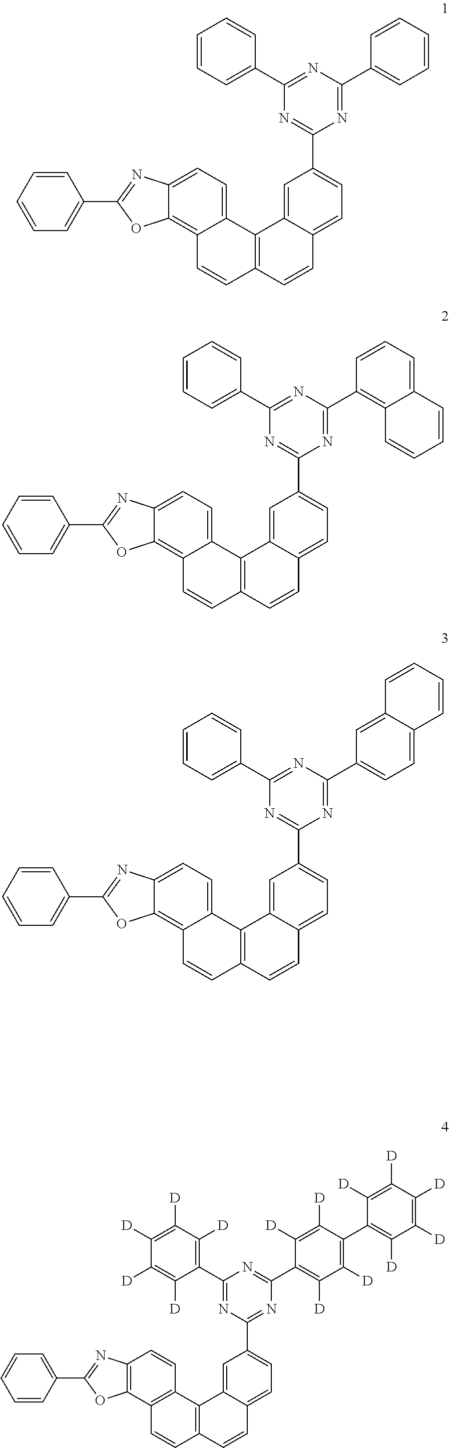 [SMILES: c1ccc(-c2nc(-c3ccccc3)nc(-c3ccc4ccc5ccc6c(ccc7nc(-c8ccccc8)oc76)c5c4c3)n2)cc1 ; c1ccc(-c2nc(-c3ccc4ccc5ccc6c(ccc7nc(-c8ccccc8)oc76)c5c4c3)nc(-c3cccc4ccccc34)n2)cc1 ; c1ccc(-c2nc(-c3ccc4ccccc4c3)nc(-c3ccc4ccc5ccc6c(ccc7nc(-c8ccccc8)oc76)c5c4c3)n2)cc1 ; [2H]c1c([2H])c([2H])c(-c2nc(-c3ccc4ccc5ccc6c(ccc7nc(-c8ccccc8)oc76)c5c4c3)nc(-c3c([2H])c([2H])c(-c4c([2H])c([2H])c([2H])c([2H])c4[2H])c([2H])c3[2H])n2)c([2H])c1[2H]]

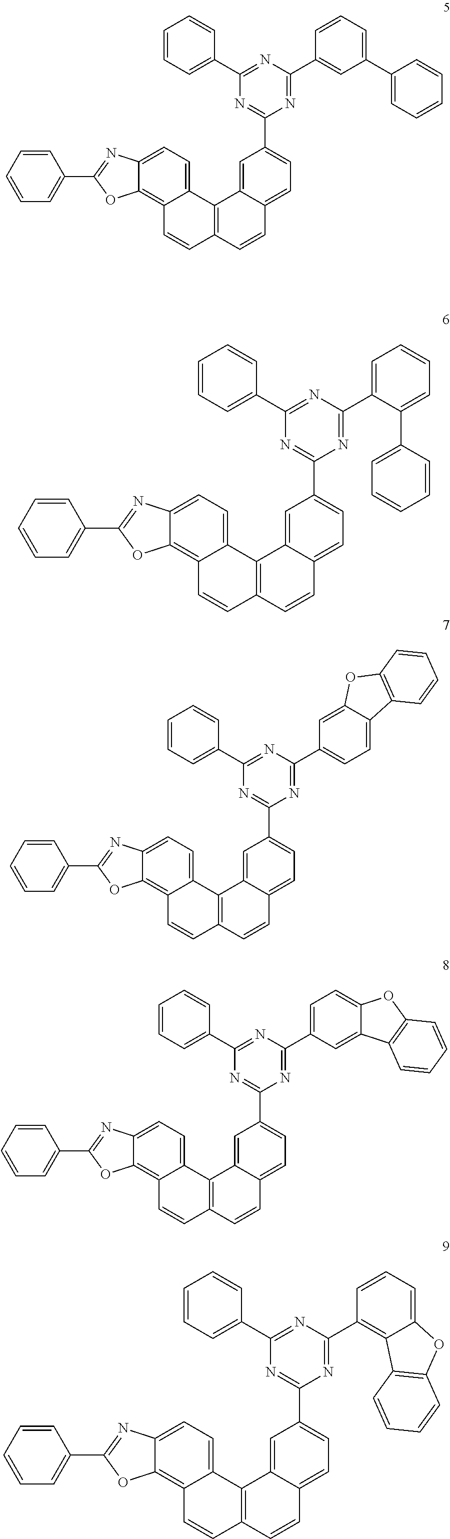 [SMILES: c1ccc(-c2cccc(-c3nc(-c4ccccc4)nc(-c4ccc5ccc6ccc7c(ccc8nc(-c9ccccc9)oc87)c6c5c4)n3)c2)cc1 ; c1ccc(-c2nc(-c3ccc4ccc5ccc6c(ccc7nc(-c8ccccc8)oc76)c5c4c3)nc(-c3ccccc3-c3ccccc3)n2)cc1 ; c1ccc(-c2nc(-c3ccc4c(c3)oc3ccccc34)nc(-c3ccc4ccc5ccc6c(ccc7nc(-c8ccccc8)oc76)c5c4c3)n2)cc1 ; c1ccc(-c2nc(-c3ccc4oc5ccccc5c4c3)nc(-c3ccc4ccc5ccc6c(ccc7nc(-c8ccccc8)oc76)c5c4c3)n2)cc1 ; c1ccc(-c2nc(-c3ccc4ccc5ccc6c(ccc7nc(-c8ccccc8)oc76)c5c4c3)nc(-c3cccc4oc5ccccc5c34)n2)cc1]

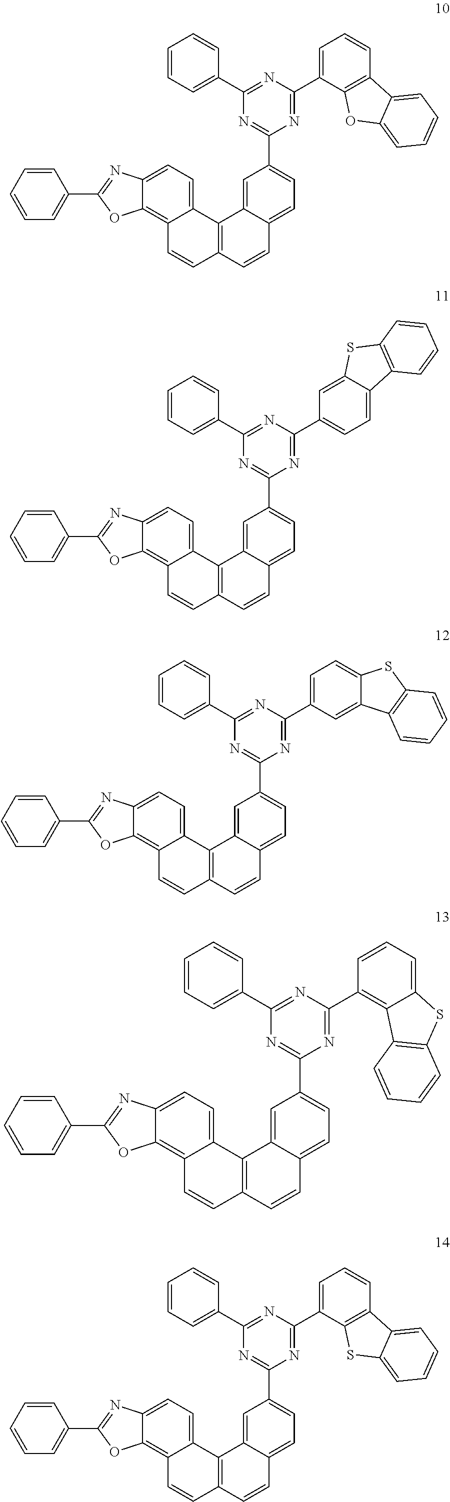 [SMILES: c1ccc(-c2nc(-c3ccc4ccc5ccc6c(ccc7nc(-c8ccccc8)oc76)c5c4c3)nc(-c3cccc4c3oc3ccccc34)n2)cc1 ; c1ccc(-c2nc(-c3ccc4c(c3)sc3ccccc34)nc(-c3ccc4ccc5ccc6c(ccc7nc(-c8ccccc8)oc76)c5c4c3)n2)cc1 ; c1ccc(-c2nc(-c3ccc4sc5ccccc5c4c3)nc(-c3ccc4ccc5ccc6c(ccc7nc(-c8ccccc8)oc76)c5c4c3)n2)cc1 ; c1ccc(-c2nc(-c3ccc4ccc5ccc6c(ccc7nc(-c8ccccc8)oc76)c5c4c3)nc(-c3cccc4sc5ccccc5c34)n2)cc1 ; c1ccc(-c2nc(-c3ccc4ccc5ccc6c(ccc7nc(-c8ccccc8)oc76)c5c4c3)nc(-c3cccc4c3sc3ccccc34)n2)cc1]

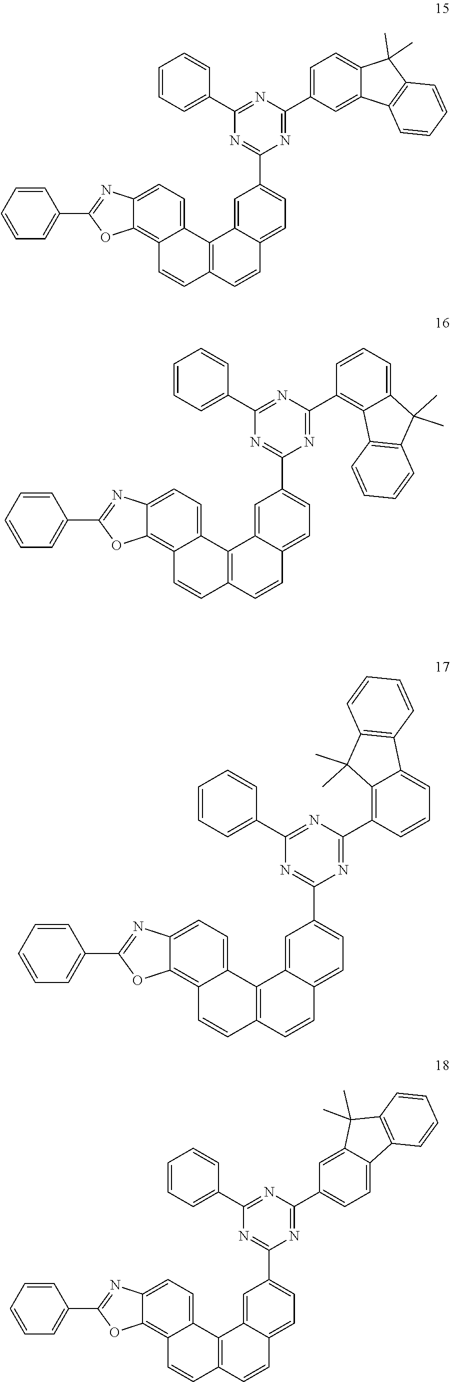 [SMILES: CC1(C)c2ccccc2-c2cc(-c3nc(-c4ccccc4)nc(-c4ccc5ccc6ccc7c(ccc8nc(-c9ccccc9)oc87)c6c5c4)n3)ccc21 ; CC1(C)c2ccccc2-c2c(-c3nc(-c4ccccc4)nc(-c4ccc5ccc6ccc7c(ccc8nc(-c9ccccc9)oc87)c6c5c4)n3)cccc21 ; CC1(C)c2ccccc2-c2cccc(-c3nc(-c4ccccc4)nc(-c4ccc5ccc6ccc7c(ccc8nc(-c9ccccc9)oc87)c6c5c4)n3)c21 ; CC1(C)c2ccccc2-c2ccc(-c3nc(-c4ccccc4)nc(-c4ccc5ccc6ccc7c(ccc8nc(-c9ccccc9)oc87)c6c5c4)n3)cc21]

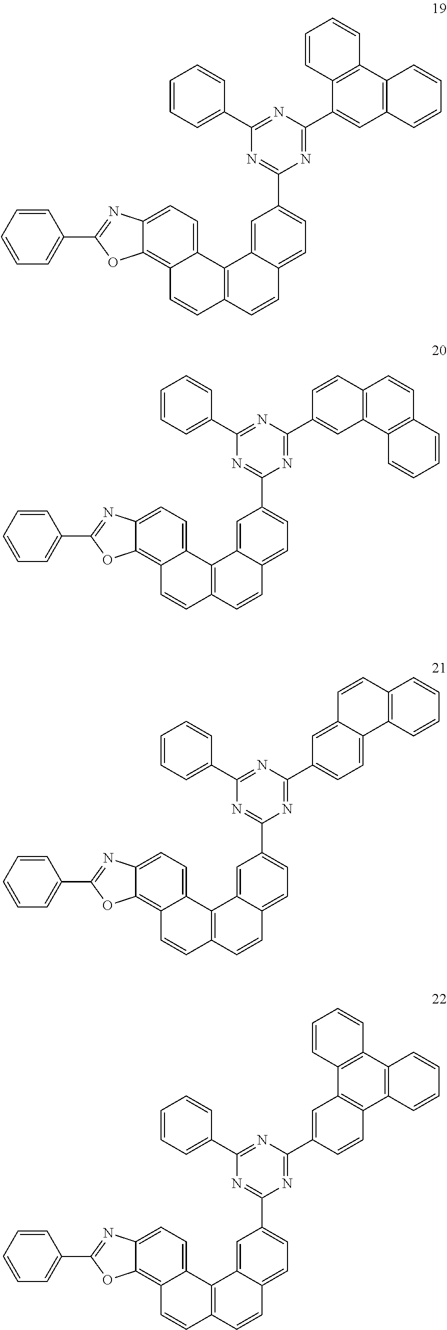 [SMILES: c1ccc(-c2nc(-c3ccc4ccc5ccc6c(ccc7nc(-c8ccccc8)oc76)c5c4c3)nc(-c3cc4ccccc4c4ccccc34)n2)cc1 ; c1ccc(-c2nc(-c3ccc4ccc5ccccc5c4c3)nc(-c3ccc4ccc5ccc6c(ccc7nc(-c8ccccc8)oc76)c5c4c3)n2)cc1 ; c1ccc(-c2nc(-c3ccc4c(ccc5ccccc54)c3)nc(-c3ccc4ccc5ccc6c(ccc7nc(-c8ccccc8)oc76)c5c4c3)n2)cc1 ; c1ccc(-c2nc(-c3ccc4c5ccccc5c5ccccc5c4c3)nc(-c3ccc4ccc5ccc6c(ccc7nc(-c8ccccc8)oc76)c5c4c3)n2)cc1]

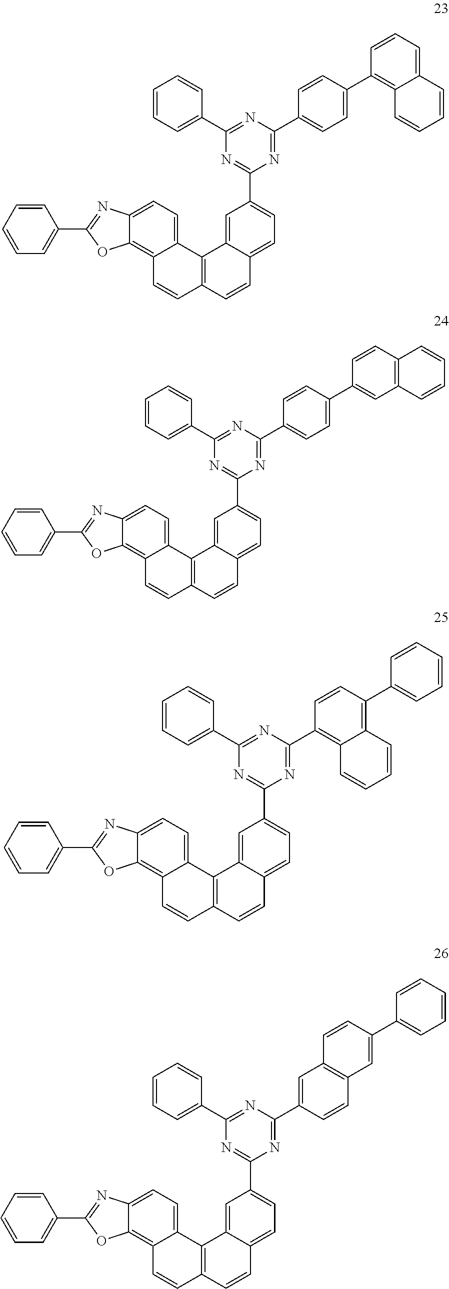 [SMILES: c1ccc(-c2nc(-c3ccc(-c4cccc5ccccc45)cc3)nc(-c3ccc4ccc5ccc6c(ccc7nc(-c8ccccc8)oc76)c5c4c3)n2)cc1 ; c1ccc(-c2nc(-c3ccc(-c4ccc5ccccc5c4)cc3)nc(-c3ccc4ccc5ccc6c(ccc7nc(-c8ccccc8)oc76)c5c4c3)n2)cc1 ; c1ccc(-c2nc(-c3ccc4ccc5ccc6c(ccc7nc(-c8ccccc8)oc76)c5c4c3)nc(-c3ccc(-c4ccccc4)c4ccccc34)n2)cc1 ; c1ccc(-c2ccc3cc(-c4nc(-c5ccccc5)nc(-c5ccc6ccc7ccc8c(ccc9nc(-c%10ccccc%10)oc98)c7c6c5)n4)ccc3c2)cc1]

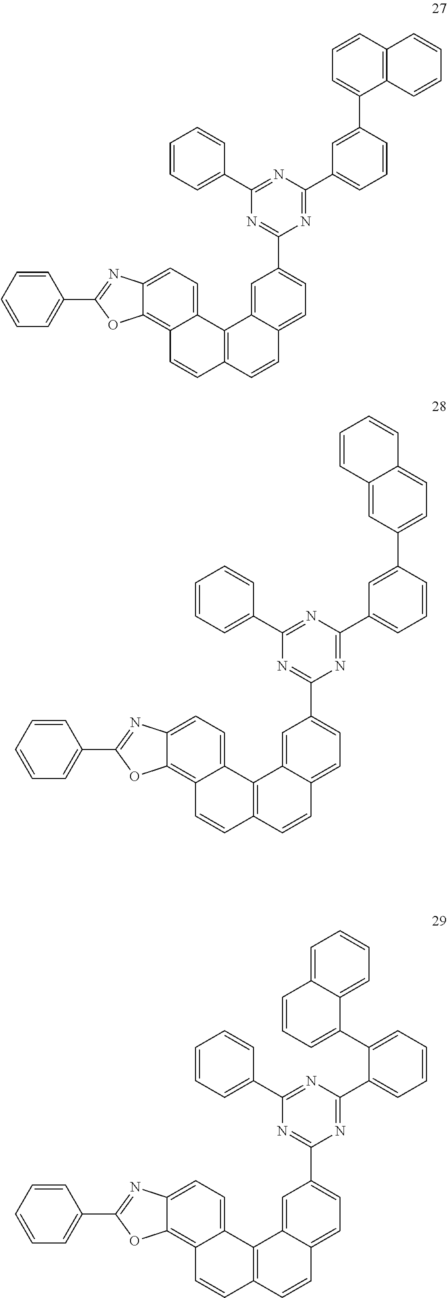 [SMILES: c1ccc(-c2nc(-c3cccc(-c4cccc5ccccc45)c3)nc(-c3ccc4ccc5ccc6c(ccc7nc(-c8ccccc8)oc76)c5c4c3)n2)cc1 ; c1ccc(-c2nc(-c3cccc(-c4ccc5ccccc5c4)c3)nc(-c3ccc4ccc5ccc6c(ccc7nc(-c8ccccc8)oc76)c5c4c3)n2)cc1 ; c1ccc(-c2nc(-c3ccc4ccc5ccc6c(ccc7nc(-c8ccccc8)oc76)c5c4c3)nc(-c3ccccc3-c3cccc4ccccc34)n2)cc1]

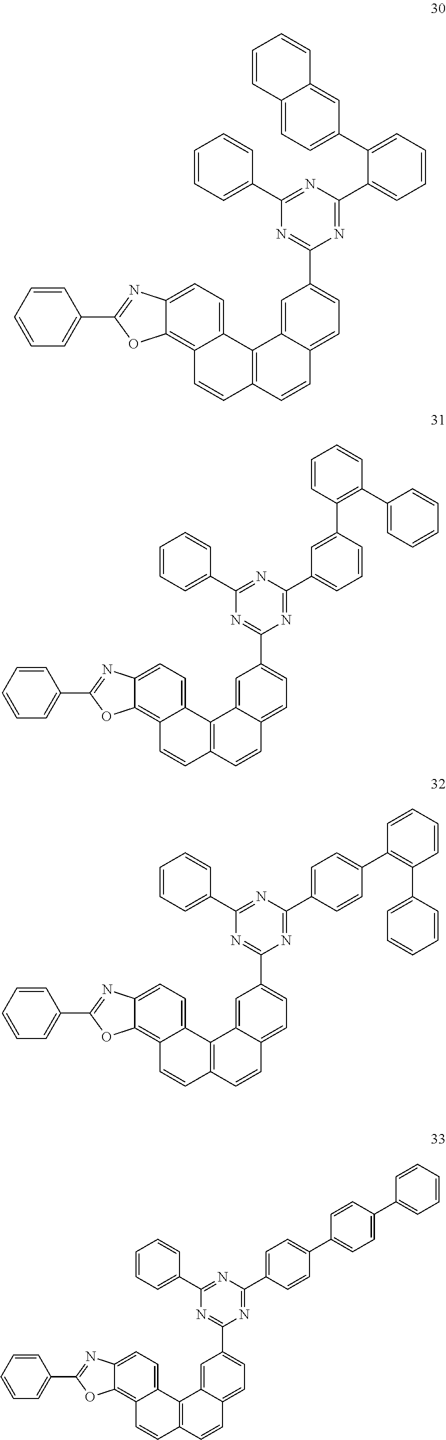 [SMILES: c1ccc(-c2nc(-c3ccc4ccc5ccc6c(ccc7nc(-c8ccccc8)oc76)c5c4c3)nc(-c3ccccc3-c3ccc4ccccc4c3)n2)cc1 ; c1ccc(-c2nc(-c3cccc(-c4ccccc4-c4ccccc4)c3)nc(-c3ccc4ccc5ccc6c(ccc7nc(-c8ccccc8)oc76)c5c4c3)n2)cc1 ; c1ccc(-c2nc(-c3ccc(-c4ccccc4-c4ccccc4)cc3)nc(-c3ccc4ccc5ccc6c(ccc7nc(-c8ccccc8)oc76)c5c4c3)n2)cc1 ; c1ccc(-c2ccc(-c3ccc(-c4nc(-c5ccccc5)nc(-c5ccc6ccc7ccc8c(ccc9nc(-c%10ccccc%10)oc98)c7c6c5)n4)cc3)cc2)cc1]

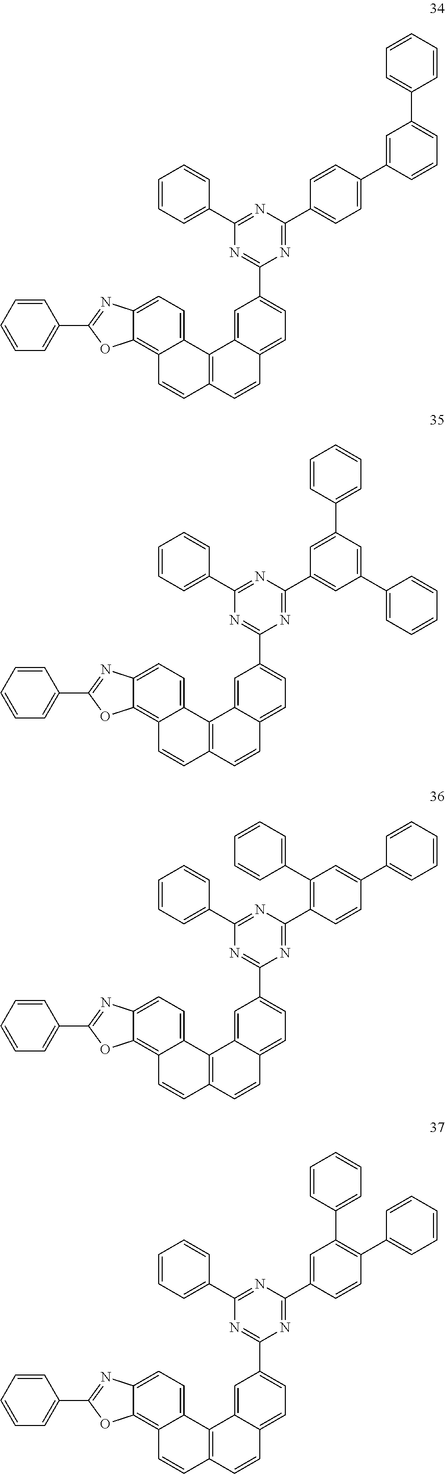 [SMILES: c1ccc(-c2cccc(-c3ccc(-c4nc(-c5ccccc5)nc(-c5ccc6ccc7ccc8c(ccc9nc(-c%10ccccc%10)oc98)c7c6c5)n4)cc3)c2)cc1 ; c1ccc(-c2cc(-c3ccccc3)cc(-c3nc(-c4ccccc4)nc(-c4ccc5ccc6ccc7c(ccc8nc(-c9ccccc9)oc87)c6c5c4)n3)c2)cc1 ; c1ccc(-c2ccc(-c3nc(-c4ccccc4)nc(-c4ccc5ccc6ccc7c(ccc8nc(-c9ccccc9)oc87)c6c5c4)n3)c(-c3ccccc3)c2)cc1 ; c1ccc(-c2nc(-c3ccc(-c4ccccc4)c(-c4ccccc4)c3)nc(-c3ccc4ccc5ccc6c(ccc7nc(-c8ccccc8)oc76)c5c4c3)n2)cc1]

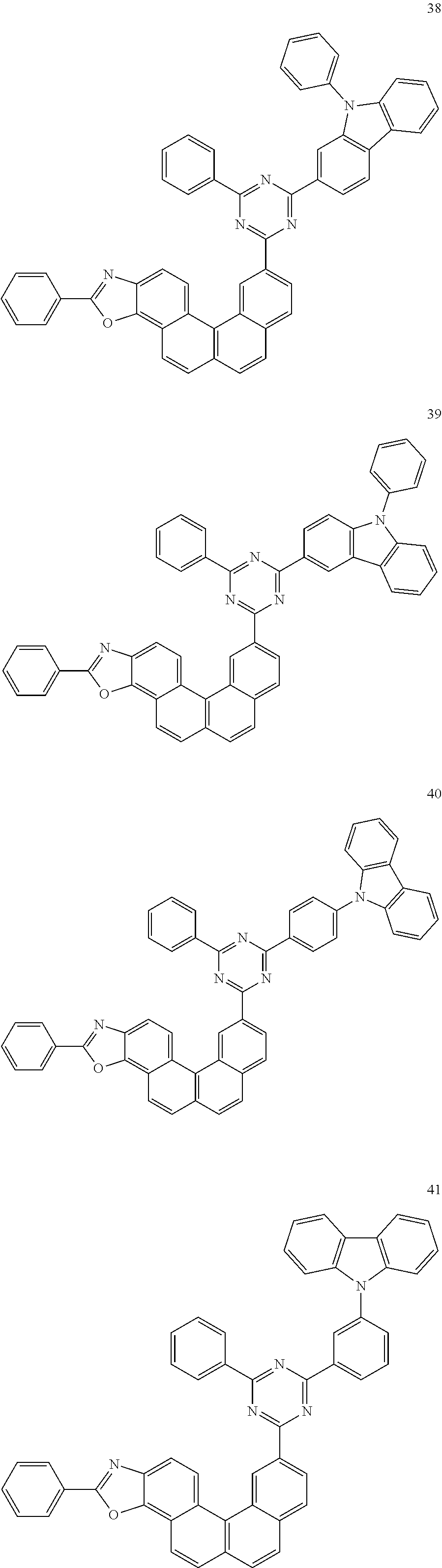 [SMILES: c1ccc(-c2nc(-c3ccc4ccc5ccc6c(ccc7nc(-c8ccccc8)oc76)c5c4c3)nc(-c3ccc4c5ccccc5n(-c5ccccc5)c4c3)n2)cc1 ; c1ccc(-c2nc(-c3ccc4ccc5ccc6c(ccc7nc(-c8ccccc8)oc76)c5c4c3)nc(-c3ccc4c(c3)c3ccccc3n4-c3ccccc3)n2)cc1 ; c1ccc(-c2nc(-c3ccc(-n4c5ccccc5c5ccccc54)cc3)nc(-c3ccc4ccc5ccc6c(ccc7nc(-c8ccccc8)oc76)c5c4c3)n2)cc1 ; c1ccc(-c2nc(-c3cccc(-n4c5ccccc5c5ccccc54)c3)nc(-c3ccc4ccc5ccc6c(ccc7nc(-c8ccccc8)oc76)c5c4c3)n2)cc1]

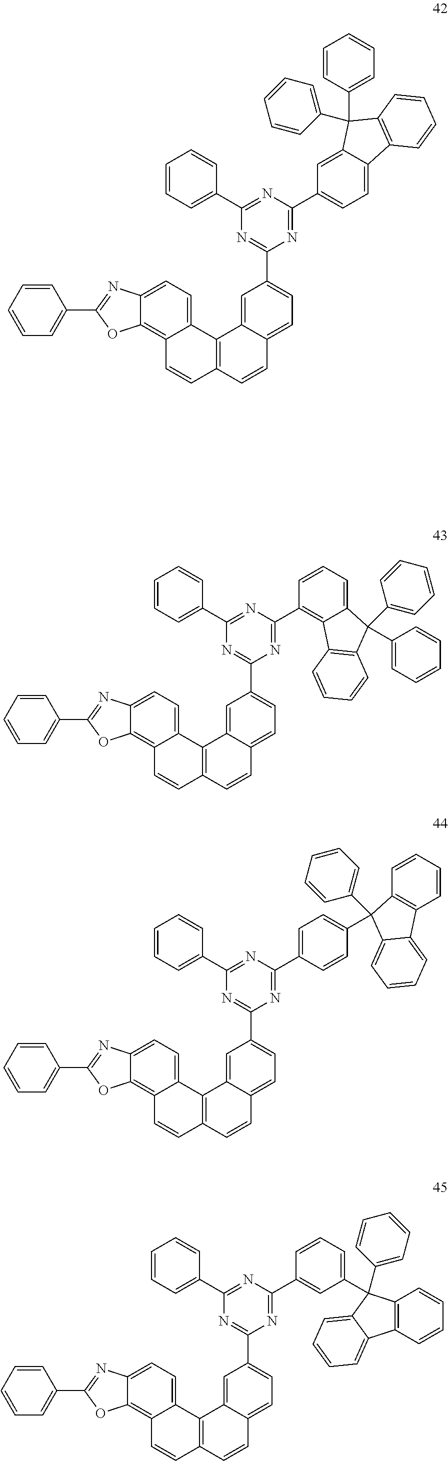 [SMILES: c1ccc(-c2nc(-c3ccc4c(c3)C(c3ccccc3)(c3ccccc3)c3ccccc3-4)nc(-c3ccc4ccc5ccc6c(ccc7nc(-c8ccccc8)oc76)c5c4c3)n2)cc1 ; c1ccc(-c2nc(-c3ccc4ccc5ccc6c(ccc7nc(-c8ccccc8)oc76)c5c4c3)nc(-c3cccc4c3-c3ccccc3C4(c3ccccc3)c3ccccc3)n2)cc1 ; c1ccc(-c2nc(-c3ccc(C4(c5ccccc5)c5ccccc5-c5ccccc54)cc3)nc(-c3ccc4ccc5ccc6c(ccc7nc(-c8ccccc8)oc76)c5c4c3)n2)cc1 ; c1ccc(-c2nc(-c3cccc(C4(c5ccccc5)c5ccccc5-c5ccccc54)c3)nc(-c3ccc4ccc5ccc6c(ccc7nc(-c8ccccc8)oc76)c5c4c3)n2)cc1]

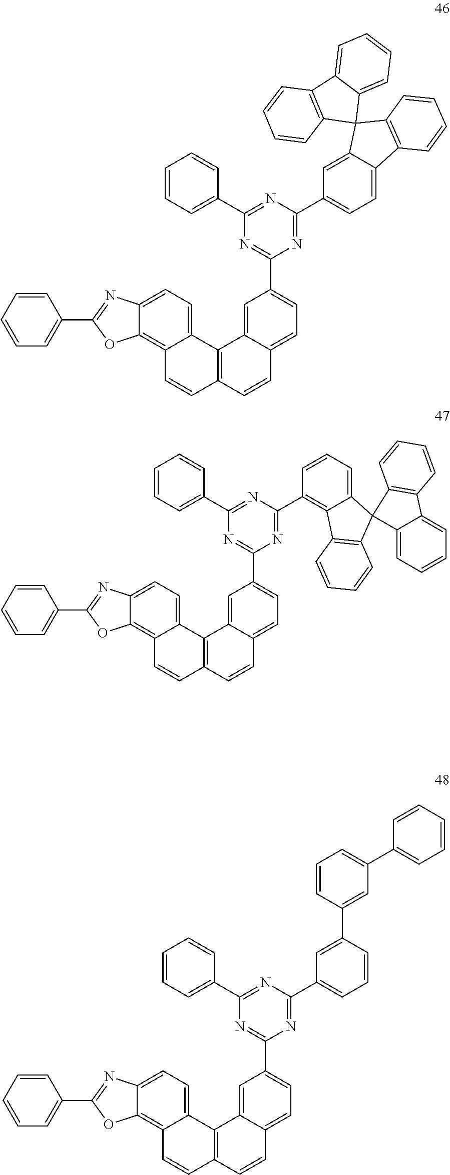 [SMILES: c1ccc(-c2nc(-c3ccc4c(c3)C3(c5ccccc5-c5ccccc53)c3ccccc3-4)nc(-c3ccc4ccc5ccc6c(ccc7nc(-c8ccccc8)oc76)c5c4c3)n2)cc1 ; c1ccc(-c2nc(-c3ccc4ccc5ccc6c(ccc7nc(-c8ccccc8)oc76)c5c4c3)nc(-c3cccc4c3-c3ccccc3C43c4ccccc4-c4ccccc43)n2)cc1 ; c1ccc(-c2cccc(-c3cccc(-c4nc(-c5ccccc5)nc(-c5ccc6ccc7ccc8c(ccc9nc(-c%10ccccc%10)oc98)c7c6c5)n4)c3)c2)cc1]

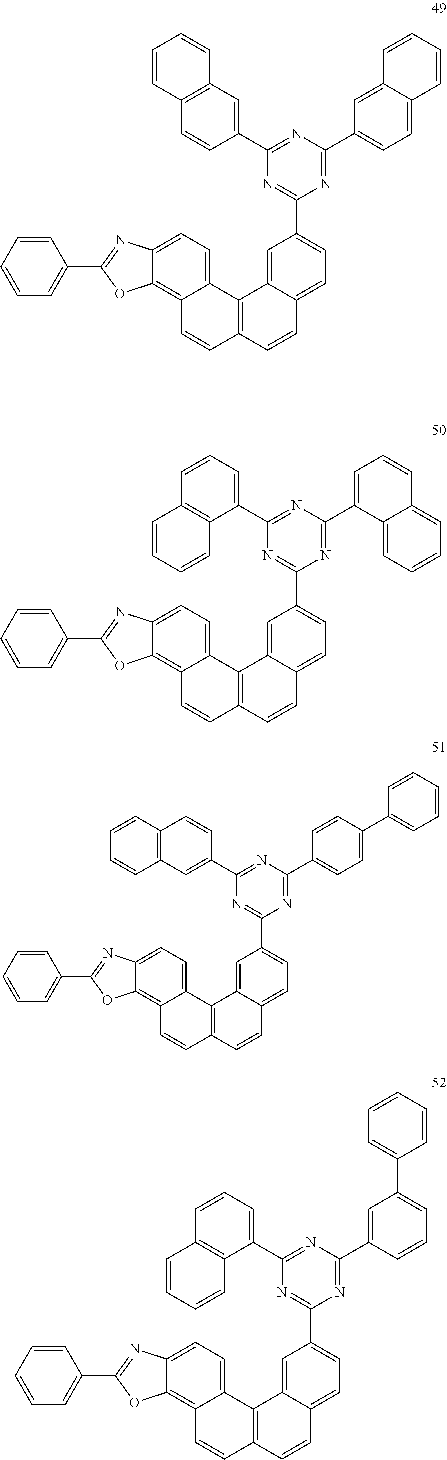 [SMILES: c1ccc(-c2nc3ccc4c(ccc5ccc6ccc(-c7nc(-c8ccc9ccccc9c8)nc(-c8ccc9ccccc9c8)n7)cc6c54)c3o2)cc1 ; c1ccc(-c2nc3ccc4c(ccc5ccc6ccc(-c7nc(-c8cccc9ccccc89)nc(-c8cccc9ccccc89)n7)cc6c54)c3o2)cc1 ; c1ccc(-c2ccc(-c3nc(-c4ccc5ccccc5c4)nc(-c4ccc5ccc6ccc7c(ccc8nc(-c9ccccc9)oc87)c6c5c4)n3)cc2)cc1 ; c1ccc(-c2cccc(-c3nc(-c4ccc5ccc6ccc7c(ccc8nc(-c9ccccc9)oc87)c6c5c4)nc(-c4cccc5ccccc45)n3)c2)cc1]

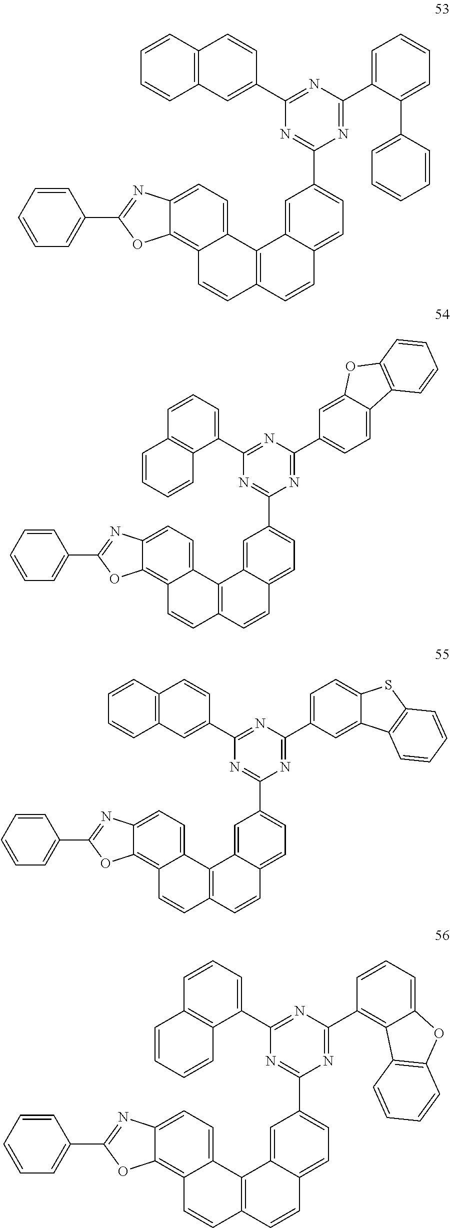 [SMILES: c1ccc(-c2nc3ccc4c(ccc5ccc6ccc(-c7nc(-c8ccc9ccccc9c8)nc(-c8ccccc8-c8ccccc8)n7)cc6c54)c3o2)cc1 ; c1ccc(-c2nc3ccc4c(ccc5ccc6ccc(-c7nc(-c8ccc9c(c8)oc8ccccc89)nc(-c8cccc9ccccc89)n7)cc6c54)c3o2)cc1 ; c1ccc(-c2nc3ccc4c(ccc5ccc6ccc(-c7nc(-c8ccc9ccccc9c8)nc(-c8ccc9sc%10ccccc%10c9c8)n7)cc6c54)c3o2)cc1 ; c1ccc(-c2nc3ccc4c(ccc5ccc6ccc(-c7nc(-c8cccc9ccccc89)nc(-c8cccc9oc%10ccccc%10c89)n7)cc6c54)c3o2)cc1]

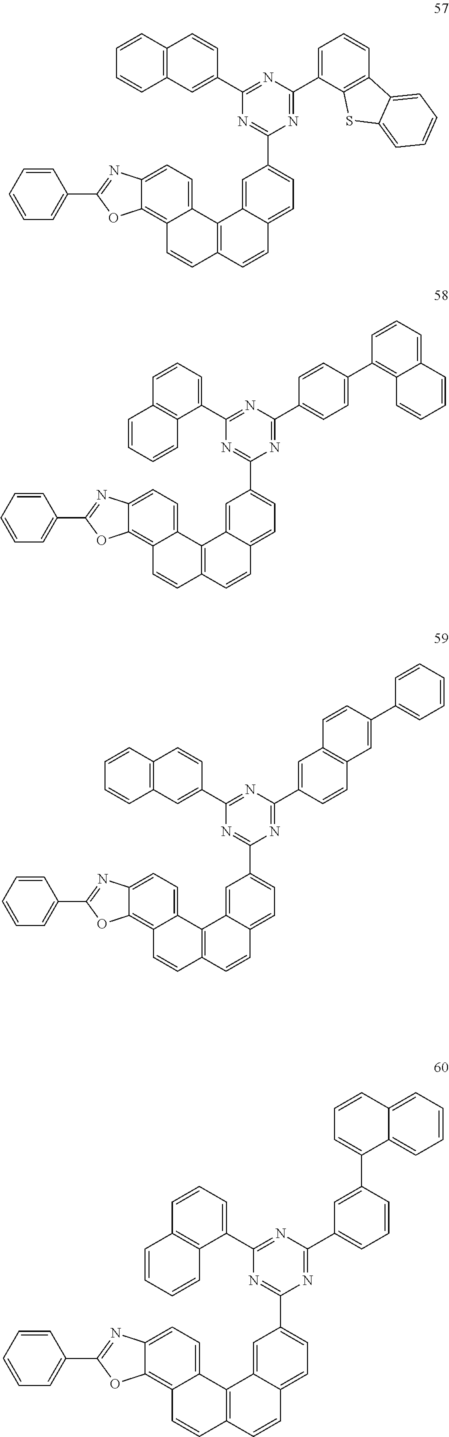 [SMILES: c1ccc(-c2nc3ccc4c(ccc5ccc6ccc(-c7nc(-c8ccc9ccccc9c8)nc(-c8cccc9c8sc8ccccc89)n7)cc6c54)c3o2)cc1 ; c1ccc(-c2nc3ccc4c(ccc5ccc6ccc(-c7nc(-c8ccc(-c9cccc%10ccccc9%10)cc8)nc(-c8cccc9ccccc89)n7)cc6c54)c3o2)cc1 ; c1ccc(-c2ccc3cc(-c4nc(-c5ccc6ccccc6c5)nc(-c5ccc6ccc7ccc8c(ccc9nc(-c%10ccccc%10)oc98)c7c6c5)n4)ccc3c2)cc1 ; c1ccc(-c2nc3ccc4c(ccc5ccc6ccc(-c7nc(-c8cccc(-c9cccc%10ccccc9%10)c8)nc(-c8cccc9ccccc89)n7)cc6c54)c3o2)cc1]

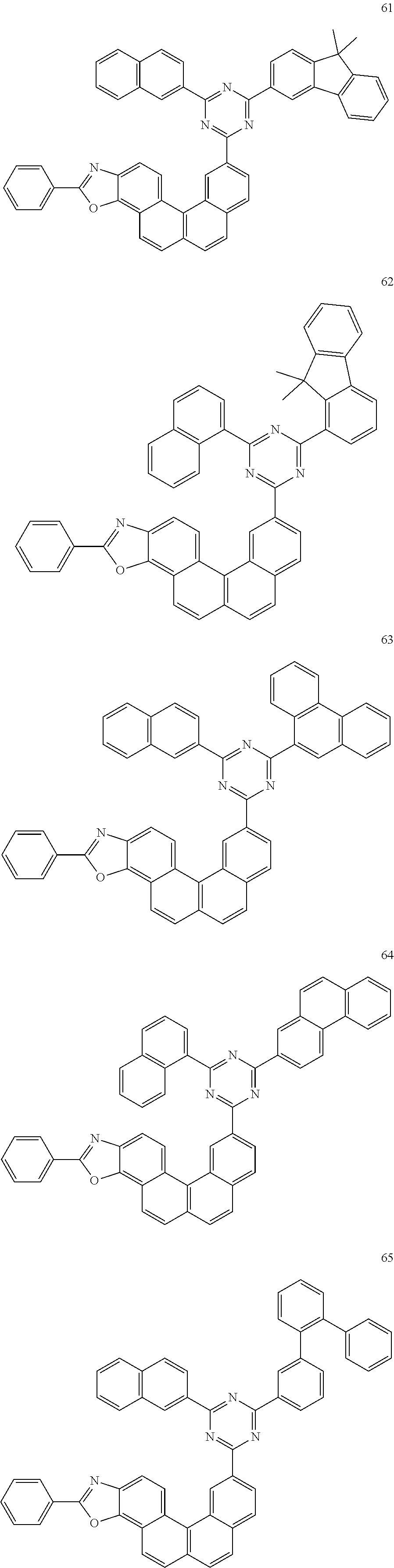

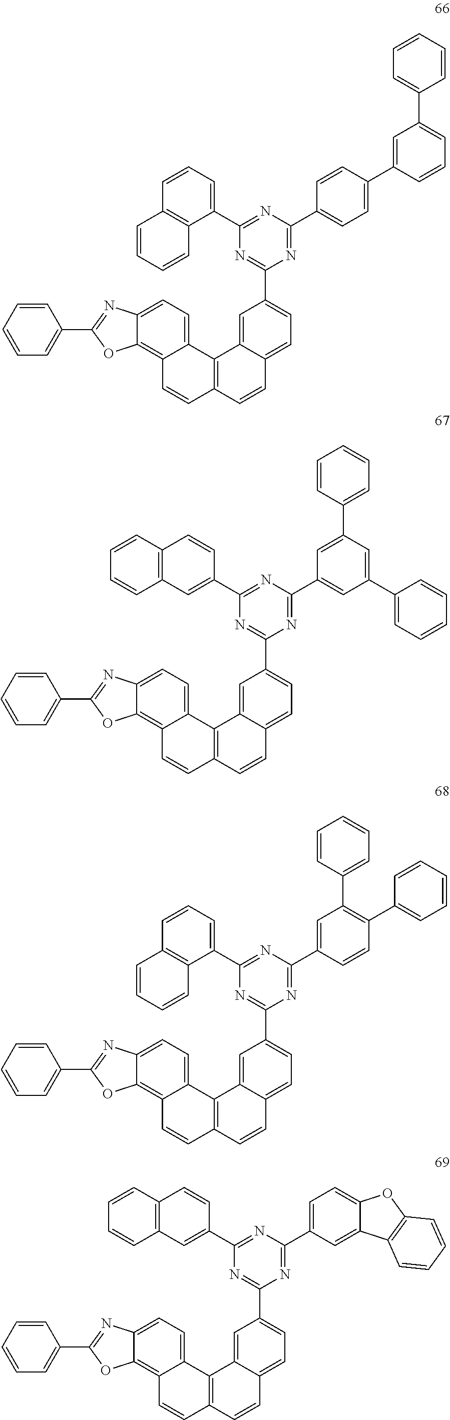 [SMILES: c1ccc(-c2cccc(-c3ccc(-c4nc(-c5ccc6ccc7ccc8c(ccc9nc(-c%10ccccc%10)oc98)c7c6c5)nc(-c5cccc6ccccc56)n4)cc3)c2)cc1 ; c1ccc(-c2cc(-c3ccccc3)cc(-c3nc(-c4ccc5ccccc5c4)nc(-c4ccc5ccc6ccc7c(ccc8nc(-c9ccccc9)oc87)c6c5c4)n3)c2)cc1 ; c1ccc(-c2nc3ccc4c(ccc5ccc6ccc(-c7nc(-c8ccc(-c9ccccc9)c(-c9ccccc9)c8)nc(-c8cccc9ccccc89)n7)cc6c54)c3o2)cc1 ; c1ccc(-c2nc3ccc4c(ccc5ccc6ccc(-c7nc(-c8ccc9ccccc9c8)nc(-c8ccc9oc%10ccccc%10c9c8)n7)cc6c54)c3o2)cc1]

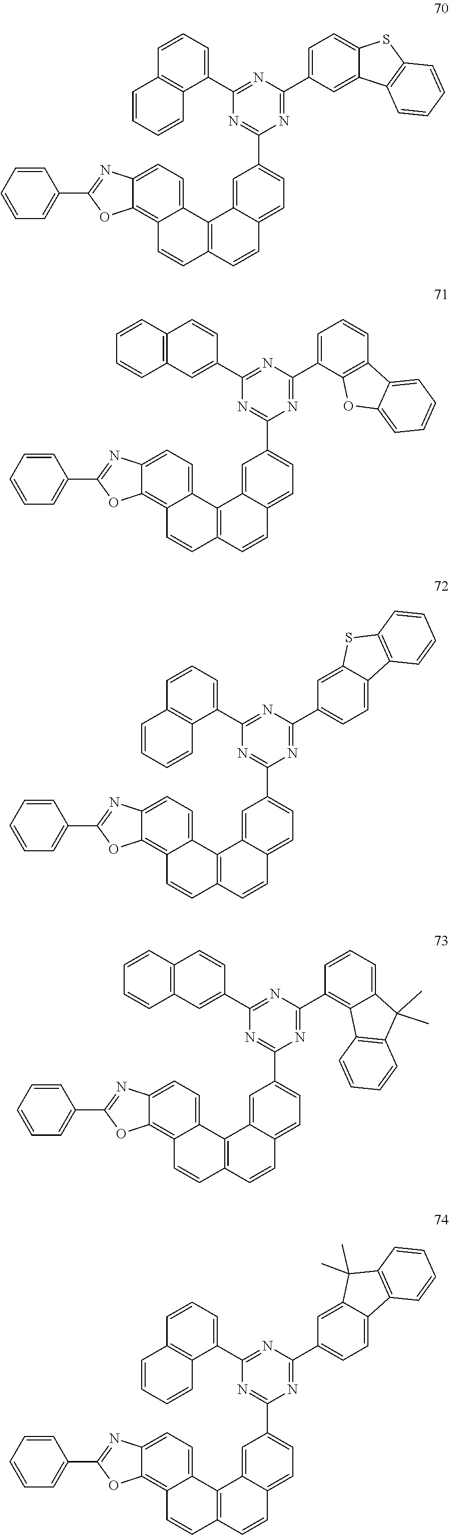 [SMILES: c1ccc(-c2nc3ccc4c(ccc5ccc6ccc(-c7nc(-c8ccc9sc%10ccccc%10c9c8)nc(-c8cccc9ccccc89)n7)cc6c54)c3o2)cc1 ; c1ccc(-c2nc3ccc4c(ccc5ccc6ccc(-c7nc(-c8ccc9ccccc9c8)nc(-c8cccc9c8oc8ccccc89)n7)cc6c54)c3o2)cc1 ; c1ccc(-c2nc3ccc4c(ccc5ccc6ccc(-c7nc(-c8ccc9c(c8)sc8ccccc89)nc(-c8cccc9ccccc89)n7)cc6c54)c3o2)cc1 ; CC1(C)c2ccccc2-c2c(-c3nc(-c4ccc5ccccc5c4)nc(-c4ccc5ccc6ccc7c(ccc8nc(-c9ccccc9)oc87)c6c5c4)n3)cccc21 ; CC1(C)c2ccccc2-c2ccc(-c3nc(-c4ccc5ccc6ccc7c(ccc8nc(-c9ccccc9)oc87)c6c5c4)nc(-c4cccc5ccccc45)n3)cc21]

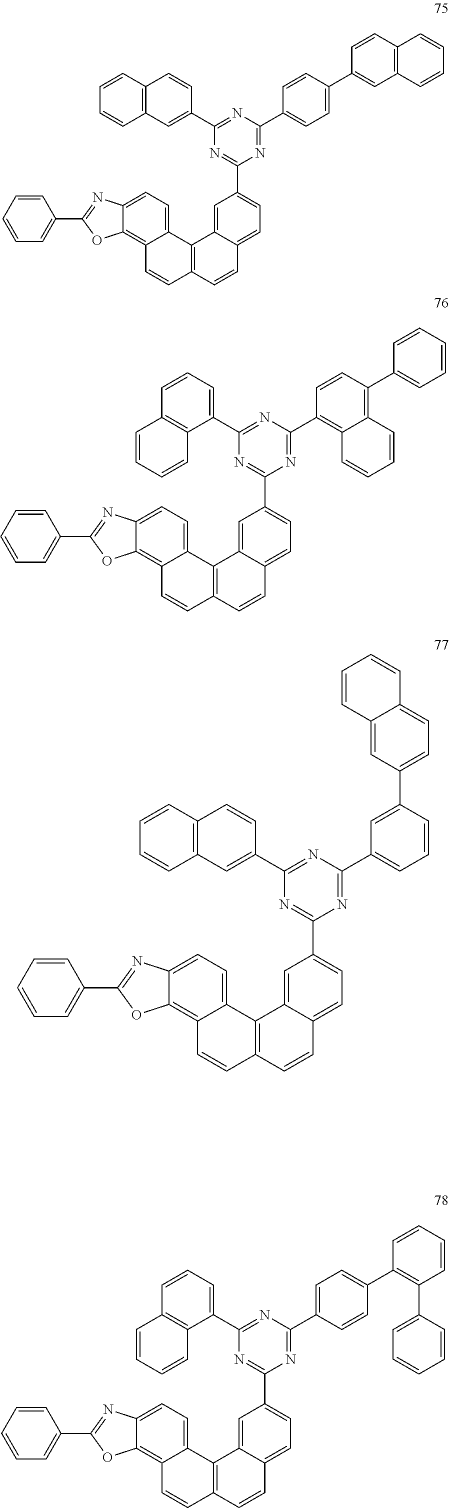 [SMILES: c1ccc(-c2nc3ccc4c(ccc5ccc6ccc(-c7nc(-c8ccc(-c9ccc%10ccccc%10c9)cc8)nc(-c8ccc9ccccc9c8)n7)cc6c54)c3o2)cc1 ; c1ccc(-c2nc3ccc4c(ccc5ccc6ccc(-c7nc(-c8cccc9ccccc89)nc(-c8ccc(-c9ccccc9)c9ccccc89)n7)cc6c54)c3o2)cc1 ; c1ccc(-c2nc3ccc4c(ccc5ccc6ccc(-c7nc(-c8cccc(-c9ccc%10ccccc%10c9)c8)nc(-c8ccc9ccccc9c8)n7)cc6c54)c3o2)cc1 ; c1ccc(-c2nc3ccc4c(ccc5ccc6ccc(-c7nc(-c8ccc(-c9ccccc9-c9ccccc9)cc8)nc(-c8cccc9ccccc89)n7)cc6c54)c3o2)cc1]

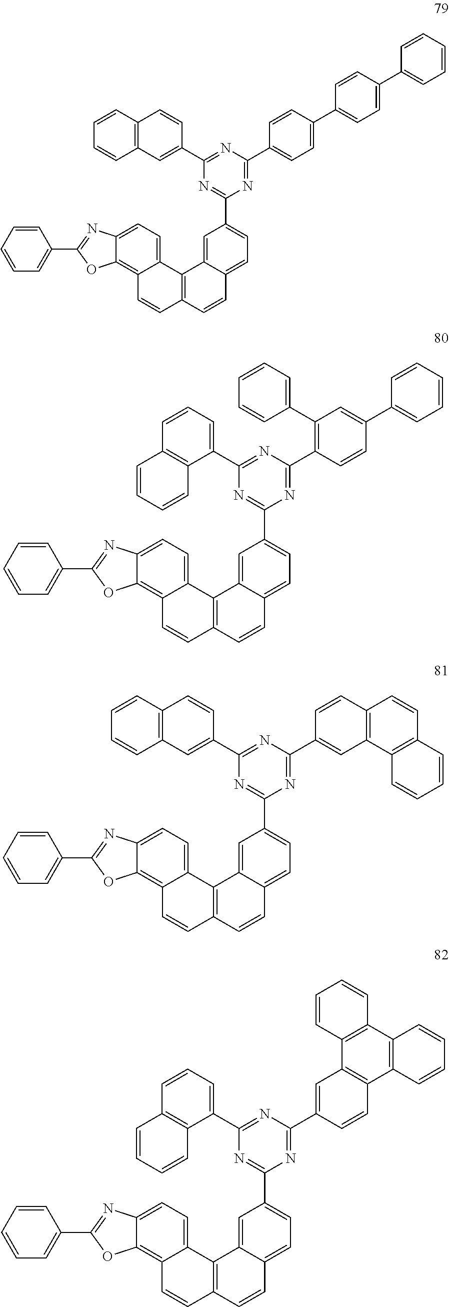 [SMILES: c1ccc(-c2ccc(-c3ccc(-c4nc(-c5ccc6ccccc6c5)nc(-c5ccc6ccc7ccc8c(ccc9nc(-c%10ccccc%10)oc98)c7c6c5)n4)cc3)cc2)cc1 ; c1ccc(-c2ccc(-c3nc(-c4ccc5ccc6ccc7c(ccc8nc(-c9ccccc9)oc87)c6c5c4)nc(-c4cccc5ccccc45)n3)c(-c3ccccc3)c2)cc1 ; c1ccc(-c2nc3ccc4c(ccc5ccc6ccc(-c7nc(-c8ccc9ccccc9c8)nc(-c8ccc9ccc%10ccccc%10c9c8)n7)cc6c54)c3o2)cc1 ; c1ccc(-c2nc3ccc4c(ccc5ccc6ccc(-c7nc(-c8ccc9c%10ccccc%10c%10ccccc%10c9c8)nc(-c8cccc9ccccc89)n7)cc6c54)c3o2)cc1]

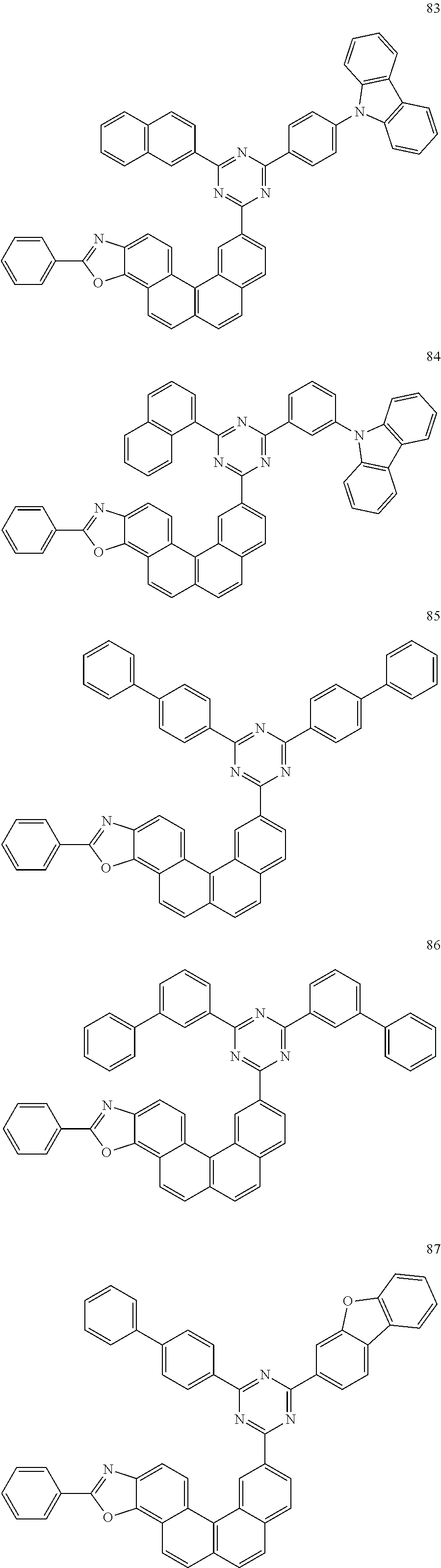

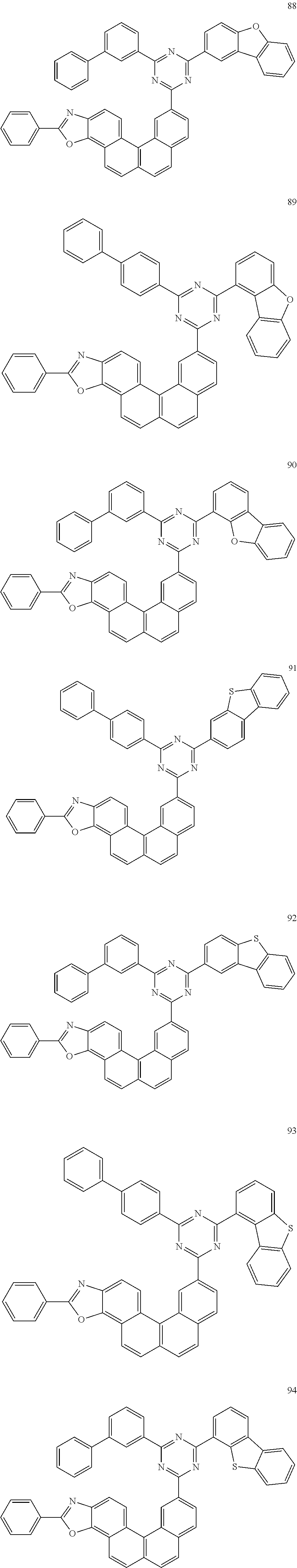

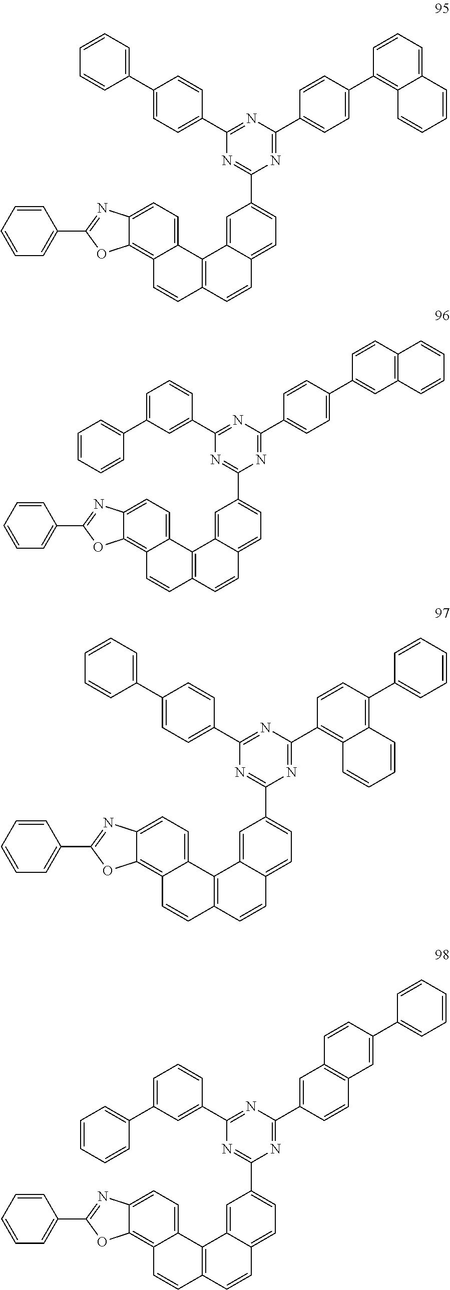 [SMILES: c1ccc(-c2ccc(-c3nc(-c4ccc(-c5cccc6ccccc56)cc4)nc(-c4ccc5ccc6ccc7c(ccc8nc(-c9ccccc9)oc87)c6c5c4)n3)cc2)cc1 ; c1ccc(-c2cccc(-c3nc(-c4ccc(-c5ccc6ccccc6c5)cc4)nc(-c4ccc5ccc6ccc7c(ccc8nc(-c9ccccc9)oc87)c6c5c4)n3)c2)cc1 ; c1ccc(-c2ccc(-c3nc(-c4ccc5ccc6ccc7c(ccc8nc(-c9ccccc9)oc87)c6c5c4)nc(-c4ccc(-c5ccccc5)c5ccccc45)n3)cc2)cc1 ; c1ccc(-c2cccc(-c3nc(-c4ccc5cc(-c6ccccc6)ccc5c4)nc(-c4ccc5ccc6ccc7c(ccc8nc(-c9ccccc9)oc87)c6c5c4)n3)c2)cc1]

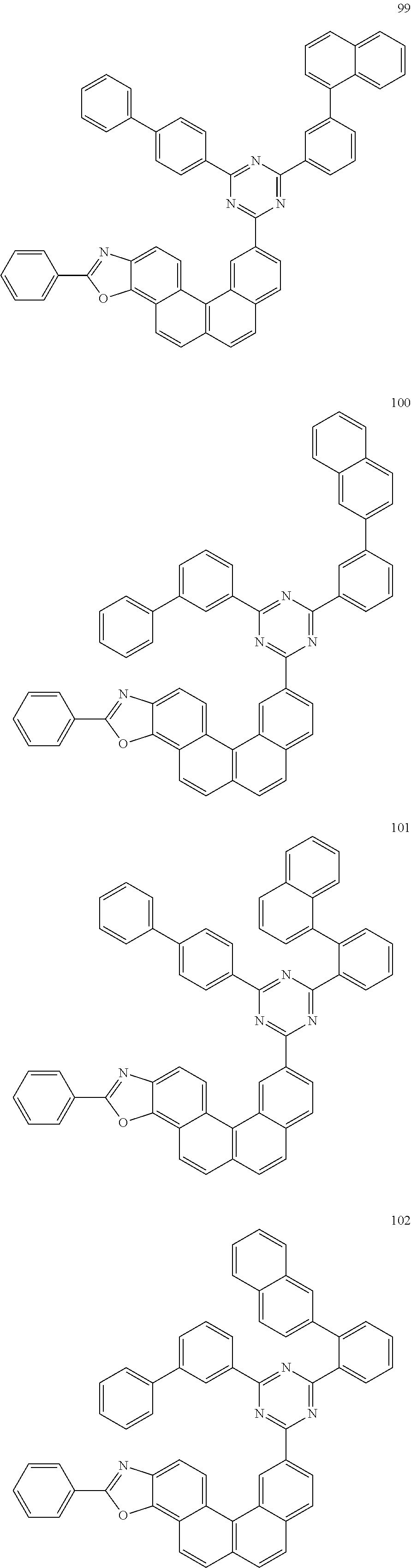 [SMILES: c1ccc(-c2ccc(-c3nc(-c4cccc(-c5cccc6ccccc56)c4)nc(-c4ccc5ccc6ccc7c(ccc8nc(-c9ccccc9)oc87)c6c5c4)n3)cc2)cc1 ; c1ccc(-c2cccc(-c3nc(-c4cccc(-c5ccc6ccccc6c5)c4)nc(-c4ccc5ccc6ccc7c(ccc8nc(-c9ccccc9)oc87)c6c5c4)n3)c2)cc1 ; c1ccc(-c2ccc(-c3nc(-c4ccc5ccc6ccc7c(ccc8nc(-c9ccccc9)oc87)c6c5c4)nc(-c4ccccc4-c4cccc5ccccc45)n3)cc2)cc1 ; c1ccc(-c2cccc(-c3nc(-c4ccc5ccc6ccc7c(ccc8nc(-c9ccccc9)oc87)c6c5c4)nc(-c4ccccc4-c4ccc5ccccc5c4)n3)c2)cc1]

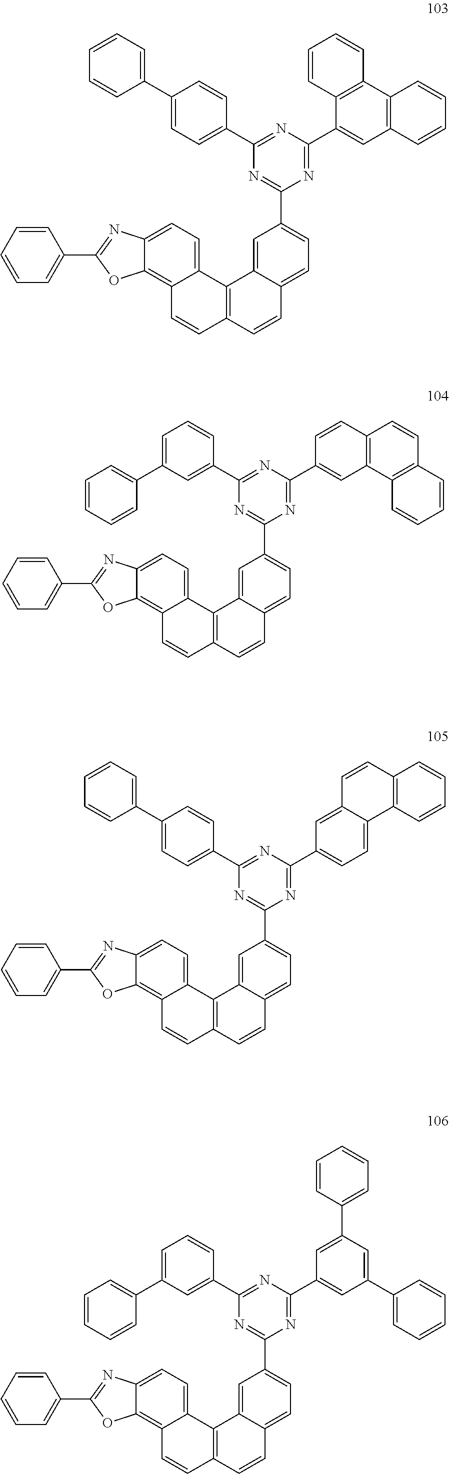 [SMILES: c1ccc(-c2ccc(-c3nc(-c4ccc5ccc6ccc7c(ccc8nc(-c9ccccc9)oc87)c6c5c4)nc(-c4cc5ccccc5c5ccccc45)n3)cc2)cc1 ; c1ccc(-c2cccc(-c3nc(-c4ccc5ccc6ccccc6c5c4)nc(-c4ccc5ccc6ccc7c(ccc8nc(-c9ccccc9)oc87)c6c5c4)n3)c2)cc1 ; c1ccc(-c2ccc(-c3nc(-c4ccc5c(ccc6ccccc65)c4)nc(-c4ccc5ccc6ccc7c(ccc8nc(-c9ccccc9)oc87)c6c5c4)n3)cc2)cc1 ; c1ccc(-c2cccc(-c3nc(-c4cc(-c5ccccc5)cc(-c5ccccc5)c4)nc(-c4ccc5ccc6ccc7c(ccc8nc(-c9ccccc9)oc87)c6c5c4)n3)c2)cc1]

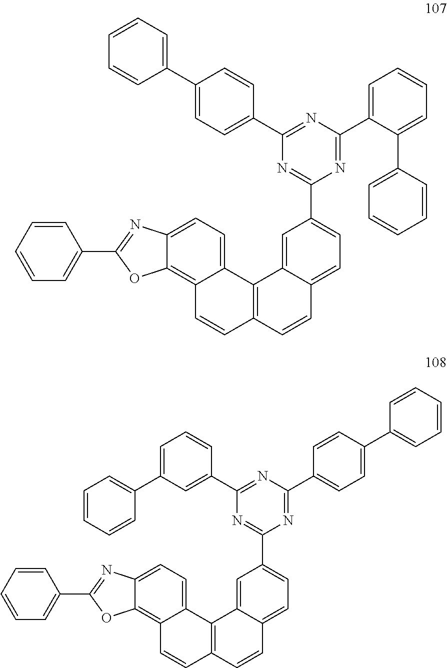 [SMILES: c1ccc(-c2ccc(-c3nc(-c4ccc5ccc6ccc7c(ccc8nc(-c9ccccc9)oc87)c6c5c4)nc(-c4ccccc4-c4ccccc4)n3)cc2)cc1 ; c1ccc(-c2ccc(-c3nc(-c4cccc(-c5ccccc5)c4)nc(-c4ccc5ccc6ccc7c(ccc8nc(-c9ccccc9)oc87)c6c5c4)n3)cc2)cc1]

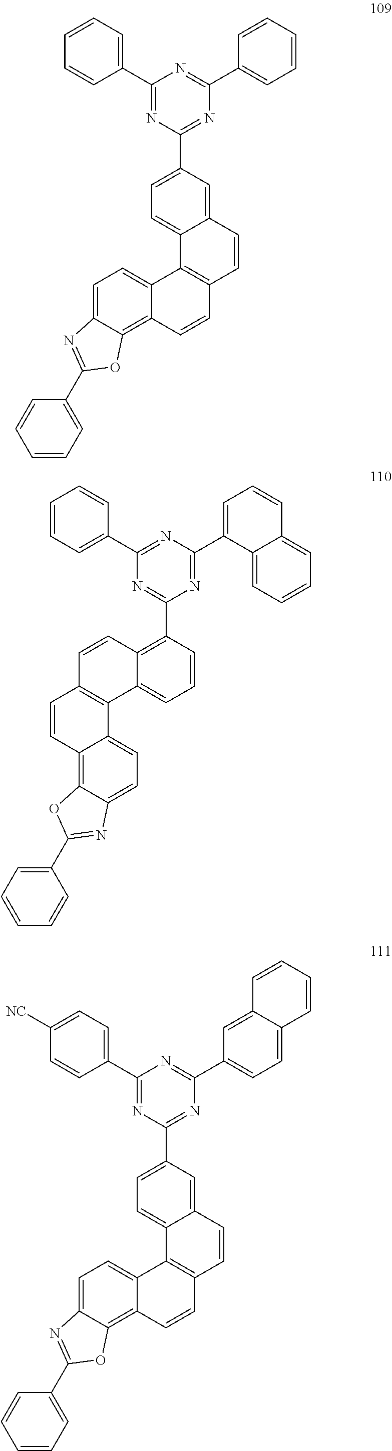

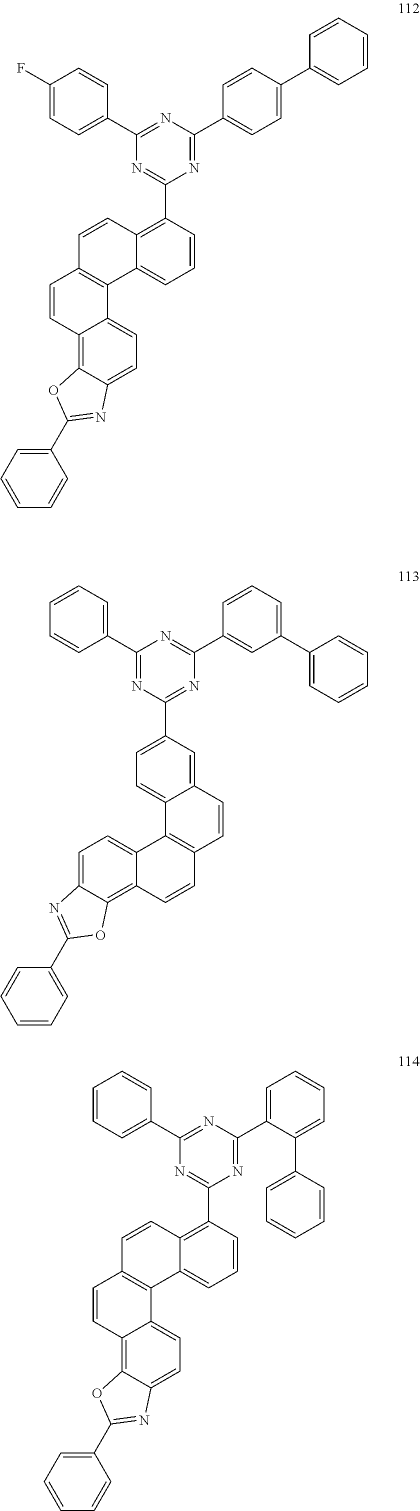 [SMILES: Fc1ccc(-c2nc(-c3ccc(-c4ccccc4)cc3)nc(-c3cccc4c3ccc3ccc5c(ccc6nc(-c7ccccc7)oc65)c34)n2)cc1 ; c1ccc(-c2cccc(-c3nc(-c4ccccc4)nc(-c4ccc5c(ccc6ccc7c(ccc8nc(-c9ccccc9)oc87)c65)c4)n3)c2)cc1 ; c1ccc(-c2nc(-c3ccccc3-c3ccccc3)nc(-c3cccc4c3ccc3ccc5c(ccc6nc(-c7ccccc7)oc65)c34)n2)cc1]

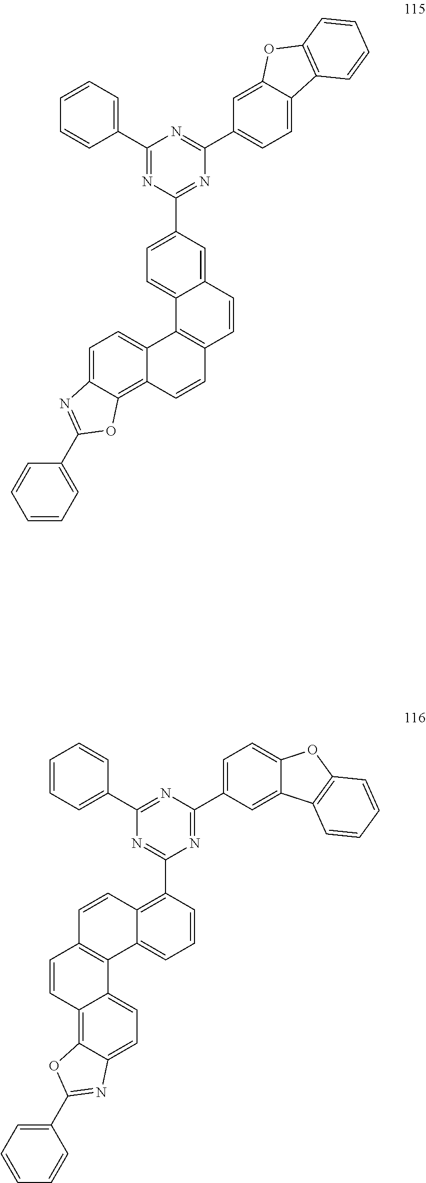 [SMILES: c1ccc(-c2nc(-c3ccc4c(ccc5ccc6c(ccc7nc(-c8ccccc8)oc76)c54)c3)nc(-c3ccc4c(c3)oc3ccccc34)n2)cc1 ; c1ccc(-c2nc(-c3ccc4oc5ccccc5c4c3)nc(-c3cccc4c3ccc3ccc5c(ccc6nc(-c7ccccc7)oc65)c34)n2)cc1]

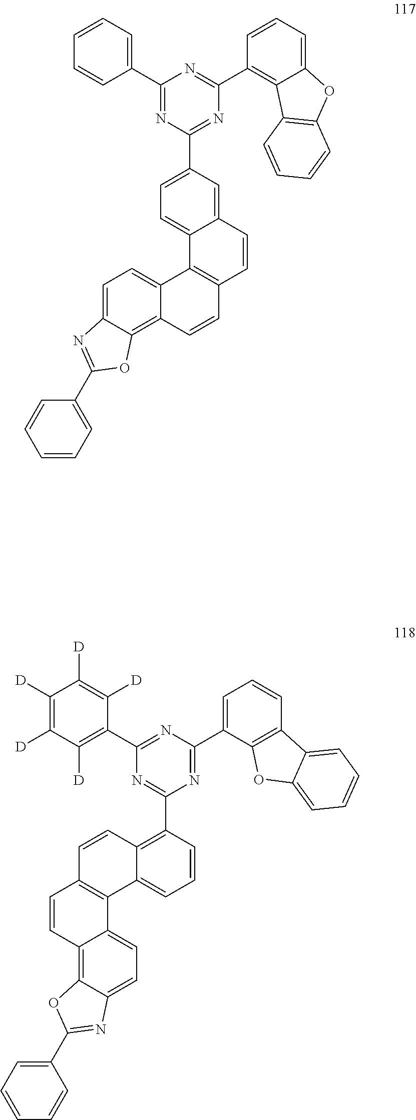 [SMILES: c1ccc(-c2nc(-c3ccc4c(ccc5ccc6c(ccc7nc(-c8ccccc8)oc76)c54)c3)nc(-c3cccc4oc5ccccc5c34)n2)cc1 ; [2H]c1c([2H])c([2H])c(-c2nc(-c3cccc4c3ccc3ccc5c(ccc6nc(-c7ccccc7)oc65)c34)nc(-c3cccc4c3oc3ccccc34)n2)c([2H])c1[2H]]

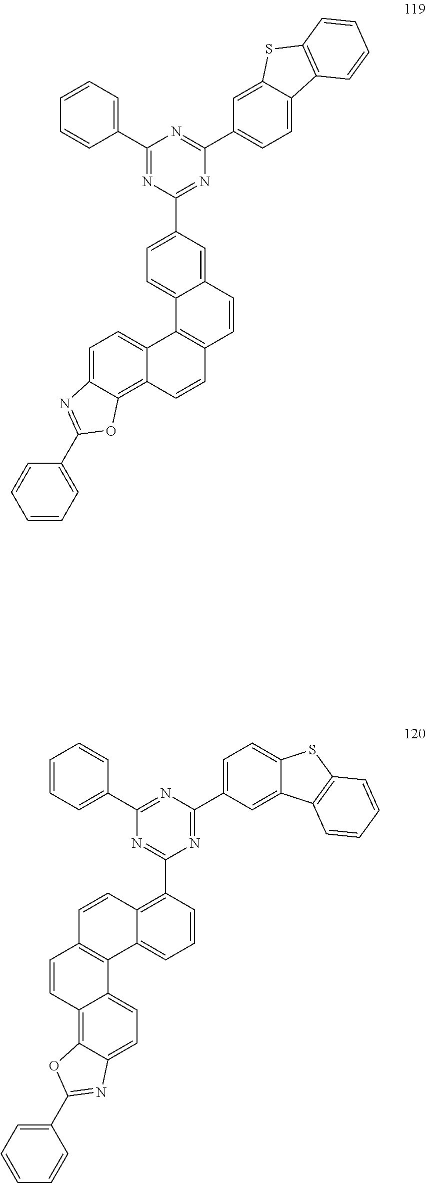 [SMILES: c1ccc(-c2nc(-c3ccc4c(ccc5ccc6c(ccc7nc(-c8ccccc8)oc76)c54)c3)nc(-c3ccc4c(c3)sc3ccccc34)n2)cc1 ; c1ccc(-c2nc(-c3ccc4sc5ccccc5c4c3)nc(-c3cccc4c3ccc3ccc5c(ccc6nc(-c7ccccc7)oc65)c34)n2)cc1]

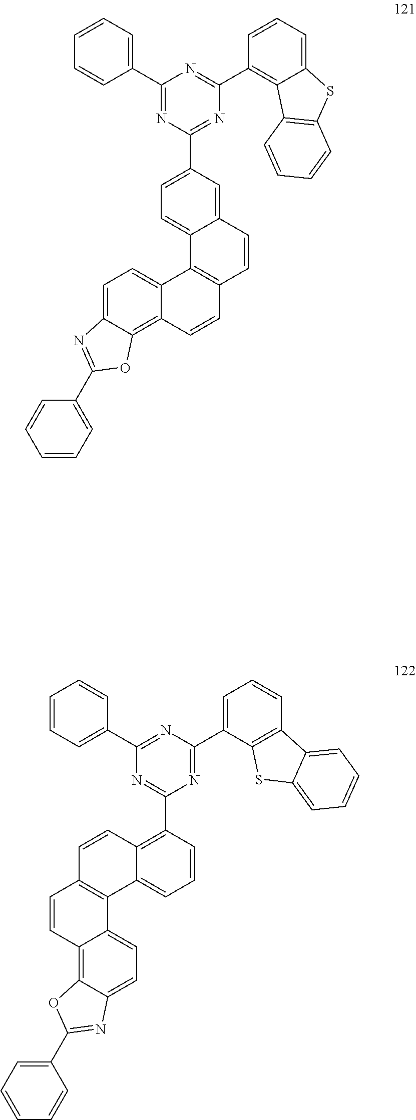 [SMILES: c1ccc(-c2nc(-c3ccc4c(ccc5ccc6c(ccc7nc(-c8ccccc8)oc76)c54)c3)nc(-c3cccc4sc5ccccc5c34)n2)cc1 ; c1ccc(-c2nc(-c3cccc4c3ccc3ccc5c(ccc6nc(-c7ccccc7)oc65)c34)nc(-c3cccc4c3sc3ccccc34)n2)cc1]

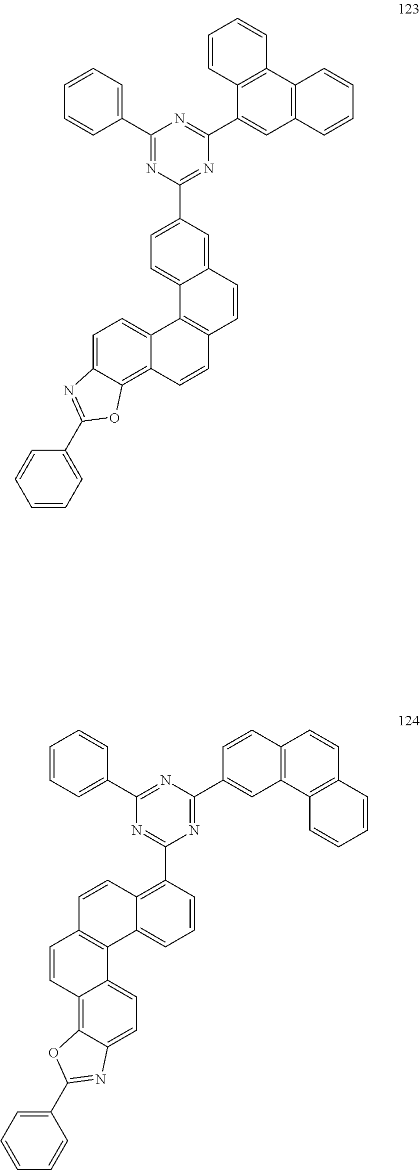 [SMILES: c1ccc(-c2nc(-c3ccc4c(ccc5ccc6c(ccc7nc(-c8ccccc8)oc76)c54)c3)nc(-c3cc4ccccc4c4ccccc34)n2)cc1 ; c1ccc(-c2nc(-c3ccc4ccc5ccccc5c4c3)nc(-c3cccc4c3ccc3ccc5c(ccc6nc(-c7ccccc7)oc65)c34)n2)cc1]

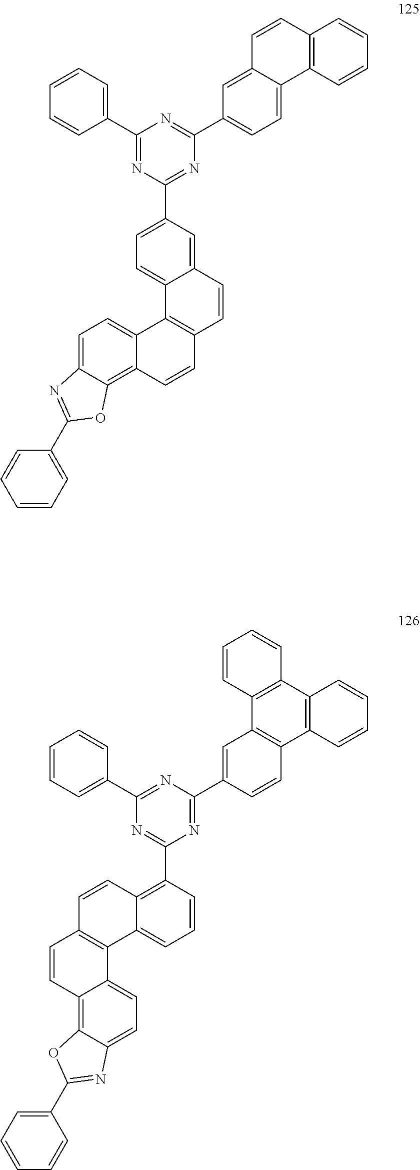 [SMILES: c1ccc(-c2nc(-c3ccc4c(ccc5ccccc54)c3)nc(-c3ccc4c(ccc5ccc6c(ccc7nc(-c8ccccc8)oc76)c54)c3)n2)cc1 ; c1ccc(-c2nc(-c3ccc4c5ccccc5c5ccccc5c4c3)nc(-c3cccc4c3ccc3ccc5c(ccc6nc(-c7ccccc7)oc65)c34)n2)cc1]

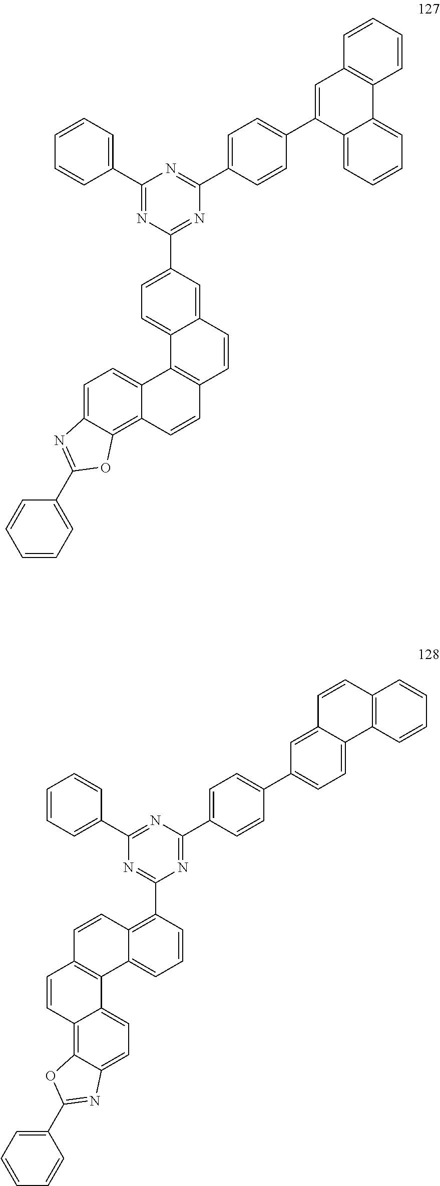 [SMILES: c1ccc(-c2nc(-c3ccc(-c4cc5ccccc5c5ccccc45)cc3)nc(-c3ccc4c(ccc5ccc6c(ccc7nc(-c8ccccc8)oc76)c54)c3)n2)cc1 ; c1ccc(-c2nc(-c3ccc(-c4ccc5c(ccc6ccccc65)c4)cc3)nc(-c3cccc4c3ccc3ccc5c(ccc6nc(-c7ccccc7)oc65)c34)n2)cc1]

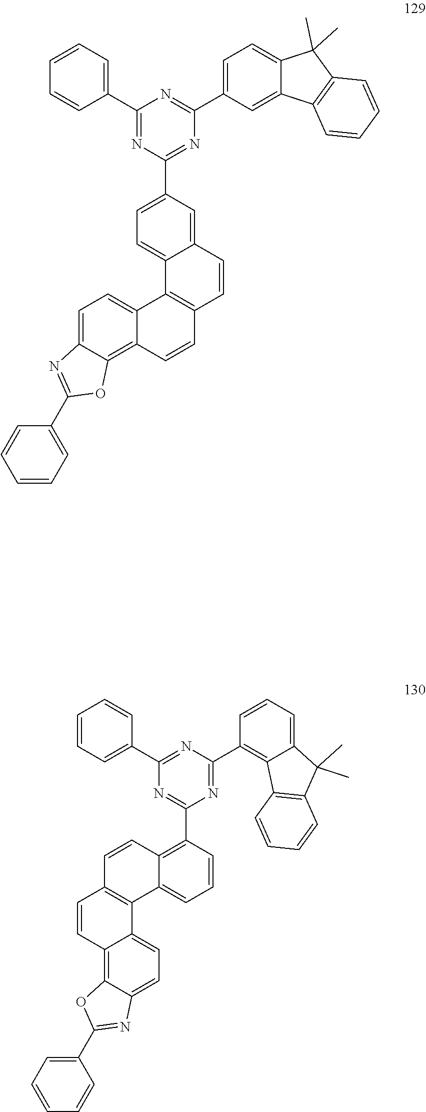 [SMILES: CC1(C)c2ccccc2-c2cc(-c3nc(-c4ccccc4)nc(-c4ccc5c(ccc6ccc7c(ccc8nc(-c9ccccc9)oc87)c65)c4)n3)ccc21 ; CC1(C)c2ccccc2-c2c(-c3nc(-c4ccccc4)nc(-c4cccc5c4ccc4ccc6c(ccc7nc(-c8ccccc8)oc76)c45)n3)cccc21]

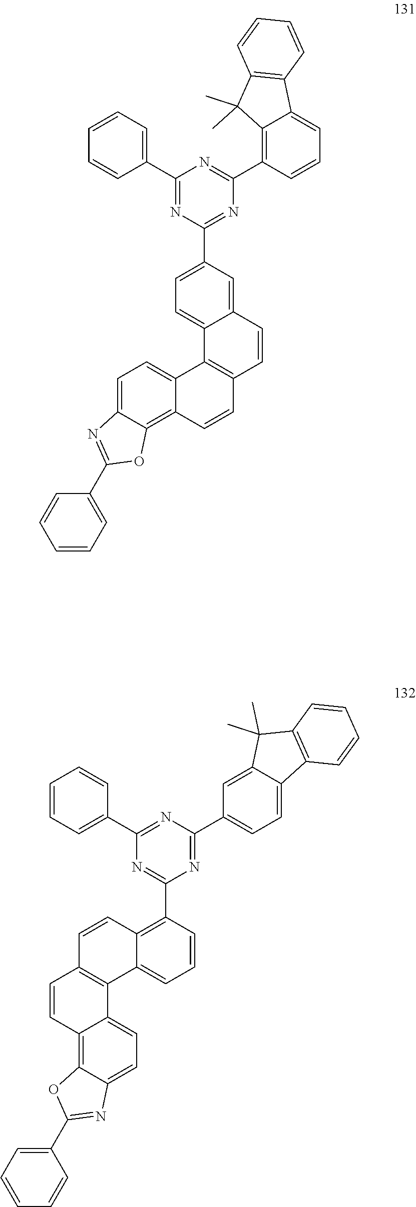 [SMILES: CC1(C)c2ccccc2-c2cccc(-c3nc(-c4ccccc4)nc(-c4ccc5c(ccc6ccc7c(ccc8nc(-c9ccccc9)oc87)c65)c4)n3)c21 ; CC1(C)c2ccccc2-c2ccc(-c3nc(-c4ccccc4)nc(-c4cccc5c4ccc4ccc6c(ccc7nc(-c8ccccc8)oc76)c45)n3)cc21]

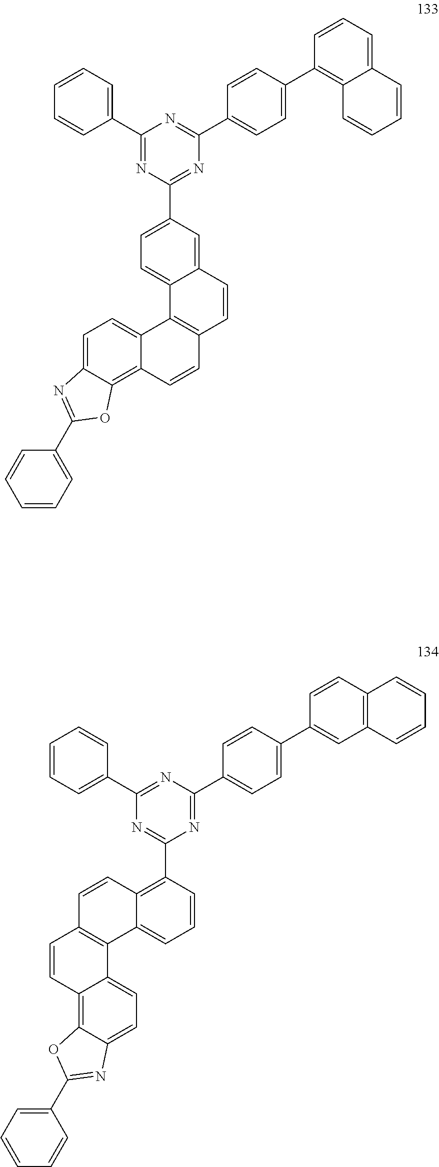 [SMILES: c1ccc(-c2nc(-c3ccc(-c4cccc5ccccc45)cc3)nc(-c3ccc4c(ccc5ccc6c(ccc7nc(-c8ccccc8)oc76)c54)c3)n2)cc1 ; c1ccc(-c2nc(-c3ccc(-c4ccc5ccccc5c4)cc3)nc(-c3cccc4c3ccc3ccc5c(ccc6nc(-c7ccccc7)oc65)c34)n2)cc1]

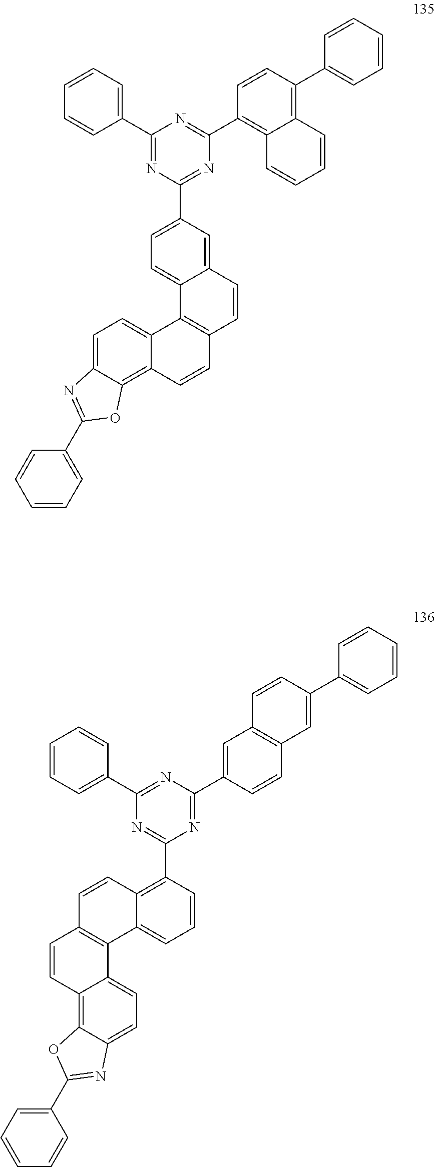 [SMILES: c1ccc(-c2nc(-c3ccc4c(ccc5ccc6c(ccc7nc(-c8ccccc8)oc76)c54)c3)nc(-c3ccc(-c4ccccc4)c4ccccc34)n2)cc1 ; c1ccc(-c2ccc3cc(-c4nc(-c5ccccc5)nc(-c5cccc6c5ccc5ccc7c(ccc8nc(-c9ccccc9)oc87)c56)n4)ccc3c2)cc1]

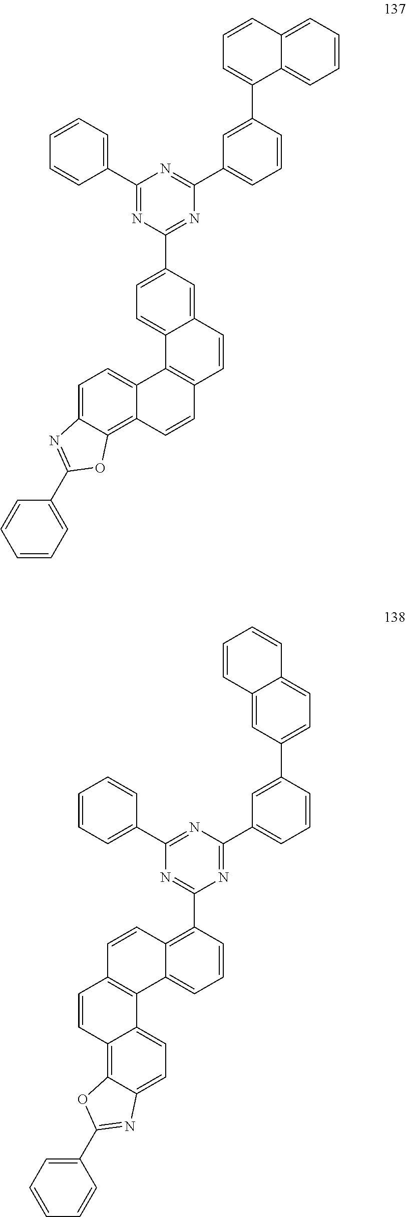 [SMILES: c1ccc(-c2nc(-c3cccc(-c4cccc5ccccc45)c3)nc(-c3ccc4c(ccc5ccc6c(ccc7nc(-c8ccccc8)oc76)c54)c3)n2)cc1 ; c1ccc(-c2nc(-c3cccc(-c4ccc5ccccc5c4)c3)nc(-c3cccc4c3ccc3ccc5c(ccc6nc(-c7ccccc7)oc65)c34)n2)cc1]

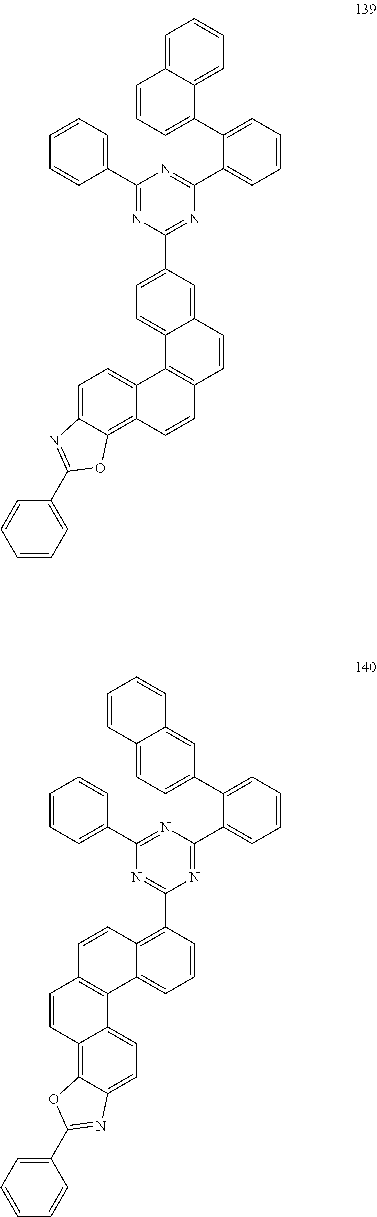 [SMILES: c1ccc(-c2nc(-c3ccc4c(ccc5ccc6c(ccc7nc(-c8ccccc8)oc76)c54)c3)nc(-c3ccccc3-c3cccc4ccccc34)n2)cc1 ; c1ccc(-c2nc(-c3ccccc3-c3ccc4ccccc4c3)nc(-c3cccc4c3ccc3ccc5c(ccc6nc(-c7ccccc7)oc65)c34)n2)cc1]

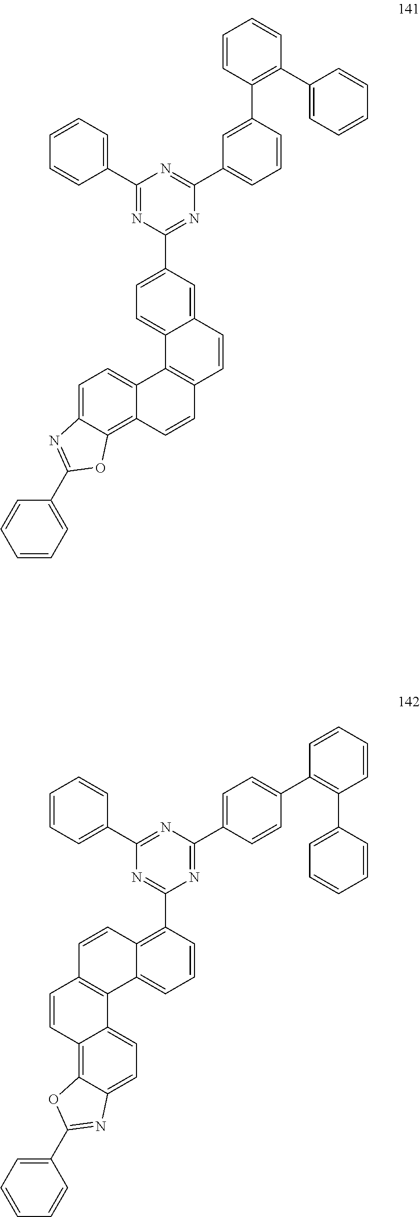 [SMILES: c1ccc(-c2nc(-c3cccc(-c4ccccc4-c4ccccc4)c3)nc(-c3ccc4c(ccc5ccc6c(ccc7nc(-c8ccccc8)oc76)c54)c3)n2)cc1 ; c1ccc(-c2nc(-c3ccc(-c4ccccc4-c4ccccc4)cc3)nc(-c3cccc4c3ccc3ccc5c(ccc6nc(-c7ccccc7)oc65)c34)n2)cc1]

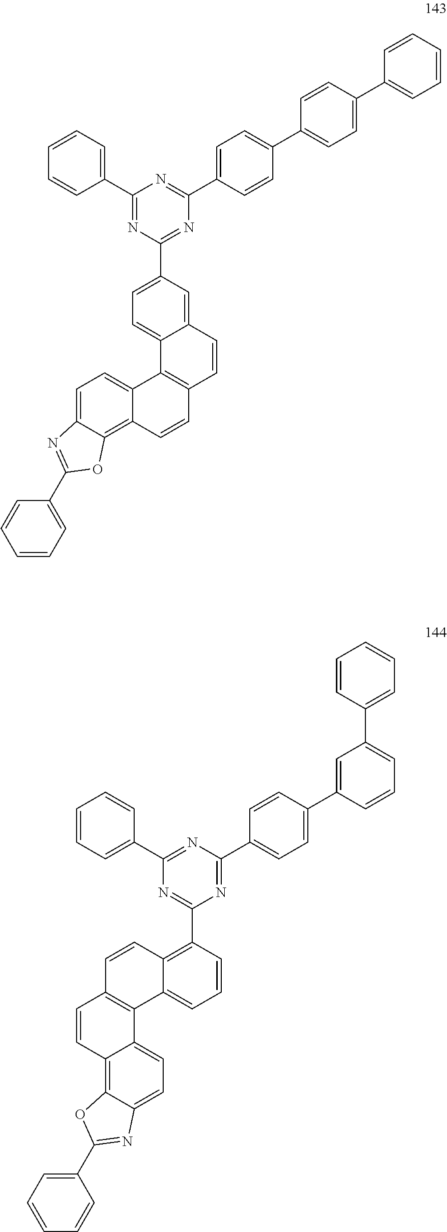 [SMILES: c1ccc(-c2ccc(-c3ccc(-c4nc(-c5ccccc5)nc(-c5ccc6c(ccc7ccc8c(ccc9nc(-c%10ccccc%10)oc98)c76)c5)n4)cc3)cc2)cc1 ; c1ccc(-c2cccc(-c3ccc(-c4nc(-c5ccccc5)nc(-c5cccc6c5ccc5ccc7c(ccc8nc(-c9ccccc9)oc87)c56)n4)cc3)c2)cc1]

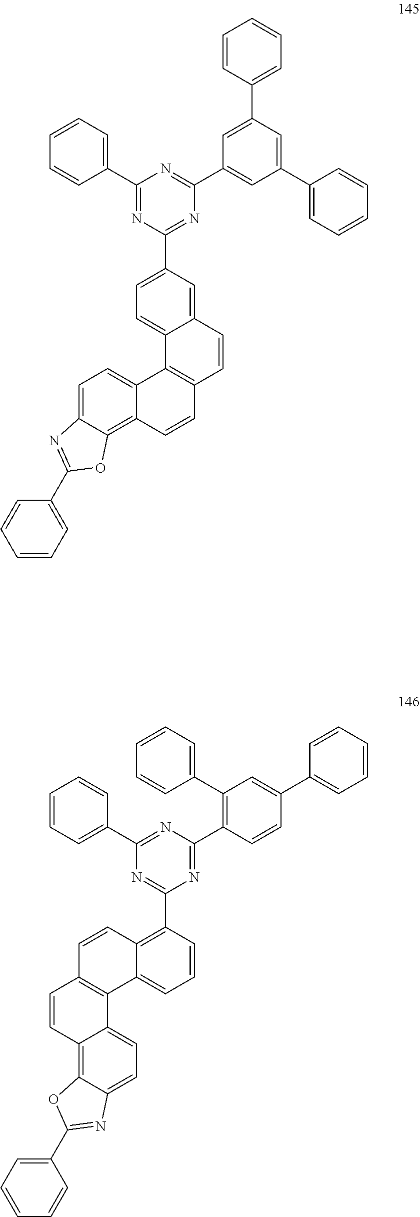 [SMILES: c1ccc(-c2cc(-c3ccccc3)cc(-c3nc(-c4ccccc4)nc(-c4ccc5c(ccc6ccc7c(ccc8nc(-c9ccccc9)oc87)c65)c4)n3)c2)cc1 ; c1ccc(-c2ccc(-c3nc(-c4ccccc4)nc(-c4cccc5c4ccc4ccc6c(ccc7nc(-c8ccccc8)oc76)c45)n3)c(-c3ccccc3)c2)cc1]

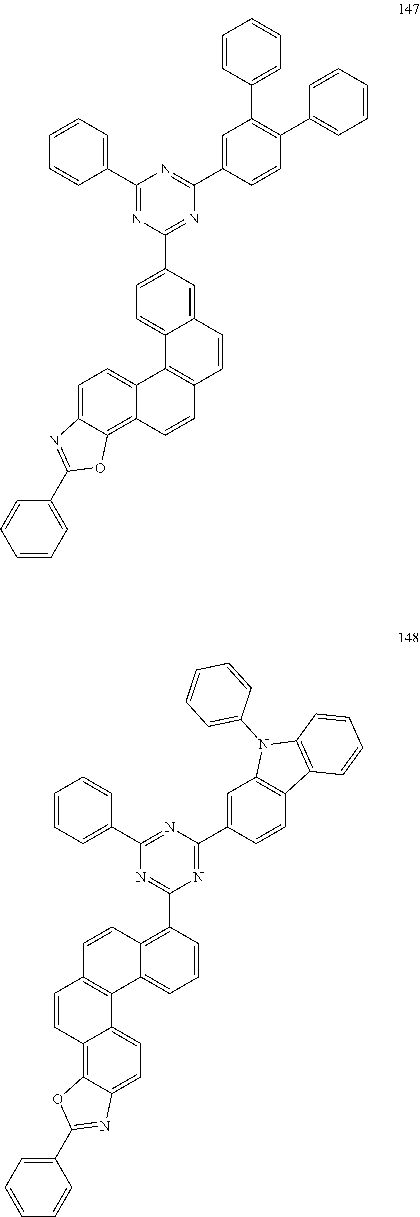 [SMILES: c1ccc(-c2nc(-c3ccc(-c4ccccc4)c(-c4ccccc4)c3)nc(-c3ccc4c(ccc5ccc6c(ccc7nc(-c8ccccc8)oc76)c54)c3)n2)cc1 ; c1ccc(-c2nc(-c3ccc4c5ccccc5n(-c5ccccc5)c4c3)nc(-c3cccc4c3ccc3ccc5c(ccc6nc(-c7ccccc7)oc65)c34)n2)cc1]

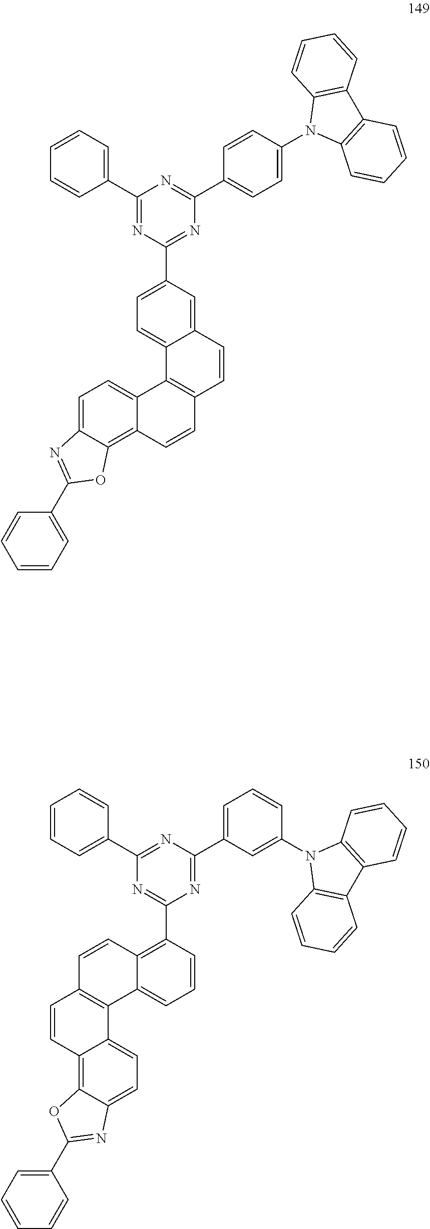 [SMILES: c1ccc(-c2nc(-c3ccc(-n4c5ccccc5c5ccccc54)cc3)nc(-c3ccc4c(ccc5ccc6c(ccc7nc(-c8ccccc8)oc76)c54)c3)n2)cc1 ; c1ccc(-c2nc(-c3cccc(-n4c5ccccc5c5ccccc54)c3)nc(-c3cccc4c3ccc3ccc5c(ccc6nc(-c7ccccc7)oc65)c34)n2)cc1]

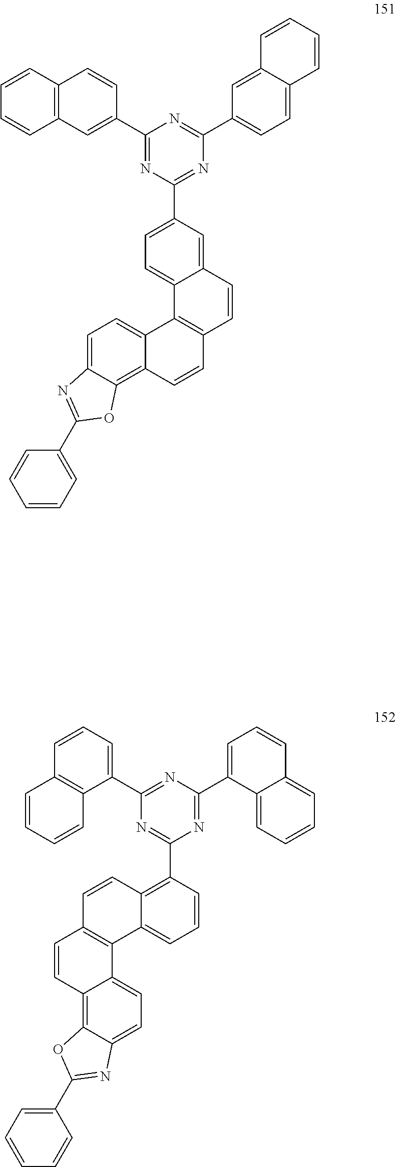 [SMILES: c1ccc(-c2nc3ccc4c(ccc5ccc6cc(-c7nc(-c8ccc9ccccc9c8)nc(-c8ccc9ccccc9c8)n7)ccc6c54)c3o2)cc1 ; c1ccc(-c2nc3ccc4c(ccc5ccc6c(-c7nc(-c8cccc9ccccc89)nc(-c8cccc9ccccc89)n7)cccc6c54)c3o2)cc1]

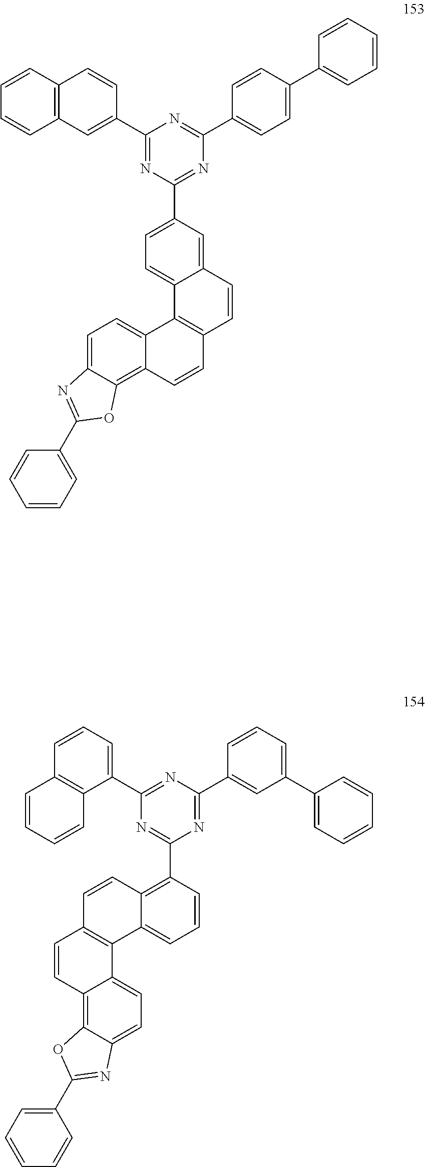 [SMILES: c1ccc(-c2ccc(-c3nc(-c4ccc5ccccc5c4)nc(-c4ccc5c(ccc6ccc7c(ccc8nc(-c9ccccc9)oc87)c65)c4)n3)cc2)cc1 ; c1ccc(-c2cccc(-c3nc(-c4cccc5ccccc45)nc(-c4cccc5c4ccc4ccc6c(ccc7nc(-c8ccccc8)oc76)c45)n3)c2)cc1]

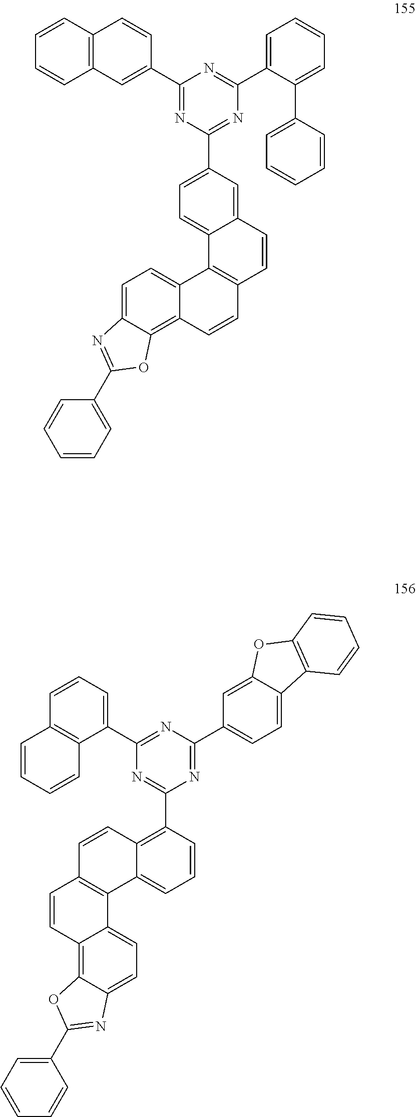 [SMILES: c1ccc(-c2nc3ccc4c(ccc5ccc6cc(-c7nc(-c8ccc9ccccc9c8)nc(-c8ccccc8-c8ccccc8)n7)ccc6c54)c3o2)cc1 ; c1ccc(-c2nc3ccc4c(ccc5ccc6c(-c7nc(-c8ccc9c(c8)oc8ccccc89)nc(-c8cccc9ccccc89)n7)cccc6c54)c3o2)cc1]

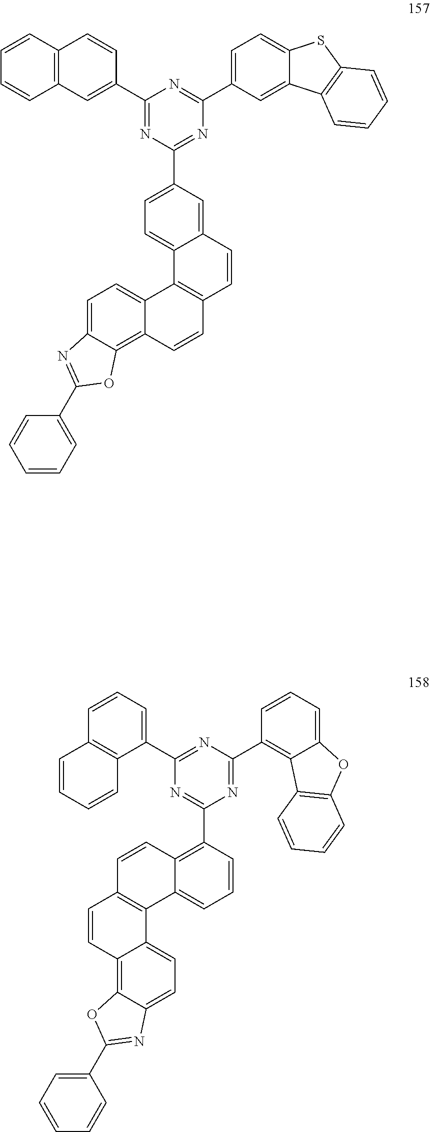 [SMILES: c1ccc(-c2nc3ccc4c(ccc5ccc6cc(-c7nc(-c8ccc9ccccc9c8)nc(-c8ccc9sc%10ccccc%10c9c8)n7)ccc6c54)c3o2)cc1 ; c1ccc(-c2nc3ccc4c(ccc5ccc6c(-c7nc(-c8cccc9ccccc89)nc(-c8cccc9oc%10ccccc%10c89)n7)cccc6c54)c3o2)cc1]

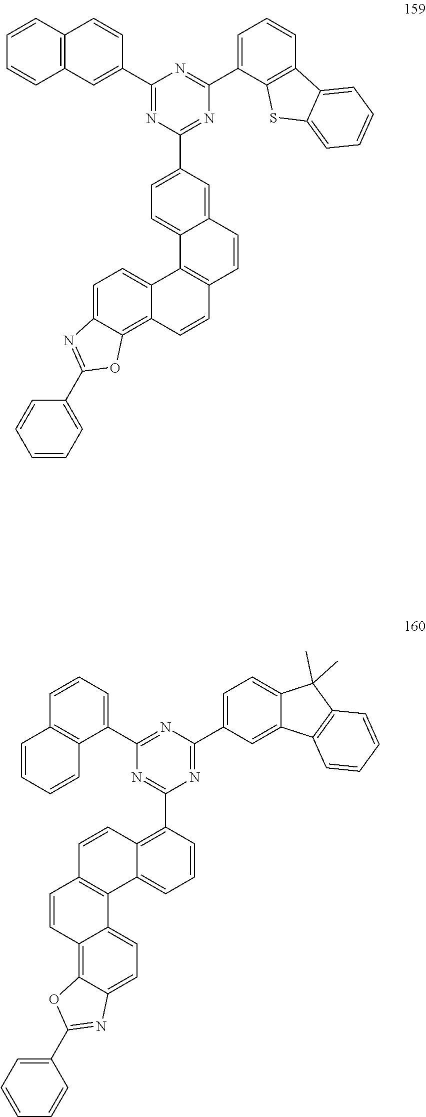 [SMILES: c1ccc(-c2nc3ccc4c(ccc5ccc6cc(-c7nc(-c8ccc9ccccc9c8)nc(-c8cccc9c8sc8ccccc89)n7)ccc6c54)c3o2)cc1 ; CC1(C)c2ccccc2-c2cc(-c3nc(-c4cccc5ccccc45)nc(-c4cccc5c4ccc4ccc6c(ccc7nc(-c8ccccc8)oc76)c45)n3)ccc21]

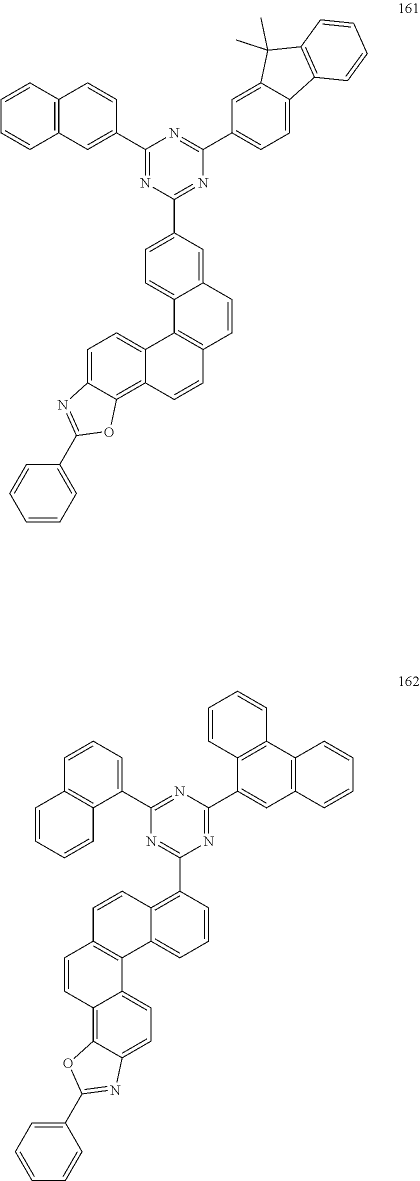 [SMILES: CC1(C)c2ccccc2-c2ccc(-c3nc(-c4ccc5ccccc5c4)nc(-c4ccc5c(ccc6ccc7c(ccc8nc(-c9ccccc9)oc87)c65)c4)n3)cc21 ; c1ccc(-c2nc3ccc4c(ccc5ccc6c(-c7nc(-c8cccc9ccccc89)nc(-c8cc9ccccc9c9ccccc89)n7)cccc6c54)c3o2)cc1]

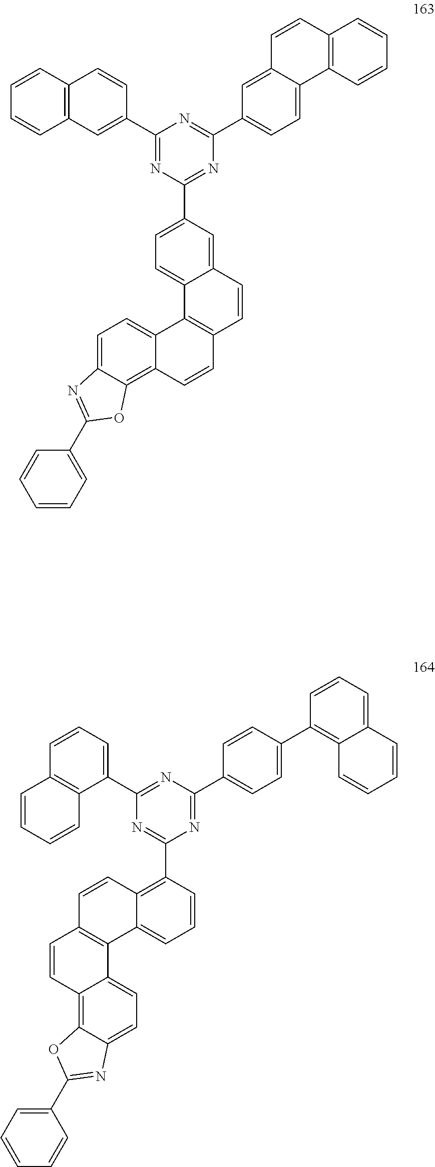 [SMILES: c1ccc(-c2nc3ccc4c(ccc5ccc6cc(-c7nc(-c8ccc9ccccc9c8)nc(-c8ccc9c(ccc%10ccccc%109)c8)n7)ccc6c54)c3o2)cc1 ; c1ccc(-c2nc3ccc4c(ccc5ccc6c(-c7nc(-c8ccc(-c9cccc%10ccccc9%10)cc8)nc(-c8cccc9ccccc89)n7)cccc6c54)c3o2)cc1]

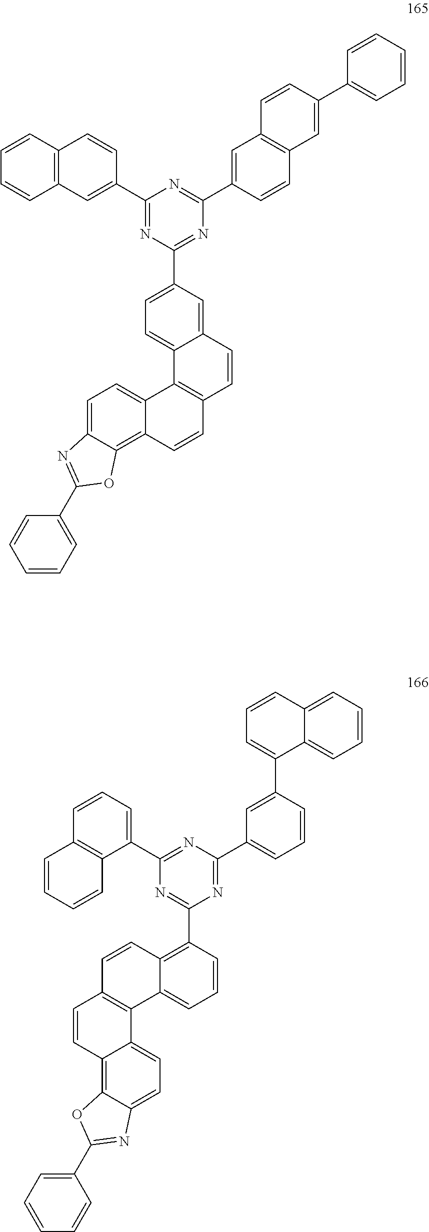 [SMILES: c1ccc(-c2ccc3cc(-c4nc(-c5ccc6ccccc6c5)nc(-c5ccc6c(ccc7ccc8c(ccc9nc(-c%10ccccc%10)oc98)c76)c5)n4)ccc3c2)cc1 ; c1ccc(-c2nc3ccc4c(ccc5ccc6c(-c7nc(-c8cccc(-c9cccc%10ccccc9%10)c8)nc(-c8cccc9ccccc89)n7)cccc6c54)c3o2)cc1]

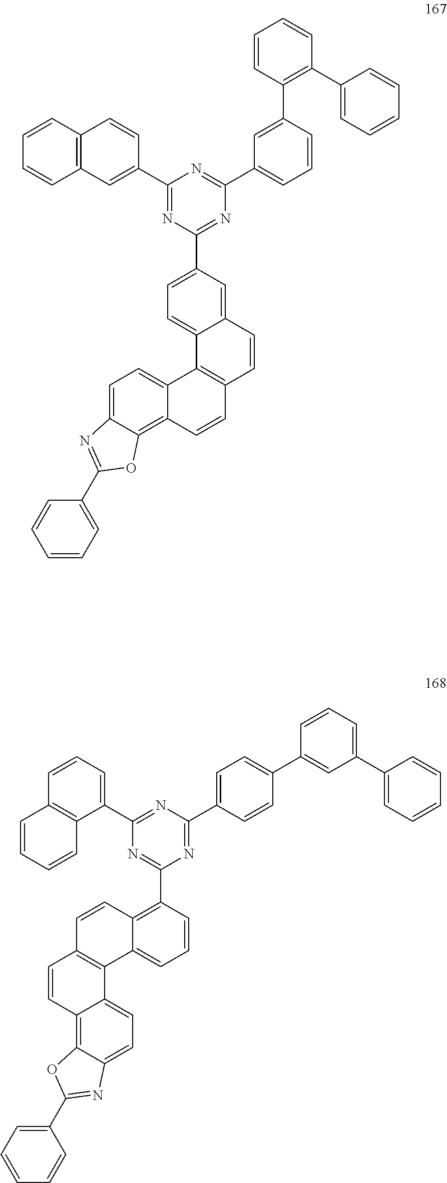 [SMILES: c1ccc(-c2nc3ccc4c(ccc5ccc6cc(-c7nc(-c8cccc(-c9ccccc9-c9ccccc9)c8)nc(-c8ccc9ccccc9c8)n7)ccc6c54)c3o2)cc1 ; c1ccc(-c2cccc(-c3ccc(-c4nc(-c5cccc6ccccc56)nc(-c5cccc6c5ccc5ccc7c(ccc8nc(-c9ccccc9)oc87)c56)n4)cc3)c2)cc1]

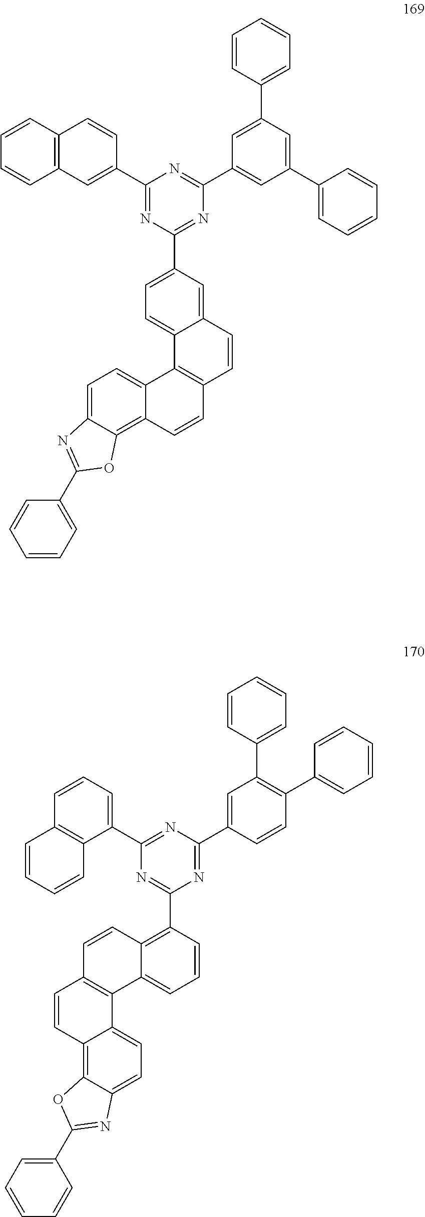 [SMILES: c1ccc(-c2cc(-c3ccccc3)cc(-c3nc(-c4ccc5ccccc5c4)nc(-c4ccc5c(ccc6ccc7c(ccc8nc(-c9ccccc9)oc87)c65)c4)n3)c2)cc1 ; c1ccc(-c2nc3ccc4c(ccc5ccc6c(-c7nc(-c8ccc(-c9ccccc9)c(-c9ccccc9)c8)nc(-c8cccc9ccccc89)n7)cccc6c54)c3o2)cc1]

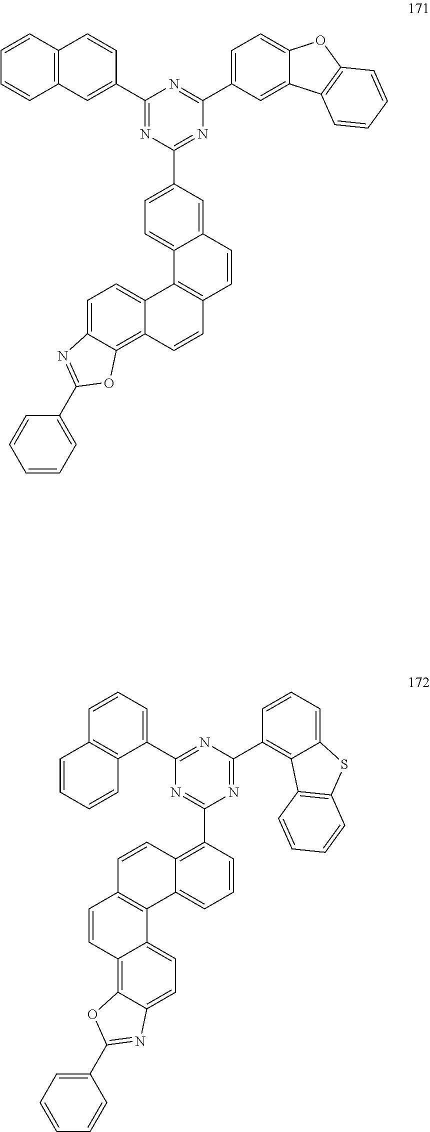 [SMILES: c1ccc(-c2nc3ccc4c(ccc5ccc6cc(-c7nc(-c8ccc9ccccc9c8)nc(-c8ccc9oc%10ccccc%10c9c8)n7)ccc6c54)c3o2)cc1 ; c1ccc(-c2nc3ccc4c(ccc5ccc6c(-c7nc(-c8cccc9ccccc89)nc(-c8cccc9sc%10ccccc%10c89)n7)cccc6c54)c3o2)cc1]

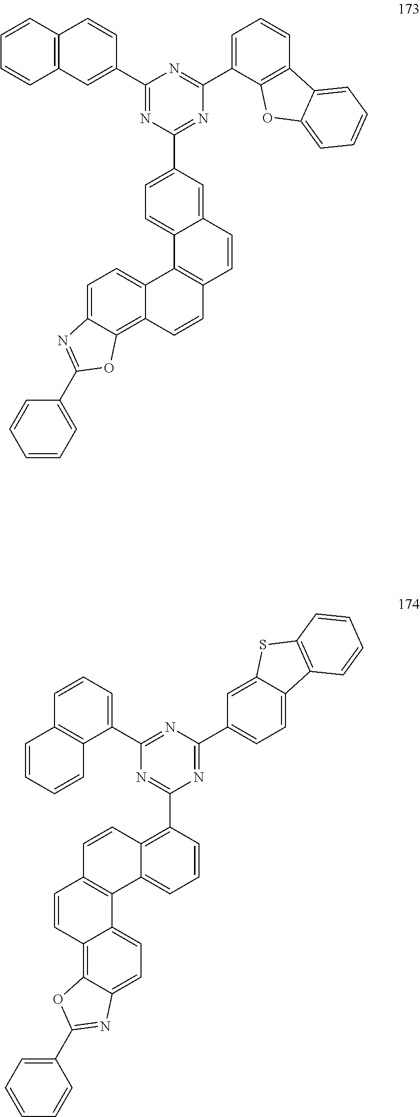 [SMILES: c1ccc(-c2nc3ccc4c(ccc5ccc6cc(-c7nc(-c8ccc9ccccc9c8)nc(-c8cccc9c8oc8ccccc89)n7)ccc6c54)c3o2)cc1 ; c1ccc(-c2nc3ccc4c(ccc5ccc6c(-c7nc(-c8ccc9c(c8)sc8ccccc89)nc(-c8cccc9ccccc89)n7)cccc6c54)c3o2)cc1]

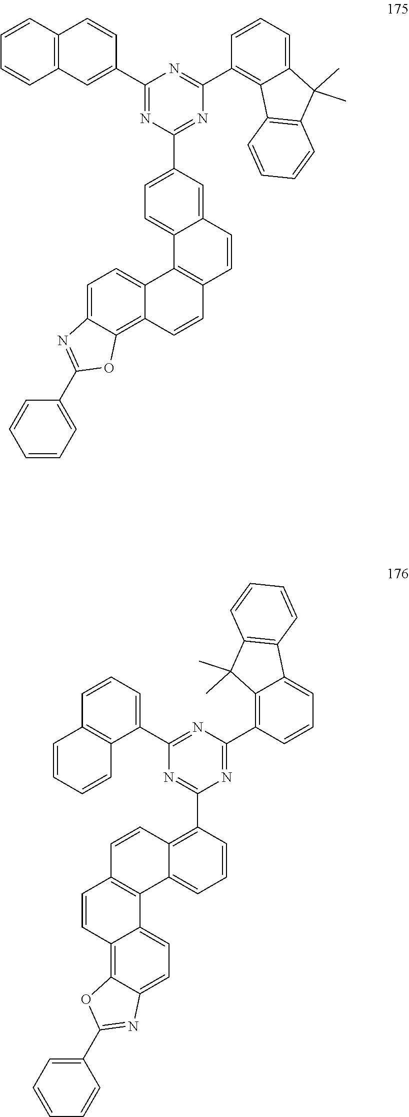 [SMILES: CC1(C)c2ccccc2-c2c(-c3nc(-c4ccc5ccccc5c4)nc(-c4ccc5c(ccc6ccc7c(ccc8nc(-c9ccccc9)oc87)c65)c4)n3)cccc21 ; CC1(C)c2ccccc2-c2cccc(-c3nc(-c4cccc5ccccc45)nc(-c4cccc5c4ccc4ccc6c(ccc7nc(-c8ccccc8)oc76)c45)n3)c21]

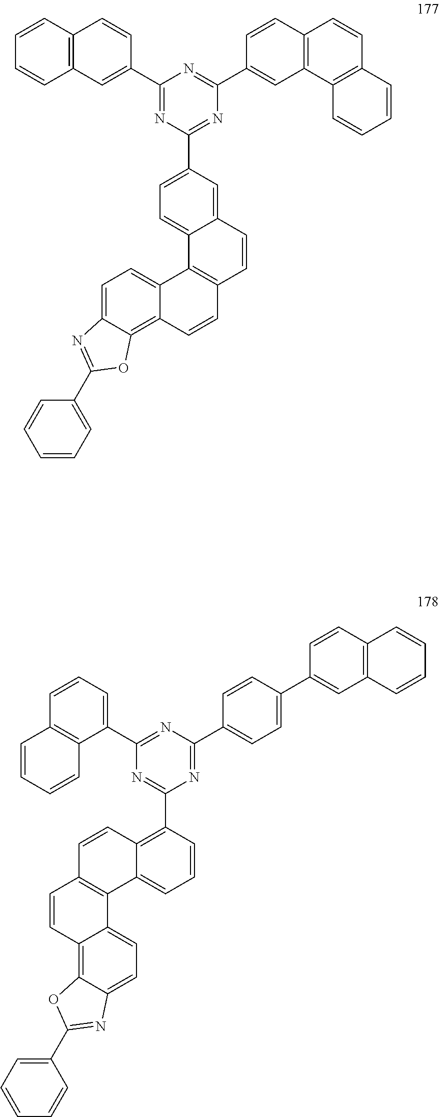 [SMILES: c1ccc(-c2nc3ccc4c(ccc5ccc6cc(-c7nc(-c8ccc9ccccc9c8)nc(-c8ccc9ccc%10ccccc%10c9c8)n7)ccc6c54)c3o2)cc1 ; c1ccc(-c2nc3ccc4c(ccc5ccc6c(-c7nc(-c8ccc(-c9ccc%10ccccc%10c9)cc8)nc(-c8cccc9ccccc89)n7)cccc6c54)c3o2)cc1]

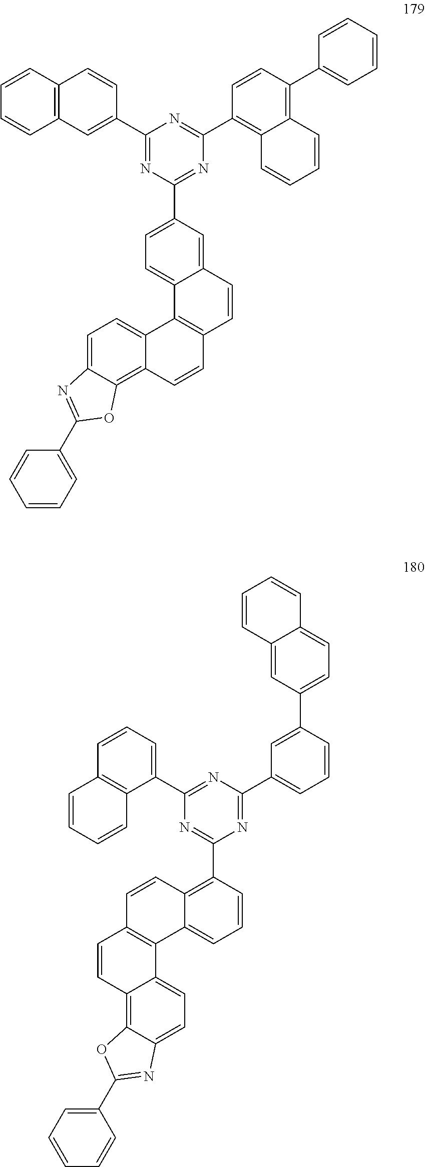 [SMILES: c1ccc(-c2nc3ccc4c(ccc5ccc6cc(-c7nc(-c8ccc9ccccc9c8)nc(-c8ccc(-c9ccccc9)c9ccccc89)n7)ccc6c54)c3o2)cc1 ; c1ccc(-c2nc3ccc4c(ccc5ccc6c(-c7nc(-c8cccc(-c9ccc%10ccccc%10c9)c8)nc(-c8cccc9ccccc89)n7)cccc6c54)c3o2)cc1]

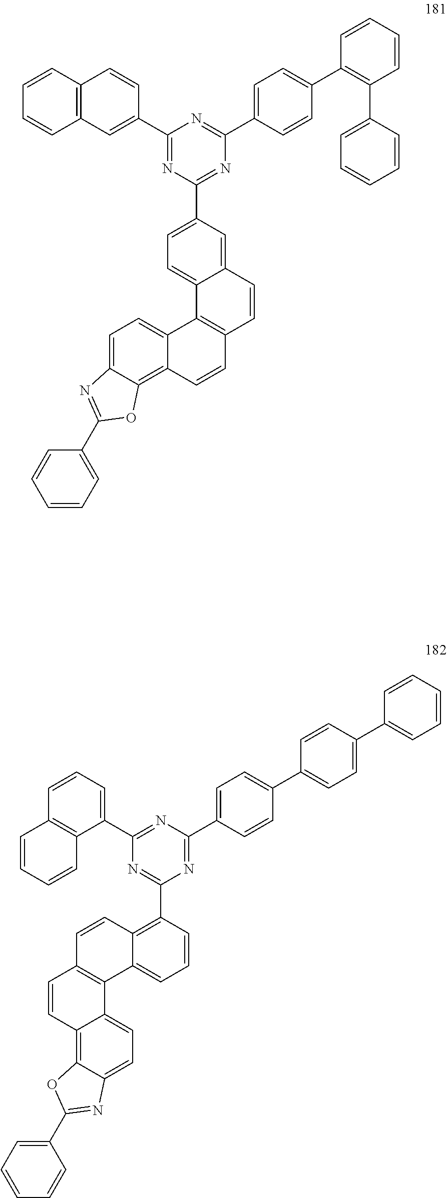 [SMILES: c1ccc(-c2nc3ccc4c(ccc5ccc6cc(-c7nc(-c8ccc(-c9ccccc9-c9ccccc9)cc8)nc(-c8ccc9ccccc9c8)n7)ccc6c54)c3o2)cc1 ; c1ccc(-c2ccc(-c3ccc(-c4nc(-c5cccc6ccccc56)nc(-c5cccc6c5ccc5ccc7c(ccc8nc(-c9ccccc9)oc87)c56)n4)cc3)cc2)cc1]

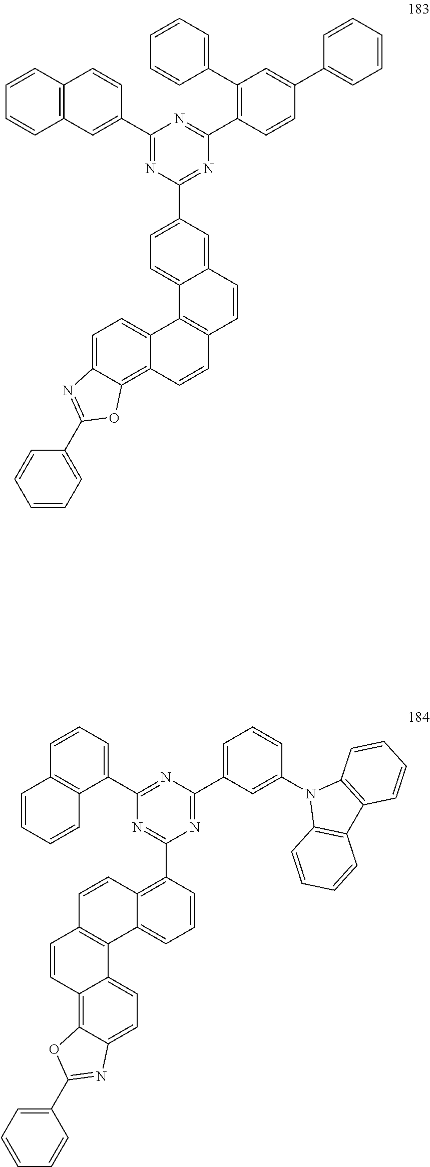 [SMILES: c1ccc(-c2ccc(-c3nc(-c4ccc5ccccc5c4)nc(-c4ccc5c(ccc6ccc7c(ccc8nc(-c9ccccc9)oc87)c65)c4)n3)c(-c3ccccc3)c2)cc1 ; c1ccc(-c2nc3ccc4c(ccc5ccc6c(-c7nc(-c8cccc(-n9c%10ccccc%10c%10ccccc%109)c8)nc(-c8cccc9ccccc89)n7)cccc6c54)c3o2)cc1]

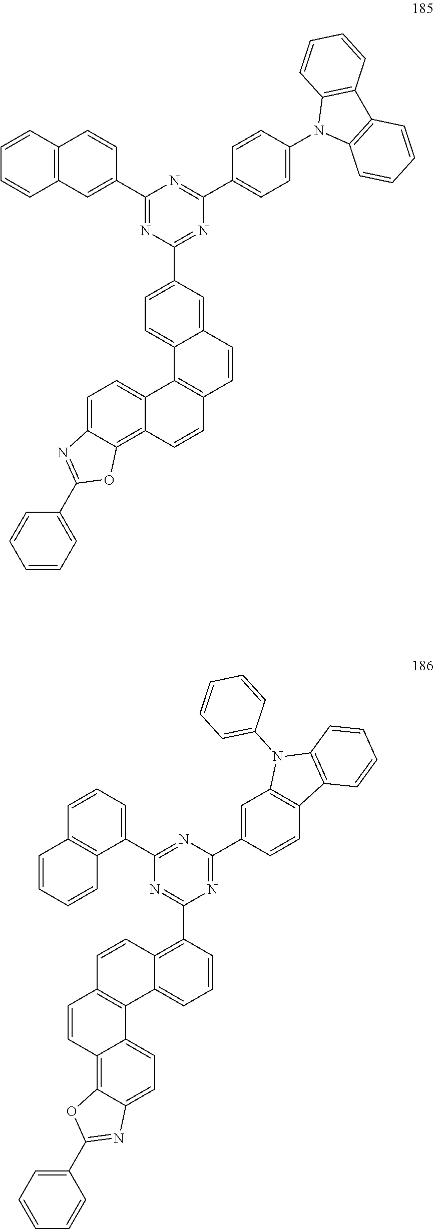 [SMILES: c1ccc(-c2nc3ccc4c(ccc5ccc6cc(-c7nc(-c8ccc(-n9c%10ccccc%10c%10ccccc%109)cc8)nc(-c8ccc9ccccc9c8)n7)ccc6c54)c3o2)cc1 ; c1ccc(-c2nc3ccc4c(ccc5ccc6c(-c7nc(-c8ccc9c%10ccccc%10n(-c%10ccccc%10)c9c8)nc(-c8cccc9ccccc89)n7)cccc6c54)c3o2)cc1]

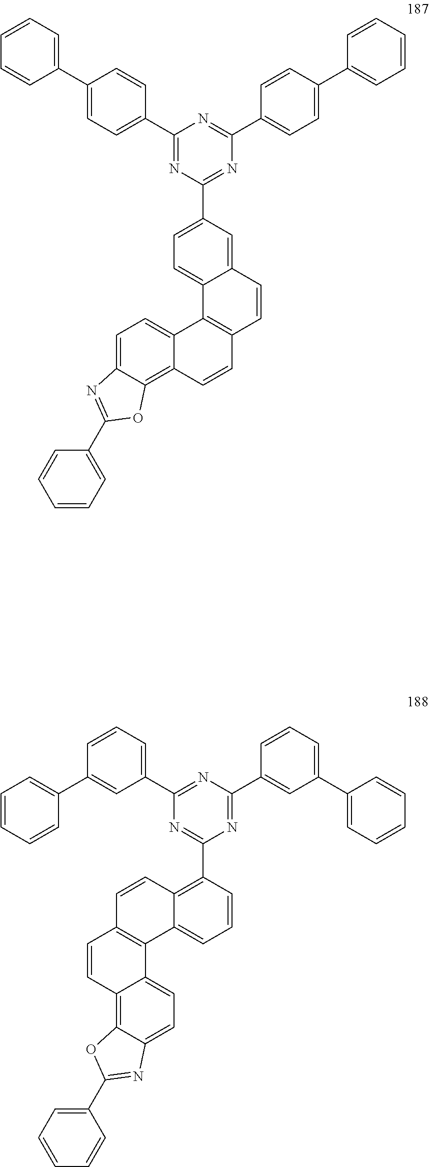 [SMILES: c1ccc(-c2ccc(-c3nc(-c4ccc(-c5ccccc5)cc4)nc(-c4ccc5c(ccc6ccc7c(ccc8nc(-c9ccccc9)oc87)c65)c4)n3)cc2)cc1 ; c1ccc(-c2cccc(-c3nc(-c4cccc(-c5ccccc5)c4)nc(-c4cccc5c4ccc4ccc6c(ccc7nc(-c8ccccc8)oc76)c45)n3)c2)cc1]

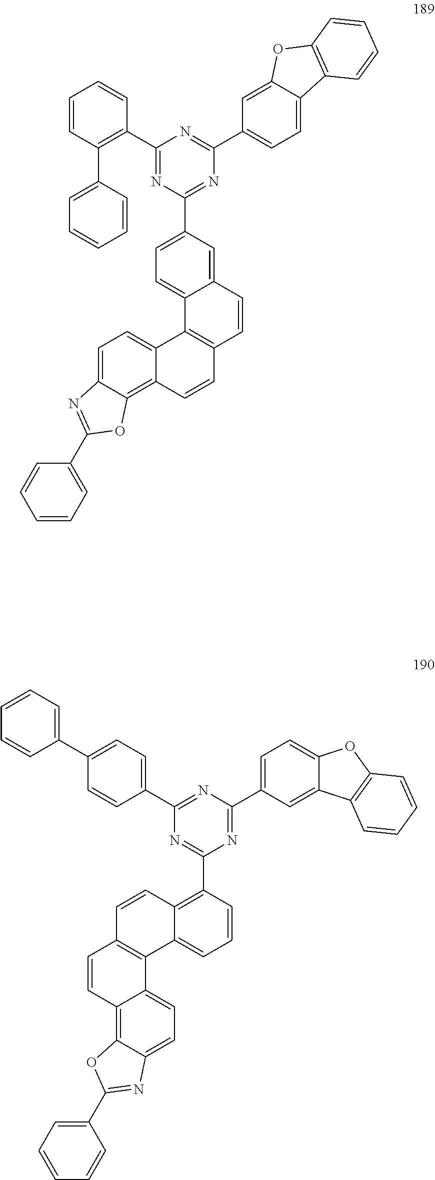 [SMILES: c1ccc(-c2nc3ccc4c(ccc5ccc6cc(-c7nc(-c8ccc9c(c8)oc8ccccc89)nc(-c8ccccc8-c8ccccc8)n7)ccc6c54)c3o2)cc1 ; c1ccc(-c2ccc(-c3nc(-c4ccc5oc6ccccc6c5c4)nc(-c4cccc5c4ccc4ccc6c(ccc7nc(-c8ccccc8)oc76)c45)n3)cc2)cc1]

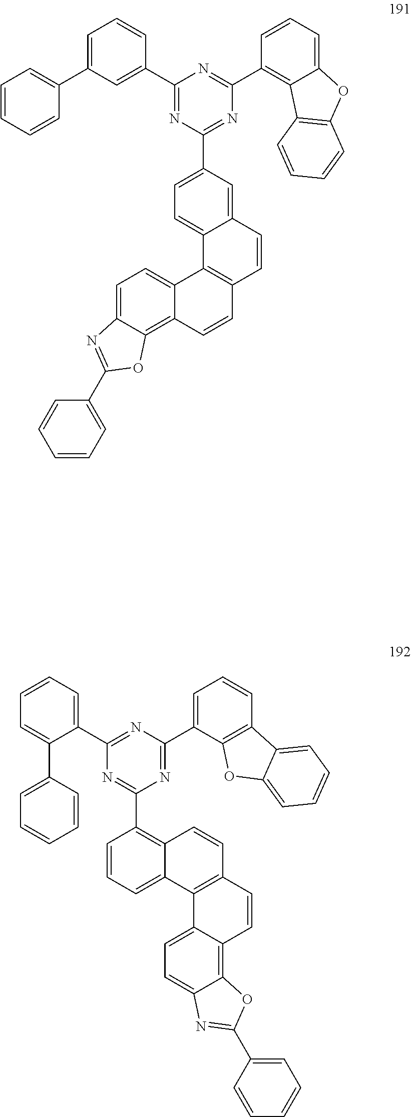 [SMILES: c1ccc(-c2cccc(-c3nc(-c4ccc5c(ccc6ccc7c(ccc8nc(-c9ccccc9)oc87)c65)c4)nc(-c4cccc5oc6ccccc6c45)n3)c2)cc1 ; c1ccc(-c2nc3ccc4c(ccc5ccc6c(-c7nc(-c8ccccc8-c8ccccc8)nc(-c8cccc9c8oc8ccccc89)n7)cccc6c54)c3o2)cc1]

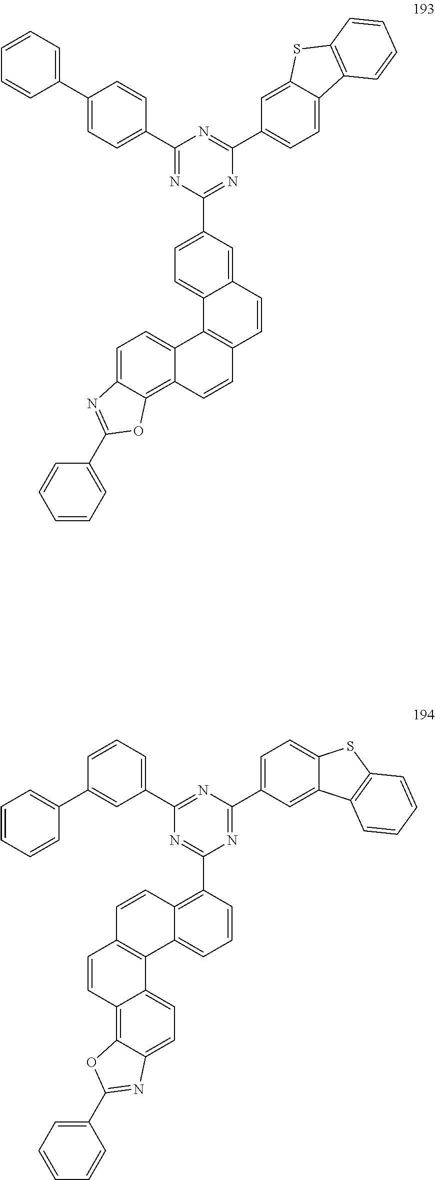 [SMILES: c1ccc(-c2ccc(-c3nc(-c4ccc5c(ccc6ccc7c(ccc8nc(-c9ccccc9)oc87)c65)c4)nc(-c4ccc5c(c4)sc4ccccc45)n3)cc2)cc1 ; c1ccc(-c2cccc(-c3nc(-c4ccc5sc6ccccc6c5c4)nc(-c4cccc5c4ccc4ccc6c(ccc7nc(-c8ccccc8)oc76)c45)n3)c2)cc1]

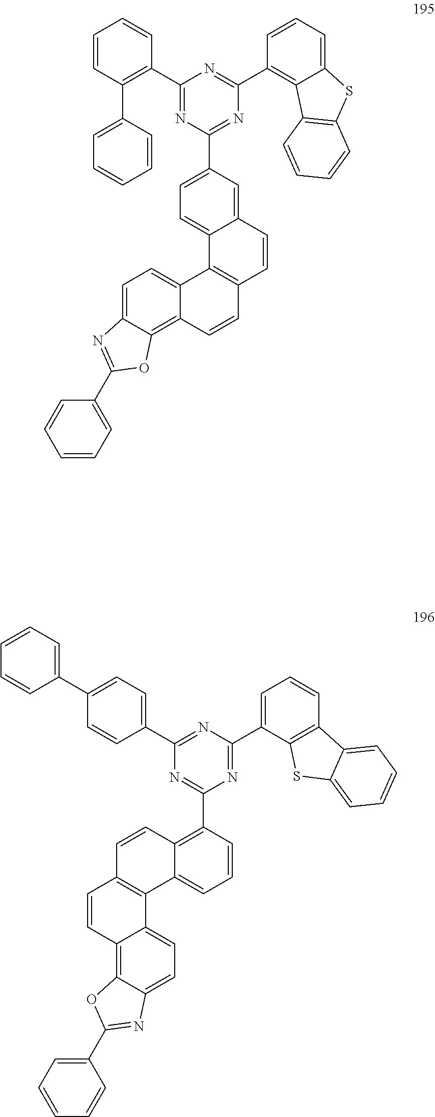 [SMILES: c1ccc(-c2nc3ccc4c(ccc5ccc6cc(-c7nc(-c8ccccc8-c8ccccc8)nc(-c8cccc9sc%10ccccc%10c89)n7)ccc6c54)c3o2)cc1 ; c1ccc(-c2ccc(-c3nc(-c4cccc5c4ccc4ccc6c(ccc7nc(-c8ccccc8)oc76)c45)nc(-c4cccc5c4sc4ccccc45)n3)cc2)cc1]

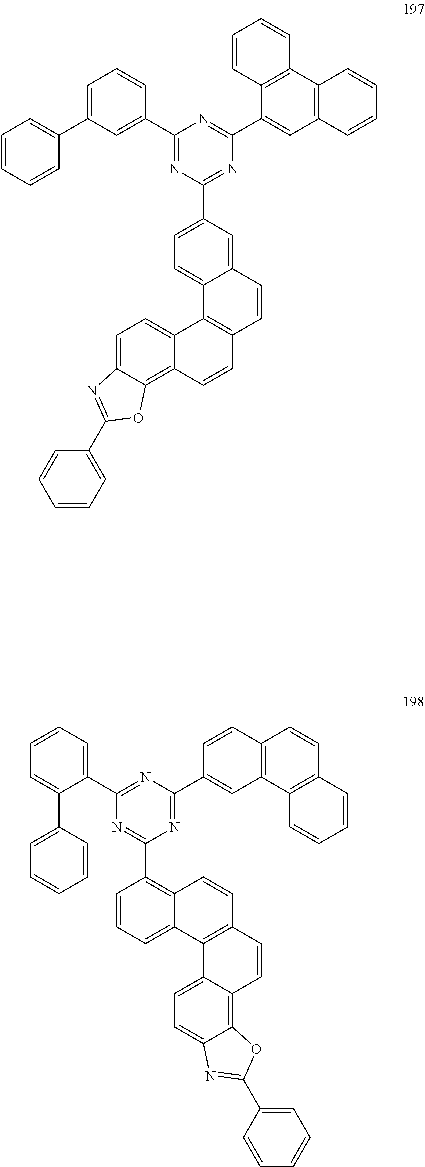 [SMILES: c1ccc(-c2cccc(-c3nc(-c4ccc5c(ccc6ccc7c(ccc8nc(-c9ccccc9)oc87)c65)c4)nc(-c4cc5ccccc5c5ccccc45)n3)c2)cc1 ; c1ccc(-c2nc3ccc4c(ccc5ccc6c(-c7nc(-c8ccc9ccc%10ccccc%10c9c8)nc(-c8ccccc8-c8ccccc8)n7)cccc6c54)c3o2)cc1]

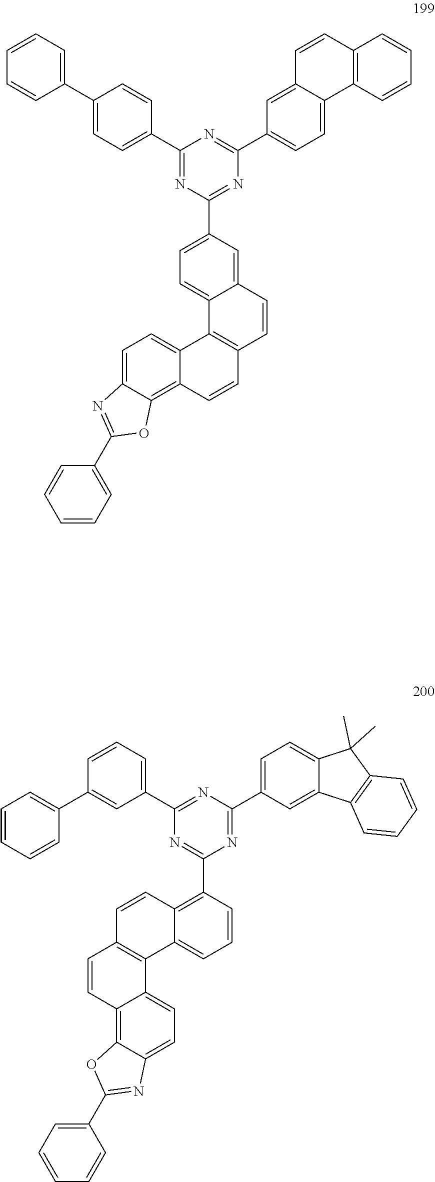 [SMILES: c1ccc(-c2ccc(-c3nc(-c4ccc5c(ccc6ccccc65)c4)nc(-c4ccc5c(ccc6ccc7c(ccc8nc(-c9ccccc9)oc87)c65)c4)n3)cc2)cc1 ; CC1(C)c2ccccc2-c2cc(-c3nc(-c4cccc(-c5ccccc5)c4)nc(-c4cccc5c4ccc4ccc6c(ccc7nc(-c8ccccc8)oc76)c45)n3)ccc21]

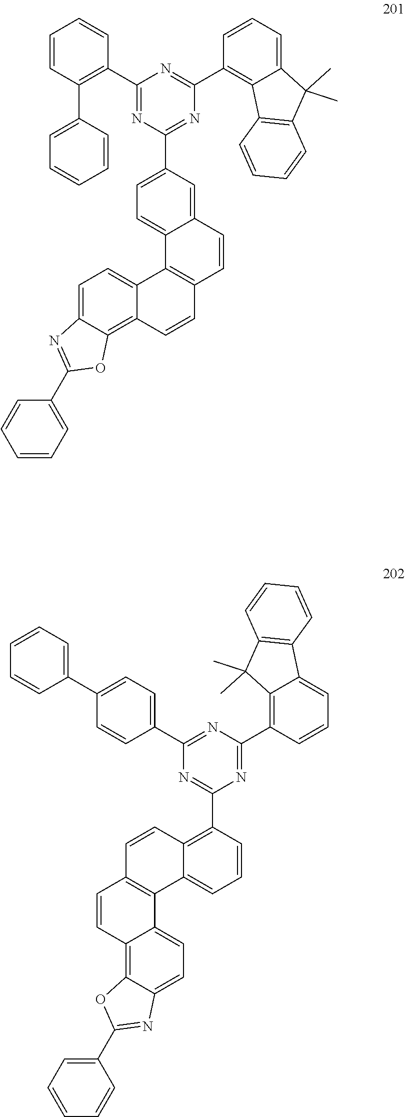 [SMILES: CC1(C)c2ccccc2-c2c(-c3nc(-c4ccc5c(ccc6ccc7c(ccc8nc(-c9ccccc9)oc87)c65)c4)nc(-c4ccccc4-c4ccccc4)n3)cccc21 ; CC1(C)c2ccccc2-c2cccc(-c3nc(-c4ccc(-c5ccccc5)cc4)nc(-c4cccc5c4ccc4ccc6c(ccc7nc(-c8ccccc8)oc76)c45)n3)c21]

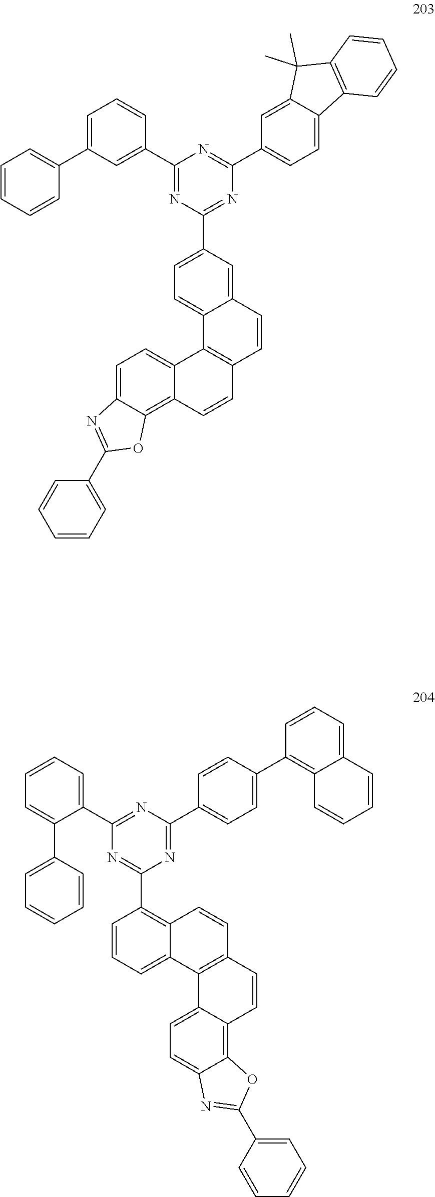 [SMILES: CC1(C)c2ccccc2-c2ccc(-c3nc(-c4cccc(-c5ccccc5)c4)nc(-c4ccc5c(ccc6ccc7c(ccc8nc(-c9ccccc9)oc87)c65)c4)n3)cc21 ; c1ccc(-c2nc3ccc4c(ccc5ccc6c(-c7nc(-c8ccc(-c9cccc%10ccccc9%10)cc8)nc(-c8ccccc8-c8ccccc8)n7)cccc6c54)c3o2)cc1]

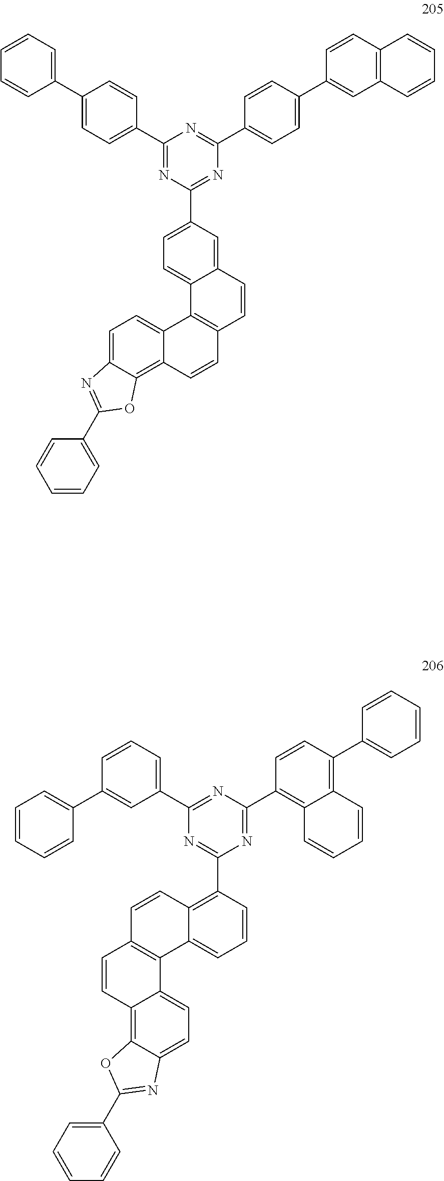 [SMILES: c1ccc(-c2ccc(-c3nc(-c4ccc(-c5ccc6ccccc6c5)cc4)nc(-c4ccc5c(ccc6ccc7c(ccc8nc(-c9ccccc9)oc87)c65)c4)n3)cc2)cc1 ; c1ccc(-c2cccc(-c3nc(-c4ccc(-c5ccccc5)c5ccccc45)nc(-c4cccc5c4ccc4ccc6c(ccc7nc(-c8ccccc8)oc76)c45)n3)c2)cc1]

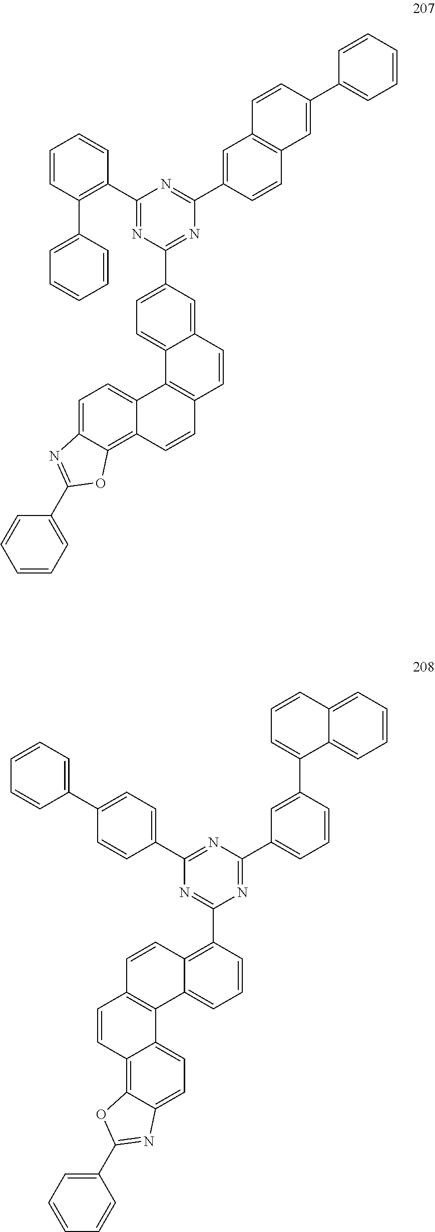 [SMILES: c1ccc(-c2ccc3cc(-c4nc(-c5ccc6c(ccc7ccc8c(ccc9nc(-c%10ccccc%10)oc98)c76)c5)nc(-c5ccccc5-c5ccccc5)n4)ccc3c2)cc1 ; c1ccc(-c2ccc(-c3nc(-c4cccc(-c5cccc6ccccc56)c4)nc(-c4cccc5c4ccc4ccc6c(ccc7nc(-c8ccccc8)oc76)c45)n3)cc2)cc1]

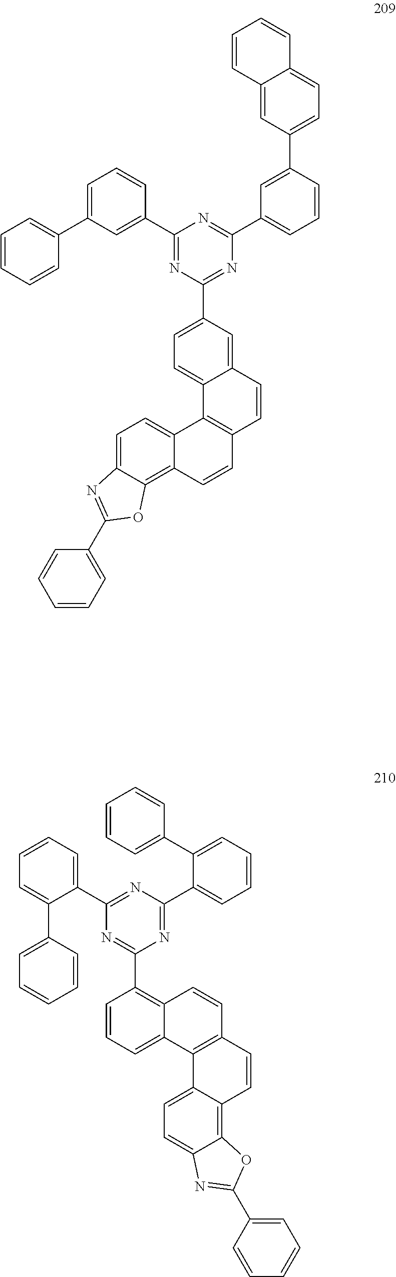 [SMILES: c1ccc(-c2cccc(-c3nc(-c4cccc(-c5ccc6ccccc6c5)c4)nc(-c4ccc5c(ccc6ccc7c(ccc8nc(-c9ccccc9)oc87)c65)c4)n3)c2)cc1 ; c1ccc(-c2nc3ccc4c(ccc5ccc6c(-c7nc(-c8ccccc8-c8ccccc8)nc(-c8ccccc8-c8ccccc8)n7)cccc6c54)c3o2)cc1]

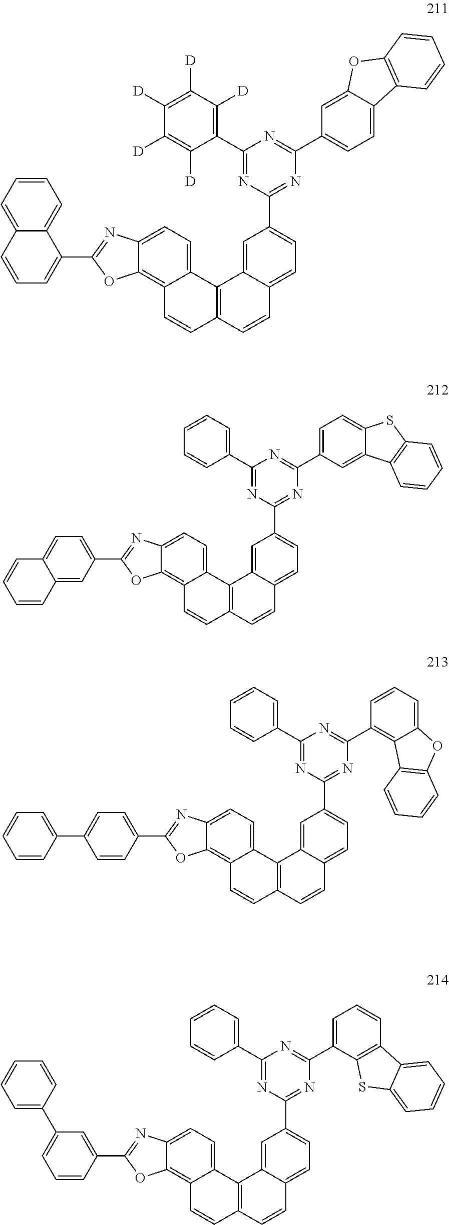 [SMILES: [2H]c1c([2H])c([2H])c(-c2nc(-c3ccc4c(c3)oc3ccccc34)nc(-c3ccc4ccc5ccc6c(ccc7nc(-c8cccc9ccccc89)oc76)c5c4c3)n2)c([2H])c1[2H] ; c1ccc(-c2nc(-c3ccc4sc5ccccc5c4c3)nc(-c3ccc4ccc5ccc6c(ccc7nc(-c8ccc9ccccc9c8)oc76)c5c4c3)n2)cc1 ; c1ccc(-c2ccc(-c3nc4ccc5c(ccc6ccc7ccc(-c8nc(-c9ccccc9)nc(-c9cccc%10oc%11ccccc%11c9%10)n8)cc7c65)c4o3)cc2)cc1 ; c1ccc(-c2cccc(-c3nc4ccc5c(ccc6ccc7ccc(-c8nc(-c9ccccc9)nc(-c9cccc%10c9sc9ccccc9%10)n8)cc7c65)c4o3)c2)cc1]

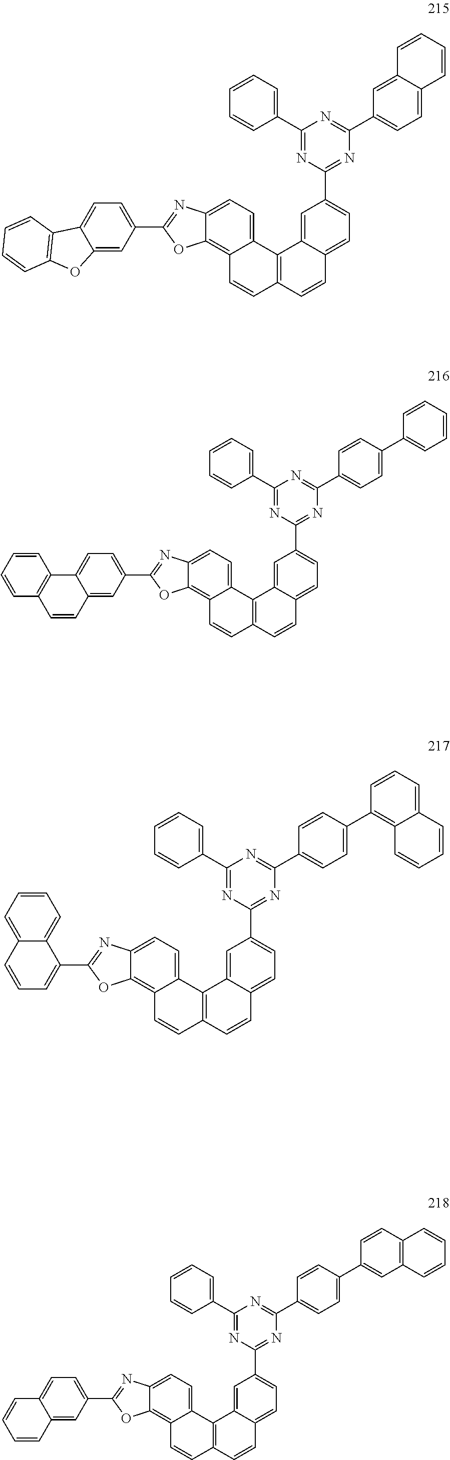 [SMILES: c1ccc(-c2nc(-c3ccc4ccccc4c3)nc(-c3ccc4ccc5ccc6c(ccc7nc(-c8ccc9c(c8)oc8ccccc89)oc76)c5c4c3)n2)cc1 ; c1ccc(-c2ccc(-c3nc(-c4ccccc4)nc(-c4ccc5ccc6ccc7c(ccc8nc(-c9ccc%10c(ccc%11ccccc%11%10)c9)oc87)c6c5c4)n3)cc2)cc1 ; c1ccc(-c2nc(-c3ccc(-c4cccc5ccccc45)cc3)nc(-c3ccc4ccc5ccc6c(ccc7nc(-c8cccc9ccccc89)oc76)c5c4c3)n2)cc1 ; c1ccc(-c2nc(-c3ccc(-c4ccc5ccccc5c4)cc3)nc(-c3ccc4ccc5ccc6c(ccc7nc(-c8ccc9ccccc9c8)oc76)c5c4c3)n2)cc1]

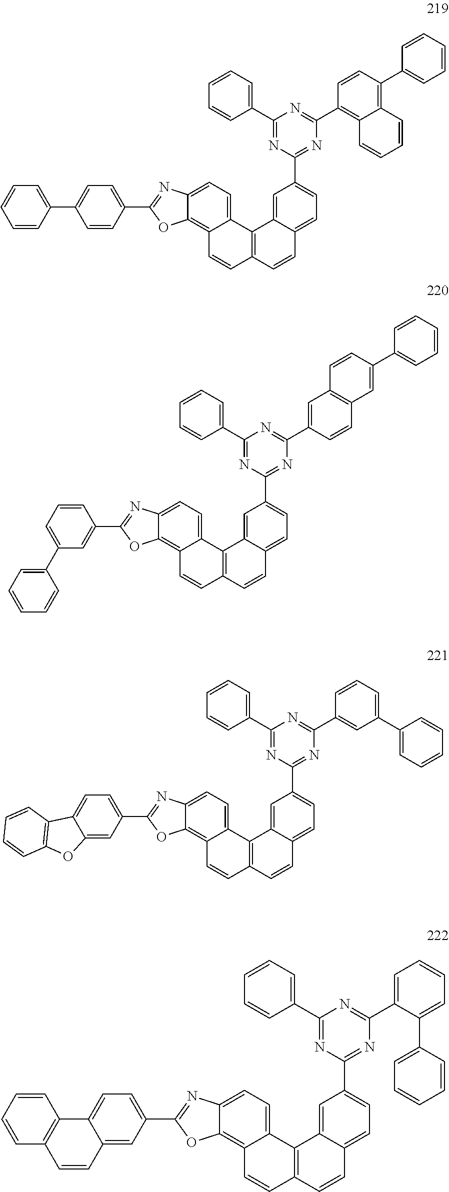 [SMILES: c1ccc(-c2ccc(-c3nc4ccc5c(ccc6ccc7ccc(-c8nc(-c9ccccc9)nc(-c9ccc(-c%10ccccc%10)c%10ccccc9%10)n8)cc7c65)c4o3)cc2)cc1 ; c1ccc(-c2cccc(-c3nc4ccc5c(ccc6ccc7ccc(-c8nc(-c9ccccc9)nc(-c9ccc%10cc(-c%11ccccc%11)ccc%10c9)n8)cc7c65)c4o3)c2)cc1 ; c1ccc(-c2cccc(-c3nc(-c4ccccc4)nc(-c4ccc5ccc6ccc7c(ccc8nc(-c9ccc%10c(c9)oc9ccccc9%10)oc87)c6c5c4)n3)c2)cc1 ; c1ccc(-c2nc(-c3ccc4ccc5ccc6c(ccc7nc(-c8ccc9c(ccc%10ccccc%109)c8)oc76)c5c4c3)nc(-c3ccccc3-c3ccccc3)n2)cc1]

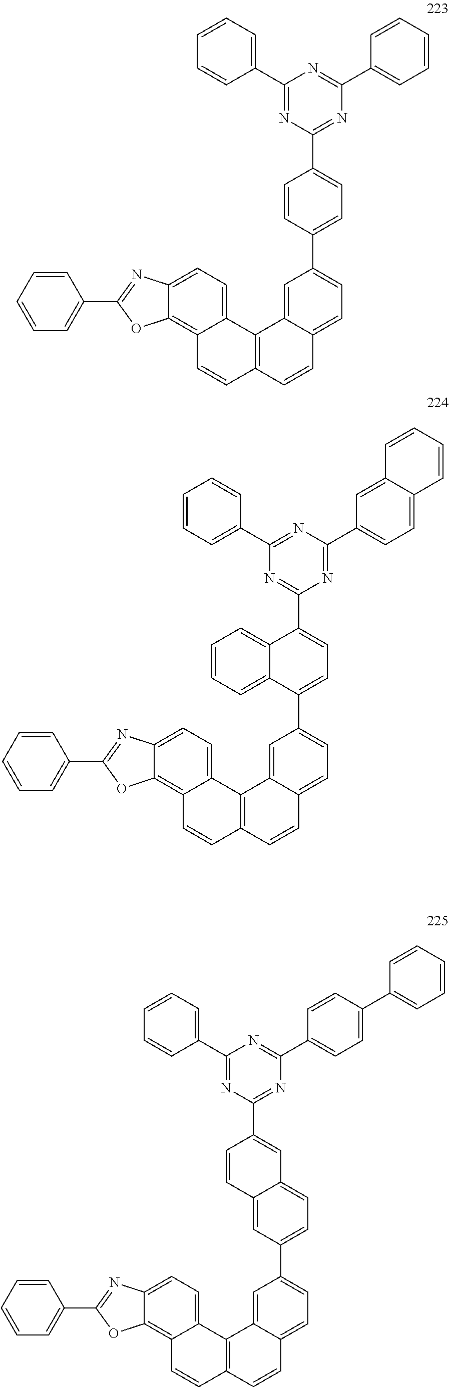 [SMILES: c1ccc(-c2nc(-c3ccccc3)nc(-c3ccc(-c4ccc5ccc6ccc7c(ccc8nc(-c9ccccc9)oc87)c6c5c4)cc3)n2)cc1 ; c1ccc(-c2nc(-c3ccc4ccccc4c3)nc(-c3ccc(-c4ccc5ccc6ccc7c(ccc8nc(-c9ccccc9)oc87)c6c5c4)c4ccccc34)n2)cc1 ; c1ccc(-c2ccc(-c3nc(-c4ccccc4)nc(-c4ccc5cc(-c6ccc7ccc8ccc9c(ccc%10nc(-c%11ccccc%11)oc%109)c8c7c6)ccc5c4)n3)cc2)cc1]

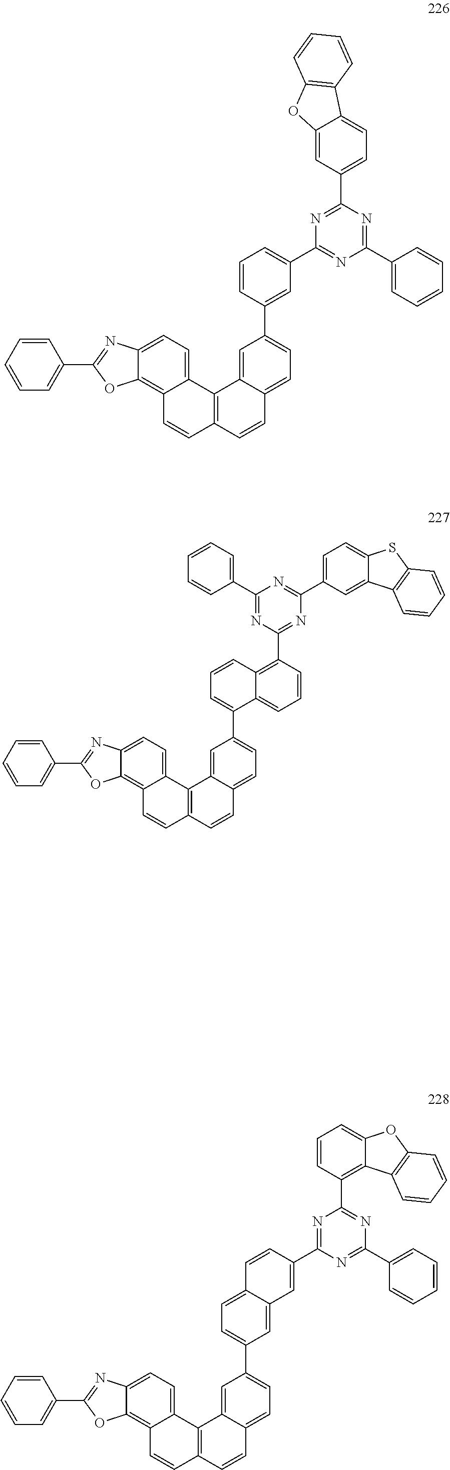 [SMILES: c1ccc(-c2nc(-c3cccc(-c4ccc5ccc6ccc7c(ccc8nc(-c9ccccc9)oc87)c6c5c4)c3)nc(-c3ccc4c(c3)oc3ccccc34)n2)cc1 ; c1ccc(-c2nc(-c3ccc4sc5ccccc5c4c3)nc(-c3cccc4c(-c5ccc6ccc7ccc8c(ccc9nc(-c%10ccccc%10)oc98)c7c6c5)cccc34)n2)cc1 ; c1ccc(-c2nc(-c3ccc4ccc(-c5ccc6ccc7ccc8c(ccc9nc(-c%10ccccc%10)oc98)c7c6c5)cc4c3)nc(-c3cccc4oc5ccccc5c34)n2)cc1]

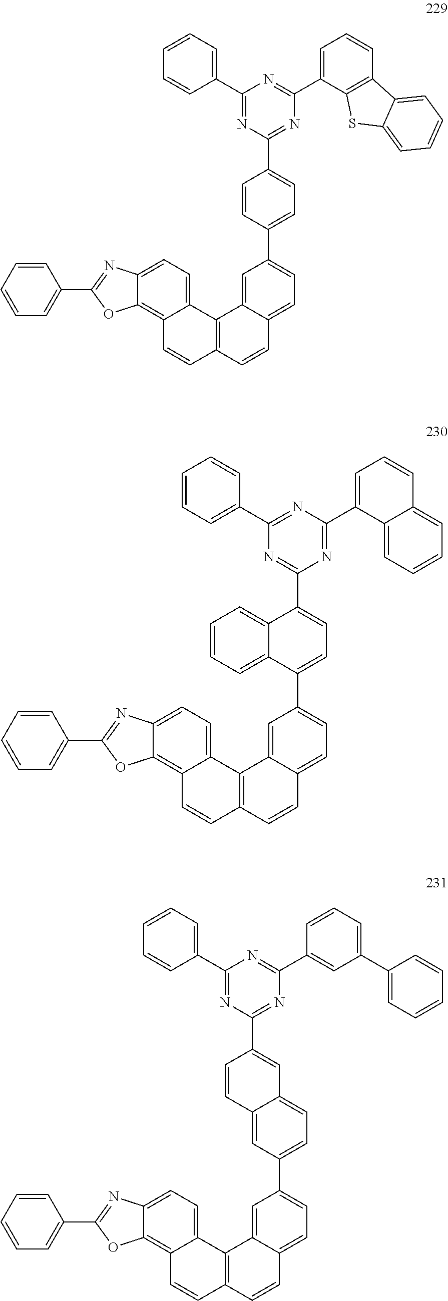 [SMILES: c1ccc(-c2nc(-c3ccc(-c4ccc5ccc6ccc7c(ccc8nc(-c9ccccc9)oc87)c6c5c4)cc3)nc(-c3cccc4c3sc3ccccc34)n2)cc1 ; c1ccc(-c2nc(-c3cccc4ccccc34)nc(-c3ccc(-c4ccc5ccc6ccc7c(ccc8nc(-c9ccccc9)oc87)c6c5c4)c4ccccc34)n2)cc1 ; c1ccc(-c2cccc(-c3nc(-c4ccccc4)nc(-c4ccc5cc(-c6ccc7ccc8ccc9c(ccc%10nc(-c%11ccccc%11)oc%109)c8c7c6)ccc5c4)n3)c2)cc1]

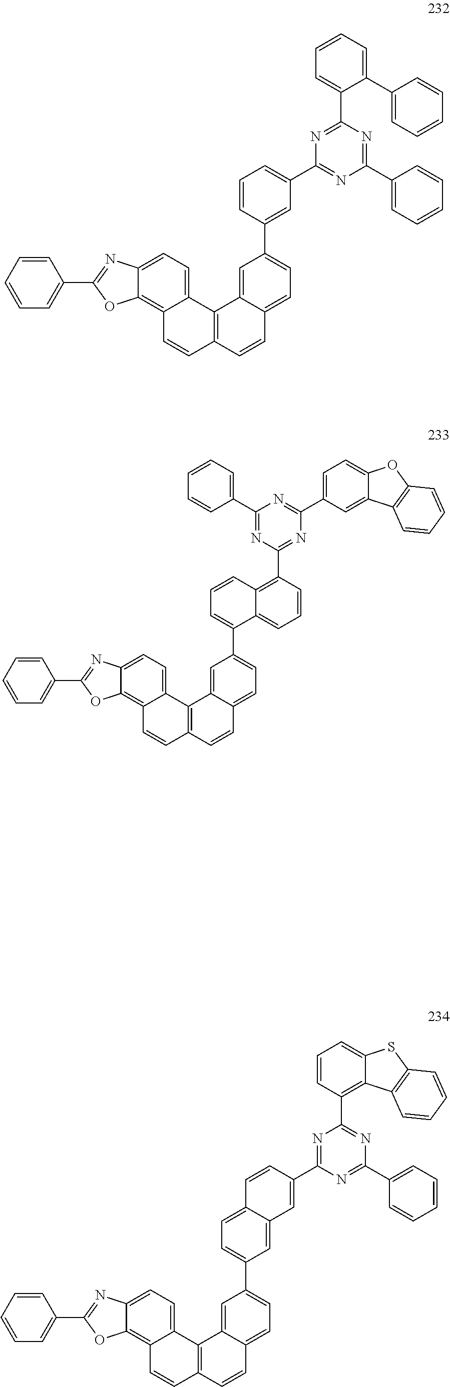 [SMILES: c1ccc(-c2nc(-c3cccc(-c4ccc5ccc6ccc7c(ccc8nc(-c9ccccc9)oc87)c6c5c4)c3)nc(-c3ccccc3-c3ccccc3)n2)cc1 ; c1ccc(-c2nc(-c3ccc4oc5ccccc5c4c3)nc(-c3cccc4c(-c5ccc6ccc7ccc8c(ccc9nc(-c%10ccccc%10)oc98)c7c6c5)cccc34)n2)cc1 ; c1ccc(-c2nc(-c3ccc4ccc(-c5ccc6ccc7ccc8c(ccc9nc(-c%10ccccc%10)oc98)c7c6c5)cc4c3)nc(-c3cccc4sc5ccccc5c34)n2)cc1]

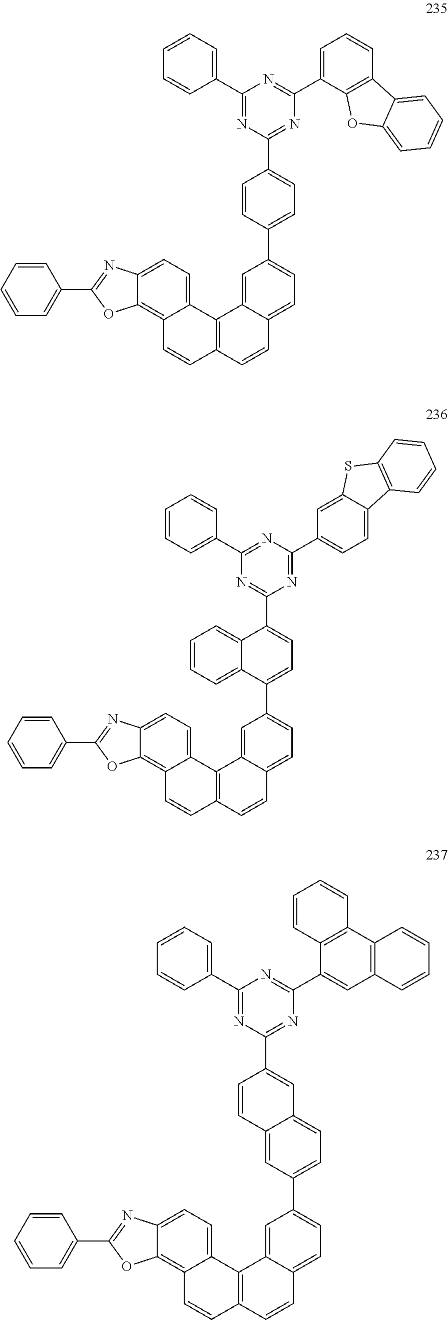 [SMILES: c1ccc(-c2nc(-c3ccc(-c4ccc5ccc6ccc7c(ccc8nc(-c9ccccc9)oc87)c6c5c4)cc3)nc(-c3cccc4c3oc3ccccc34)n2)cc1 ; c1ccc(-c2nc(-c3ccc4c(c3)sc3ccccc34)nc(-c3ccc(-c4ccc5ccc6ccc7c(ccc8nc(-c9ccccc9)oc87)c6c5c4)c4ccccc34)n2)cc1 ; c1ccc(-c2nc(-c3ccc4cc(-c5ccc6ccc7ccc8c(ccc9nc(-c%10ccccc%10)oc98)c7c6c5)ccc4c3)nc(-c3cc4ccccc4c4ccccc34)n2)cc1]

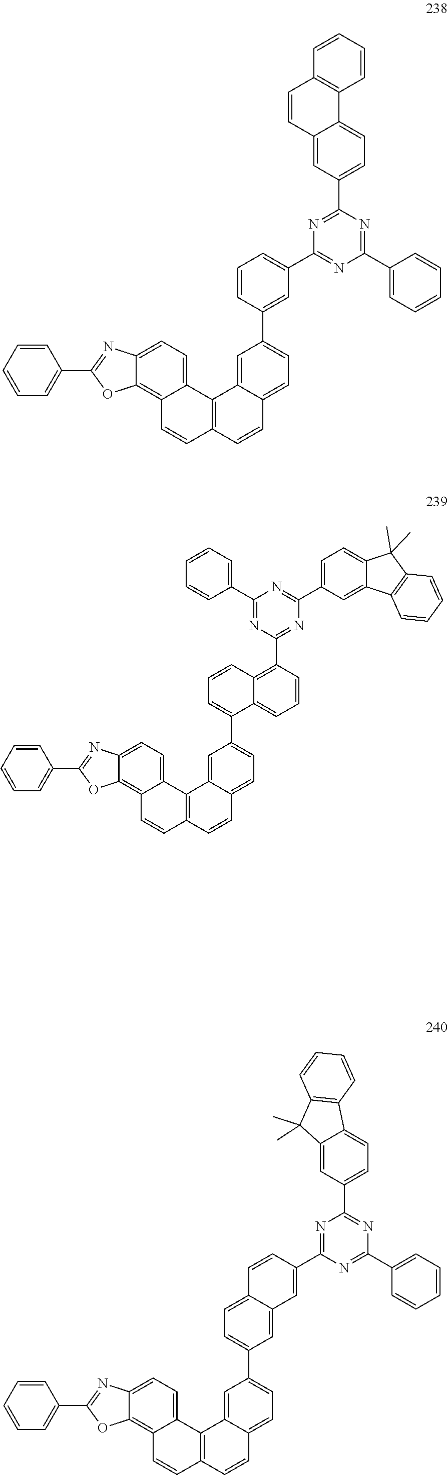 [SMILES: c1ccc(-c2nc(-c3cccc(-c4ccc5ccc6ccc7c(ccc8nc(-c9ccccc9)oc87)c6c5c4)c3)nc(-c3ccc4c(ccc5ccccc54)c3)n2)cc1 ; CC1(C)c2ccccc2-c2cc(-c3nc(-c4ccccc4)nc(-c4cccc5c(-c6ccc7ccc8ccc9c(ccc%10nc(-c%11ccccc%11)oc%109)c8c7c6)cccc45)n3)ccc21 ; CC1(C)c2ccccc2-c2ccc(-c3nc(-c4ccccc4)nc(-c4ccc5ccc(-c6ccc7ccc8ccc9c(ccc%10nc(-c%11ccccc%11)oc%109)c8c7c6)cc5c4)n3)cc21]

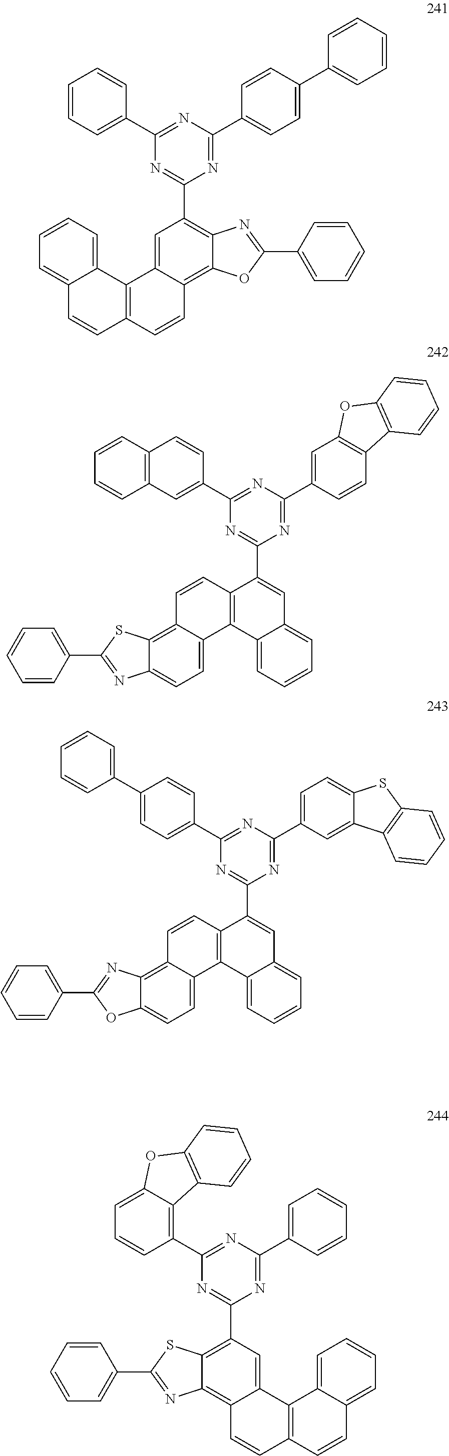 [SMILES: c1ccc(-c2ccc(-c3nc(-c4ccccc4)nc(-c4cc5c(ccc6ccc7ccccc7c65)c5oc(-c6ccccc6)nc45)n3)cc2)cc1 ; c1ccc(-c2nc3ccc4c(ccc5c(-c6nc(-c7ccc8ccccc8c7)nc(-c7ccc8c(c7)oc7ccccc78)n6)cc6ccccc6c54)c3s2)cc1 ; c1ccc(-c2ccc(-c3nc(-c4ccc5sc6ccccc6c5c4)nc(-c4cc5ccccc5c5c4ccc4c5ccc5oc(-c6ccccc6)nc54)n3)cc2)cc1 ; c1ccc(-c2nc(-c3cc4c(ccc5ccc6ccccc6c54)c4nc(-c5ccccc5)sc34)nc(-c3cccc4oc5ccccc5c34)n2)cc1]

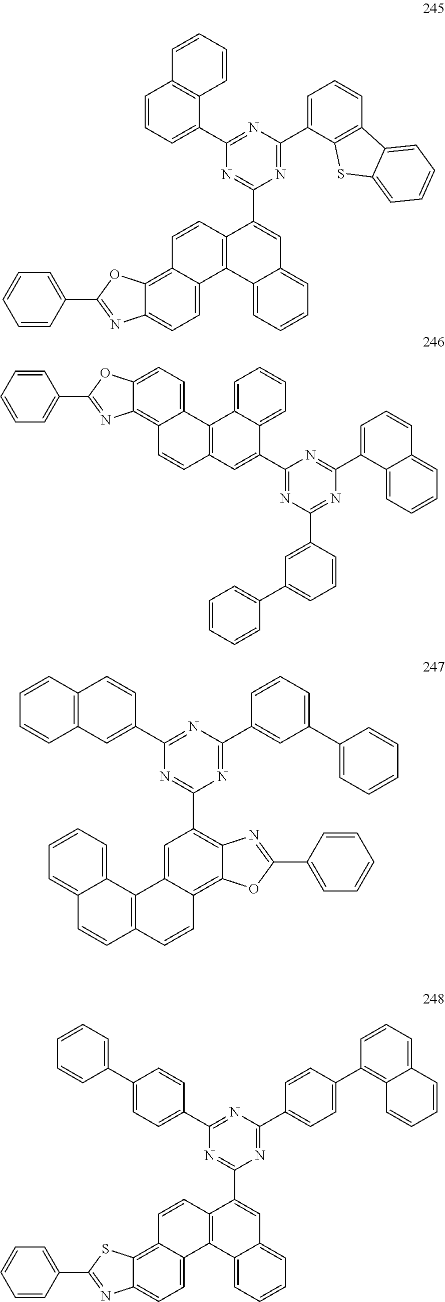

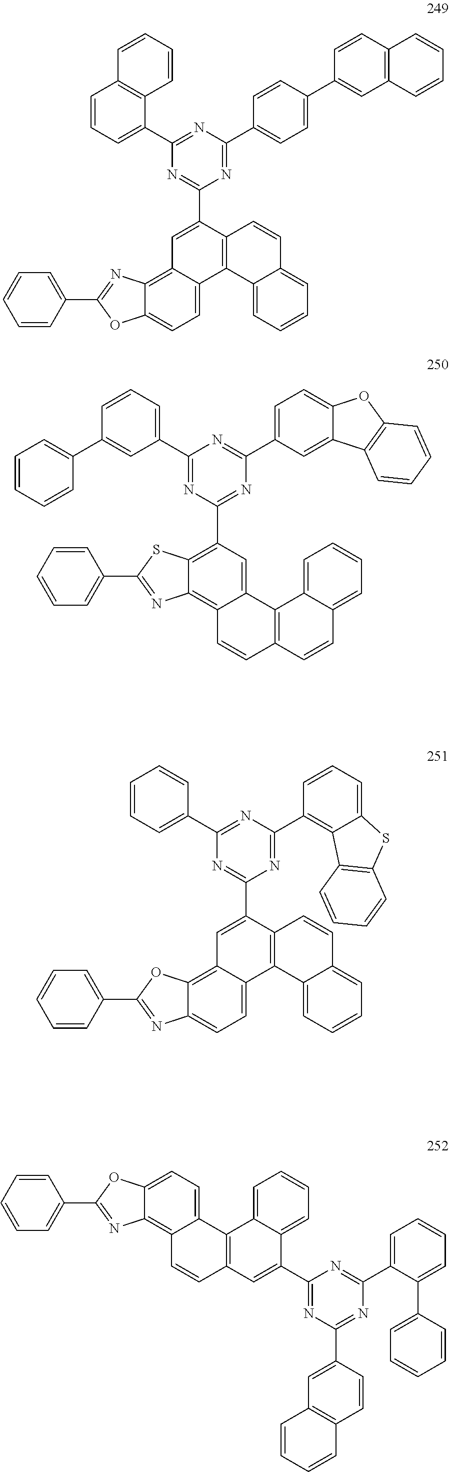 [SMILES: c1ccc(-c2nc3c(ccc4c3cc(-c3nc(-c5ccc(-c6ccc7ccccc7c6)cc5)nc(-c5cccc6ccccc56)n3)c3ccc5ccccc5c34)o2)cc1 ; c1ccc(-c2cccc(-c3nc(-c4ccc5oc6ccccc6c5c4)nc(-c4cc5c(ccc6ccc7ccccc7c65)c5nc(-c6ccccc6)sc45)n3)c2)cc1 ; c1ccc(-c2nc(-c3cc4c(ccc5nc(-c6ccccc6)oc54)c4c3ccc3ccccc34)nc(-c3cccc4sc5ccccc5c34)n2)cc1 ; c1ccc(-c2nc3c(ccc4c3ccc3cc(-c5nc(-c6ccc7ccccc7c6)nc(-c6ccccc6-c6ccccc6)n5)c5ccccc5c34)o2)cc1]

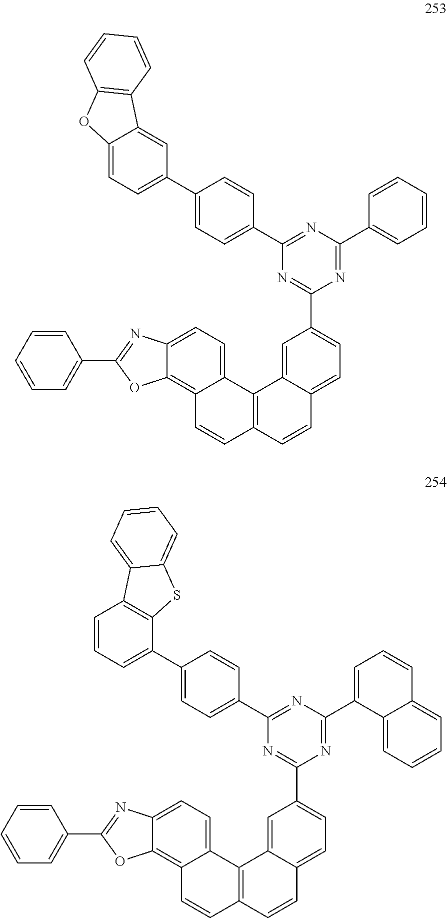 [SMILES: c1ccc(-c2nc(-c3ccc(-c4ccc5oc6ccccc6c5c4)cc3)nc(-c3ccc4ccc5ccc6c(ccc7nc(-c8ccccc8)oc76)c5c4c3)n2)cc1 ; c1ccc(-c2nc3ccc4c(ccc5ccc6ccc(-c7nc(-c8ccc(-c9cccc%10c9sc9ccccc9%10)cc8)nc(-c8cccc9ccccc89)n7)cc6c54)c3o2)cc1]

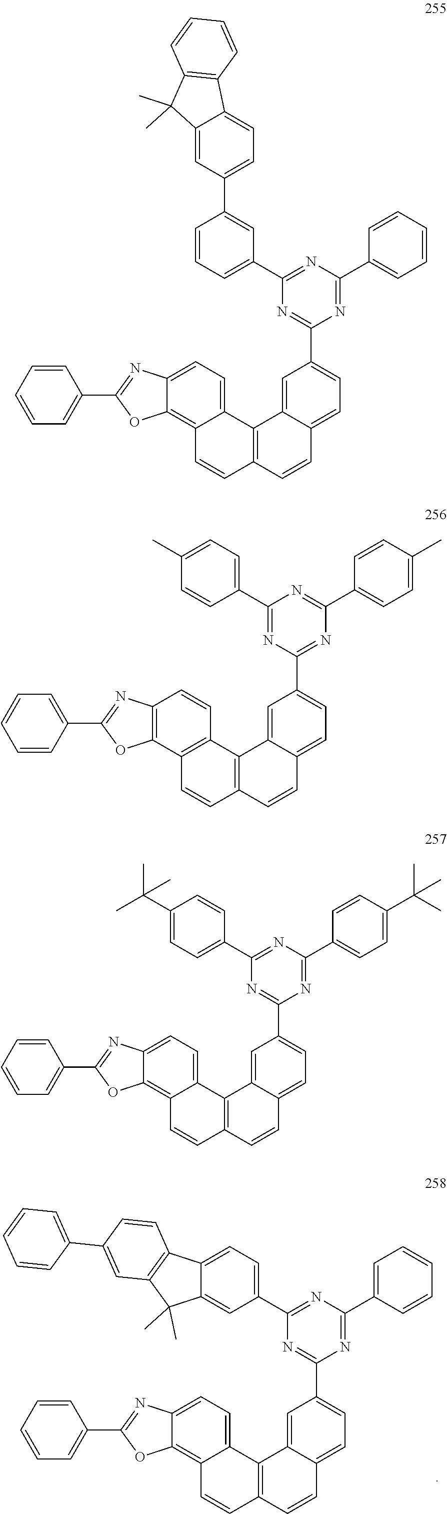 [SMILES: CC1(C)c2ccccc2-c2ccc(-c3cccc(-c4nc(-c5ccccc5)nc(-c5ccc6ccc7ccc8c(ccc9nc(-c%10ccccc%10)oc98)c7c6c5)n4)c3)cc21 ; Cc1ccc(-c2nc(-c3ccc(C)cc3)nc(-c3ccc4ccc5ccc6c(ccc7nc(-c8ccccc8)oc76)c5c4c3)n2)cc1 ; CC(C)(C)c1ccc(-c2nc(-c3ccc(C(C)(C)C)cc3)nc(-c3ccc4ccc5ccc6c(ccc7nc(-c8ccccc8)oc76)c5c4c3)n2)cc1 ; CC1(C)c2cc(-c3ccccc3)ccc2-c2ccc(-c3nc(-c4ccccc4)nc(-c4ccc5ccc6ccc7c(ccc8nc(-c9ccccc9)oc87)c6c5c4)n3)cc21]

In a second aspect, the present application provides an organic electroluminescent device comprising an anode, a cathode, and a functional layer disposed between the anode and the cathode; wherein the functional layer comprises an organic compound as described in the first aspect of the present application.

The organic compounds provided in the present application can be used to form at least one organic film layer in a functional layer to improve characteristics such as luminescence efficiency and lifetime of an organic electroluminescent device.

Alternatively, the functional layer comprises an organic light-emitting layer, the organic light-emitting layer comprising the organic compound. Wherein, the organic light-emitting layer may either be composed of the organic compound provided in the present application or be composed of the organic compound provided in the present application together with other materials.

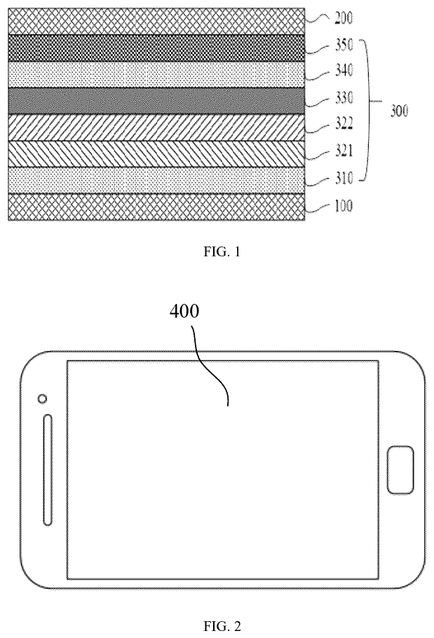

According to one specific embodiment, the organic electroluminescent device is shown in FIG. 1, and the organic electroluminescent device may include an anode 100, a hole injection layer 310, a hole transport layer 321, a hole adjustment layer (also known as a hole auxiliary layer, a second hole transport layer, or an electron blocking layer) 322, an organic light-emitting layer 330, an electron transport layer 340, an electron injection layer 350, and cathode 200 that are stacked sequentially.

In the present application, the anode 100 comprises an anode material, which is preferably a high work function material contributing to injection of holes into the functional layer. The specific examples of the anode material include: metals such as nickel, platinum, vanadium, chromium, copper, zinc, and gold, or alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly[3,4-(ethylene-1,2-dioxy)thiophene](PEDT), polypyrrole, and polyaniline, but are not limited thereto. Preferably, a transparent electrode comprising indium tin oxide (ITO) as the anode is included.

In the present application, the hole transport layer may include one or more hole transport materials, and the hole transport layer material may be selected from the group consisting of carbazole multimers, carbazole-connected triarylamine based compounds, and other types of compounds. Specifically, the hole transport materials may be selected from the group consisting of the following compounds or any combination thereof:

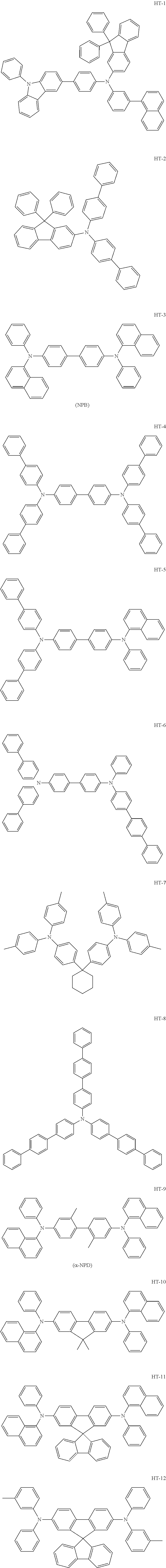

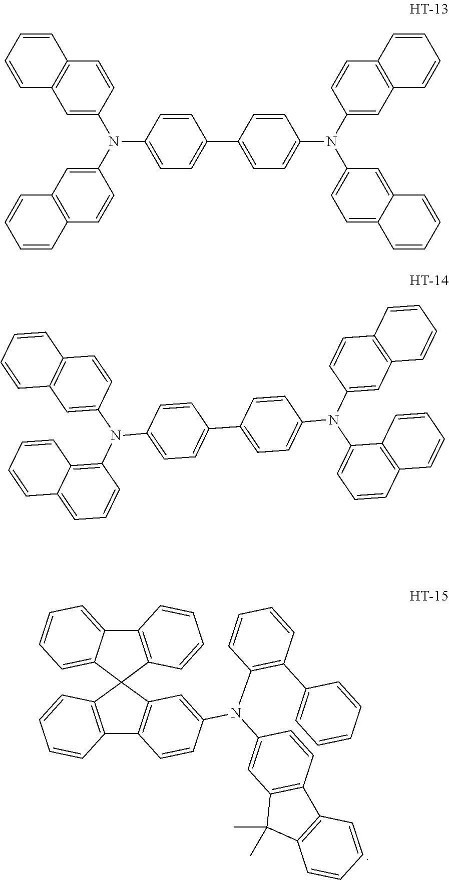 [SMILES: c1ccc2cc(N(c3ccc(-c4ccc(N(c5ccc6ccccc6c5)c5ccc6ccccc6c5)cc4)cc3)c3ccc4ccccc4c3)ccc2c1 ; c1ccc2cc(N(c3ccc(-c4ccc(N(c5ccc6ccccc6c5)c5cccc6ccccc56)cc4)cc3)c3cccc4ccccc34)ccc2c1 ; CC1(C)c2ccccc2-c2ccc(N(c3ccc4c(c3)C3(c5ccccc5-c5ccccc53)c3ccccc3-4)c3ccccc3-c3ccccc3)cc21]

In one embodiment, the hole transport layer 321 is composed of HT-5.

Alternatively, the hole adjustment layer 322 may include one or more hole transport materials, and the hole transport material may be selected from the group consisting of carbazole multimers, carbazole-connected triarylamine based compounds, and other types of compounds. It is not particularly limited in the present application. For example, in some embodiments of the present application, the hole adjustment layer 322 is composed of HT-2.

In one embodiment, the hole adjustment layer 322 is composed of HT-2. Alternatively, a hole injection layer 310 may be further provided between the anode 100 and the hole transport layer 321 to enhance the ability to inject holes into the hole transport layer 321. The hole injection layer 310 may choose to use a benzidine derivative, a starburst arylamine-based compound, a phthalocyanine derivative or other materials. It is not particularly limited in the present application. The material of the hole injection layer 310 is selected, for example, from the group consisting of the following compounds or any combination thereof;

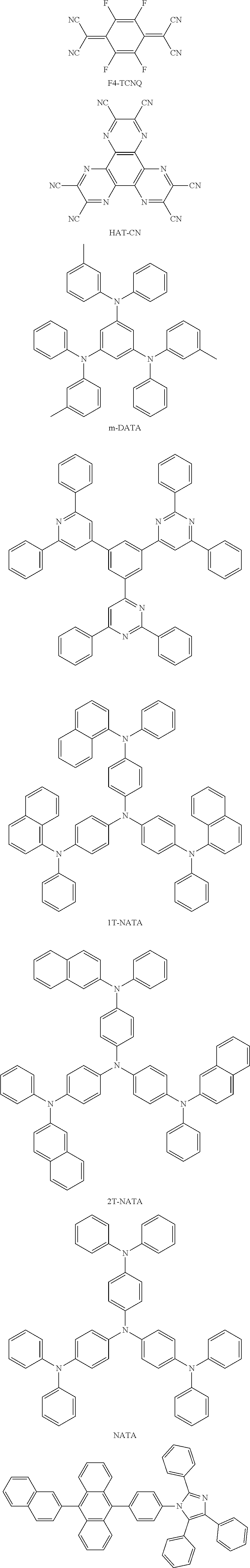

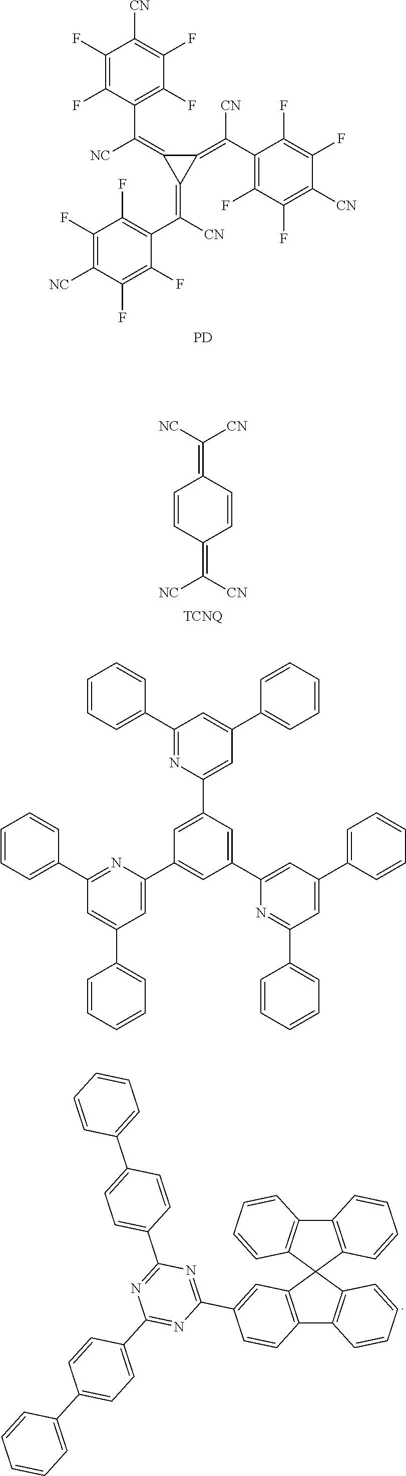 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "PD" ; N#CC(C#N)=c1ccc(=C(C#N)C#N)cc1 ; c1ccc(-c2cc(-c3ccccc3)nc(-c3cc(-c4cc(-c5ccccc5)cc(-c5ccccc5)n4)cc(-c4cc(-c5ccccc5)cc(-c5ccccc5)n4)c3)c2)cc1 ; c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In one embodiment, the hole injection layer 310 is composed of PD and H T-5.

In the present application, the organic light-emitting layer 330 may be composed of a single light-emitting material, or may comprise a host material and a guest material. Alternatively, the organic light-emitting layer 330 is composed of a host material and a guest material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the guest material, thereby enabling the guest material to emit light.

The host material of the organic light-emitting layer 330 may comprise metal chelating compounds, stilbene-based derivatives, aromatic amine derivatives, dibenzofuran derivatives, or other types of materials. Alternatively, the host material comprises the organic compounds of the present application.

The guest material of the organic light-emitting layer 330 may be a compound having a condensed aryl ring or its derivative, a compound having a heteroaryl ring or its derivative, an aromatic amine derivative, or other materials. It is not particularly limited in the present application. The guest material is also known as doping material or dopant. The dopant can be categorized into fluorescent dopants and phosphorescent dopants depending on the type of luminescence. The specific examples of the phosphorescent dopant include but are not limited to,

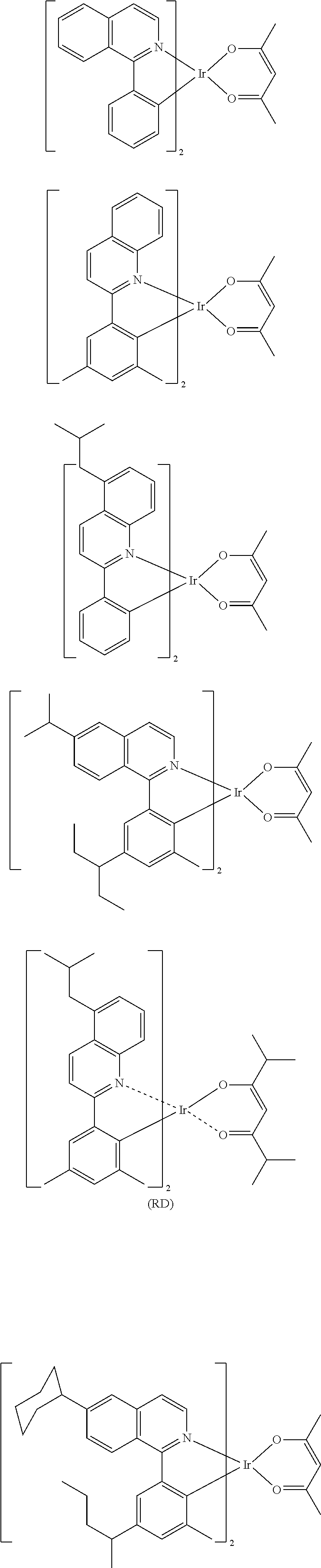

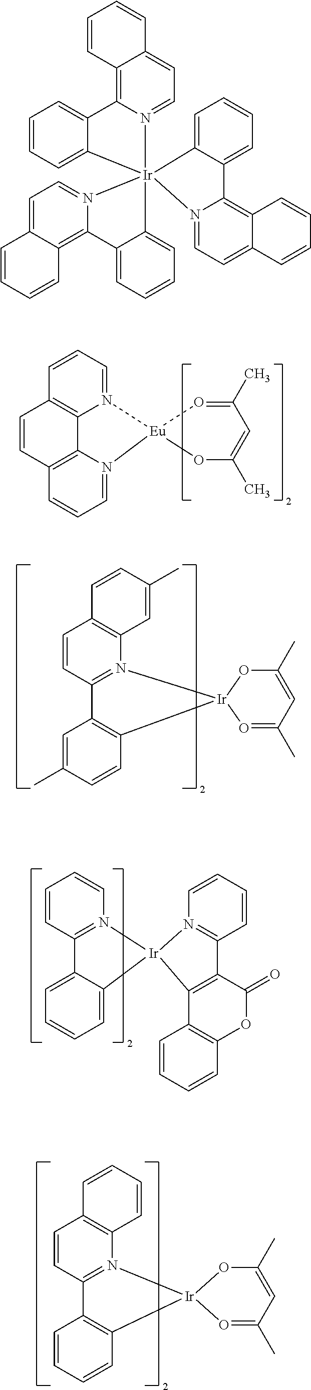

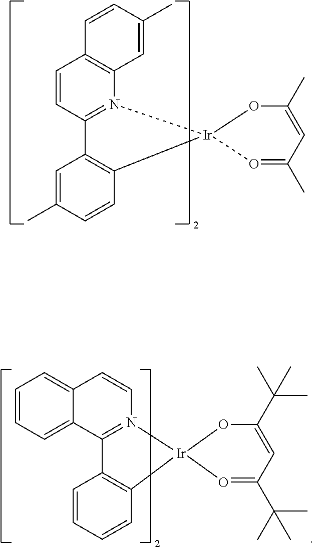

In one embodiment of the present application, the organic electroluminescent device is a red organic electroluminescent device. In one embodiment, the host material of the organic light-emitting layer 330 comprises the organic compounds of the present application. The guest material is, for example, RD. In another embodiment, the host material of the organic light-emitting layer 330 comprises the organic compounds of the present application and RH-P

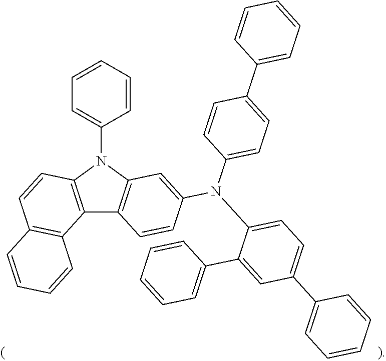

The guest material is, for example, RD.

The electron transport layer 340 may be a single-layer structure or a multi-layer structure and may comprise one or more electron transport material(s). The electron transport materials may be selected from, but not limited to, BTB, LiQ, benzimidazole derivatives, oxadiazole derivatives, quinoxaline derivatives, and other electron transport materials, and it is not particularly limited in the present application. The material of the electron transport layer 340 includes but is not limited to the following compounds:

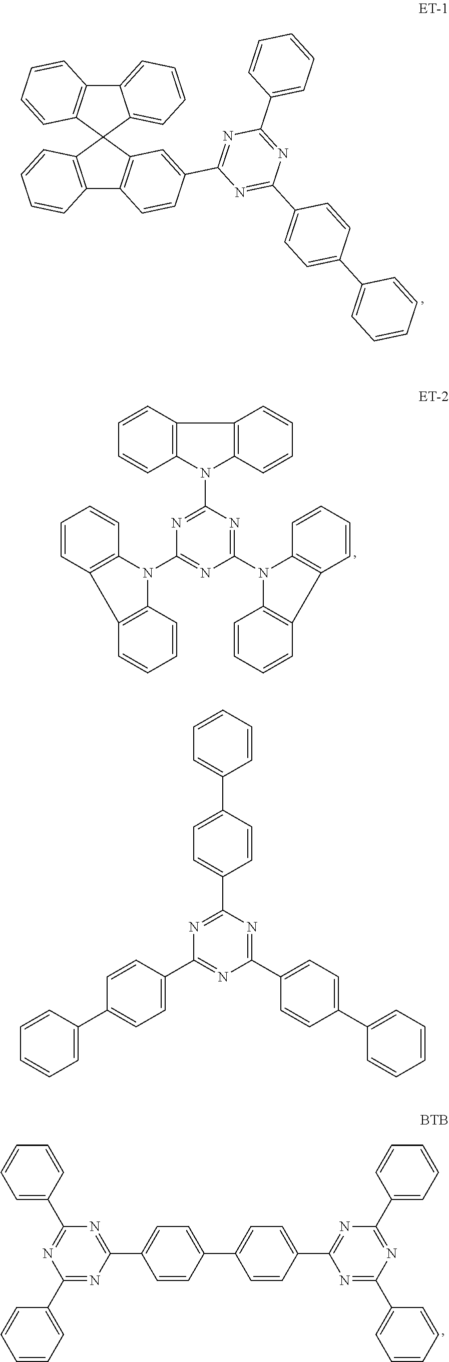 [SMILES: c1ccc(-c2ccc(-c3nc(-c4ccccc4)nc(-c4ccc5c(c4)C4(c6ccccc6-c6ccccc64)c4ccccc4-5)n3)cc2)cc1 ; c1ccc2c(c1)c1ccccc1n2-c1nc(-n2c3ccccc3c3ccccc32)nc(-n2c3ccccc3c3ccccc32)n1 ; c1ccc(-c2ccc(-c3nc(-c4ccc(-c5ccccc5)cc4)nc(-c4ccc(-c5ccccc5)cc4)n3)cc2)cc1 ; c1ccc(-c2nc(-c3ccccc3)nc(-c3ccc(-c4ccc(-c5nc(-c6ccccc6)nc(-c6ccccc6)n5)cc4)cc3)n2)cc1]

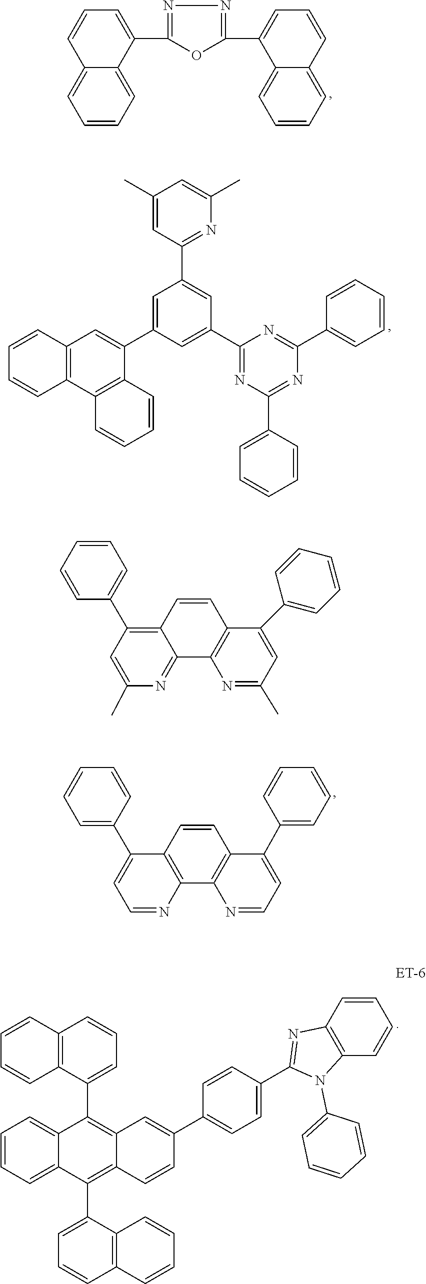 [SMILES: c1ccc2c(-c3nnc(-c4cccc5ccccc45)o3)cccc2c1 ; Cc1cc(C)nc(-c2cc(-c3nc(-c4ccccc4)nc(-c4ccccc4)n3)cc(-c3cc4ccccc4c4ccccc34)c2)c1 ; Cc1cc(-c2ccccc2)c2ccc3c(-c4ccccc4)cc(C)nc3c2n1 ; c1ccc(-c2ccnc3c2ccc2c(-c4ccccc4)ccnc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

In one embodiment of the present application, the electronic transport layer 340 may be composed of ET-1 and LiQ.

In the present application, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof; or multilayer materials such as LiF/Al, Liq/Al, LiO₂/Al, LiF/Ca, LiF/Al, and BaF₂/Ca. Alternatively, a metal electrode comprising magnesium and silver as the cathode is included.

Alternatively, an electron injection layer 350 may be further provided between the cathode 200 and the electron transport layer 340 to enhance the ability to inject electrons into the electron transport layer 340. The electron injection layer 350 may comprise an inorganic material such as an alkali metal sulfide and an alkali metal halide or may comprise a complex of an alkali metal and an organic compound. In one embodiment of the present application, the electron injection layer 350 may comprise ytterbium (Yb). In a third aspect, the present application provides an electronic apparatus, comprising the organic electroluminescent device described in the second aspect of the present application.

According to one embodiment, as shown in FIG. 2, the electronic apparatus provided is an electronic apparatus 400 comprising the above-described organic electroluminescent device. The electronic apparatus 400 may be for example, a display apparatus, a lighting apparatus, an optical communication apparatus, or other type of electronic apparatus, examples of which may include, but be not limited to, computer screens, mobile phone screens, televisions, electronic paper, emergency lamps, optical modules, etc.

The synthesis method of the organic compounds of the present application will be demonstrated in detail with the following synthetic examples, but the present application is not limited in any way by this.

### Synthetic Example

Those skilled in the art should recognize that the chemical reactions described in the present application can be used to properly prepare various organic compounds in the present application, and other methods used to prepare the compounds in the present application are all considered to be within the scope of the present application. For example, according to the present application, the synthesis of those non-exemplified compounds can be successfully completed by those skilled in the art through modification methods, such as appropriate protection of interfering groups, using other known reagents in addition to the ones described in the present application, or making some conventional adjustments to the reaction conditions. Compounds for which synthesis methods are not mentioned in the present application are all commercially available as raw materials.

### SYNTHESIS OF INTERMEDIATES

### Synthesis of Intermediate Sub-b1

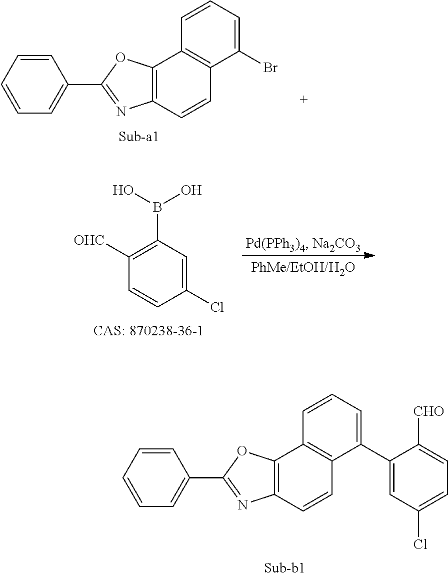 [SMILES: Brc1cccc2c1ccc1nc(-c3ccccc3)oc12 ; O=Cc1ccc(Cl)cc1B(O)O ; O=Cc1ccc(Cl)cc1-c1cccc2c1ccc1nc(-c3ccccc3)oc12]

Under a nitrogen atmosphere, Sub-a1 (16.20 g, 50 mmol), 5-chloro-2-formylphenylboronic acid (CAS: 870238-36-1, 10.14 g, 55 mmol), tetrakis(triphenylphosphine)palladium (Pd(PPh₃)₄, 0.58 g, 0.5 mmol), anhydrous sodium carbonate (Na₂CO₃, 10.60 g, 100 mmol), toluene (PhMe, 160 mL), anhydrous ethanol (EtOH, 40 mL), and deionized water (40 mL) were added into a 500 mL three-neck flask successively. The mixture was stirred and heated to reflux for 8 h of reaction. After the system cooled to room temperature, the mixture was extracted with dichloromethane (100 mL×3 times). The organic phases were combined and dried over anhydrous magnesium sulfate, followed by filtration and the removal of the solvent via distillation under reduced pressure to afford a crude product. The crude product was purified by silica gel column chromatography using dichloromethane/n-heptane as mobile phase to afford Sub-b1 (10.94 g, 57% yield) as a white solid.

Following the synthetic procedure of Intermediate Sub-b1, Intermediates Sub-b2 to Sub-b8 were synthesized by using Reactant B instead of Sub-a1, and Reactant C instead of 5-chloro-2-formylphenylboronic acid as listed in Table 1.

TABLE 1
Synthesis of Intermediate Sub-b2 to Intermediate Sub-b8

| Sub- | | |
|---|---|---|
| b | | |
| No. | Reactant B | Reactant C |
| Sub- b2 Sub- b3 | 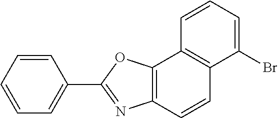 [SMILES: Brc1cccc2c1ccc1nc(-c3ccccc3)oc12] Sub-a1 | 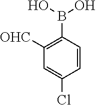 [SMILES: O=Cc1cc(Cl)ccc1B(O)O] CAS: 913835- 76-4 |
| | | 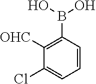 [SMILES: O=Cc1c(Cl)cccc1B(O)O] |
| | | CAS: 928048- |
| | | 11-7 |
| Sub- b4 | 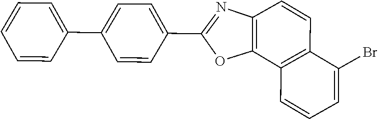 [SMILES: Brc1cccc2c1ccc1nc(-c3ccc(-c4ccccc4)cc3)oc12] | 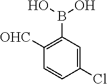 [SMILES: O=Cc1ccc(Cl)cc1B(O)O] CAS: 870238-36-1 |
| Sub- b5 | 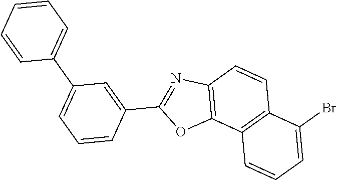 [SMILES: Brc1cccc2c1ccc1nc(-c3cccc(-c4ccccc4)c3)oc12] | |
| Sub- b6 | 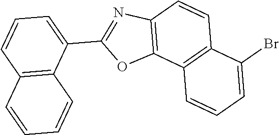 [SMILES: Brc1cccc2c1ccc1nc(-c3cccc4ccccc34)oc12] | |
| Sub- b7 | 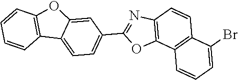 [SMILES: Brc1cccc2c1ccc1nc(-c3ccc4c(c3)oc3ccccc34)oc12] | |
| Sub- b8 | 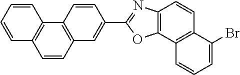 [SMILES: Brc1cccc2c1ccc1nc(-c3ccc4c(ccc5ccccc54)c3)oc12] | |

Synthesis of Intermediate Sub-b2 to Intermediate Sub-b8

| Sub- | | |
|---|---|---|
| b | | Yield |
| No. | Sub-b structure | (%) |
| Sub- b2 Sub- b3 | 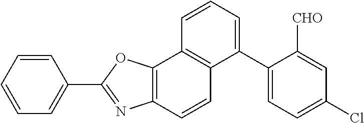 [SMILES: O=Cc1cc(Cl)ccc1-c1cccc2c1ccc1nc(-c3ccccc3)oc12] | 53 |
| | 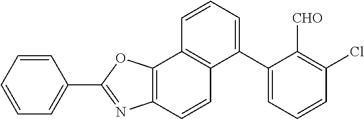 [SMILES: O=Cc1c(Cl)cccc1-c1cccc2c1ccc1nc(-c3ccccc3)oc12] | 51 |
| Sub- b4 | 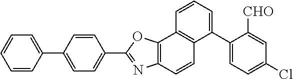 [SMILES: O=Cc1cc(Cl)ccc1-c1cccc2c1ccc1nc(-c3ccc(-c4ccccc4)cc3)oc12] | 60 |
| Sub- b5 | 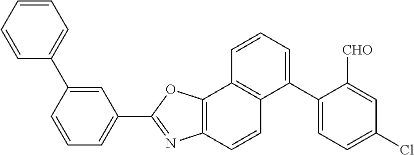 [SMILES: O=Cc1cc(Cl)ccc1-c1cccc2c1ccc1nc(-c3cccc(-c4ccccc4)c3)oc12] | 51 |
| Sub- b6 | 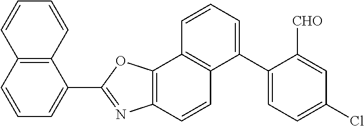 [SMILES: O=Cc1cc(Cl)ccc1-c1cccc2c1ccc1nc(-c3cccc4ccccc34)oc12] | 58 |
| Sub- b7 | 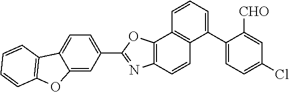 [SMILES: O=Cc1cc(Cl)ccc1-c1cccc2c1ccc1nc(-c3ccc4c(c3)oc3ccccc34)oc12] | 57 |
| Sub- b8 | 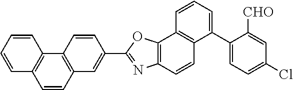 [SMILES: O=Cc1cc(Cl)ccc1-c1cccc2c1ccc1nc(-c3ccc4c(ccc5ccccc54)c3)oc12] | 60 |

### Synthesis of Intermediate Sub-c1

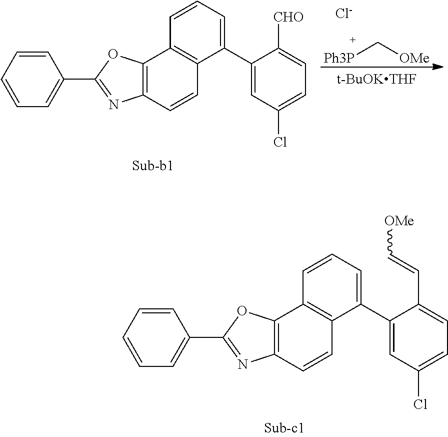

Under nitrogen protection, Sub-b1 (49.90 g, 130 mmol), (methoxymethyl)triphenylphosphonium chloride (74.38 g, 217 mmol) and anhydrous tetrahydrofuran (THF, 500 mL) were added into a 1000 mL three-necked flask successively, and the system was cooled down to 0° C. using an ice-water bath; Potassium tert-butoxide (t-BuOK) in anhydrous tetrahydrofuran (1 M, 220 mL) was then dropwise added slowly to the system; After the dropwise addition was completed, the system was allowed to warm up slowly to room temperature and the reaction was continued with stirring for 6 h. The reaction solution was poured into 1000 mL of deionized water, and was extracted with ethyl acetate (250 mL×3 times), the organic phases were combined and dried over anhydrous magnesium sulfate, followed by filtration and the removal of the solvent via distillation under reduced pressure to afford a crude product. The crude product was purified by silica gel column chromatography using n-heptane as mobile phase to afford Sub-c1 (39.10 g, 73% yield) as a white solid.

Following the synthetic procedure of Intermediate Sub-c1, Intermediates Sub-c2 to Sub-c8 were synthesized by using Reactant D instead of Sub-b1 as listed in Table 2.

TABLE 2
Synthesis of Intermediate Sub-c2 to Intermediate Sub-c8

| Sub- | | | |
|---|---|---|---|
| c | | | Yield |
| No. | Reactor D | Sub-c structure | (%) |
| Sub- c2 | 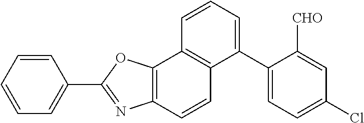 [SMILES: O=Cc1cc(Cl)ccc1-c1cccc2c1ccc1nc(-c3ccccc3)oc12] | 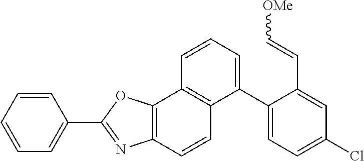 [SMILES: COC=Cc1cc(Cl)ccc1-c1cccc2c1ccc1nc(-c3ccccc3)oc12] | 65 |
| Sub- c3 | 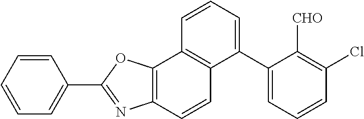 [SMILES: O=Cc1c(Cl)cccc1-c1cccc2c1ccc1nc(-c3ccccc3)oc12] | 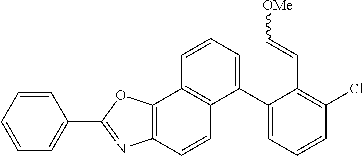 [SMILES: COC=Cc1c(Cl)cccc1-c1cccc2c1ccc1nc(-c3ccccc3)oc12] | 66 |
| Sub- c4 | 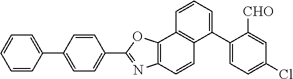 [SMILES: O=Cc1cc(Cl)ccc1-c1cccc2c1ccc1nc(-c3ccc(-c4ccccc4)cc3)oc12] | 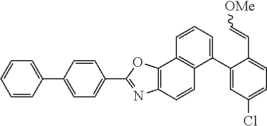 [SMILES: COC=Cc1ccc(Cl)cc1-c1cccc2c1ccc1nc(-c3ccc(-c4ccccc4)cc3)oc12] | 65 |
| Sub- c5 | 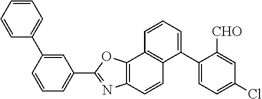 [SMILES: O=Cc1cc(Cl)ccc1-c1cccc2c1ccc1nc(-c3cccc(-c4ccccc4)c3)oc12] | 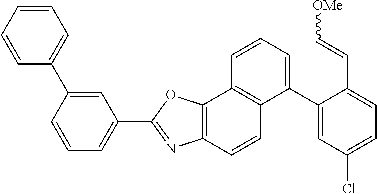 [SMILES: COC=Cc1ccc(Cl)cc1-c1cccc2c1ccc1nc(-c3cccc(-c4ccccc4)c3)oc12] | 66 |
| Sub- c6 | 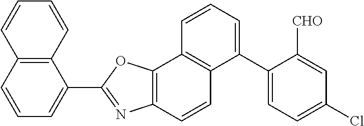 [SMILES: O=Cc1cc(Cl)ccc1-c1cccc2c1ccc1nc(-c3cccc4ccccc34)oc12] | 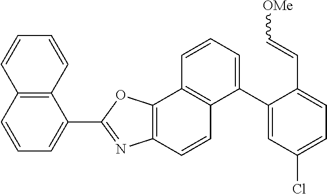 [SMILES: COC=Cc1ccc(Cl)cc1-c1cccc2c1ccc1nc(-c3cccc4ccccc34)oc12] | 74 |
| Sub- c7 | 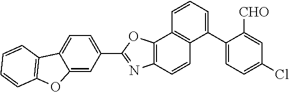 [SMILES: O=Cc1cc(Cl)ccc1-c1cccc2c1ccc1nc(-c3ccc4c(c3)oc3ccccc34)oc12] | 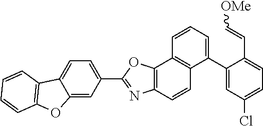 [SMILES: COC=Cc1ccc(Cl)cc1-c1cccc2c1ccc1nc(-c3ccc4c(c3)oc3ccccc34)oc12] | 67 |
| Sub- c8 | 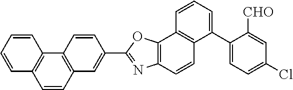 [SMILES: O=Cc1cc(Cl)ccc1-c1cccc2c1ccc1nc(-c3ccc4c(ccc5ccccc54)c3)oc12] | 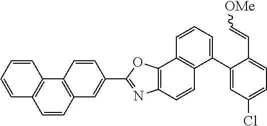 [SMILES: COC=Cc1ccc(Cl)cc1-c1cccc2c1ccc1nc(-c3ccc4c(ccc5ccccc54)c3)oc12] | 69 |

### Synthesis of Intermediate Sub-d1

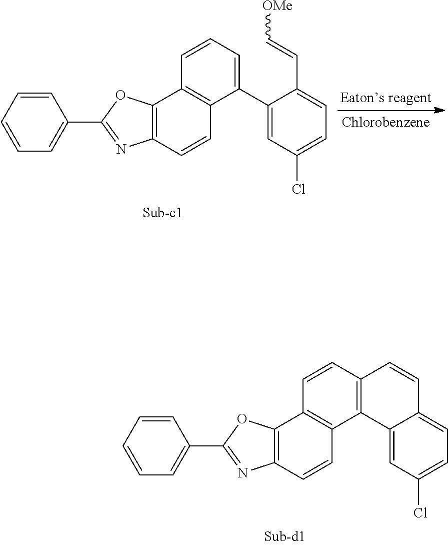 [SMILES: COC=Cc1ccc(Cl)cc1-c1cccc2c1ccc1nc(-c3ccccc3)oc12 ; Clc1ccc2ccc3ccc4c(ccc5nc(-c6ccccc6)oc54)c3c2c1]

Under nitrogen protection, Sub-c1 (49.00 g, 119 mmol), Baton's reagent (4.5 mL) and chlorobenzene (500 mL) were added into a 1000 mL three-necked flask successively, and the mixture was warmed up to reflux with continuously stirring for 4h of reaction. After the reaction system was cooled down to room temperature, the reaction solution was poured into 1000 mL of deionized water, and neutralized with saturated sodium hydroxide solution, then extracted with dichloromethane (250 mL×3 times). The organic phases were combined and dried over anhydrous magnesium sulfate, followed by the filtration and the removal of the solvent via distillation under reduced pressure to afford a crude product. The crude product was purified by silica gel column chromatography using dichloromethane/n-heptane as mobile phase to afford Sub-d1 (21.70 g, 48% yield) as a solid.

Following the synthetic procedure of Intermediate Sub-d1, Intermediates Sub-d2 to Sub-d8 were synthesized by—using Reactant E instead of Sub-c1 as listed in Table 3.

TABLE 3
Synthesis of Intermediate Sub-d2 to Intermediate Sub-d8

| Sub- | | | |
|---|---|---|---|
| d | | | Yield |
| No. | Reactant E | Sub-d structure | (%) |
| Sub- d2 | 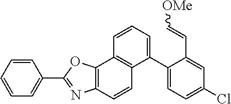 [SMILES: COC=Cc1cc(Cl)ccc1-c1cccc2c1ccc1nc(-c3ccccc3)oc12] | 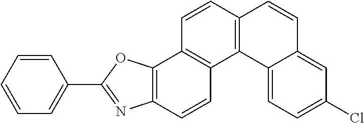 [SMILES: Clc1ccc2c(ccc3ccc4c(ccc5nc(-c6ccccc6)oc54)c32)c1] | 42 |
| Sub- d3 | 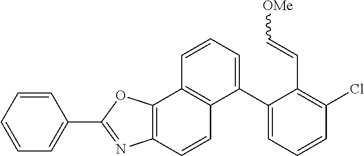 [SMILES: COC=Cc1c(Cl)cccc1-c1cccc2c1ccc1nc(-c3ccccc3)oc12] | 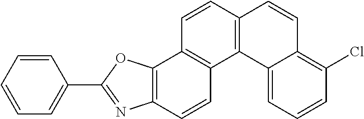 [SMILES: Clc1cccc2c1ccc1ccc3c(ccc4nc(-c5ccccc5)oc43)c12] | 46 |
| Sub- d4 | 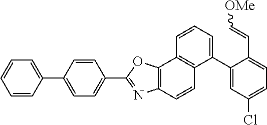 [SMILES: COC=Cc1ccc(Cl)cc1-c1cccc2c1ccc1nc(-c3ccc(-c4ccccc4)cc3)oc12] | 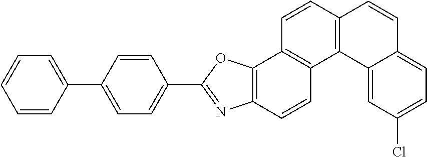 [SMILES: Clc1ccc2ccc3ccc4c(ccc5nc(-c6ccc(-c7ccccc7)cc6)oc54)c3c2c1] | 40 |
| Sub- d5 | 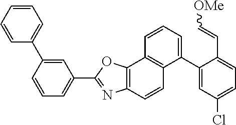 [SMILES: COC=Cc1ccc(Cl)cc1-c1cccc2c1ccc1nc(-c3cccc(-c4ccccc4)c3)oc12] | 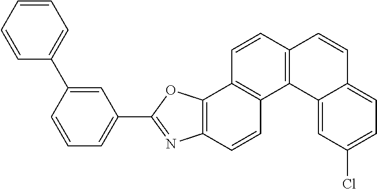 [SMILES: Clc1ccc2ccc3ccc4c(ccc5nc(-c6cccc(-c7ccccc7)c6)oc54)c3c2c1] | 43 |
| Sub- d6 | 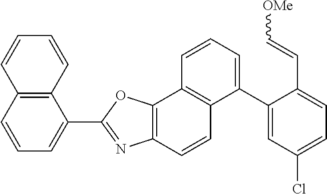 [SMILES: COC=Cc1ccc(Cl)cc1-c1cccc2c1ccc1nc(-c3cccc4ccccc34)oc12] | 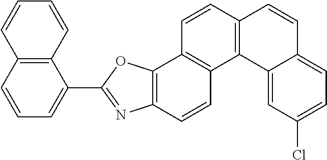 [SMILES: Clc1ccc2ccc3ccc4c(ccc5nc(-c6cccc7ccccc67)oc54)c3c2c1] | 44 |
| Sub- d7 | 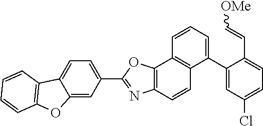 [SMILES: COC=Cc1ccc(Cl)cc1-c1cccc2c1ccc1nc(-c3ccc4c(c3)oc3ccccc34)oc12] | 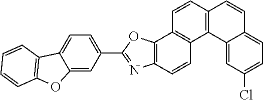 [SMILES: Clc1ccc2ccc3ccc4c(ccc5nc(-c6ccc7c(c6)oc6ccccc67)oc54)c3c2c1] | 42 |
| Sub- d8 | 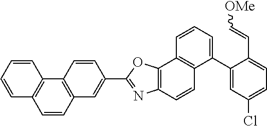 [SMILES: COC=Cc1ccc(Cl)cc1-c1cccc2c1ccc1nc(-c3ccc4c(ccc5ccccc54)c3)oc12] | 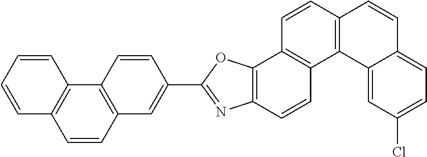 [SMILES: Clc1ccc2ccc3ccc4c(ccc5nc(-c6ccc7c(ccc8ccccc87)c6)oc54)c3c2c1] | 44 |

### Synthesis of Intermediate Sub-e1

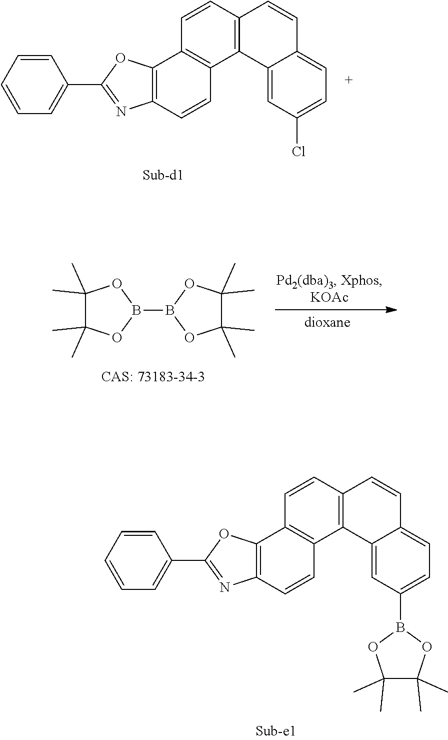

Under nitrogen protection, Sub-d1 (19.00 g, 50 mmol), bis(pinacolato)diboron (CAS: 73183-34-3, 14.00 g, 55 mmol), potassium acetate (KOAc, 10.80 g, 110 mmol), and 1,4-dioxane (160 mL) were added into a 500 mL three-necked flask successively. The mixture was stirred and heated to 40° C., followed by the rapid addition of tris(dibenzylideneacetone)dipalladium (Pd₂(dba)₃, 0.46 g, 0.50 mmol) and 2-dicyclohexylphosphino-2′,4′,6′-triisopropylbiphenyl (XPhos, 0.48 g, 1.0 mmol). The reaction mixture was then heated to reflux and stirred overnight. After the system was cooled to room temperature, 200 mL of water was added to the mixture, followed by vigorous stirring for 30 min. The resultant was filtered under reduced pressure. The filter cake was washed with deionized water until neutral and subsequently rinsed with 100 mL of anhydrous ethanol to afford a gray solid. The crude product was slurried once with n-heptane, then dissolved in toluene (200 mL) to form a clear solution and passed through a silica gel column to remove residual catalysts, followed by concentration to afford Sub-e1 as a white solid (15.79 g, 67% yield).

Following the synthetic procedure of Intermediate Sub-e1, Intermediates Sub-e2 to Sub-e7 were synthesized by using Reactant F instead of Sub-d1 as listed in Table 4.

TABLE 4
Synthesis of Intermediates Sub-e2 to Sub-e7

| Sub- | | | |
|---|---|---|---|
| e | | | Yield |
| No. | Reactant F structure | Sub-e structure | (%) |
| Sub- e2 | 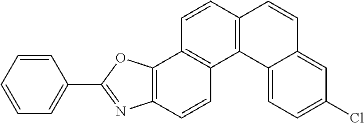 [SMILES: Clc1ccc2c(ccc3ccc4c(ccc5nc(-c6ccccc6)oc54)c32)c1] | 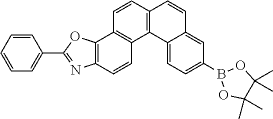 [SMILES: CC1(C)OB(c2ccc3c(ccc4ccc5c(ccc6nc(-c7ccccc7)oc65)c43)c2)OC1(C)C] | 64 |
| Sub- e3 | 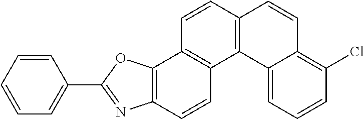 [SMILES: Clc1cccc2c1ccc1ccc3c(ccc4nc(-c5ccccc5)oc43)c12] | 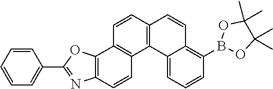 [SMILES: CC1(C)OB(c2cccc3c2ccc2ccc4c(ccc5nc(-c6ccccc6)oc54)c23)OC1(C)C] | 60 |
| Sub- e4 | 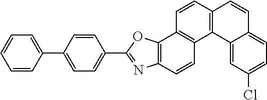 [SMILES: Clc1ccc2ccc3ccc4c(ccc5nc(-c6ccc(-c7ccccc7)cc6)oc54)c3c2c1] | 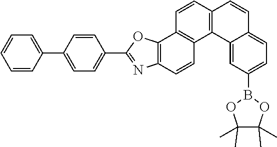 [SMILES: CC1(C)OB(c2ccc3ccc4ccc5c(ccc6nc(-c7ccc(-c8ccccc8)cc7)oc65)c4c3c2)OC1(C)C] | 65 |
| Sub- e5 | 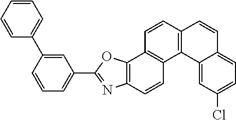 [SMILES: Clc1ccc2ccc3ccc4c(ccc5nc(-c6cccc(-c7ccccc7)c6)oc54)c3c2c1] | 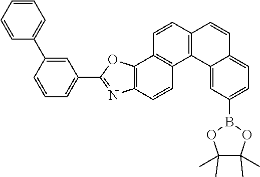 [SMILES: CC1(C)OB(c2ccc3ccc4ccc5c(ccc6nc(-c7cccc(-c8ccccc8)c7)oc65)c4c3c2)OC1(C)C] | 66 |
| Sub- e6 | 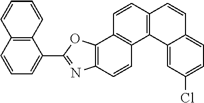 [SMILES: Clc1ccc2ccc3ccc4c(ccc5nc(-c6cccc7ccccc67)oc54)c3c2c1] | 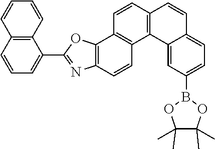 [SMILES: CC1(C)OB(c2ccc3ccc4ccc5c(ccc6nc(-c7cccc8ccccc78)oc65)c4c3c2)OC1(C)C] | 55 |
| Sub- e7 | 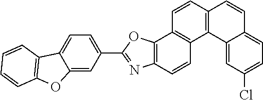 [SMILES: Clc1ccc2ccc3ccc4c(ccc5nc(-c6ccc7c(c6)oc6ccccc67)oc54)c3c2c1] | 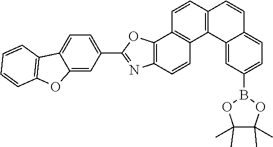 [SMILES: CC1(C)OB(c2ccc3ccc4ccc5c(ccc6nc(-c7ccc8c(c7)oc7ccccc78)oc65)c4c3c2)OC1(C)C] | 58 |
| Sub- e8 | 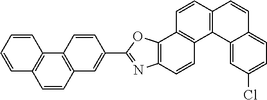 [SMILES: Clc1ccc2ccc3ccc4c(ccc5nc(-c6ccc7c(ccc8ccccc87)c6)oc54)c3c2c1] | 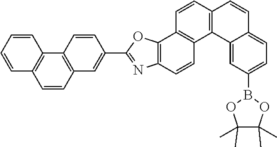 [SMILES: CC1(C)OB(c2ccc3ccc4ccc5c(ccc6nc(-c7ccc8c(ccc9ccccc98)c7)oc65)c4c3c2)OC1(C)C] | 57 |

### Synthesis of Compound

### Synthesis of Compound 4

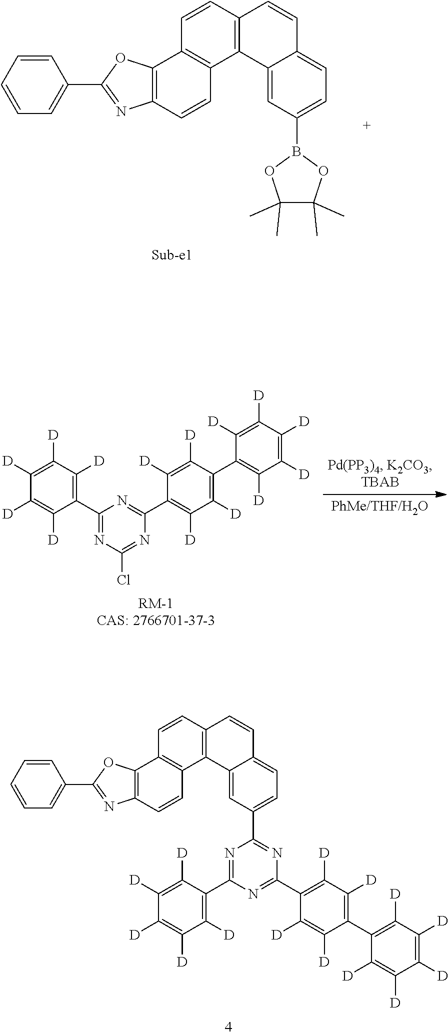 [SMILES: CC1(C)OB(c2ccc3ccc4ccc5c(ccc6nc(-c7ccccc7)oc65)c4c3c2)OC1(C)C ; [2H]c1c([2H])c([2H])c(-c2nc(Cl)nc(-c3c([2H])c([2H])c(-c4c([2H])c([2H])c([2H])c([2H])c4[2H])c([2H])c3[2H])n2)c([2H])c1[2H] ; [2H]c1c([2H])c([2H])c(-c2nc(-c3ccc4ccc5ccc6c(ccc7nc(-c8ccccc8)oc76)c5c4c3)nc(-c3c([2H])c([2H])c(-c4c([2H])c([2H])c([2H])c([2H])c4[2H])c([2H])c3[2H])n2)c([2H])c1[2H]]

Under nitrogen protection, Sub-e1 (11.78 g, 25 mmol), RM-1 (8.95 g, 25 mmol), tetrakis(triphenylphosphine)palladium (Pd(PP₃)₄, 0.29 g, 0.25 mmol), anhydrous potassium carbonate (K₂CO₃, 0.9 g, 50 mmol), tetrabutylammonium bromide (0.8 g, 2.5 mmol), toluene (120 mL), tetrahydrofuran (30 mL), and deionized water (30 mL) were added into a 250 mL three-necked flask successively. The mixture was stirred and heated to reflux for 16 h of reaction. After the system cooled to room temperature, the mixture was extracted with dichloromethane (100 mL×3 times), the organic phases were combined and dried over anhydrous magnesium sulfate, followed by filtration and the removal of the solvent via distillation under reduced pressure to afford a crude product. The crude product was purified by silica gel column chromatography using dichloromethane/n-heptane as mobile phase to afford Compound 4 (10.50 g, 63% yield, m/z=667.32[M+H]⁺) as a white solid.

Following the synthetic procedure of Compound 4, the compounds in the present application as listed in Table 5 were synthesized by using Reactant G instead of Sub-e1, and Reactant H instead of RM-1 as listed in Table 5.

TABLE 5
Synthesis of the compounds in the present application

| Reactant G | Reactant H |
|---|---|
| 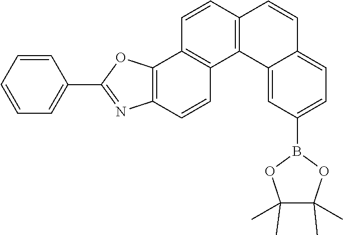 [SMILES: CC1(C)OB(c2ccc3ccc4ccc5c(ccc6nc(-c7ccccc7)oc65)c4c3c2)OC1(C)C] | 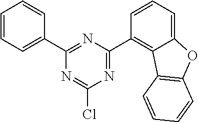 [SMILES: Clc1nc(-c2ccccc2)nc(-c2cccc3oc4ccccc4c23)n1] CAS: 1883265-32-4 |
| 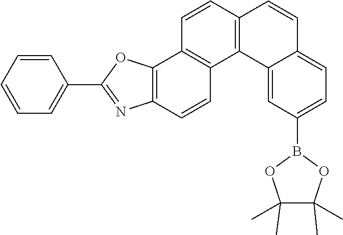 [SMILES: CC1(C)OB(c2ccc3ccc4ccc5c(ccc6nc(-c7ccccc7)oc65)c4c3c2)OC1(C)C] | 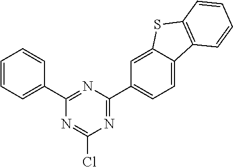 [SMILES: Clc1nc(-c2ccccc2)nc(-c2ccc3c(c2)sc2ccccc23)n1] CAS: 2172889-29-9 |
| 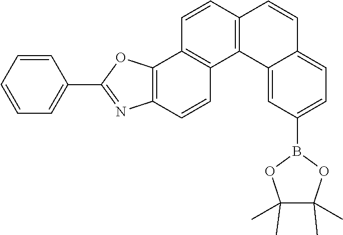 [SMILES: CC1(C)OB(c2ccc3ccc4ccc5c(ccc6nc(-c7ccccc7)oc65)c4c3c2)OC1(C)C] | 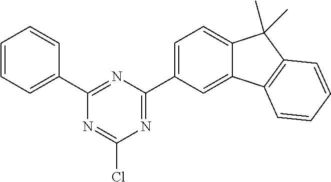 [SMILES: CC1(C)c2ccccc2-c2cc(-c3nc(Cl)nc(-c4ccccc4)n3)ccc21] CAS: 1835683-72-1 |
| 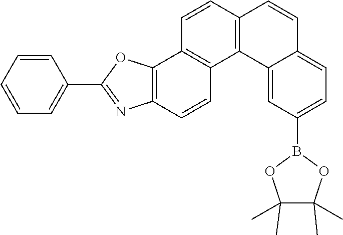 [SMILES: CC1(C)OB(c2ccc3ccc4ccc5c(ccc6nc(-c7ccccc7)oc65)c4c3c2)OC1(C)C] | 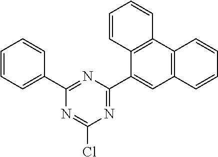 [SMILES: Clc1nc(-c2ccccc2)nc(-c2cc3ccccc3c3ccccc23)n1] CAS: 1821147-80-1 |
| 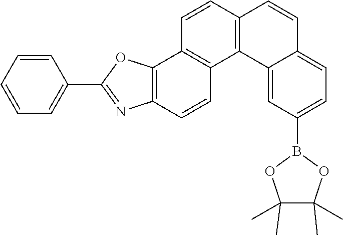 [SMILES: CC1(C)OB(c2ccc3ccc4ccc5c(ccc6nc(-c7ccccc7)oc65)c4c3c2)OC1(C)C] | 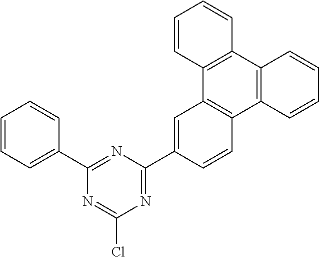 [SMILES: Clc1nc(-c2ccccc2)nc(-c2ccc3c4ccccc4c4ccccc4c3c2)n1] |
| | CAS: 1836145-06-2 |
| 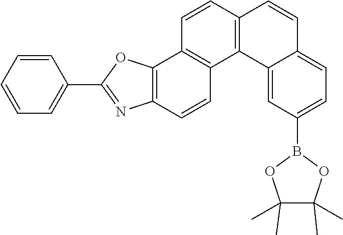 [SMILES: CC1(C)OB(c2ccc3ccc4ccc5c(ccc6nc(-c7ccccc7)oc65)c4c3c2)OC1(C)C] | 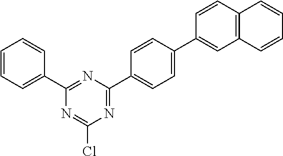 [SMILES: Clc1nc(-c2ccccc2)nc(-c2ccc(-c3ccc4ccccc4c3)cc2)n1] CAS: 2396648-13-6 |
| 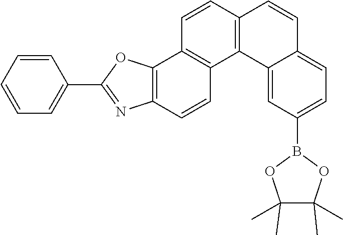 [SMILES: CC1(C)OB(c2ccc3ccc4ccc5c(ccc6nc(-c7ccccc7)oc65)c4c3c2)OC1(C)C] | 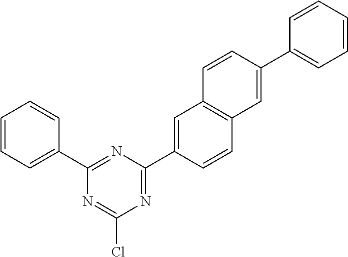 [SMILES: Clc1nc(-c2ccccc2)nc(-c2ccc3cc(-c4ccccc4)ccc3c2)n1] |
| | CAS: 2476829-04-4 |
| 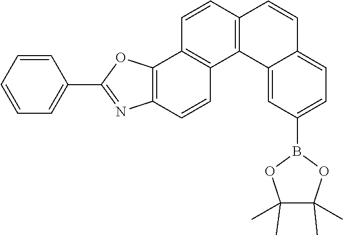 [SMILES: CC1(C)OB(c2ccc3ccc4ccc5c(ccc6nc(-c7ccccc7)oc65)c4c3c2)OC1(C)C] | 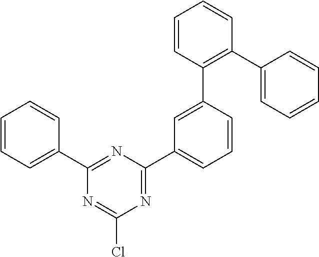 [SMILES: Clc1nc(-c2ccccc2)nc(-c2cccc(-c3ccccc3-c3ccccc3)c2)n1] CAS: 2173555-94-5 |
| 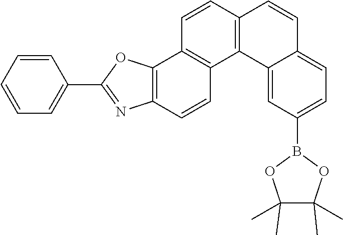 [SMILES: CC1(C)OB(c2ccc3ccc4ccc5c(ccc6nc(-c7ccccc7)oc65)c4c3c2)OC1(C)C] | 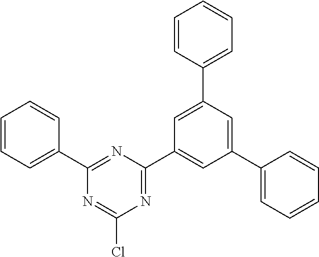 [SMILES: Clc1nc(-c2ccccc2)nc(-c2cc(-c3ccccc3)cc(-c3ccccc3)c2)n1] |
| | CAS: 1616231-57-2 |
| 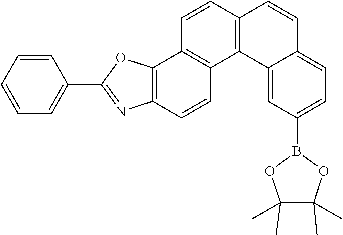 [SMILES: CC1(C)OB(c2ccc3ccc4ccc5c(ccc6nc(-c7ccccc7)oc65)c4c3c2)OC1(C)C] | 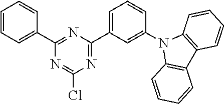 [SMILES: Clc1nc(-c2ccccc2)nc(-c2cccc(-n3c4ccccc4c4ccccc43)c2)n1] CAS: 2260561-80-4 |
| 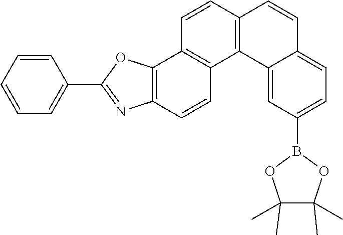 [SMILES: CC1(C)OB(c2ccc3ccc4ccc5c(ccc6nc(-c7ccccc7)oc65)c4c3c2)OC1(C)C] | 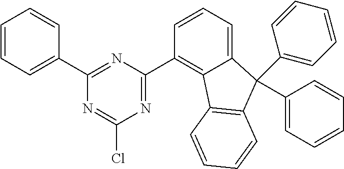 [SMILES: Clc1nc(-c2ccccc2)nc(-c2cccc3c2-c2ccccc2C3(c2ccccc2)c2ccccc2)n1] CAS: 1835683-70-9 |
| 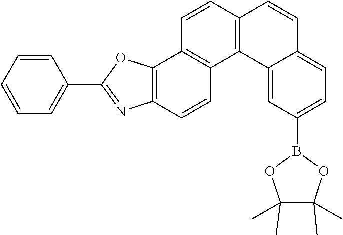 [SMILES: CC1(C)OB(c2ccc3ccc4ccc5c(ccc6nc(-c7ccccc7)oc65)c4c3c2)OC1(C)C] | 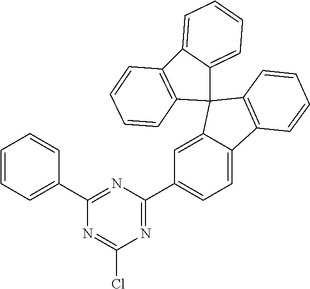 [SMILES: Clc1nc(-c2ccccc2)nc(-c2ccc3c(c2)C2(c4ccccc4-c4ccccc42)c2ccccc2-3)n1] |
| | CAS: 1870820-31-7 |
| 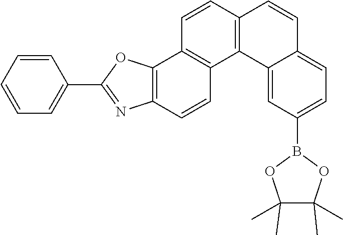 [SMILES: CC1(C)OB(c2ccc3ccc4ccc5c(ccc6nc(-c7ccccc7)oc65)c4c3c2)OC1(C)C] | 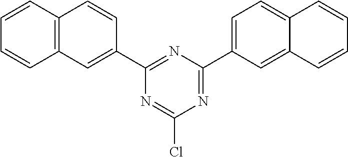 [SMILES: Clc1nc(-c2ccc3ccccc3c2)nc(-c2ccc3ccccc3c2)n1] CAS: 1247124-77-1 |
| 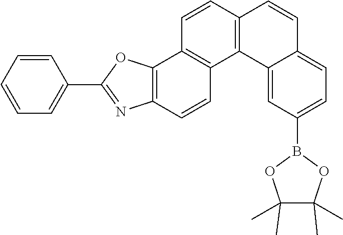 [SMILES: CC1(C)OB(c2ccc3ccc4ccc5c(ccc6nc(-c7ccccc7)oc65)c4c3c2)OC1(C)C] | 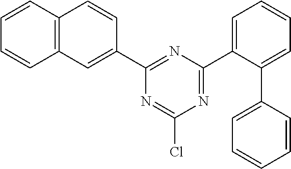 [SMILES: Clc1nc(-c2ccc3ccccc3c2)nc(-c2ccccc2-c2ccccc2)n1] CAS: 2440106-70-5 |
| 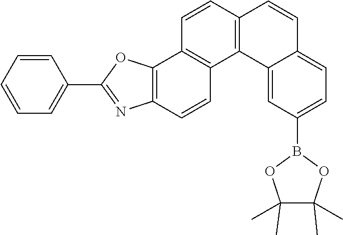 [SMILES: CC1(C)OB(c2ccc3ccc4ccc5c(ccc6nc(-c7ccccc7)oc65)c4c3c2)OC1(C)C] | 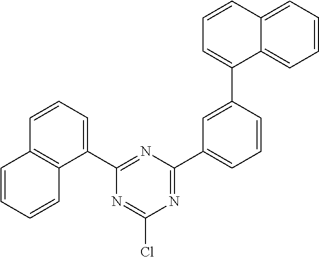 [SMILES: Clc1nc(-c2cccc(-c3cccc4ccccc34)c2)nc(-c2cccc3ccccc23)n1] |
| | CAS: 1858269-61-0 |
| 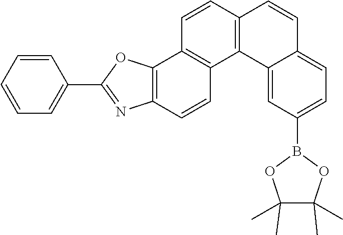 [SMILES: CC1(C)OB(c2ccc3ccc4ccc5c(ccc6nc(-c7ccccc7)oc65)c4c3c2)OC1(C)C] | 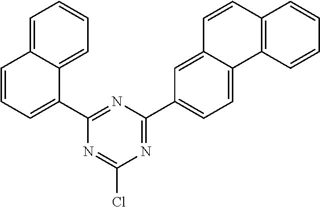 [SMILES: Clc1nc(-c2ccc3c(ccc4ccccc43)c2)nc(-c2cccc3ccccc23)n1] CAS: 2691136-17-9 |
| 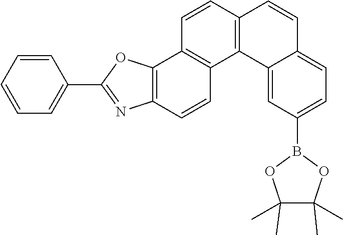 [SMILES: CC1(C)OB(c2ccc3ccc4ccc5c(ccc6nc(-c7ccccc7)oc65)c4c3c2)OC1(C)C] | 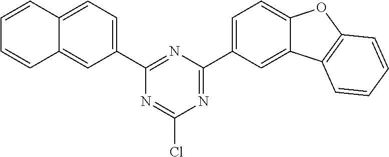 [SMILES: Clc1nc(-c2ccc3ccccc3c2)nc(-c2ccc3oc4ccccc4c3c2)n1] CAS: 2412580-40-4 |
| 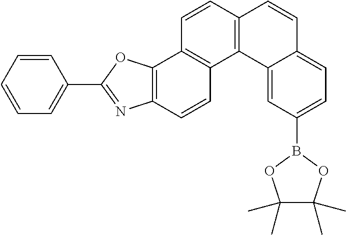 [SMILES: CC1(C)OB(c2ccc3ccc4ccc5c(ccc6nc(-c7ccccc7)oc65)c4c3c2)OC1(C)C] | 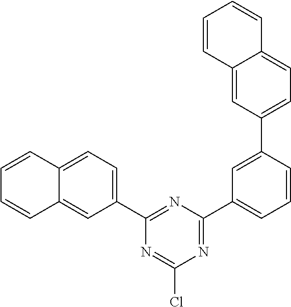 [SMILES: Clc1nc(-c2cccc(-c3ccc4ccccc4c3)c2)nc(-c2ccc3ccccc3c2)n1] |
| | CAS: 2822576-97-4 |
| 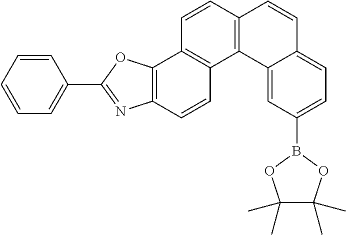 [SMILES: CC1(C)OB(c2ccc3ccc4ccc5c(ccc6nc(-c7ccccc7)oc65)c4c3c2)OC1(C)C] | 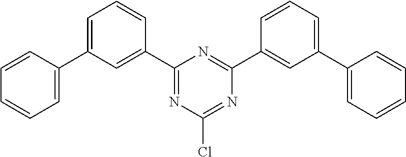 [SMILES: Clc1nc(-c2cccc(-c3ccccc3)c2)nc(-c2cccc(-c3ccccc3)c2)n1] CAS: 1205748-61-3 |
| 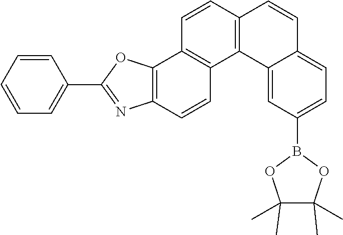 [SMILES: CC1(C)OB(c2ccc3ccc4ccc5c(ccc6nc(-c7ccccc7)oc65)c4c3c2)OC1(C)C] | 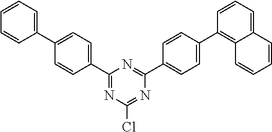 [SMILES: Clc1nc(-c2ccc(-c3ccccc3)cc2)nc(-c2ccc(-c3cccc4ccccc34)cc2)n1] CAS: 2468040-63-1 |
| 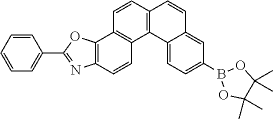 [SMILES: CC1(C)OB(c2ccc3c(ccc4ccc5c(ccc6nc(-c7ccccc7)oc65)c43)c2)OC1(C)C] | 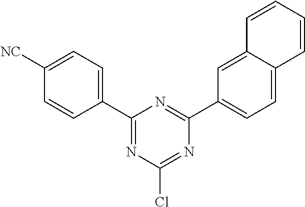 [SMILES: N#Cc1ccc(-c2nc(Cl)nc(-c3ccc4ccccc4c3)n2)cc1] |
| | CAS: 2568464-79-7 |
| 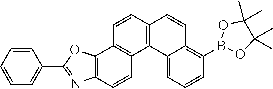 [SMILES: CC1(C)OB(c2cccc3c2ccc2ccc4c(ccc5nc(-c6ccccc6)oc54)c23)OC1(C)C] | 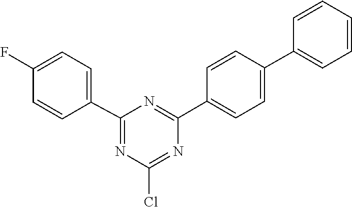 [SMILES: Fc1ccc(-c2nc(Cl)nc(-c3ccc(-c4ccccc4)cc3)n2)cc1] |
| | CAS: 2737218-48-1 |
| 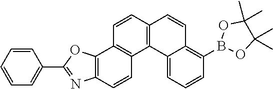 [SMILES: CC1(C)OB(c2cccc3c2ccc2ccc4c(ccc5nc(-c6ccccc6)oc54)c23)OC1(C)C] | 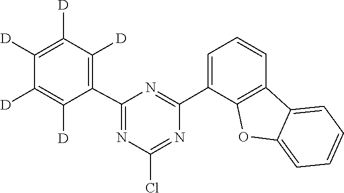 [SMILES: [2H]c1c([2H])c([2H])c(-c2nc(Cl)nc(-c3cccc4c3oc3ccccc34)n2)c([2H])c1[2H]] |
| | CAS: 2408705-74-6 |
| 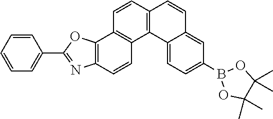 [SMILES: CC1(C)OB(c2ccc3c(ccc4ccc5c(ccc6nc(-c7ccccc7)oc65)c43)c2)OC1(C)C] | 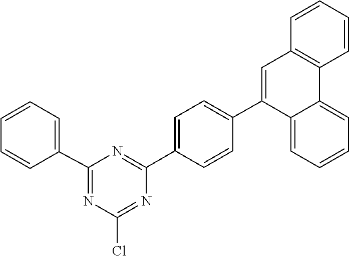 [SMILES: Clc1nc(-c2ccccc2)nc(-c2ccc(-c3cc4ccccc4c4ccccc34)cc2)n1] |
| | CAS: 2305965-24-4 |
| 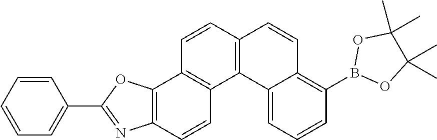 [SMILES: CC1(C)OB(c2cccc3c2ccc2ccc4c(ccc5nc(-c6ccccc6)oc54)c23)OC1(C)C] | 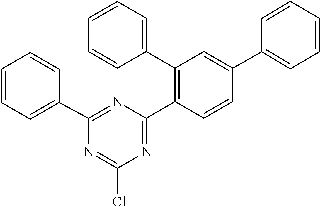 [SMILES: Clc1nc(-c2ccccc2)nc(-c2ccc(-c3ccccc3)cc2-c2ccccc2)n1] CAS: 2359655-66-4 |
| 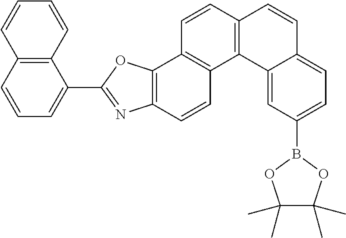 [SMILES: CC1(C)OB(c2ccc3ccc4ccc5c(ccc6nc(-c7cccc8ccccc78)oc65)c4c3c2)OC1(C)C] | 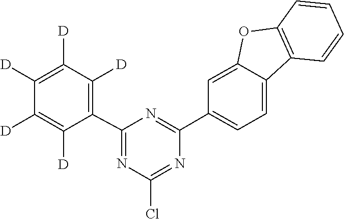 [SMILES: [2H]c1c([2H])c([2H])c(-c2nc(Cl)nc(-c3ccc4c(c3)oc3ccccc34)n2)c([2H])c1[2H]] CAS: 2226747-65-3 |
| 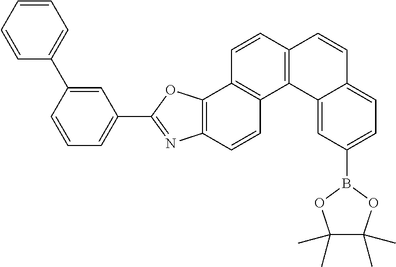 [SMILES: CC1(C)OB(c2ccc3ccc4ccc5c(ccc6nc(-c7cccc(-c8ccccc8)c7)oc65)c4c3c2)OC1(C)C] | 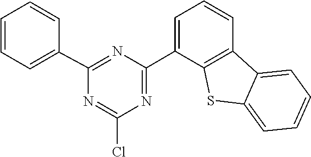 [SMILES: Clc1nc(-c2ccccc2)nc(-c2cccc3c2sc2ccccc23)n1] CAS: 1476735-48-4 |
| 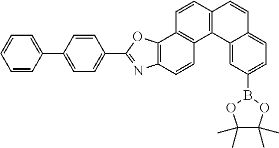 [SMILES: CC1(C)OB(c2ccc3ccc4ccc5c(ccc6nc(-c7ccc(-c8ccccc8)cc7)oc65)c4c3c2)OC1(C)C] | 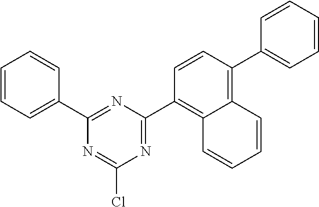 [SMILES: Clc1nc(-c2ccccc2)nc(-c2ccc(-c3ccccc3)c3ccccc23)n1] |
| | CAS: 2418702-67-5 |
| 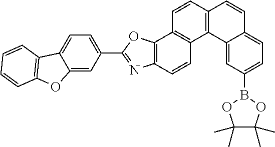 [SMILES: CC1(C)OB(c2ccc3ccc4ccc5c(ccc6nc(-c7ccc8c(c7)oc7ccccc78)oc65)c4c3c2)OC1(C)C] | 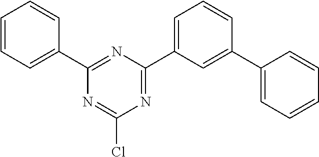 [SMILES: Clc1nc(-c2ccccc2)nc(-c2cccc(-c3ccccc3)c2)n1] CAS: 1689576-03-1 |
| 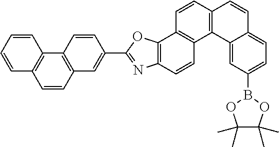 [SMILES: CC1(C)OB(c2ccc3ccc4ccc5c(ccc6nc(-c7ccc8c(ccc9ccccc98)c7)oc65)c4c3c2)OC1(C)C] | 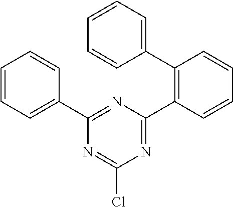 [SMILES: Clc1nc(-c2ccccc2)nc(-c2ccccc2-c2ccccc2)n1] |
| | CAS: 1835683-68-5 |
| 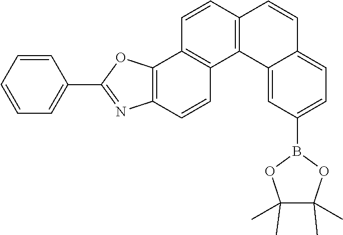 [SMILES: CC1(C)OB(c2ccc3ccc4ccc5c(ccc6nc(-c7ccccc7)oc65)c4c3c2)OC1(C)C] | 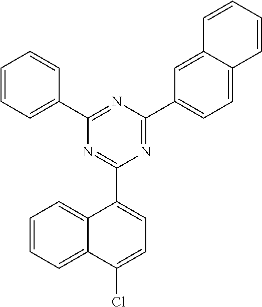 [SMILES: Clc1ccc(-c2nc(-c3ccccc3)nc(-c3ccc4ccccc4c3)n2)c2ccccc12] |
| | CAS: 2639900-27-7 |
| 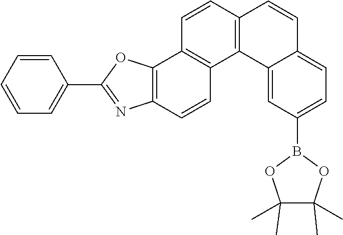 [SMILES: CC1(C)OB(c2ccc3ccc4ccc5c(ccc6nc(-c7ccccc7)oc65)c4c3c2)OC1(C)C] | 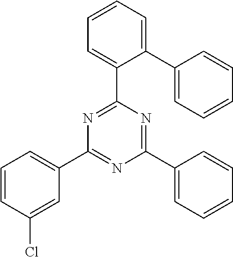 [SMILES: Clc1cccc(-c2nc(-c3ccccc3)nc(-c3ccccc3-c3ccccc3)n2)c1] |
| | CAS: 2246950-40-1 |

Synthesis of the compounds in the present application

| Reactant G | Compound structure and No. |
|---|---|
| 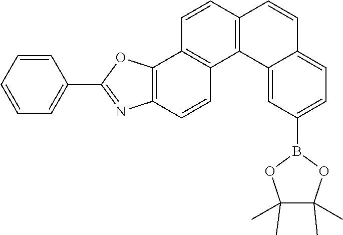 [SMILES: CC1(C)OB(c2ccc3ccc4ccc5c(ccc6nc(-c7ccccc7)oc65)c4c3c2)OC1(C)C] | 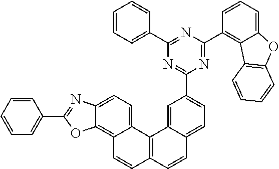 [SMILES: c1ccc(-c2nc(-c3ccc4ccc5ccc6c(ccc7nc(-c8ccccc8)oc76)c5c4c3)nc(-c3cccc4oc5ccccc5c34)n2)cc1] 9 |
| 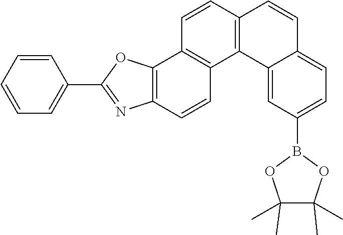 [SMILES: CC1(C)OB(c2ccc3ccc4ccc5c(ccc6nc(-c7ccccc7)oc65)c4c3c2)OC1(C)C] | 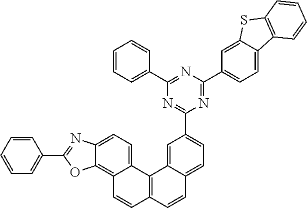 [SMILES: c1ccc(-c2nc(-c3ccc4c(c3)sc3ccccc34)nc(-c3ccc4ccc5ccc6c(ccc7nc(-c8ccccc8)oc76)c5c4c3)n2)cc1] 11 |
| 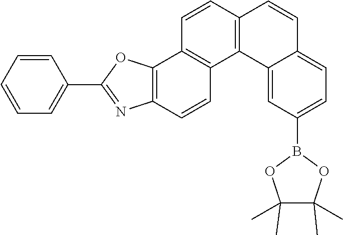 [SMILES: CC1(C)OB(c2ccc3ccc4ccc5c(ccc6nc(-c7ccccc7)oc65)c4c3c2)OC1(C)C] | 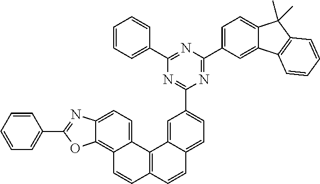 [SMILES: CC1(C)c2ccccc2-c2cc(-c3nc(-c4ccccc4)nc(-c4ccc5ccc6ccc7c(ccc8nc(-c9ccccc9)oc87)c6c5c4)n3)ccc21] 15 |
| 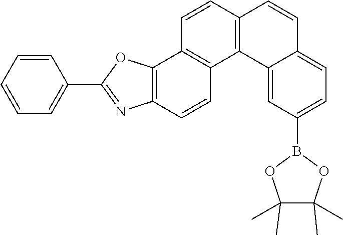 [SMILES: CC1(C)OB(c2ccc3ccc4ccc5c(ccc6nc(-c7ccccc7)oc65)c4c3c2)OC1(C)C] | 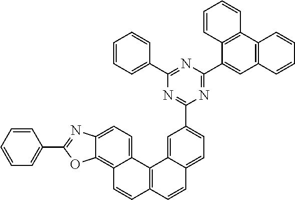 [SMILES: c1ccc(-c2nc(-c3ccc4ccc5ccc6c(ccc7nc(-c8ccccc8)oc76)c5c4c3)nc(-c3cc4ccccc4c4ccccc34)n2)cc1] 19 |
| 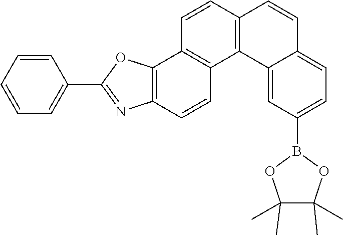 [SMILES: CC1(C)OB(c2ccc3ccc4ccc5c(ccc6nc(-c7ccccc7)oc65)c4c3c2)OC1(C)C] | 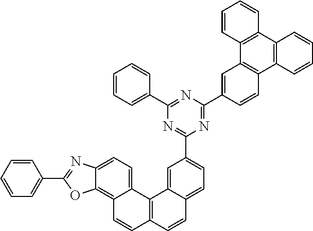 [SMILES: c1ccc(-c2nc(-c3ccc4c5ccccc5c5ccccc5c4c3)nc(-c3ccc4ccc5ccc6c(ccc7nc(-c8ccccc8)oc76)c5c4c3)n2)cc1] |
| | 22 |
| 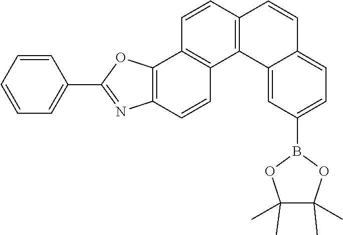 [SMILES: CC1(C)OB(c2ccc3ccc4ccc5c(ccc6nc(-c7ccccc7)oc65)c4c3c2)OC1(C)C] | 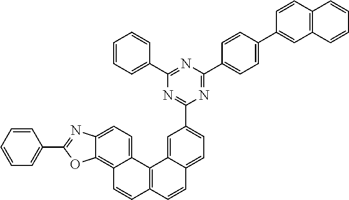 [SMILES: c1ccc(-c2nc(-c3ccc(-c4ccc5ccccc5c4)cc3)nc(-c3ccc4ccc5ccc6c(ccc7nc(-c8ccccc8)oc76)c5c4c3)n2)cc1] 24 |
| 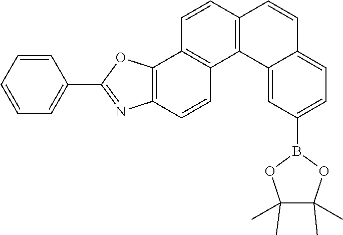 [SMILES: CC1(C)OB(c2ccc3ccc4ccc5c(ccc6nc(-c7ccccc7)oc65)c4c3c2)OC1(C)C] | 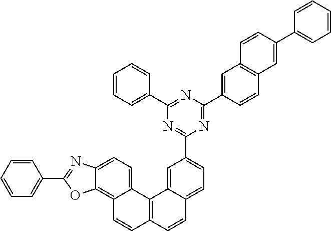 [SMILES: c1ccc(-c2ccc3cc(-c4nc(-c5ccccc5)nc(-c5ccc6ccc7ccc8c(ccc9nc(-c%10ccccc%10)oc98)c7c6c5)n4)ccc3c2)cc1] |
| | 26 |
| 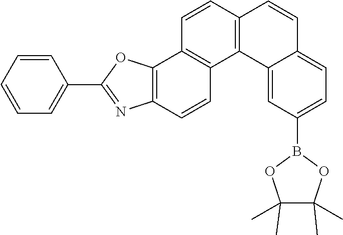 [SMILES: CC1(C)OB(c2ccc3ccc4ccc5c(ccc6nc(-c7ccccc7)oc65)c4c3c2)OC1(C)C] | 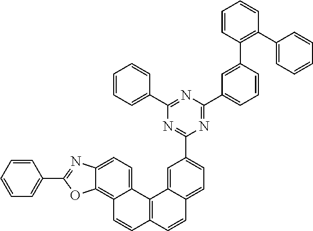 [SMILES: c1ccc(-c2nc(-c3cccc(-c4ccccc4-c4ccccc4)c3)nc(-c3ccc4ccc5ccc6c(ccc7nc(-c8ccccc8)oc76)c5c4c3)n2)cc1] |
| | 31 |
| 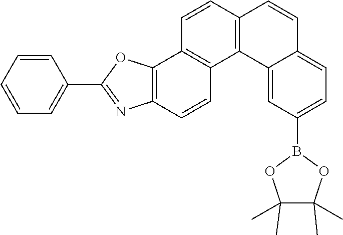 [SMILES: CC1(C)OB(c2ccc3ccc4ccc5c(ccc6nc(-c7ccccc7)oc65)c4c3c2)OC1(C)C] | 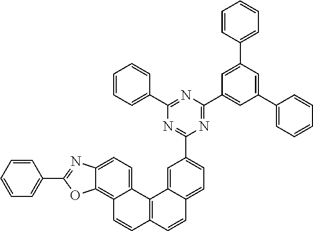 [SMILES: c1ccc(-c2cc(-c3ccccc3)cc(-c3nc(-c4ccccc4)nc(-c4ccc5ccc6ccc7c(ccc8nc(-c9ccccc9)oc87)c6c5c4)n3)c2)cc1] |
| | 35 |
| 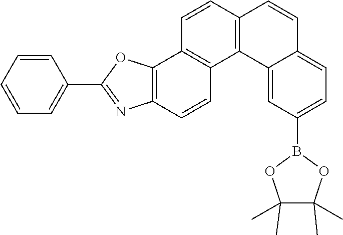 [SMILES: CC1(C)OB(c2ccc3ccc4ccc5c(ccc6nc(-c7ccccc7)oc65)c4c3c2)OC1(C)C] | 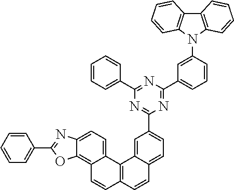 [SMILES: c1ccc(-c2nc(-c3cccc(-n4c5ccccc5c5ccccc54)c3)nc(-c3ccc4ccc5ccc6c(ccc7nc(-c8ccccc8)oc76)c5c4c3)n2)cc1] 41 |
| 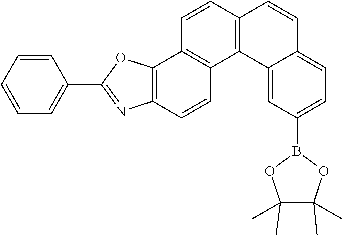 [SMILES: CC1(C)OB(c2ccc3ccc4ccc5c(ccc6nc(-c7ccccc7)oc65)c4c3c2)OC1(C)C] | 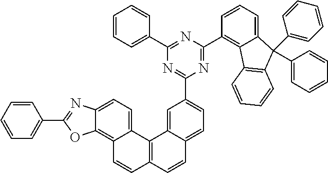 [SMILES: c1ccc(-c2nc(-c3ccc4ccc5ccc6c(ccc7nc(-c8ccccc8)oc76)c5c4c3)nc(-c3cccc4c3-c3ccccc3C4(c3ccccc3)c3ccccc3)n2)cc1] 43 |
| 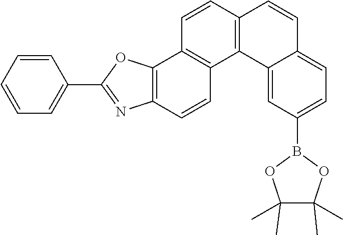 [SMILES: CC1(C)OB(c2ccc3ccc4ccc5c(ccc6nc(-c7ccccc7)oc65)c4c3c2)OC1(C)C] | 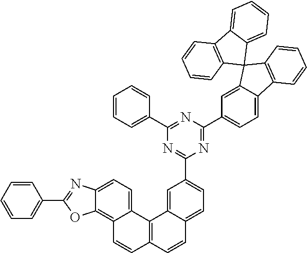 [SMILES: c1ccc(-c2nc(-c3ccc4c(c3)C3(c5ccccc5-c5ccccc53)c3ccccc3-4)nc(-c3ccc4ccc5ccc6c(ccc7nc(-c8ccccc8)oc76)c5c4c3)n2)cc1] 46 |
| 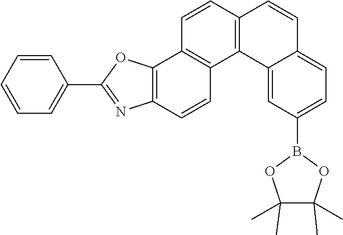 [SMILES: CC1(C)OB(c2ccc3ccc4ccc5c(ccc6nc(-c7ccccc7)oc65)c4c3c2)OC1(C)C] | 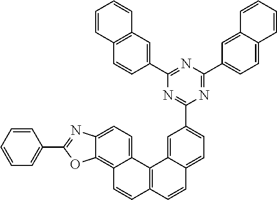 [SMILES: c1ccc(-c2nc3ccc4c(ccc5ccc6ccc(-c7nc(-c8ccc9ccccc9c8)nc(-c8ccc9ccccc9c8)n7)cc6c54)c3o2)cc1] 49 |
| 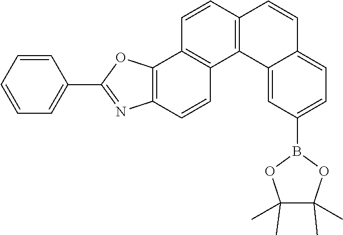 [SMILES: CC1(C)OB(c2ccc3ccc4ccc5c(ccc6nc(-c7ccccc7)oc65)c4c3c2)OC1(C)C] | 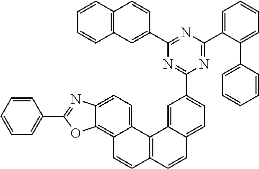 [SMILES: c1ccc(-c2nc3ccc4c(ccc5ccc6ccc(-c7nc(-c8ccc9ccccc9c8)nc(-c8ccccc8-c8ccccc8)n7)cc6c54)c3o2)cc1] 53 |
| 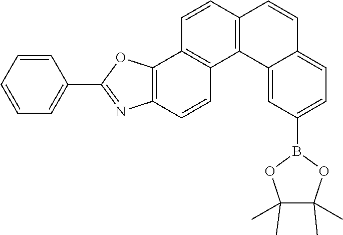 [SMILES: CC1(C)OB(c2ccc3ccc4ccc5c(ccc6nc(-c7ccccc7)oc65)c4c3c2)OC1(C)C] | 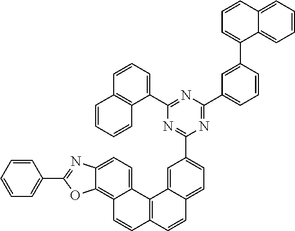 [SMILES: c1ccc(-c2nc3ccc4c(ccc5ccc6ccc(-c7nc(-c8cccc(-c9cccc%10ccccc9%10)c8)nc(-c8cccc9ccccc89)n7)cc6c54)c3o2)cc1] |
| | 60 |
| 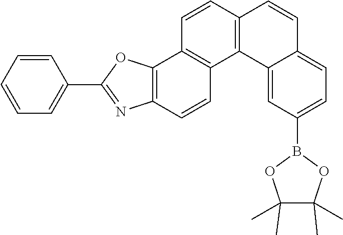 [SMILES: CC1(C)OB(c2ccc3ccc4ccc5c(ccc6nc(-c7ccccc7)oc65)c4c3c2)OC1(C)C] | 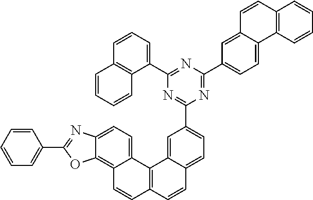 [SMILES: c1ccc(-c2nc3ccc4c(ccc5ccc6ccc(-c7nc(-c8ccc9c(ccc%10ccccc%109)c8)nc(-c8cccc9ccccc89)n7)cc6c54)c3o2)cc1] 64 |
| 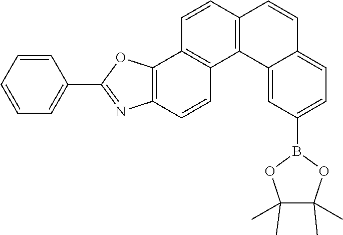 [SMILES: CC1(C)OB(c2ccc3ccc4ccc5c(ccc6nc(-c7ccccc7)oc65)c4c3c2)OC1(C)C] | 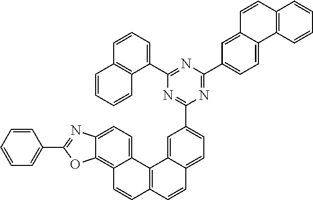 [SMILES: c1ccc(-c2nc3ccc4c(ccc5ccc6ccc(-c7nc(-c8ccc9c(ccc%10ccccc%109)c8)nc(-c8cccc9ccccc89)n7)cc6c54)c3o2)cc1] 69 |
| 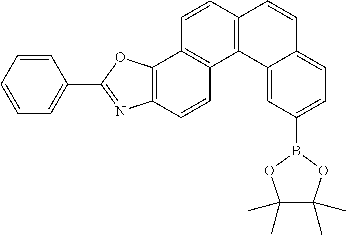 [SMILES: CC1(C)OB(c2ccc3ccc4ccc5c(ccc6nc(-c7ccccc7)oc65)c4c3c2)OC1(C)C] | 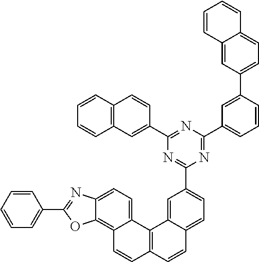 [SMILES: c1ccc(-c2nc3ccc4c(ccc5ccc6ccc(-c7nc(-c8cccc(-c9ccc%10ccccc%10c9)c8)nc(-c8ccc9ccccc9c8)n7)cc6c54)c3o2)cc1] |
| | 77 |
| 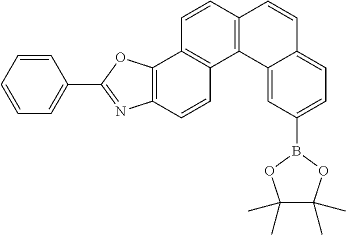 [SMILES: CC1(C)OB(c2ccc3ccc4ccc5c(ccc6nc(-c7ccccc7)oc65)c4c3c2)OC1(C)C] | 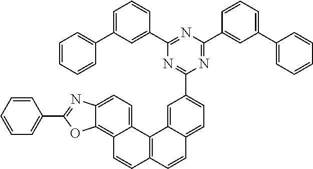 [SMILES: c1ccc(-c2cccc(-c3nc(-c4cccc(-c5ccccc5)c4)nc(-c4ccc5ccc6ccc7c(ccc8nc(-c9ccccc9)oc87)c6c5c4)n3)c2)cc1] 86 |
| 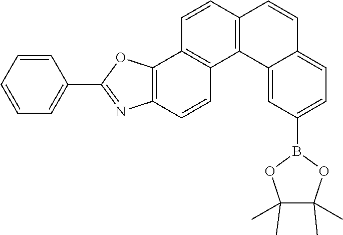 [SMILES: CC1(C)OB(c2ccc3ccc4ccc5c(ccc6nc(-c7ccccc7)oc65)c4c3c2)OC1(C)C] | 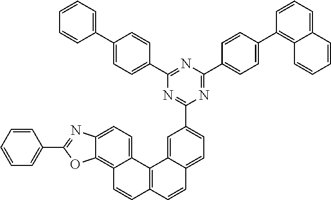 [SMILES: c1ccc(-c2ccc(-c3nc(-c4ccc(-c5cccc6ccccc56)cc4)nc(-c4ccc5ccc6ccc7c(ccc8nc(-c9ccccc9)oc87)c6c5c4)n3)cc2)cc1] 95 |
| 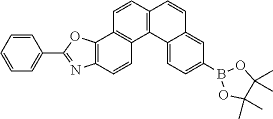 [SMILES: CC1(C)OB(c2ccc3c(ccc4ccc5c(ccc6nc(-c7ccccc7)oc65)c43)c2)OC1(C)C] | 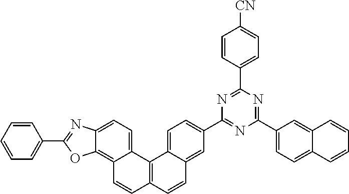 [SMILES: N#Cc1ccc(-c2nc(-c3ccc4ccccc4c3)nc(-c3ccc4c(ccc5ccc6c(ccc7nc(-c8ccccc8)oc76)c54)c3)n2)cc1] |
| | 111 |
| 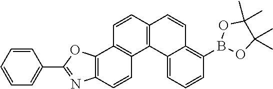 [SMILES: CC1(C)OB(c2cccc3c2ccc2ccc4c(ccc5nc(-c6ccccc6)oc54)c23)OC1(C)C] | 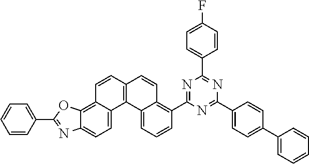 [SMILES: Fc1ccc(-c2nc(-c3ccc(-c4ccccc4)cc3)nc(-c3cccc4c3ccc3ccc5c(ccc6nc(-c7ccccc7)oc65)c34)n2)cc1] |
| | 112 |
| 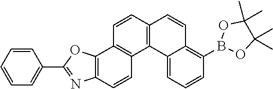 [SMILES: CC1(C)OB(c2cccc3c2ccc2ccc4c(ccc5nc(-c6ccccc6)oc54)c23)OC1(C)C] | 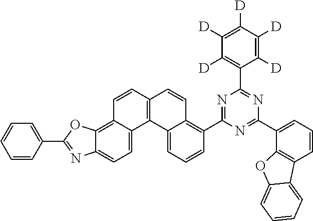 [SMILES: [2H]c1c([2H])c([2H])c(-c2nc(-c3cccc4c3ccc3ccc5c(ccc6nc(-c7ccccc7)oc65)c34)nc(-c3cccc4c3oc3ccccc34)n2)c([2H])c1[2H]] |
| | 118 |
| 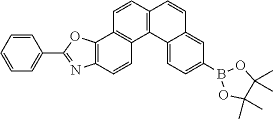 [SMILES: CC1(C)OB(c2ccc3c(ccc4ccc5c(ccc6nc(-c7ccccc7)oc65)c43)c2)OC1(C)C] | 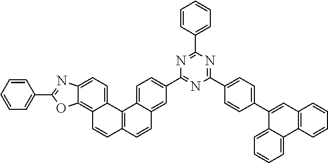 [SMILES: c1ccc(-c2nc(-c3ccc(-c4cc5ccccc5c5ccccc45)cc3)nc(-c3ccc4c(ccc5ccc6c(ccc7nc(-c8ccccc8)oc76)c54)c3)n2)cc1] |
| | 127 |
| 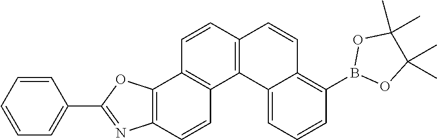 [SMILES: CC1(C)OB(c2cccc3c2ccc2ccc4c(ccc5nc(-c6ccccc6)oc54)c23)OC1(C)C] | 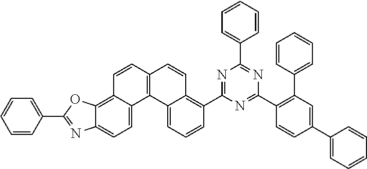 [SMILES: c1ccc(-c2ccc(-c3nc(-c4ccccc4)nc(-c4cccc5c4ccc4ccc6c(ccc7nc(-c8ccccc8)oc76)c45)n3)c(-c3ccccc3)c2)cc1] 146 |
| 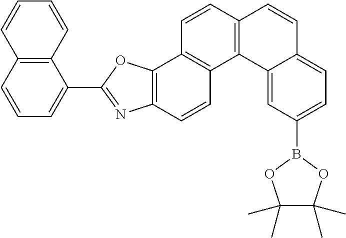 [SMILES: CC1(C)OB(c2ccc3ccc4ccc5c(ccc6nc(-c7cccc8ccccc78)oc65)c4c3c2)OC1(C)C] | 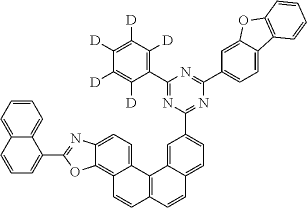 [SMILES: [2H]c1c([2H])c([2H])c(-c2nc(-c3ccc4c(c3)oc3ccccc34)nc(-c3ccc4ccc5ccc6c(ccc7nc(-c8cccc9ccccc89)oc76)c5c4c3)n2)c([2H])c1[2H]] 211 |
| 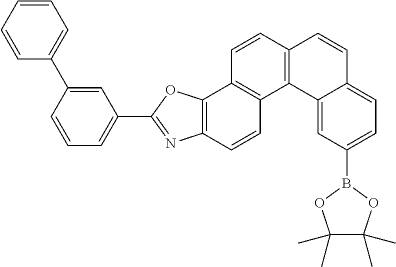 [SMILES: CC1(C)OB(c2ccc3ccc4ccc5c(ccc6nc(-c7cccc(-c8ccccc8)c7)oc65)c4c3c2)OC1(C)C] | 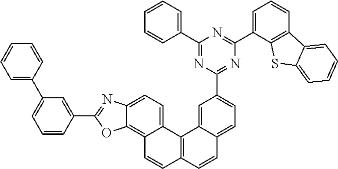 [SMILES: c1ccc(-c2cccc(-c3nc4ccc5c(ccc6ccc7ccc(-c8nc(-c9ccccc9)nc(-c9cccc%10c9sc9ccccc9%10)n8)cc7c65)c4o3)c2)cc1] 214 |
| 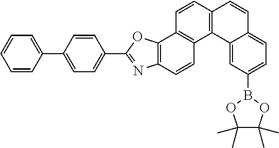 [SMILES: CC1(C)OB(c2ccc3ccc4ccc5c(ccc6nc(-c7ccc(-c8ccccc8)cc7)oc65)c4c3c2)OC1(C)C] | 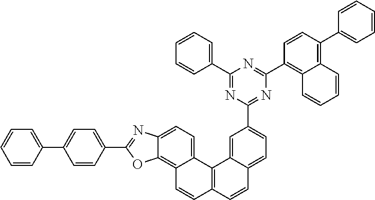 [SMILES: c1ccc(-c2ccc(-c3nc4ccc5c(ccc6ccc7ccc(-c8nc(-c9ccccc9)nc(-c9ccc(-c%10ccccc%10)c%10ccccc9%10)n8)cc7c65)c4o3)cc2)cc1] |
| | 219 |
| 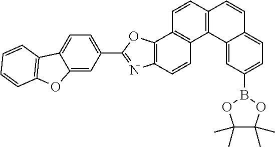 [SMILES: CC1(C)OB(c2ccc3ccc4ccc5c(ccc6nc(-c7ccc8c(c7)oc7ccccc78)oc65)c4c3c2)OC1(C)C] | 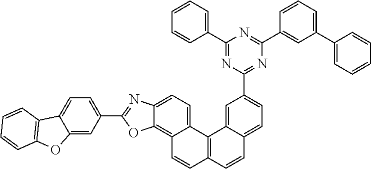 [SMILES: c1ccc(-c2cccc(-c3nc(-c4ccccc4)nc(-c4ccc5ccc6ccc7c(ccc8nc(-c9ccc%10c(c9)oc9ccccc9%10)oc87)c6c5c4)n3)c2)cc1] |
| | 221 |
| 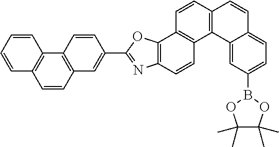 [SMILES: CC1(C)OB(c2ccc3ccc4ccc5c(ccc6nc(-c7ccc8c(ccc9ccccc98)c7)oc65)c4c3c2)OC1(C)C] | 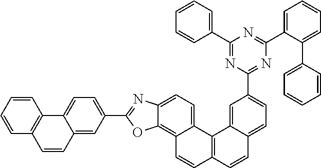 [SMILES: c1ccc(-c2nc(-c3ccc4ccc5ccc6c(ccc7nc(-c8ccc9c(ccc%10ccccc%109)c8)oc76)c5c4c3)nc(-c3ccccc3-c3ccccc3)n2)cc1] |
| | 222 |
| 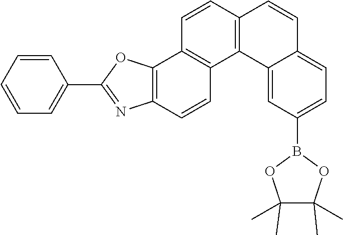 [SMILES: CC1(C)OB(c2ccc3ccc4ccc5c(ccc6nc(-c7ccccc7)oc65)c4c3c2)OC1(C)C] | 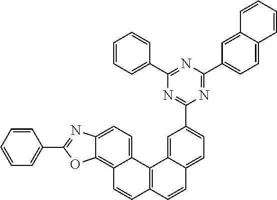 [SMILES: c1ccc(-c2nc(-c3ccc4ccccc4c3)nc(-c3ccc4ccc5ccc6c(ccc7nc(-c8ccccc8)oc76)c5c4c3)n2)cc1] 224 |
| 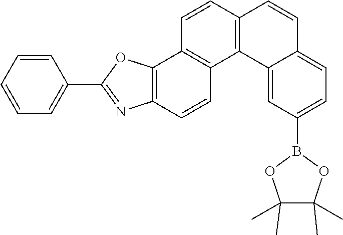 [SMILES: CC1(C)OB(c2ccc3ccc4ccc5c(ccc6nc(-c7ccccc7)oc65)c4c3c2)OC1(C)C] | 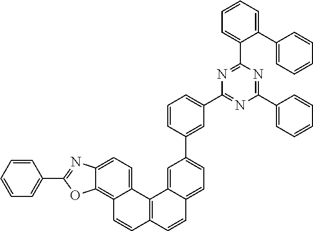 [SMILES: c1ccc(-c2nc(-c3cccc(-c4ccc5ccc6ccc7c(ccc8nc(-c9ccccc9)oc87)c6c5c4)c3)nc(-c3ccccc3-c3ccccc3)n2)cc1] 232 |

Synthesis of the compounds in the present application

| | m/z | Yield |
|---|---|---|
| Reactant G | ([M + H]⁺) | % |
| 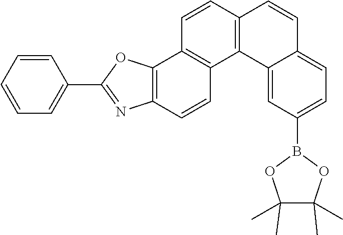 [SMILES: CC1(C)OB(c2ccc3ccc4ccc5c(ccc6nc(-c7ccccc7)oc65)c4c3c2)OC1(C)C] | 667.21 | 62 |
| 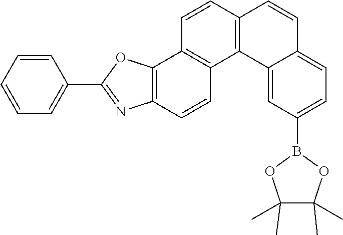 [SMILES: CC1(C)OB(c2ccc3ccc4ccc5c(ccc6nc(-c7ccccc7)oc65)c4c3c2)OC1(C)C] | 683.18 | 54 |
| 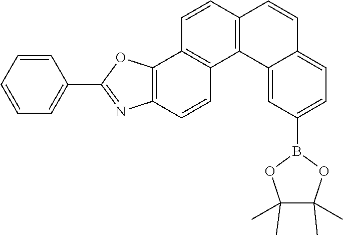 [SMILES: CC1(C)OB(c2ccc3ccc4ccc5c(ccc6nc(-c7ccccc7)oc65)c4c3c2)OC1(C)C] | 693.26 | 54 |
| 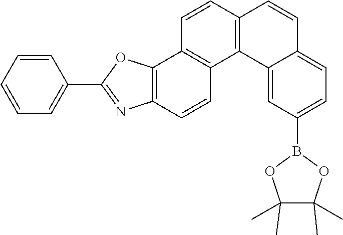 [SMILES: CC1(C)OB(c2ccc3ccc4ccc5c(ccc6nc(-c7ccccc7)oc65)c4c3c2)OC1(C)C] | 677.23 | 58 |
| 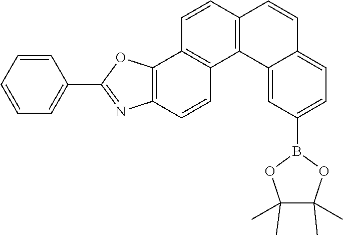 [SMILES: CC1(C)OB(c2ccc3ccc4ccc5c(ccc6nc(-c7ccccc7)oc65)c4c3c2)OC1(C)C] | 727.25 | 51 |
| 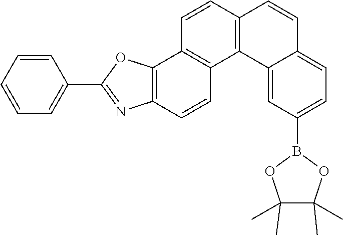 [SMILES: CC1(C)OB(c2ccc3ccc4ccc5c(ccc6nc(-c7ccccc7)oc65)c4c3c2)OC1(C)C] | 703.25 | 65 |
| 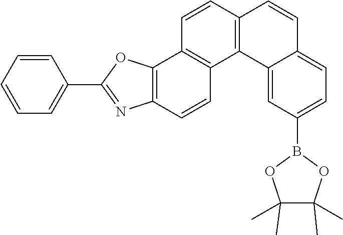 [SMILES: CC1(C)OB(c2ccc3ccc4ccc5c(ccc6nc(-c7ccccc7)oc65)c4c3c2)OC1(C)C] | 703.25 | 57 |
| 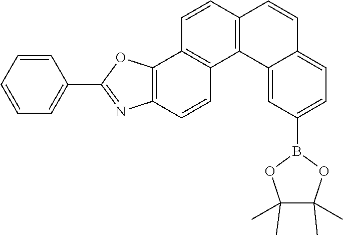 [SMILES: CC1(C)OB(c2ccc3ccc4ccc5c(ccc6nc(-c7ccccc7)oc65)c4c3c2)OC1(C)C] | 729.26 | 53 |
| 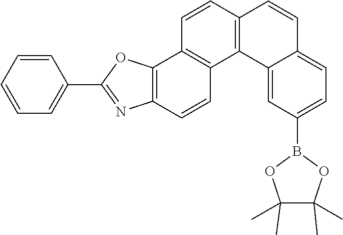 [SMILES: CC1(C)OB(c2ccc3ccc4ccc5c(ccc6nc(-c7ccccc7)oc65)c4c3c2)OC1(C)C] | 729.26 | 50 |
| 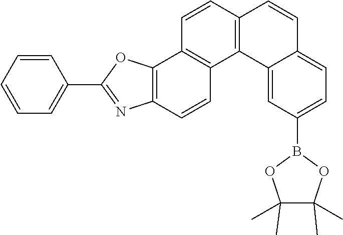 [SMILES: CC1(C)OB(c2ccc3ccc4ccc5c(ccc6nc(-c7ccccc7)oc65)c4c3c2)OC1(C)C] | 741.26 | 58 |
| 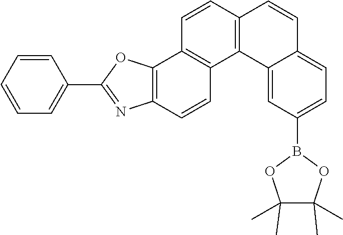 [SMILES: CC1(C)OB(c2ccc3ccc4ccc5c(ccc6nc(-c7ccccc7)oc65)c4c3c2)OC1(C)C] | 817.30 | 58 |
| 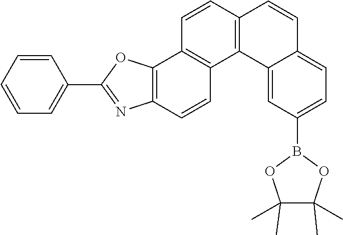 [SMILES: CC1(C)OB(c2ccc3ccc4ccc5c(ccc6nc(-c7ccccc7)oc65)c4c3c2)OC1(C)C] | 815.28 | 65 |
| 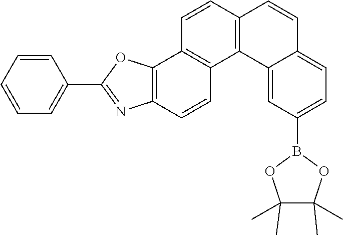 [SMILES: CC1(C)OB(c2ccc3ccc4ccc5c(ccc6nc(-c7ccccc7)oc65)c4c3c2)OC1(C)C] | 677.23 | 57 |
| 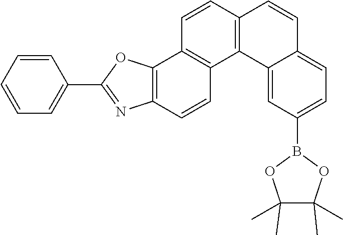 [SMILES: CC1(C)OB(c2ccc3ccc4ccc5c(ccc6nc(-c7ccccc7)oc65)c4c3c2)OC1(C)C] | 704.25 | 63 |
| 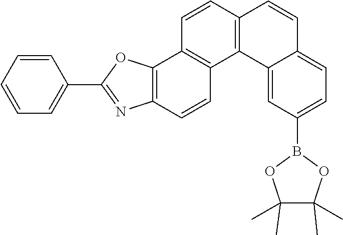 [SMILES: CC1(C)OB(c2ccc3ccc4ccc5c(ccc6nc(-c7ccccc7)oc65)c4c3c2)OC1(C)C] | 753.26 | 65 |
| 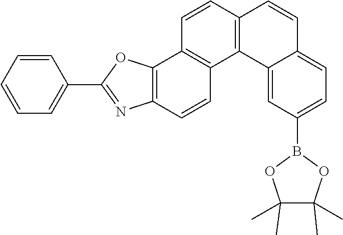 [SMILES: CC1(C)OB(c2ccc3ccc4ccc5c(ccc6nc(-c7ccccc7)oc65)c4c3c2)OC1(C)C] | 727.25 | 65 |
| 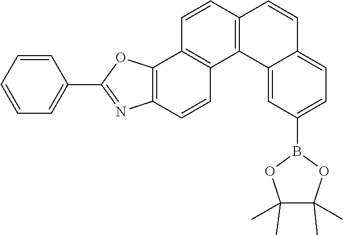 [SMILES: CC1(C)OB(c2ccc3ccc4ccc5c(ccc6nc(-c7ccccc7)oc65)c4c3c2)OC1(C)C] | 717.23 | 52 |
| 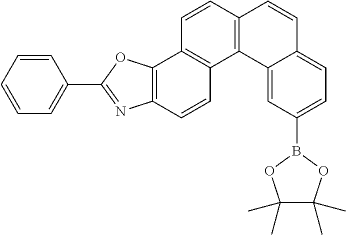 [SMILES: CC1(C)OB(c2ccc3ccc4ccc5c(ccc6nc(-c7ccccc7)oc65)c4c3c2)OC1(C)C] | 753.26 | 57 |
| 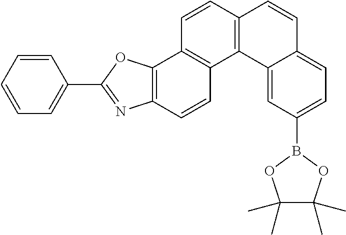 [SMILES: CC1(C)OB(c2ccc3ccc4ccc5c(ccc6nc(-c7ccccc7)oc65)c4c3c2)OC1(C)C] | 729.26 | 51 |
| 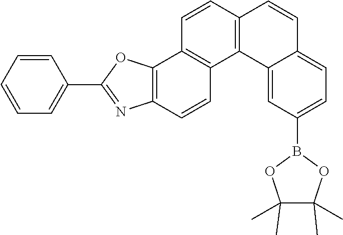 [SMILES: CC1(C)OB(c2ccc3ccc4ccc5c(ccc6nc(-c7ccccc7)oc65)c4c3c2)OC1(C)C] | 779.28 | 57 |
| 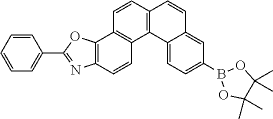 [SMILES: CC1(C)OB(c2ccc3c(ccc4ccc5c(ccc6nc(-c7ccccc7)oc65)c43)c2)OC1(C)C] | 652.21 | 59 |
| 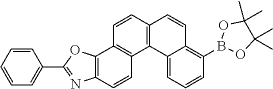 [SMILES: CC1(C)OB(c2cccc3c2ccc2ccc4c(ccc5nc(-c6ccccc6)oc54)c23)OC1(C)C] | 671.22 | 56 |
| 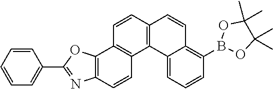 [SMILES: CC1(C)OB(c2cccc3c2ccc2ccc4c(ccc5nc(-c6ccccc6)oc54)c23)OC1(C)C] | 672.24 | 59 |
| 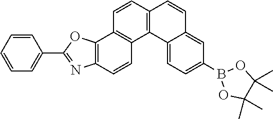 [SMILES: CC1(C)OB(c2ccc3c(ccc4ccc5c(ccc6nc(-c7ccccc7)oc65)c43)c2)OC1(C)C] | 753.26 | 63 |
| 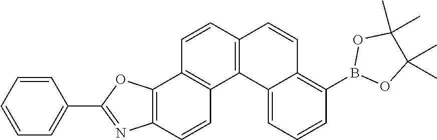 [SMILES: CC1(C)OB(c2cccc3c2ccc2ccc4c(ccc5nc(-c6ccccc6)oc54)c23)OC1(C)C] | 729.26 | 64 |
| 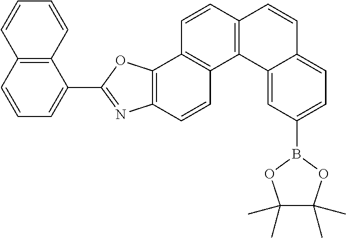 [SMILES: CC1(C)OB(c2ccc3ccc4ccc5c(ccc6nc(-c7cccc8ccccc78)oc65)c4c3c2)OC1(C)C] | 722.26 | 63 |
| 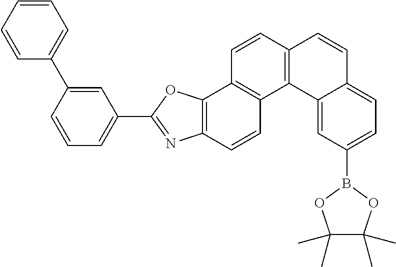 [SMILES: CC1(C)OB(c2ccc3ccc4ccc5c(ccc6nc(-c7cccc(-c8ccccc8)c7)oc65)c4c3c2)OC1(C)C] | 759.22 | 60 |
| 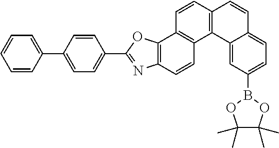 [SMILES: CC1(C)OB(c2ccc3ccc4ccc5c(ccc6nc(-c7ccc(-c8ccccc8)cc7)oc65)c4c3c2)OC1(C)C] | 779.28 | 60 |
| 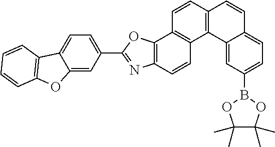 [SMILES: CC1(C)OB(c2ccc3ccc4ccc5c(ccc6nc(-c7ccc8c(c7)oc7ccccc78)oc65)c4c3c2)OC1(C)C] | 743.24 | 61 |
| 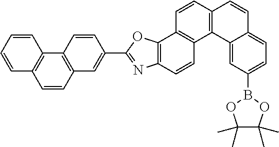 [SMILES: CC1(C)OB(c2ccc3ccc4ccc5c(ccc6nc(-c7ccc8c(ccc9ccccc98)c7)oc65)c4c3c2)OC1(C)C] | 743.28 | 61 |
| 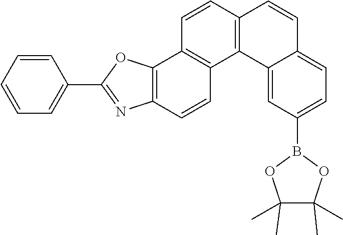 [SMILES: CC1(C)OB(c2ccc3ccc4ccc5c(ccc6nc(-c7ccccc7)oc65)c4c3c2)OC1(C)C] | 753.26 | 58 |
| 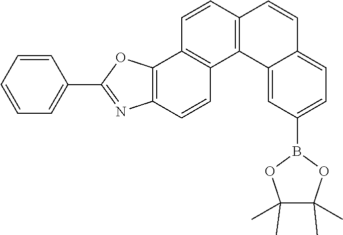 [SMILES: CC1(C)OB(c2ccc3ccc4ccc5c(ccc6nc(-c7ccccc7)oc65)c4c3c2)OC1(C)C] | 729.26 | 58 |

### Characterization of Compounds

NMR data for some of the compounds are shown below:

Compound 69: ¹H-NMR (400 MHz, Methylene-Chloride-D₂) δ ppm 9.51 (s, 1H), 9.21 (d, 1H), 8.83 (sm 1H) 8.81 (s, 1H), 8.69 (d, 1H), 8.61 (d, 1H), 8.53 (d, 1H), 8.34-8.30 (m, 3H), 8.25 (d, 1H), 8.18 (d, 1H), 8.13 (d, 1H) 8.08 (d, 1H), 8.05-7.78 (m, 3H), 7.93 (d, 1H), 7.81 (d, 1H), 7.76 (d, 1H), 7.64 (t, 1H), 7.57 (t, 2H), 7.52-7047 (m, 2H0, 7.40-7.32 (m, 3H).

### Fabrication and Evaluation of Organic Electroluminescent Device

In the following, the organic electroluminescent device of the present application is described in detail by means of examples. However, the following examples are merely exemplary of, and not limiting, the present application.

### Example 1: Red organic electroluminescent device

The anode was first pretreated by the following process: On ITO/Ag/ITO substrates with sequential thickness of 100 ∪/100 ∪/100 ∪, UV-ozone and O₂:N₂ plasma were employed for surface treatment to increase the anode work function. Alternatively, organic solvent may be used to clean the ITO substrate to remove impurities and oil contaminants from the ITP substrate surface.

On the experimental substrate (anode), PD and HT-5 were co-evaporated at an evaporation rate ratio of 2%-94% to form a hole injection layer (HIL) with a thickness of 100 Å. Then HT-5 was vacuum evaporation layer the hole injection layer to form a hole transport layer with a thickness of 1065 Å.

The compound HT-2 was vacuum-evaporated on the hole transport layer to form a hole adjustment layer with a thickness of 890 Å.

Compound 4, RH-P, and RD were co-evaporated on the hole adjustment layer at an evaporation rate ratio of 49%:49%:2% to form an organic light-emitting layer (EML) with a thickness of 400 Å.

On the organic light-emitting layer, the compounds ET-1 and LiQ were co-evaporated at an evaporation rate ratio of 1:1 to form an electron transport layer (ETL) with a thickness of 350 Å. Yb is evaporated on the electron transport layer to form an electron injection layer (EIL) with a thickness of 10 Å. Then, magnesium (Mg) and silver (Ag) were mixed at an evaporation rate ratio of 1:9 and vacuum-evaporated on the electron injection layer to form a cathode with a thickness of 130 Å.

In addition, Compound CP-1 was vacuum evaporated on the top of cathode to form a capping layer (CPL) with a thickness of 800 Å, thereby completing the fabrication of red organic electroluminescent device.

### Examples 2-33

An organic electroluminescent device was prepared using the same method as in Example 1 except that Compounds in Table 6 below was used instead of Compound 4 in Example 1 in the fabrication of the light-emitting layer.

### Comparative Examples 1-3

An organic electroluminescent device was prepared using the same method as in Example 1 except that Compound A, Compound B, or Compound C was used instead of Compound 4 in Example 1 in the fabrication of the light-emitting layer, respectively.

Among them, the structures of the compounds used in preparation of the various examples and comparative examples are as follows:

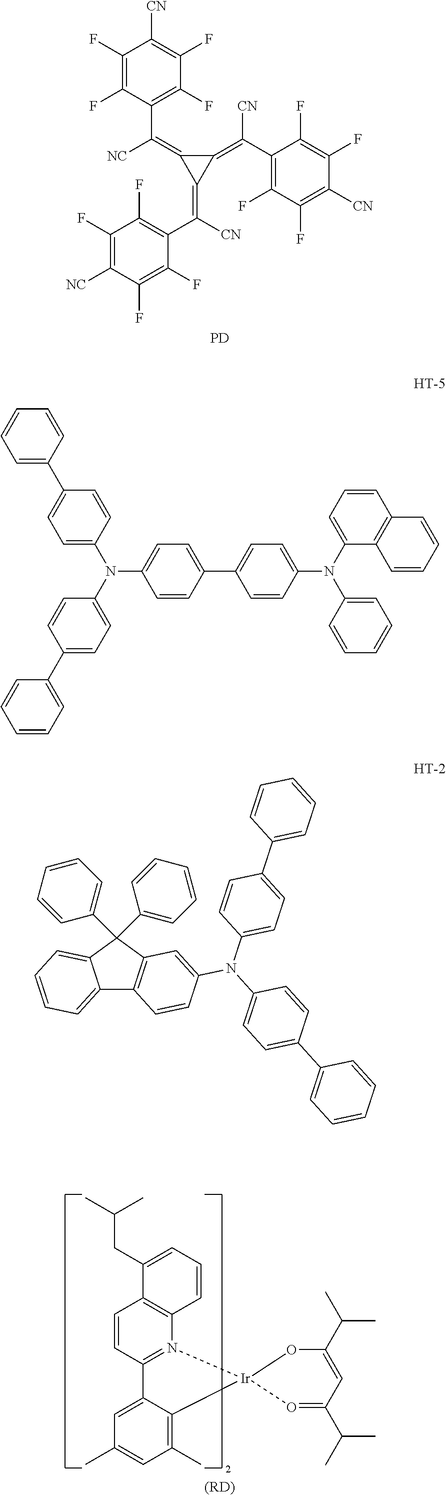

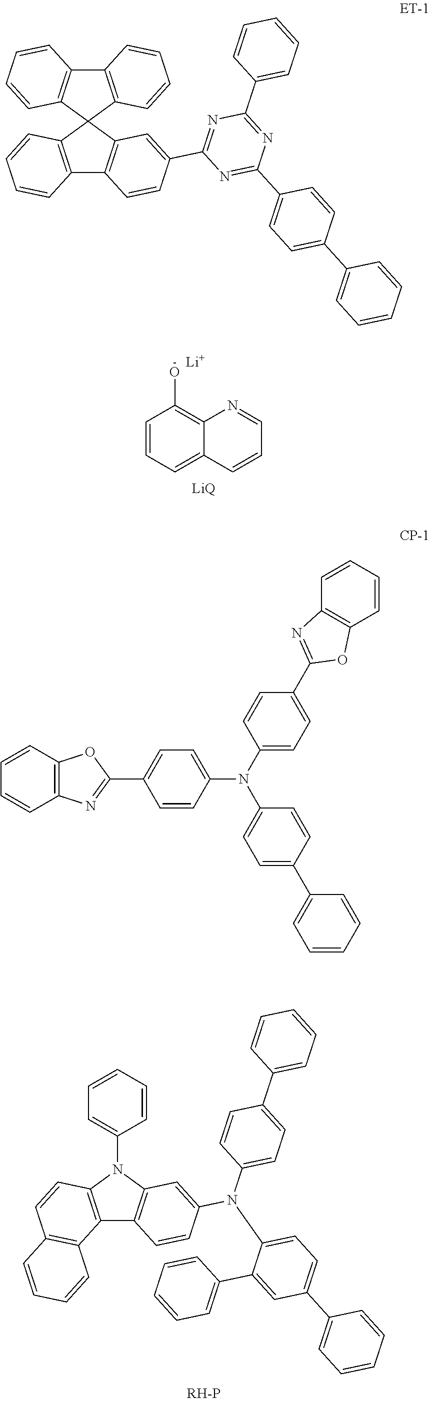

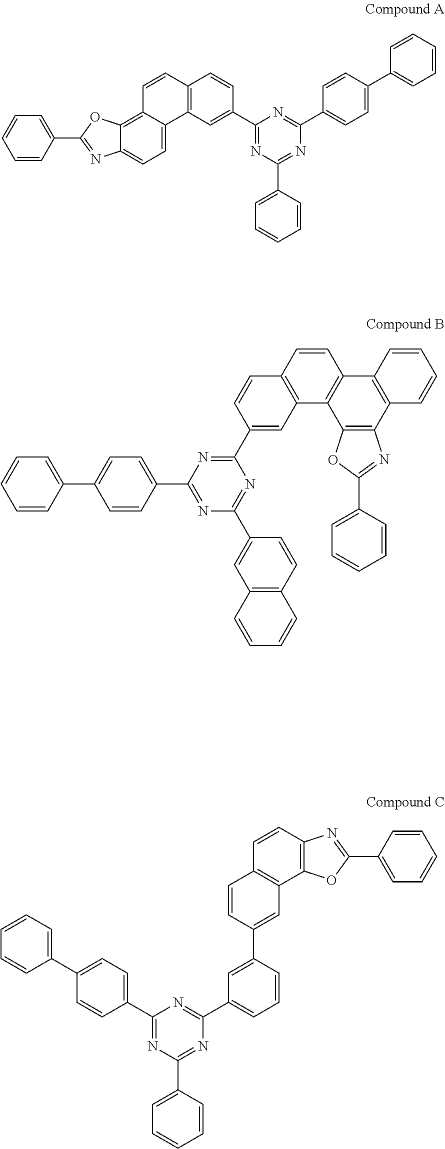 [SMILES: c1ccc(-c2ccc(-c3nc(-c4ccccc4)nc(-c4ccc5ccc6c(ccc7nc(-c8ccccc8)oc76)c5c4)n3)cc2)cc1 ; c1ccc(-c2ccc(-c3nc(-c4ccc5ccccc5c4)nc(-c4ccc5ccc6c7ccccc7c7nc(-c8ccccc8)oc7c6c5c4)n3)cc2)cc1 ; c1ccc(-c2ccc(-c3nc(-c4ccccc4)nc(-c4cccc(-c5ccc6ccc7nc(-c8ccccc8)oc7c6c5)c4)n3)cc2)cc1]

The performances of the red organic electroluminescent devices fabricated in Examples 1 to 33 and Comparative Examples 1 to 3 were tested. Specifically, the IVL performance of the devices was tested at 10 mA/cm², and the T95 device lifetime was tested at 20 mA/cm², and the results of the tests are shown in Table 6.

TABLE 6

| | Light-emitting layer | Operating | | | | |
|---|---|---|---|---|---|---|
| | Compound X: | voltage | | | | T95 (hrs) |
| Example No. | RH-:RD | Volt (V) | Cd/A | CIEx | CIEy | @20 mA/cm² |
| Example 1 | Compound 4 | 3.41 | 59.11 | 0.68 | 0.32 | 507 |
| Example 2 | Compound 9 | 3.41 | 58.03 | 0.68 | 0.32 | 517 |
| Example 3 | Compound 11 | 3.43 | 58.44 | 0.68 | 0.32 | 516 |
| Example 4 | Compound 15 | 3.42 | 58.48 | 0.68 | 0.32 | 527 |
| Example 5 | Compound 19 | 3.41 | 58.12 | 0.68 | 0.32 | 527 |
| Example 6 | Compound 22 | 3.44 | 59.08 | 0.68 | 0.32 | 523 |
| Example 7 | Compound 24 | 3.43 | 58.20 | 0.68 | 0.32 | 522 |
| Example 8 | Compound 26 | 3.46 | 58.81 | 0.68 | 0.32 | 516 |
| Example 9 | Compound 31 | 3.46 | 58.07 | 0.68 | 0.32 | 518 |
| Example 10 | Compound 35 | 3.46 | 59.00 | 0.68 | 0.32 | 515 |
| Example 11 | Compound 41 | 3.43 | 58.40 | 0.68 | 0.32 | 510 |
| Example 12 | Compound 43 | 3.42 | 58.50 | 0.68 | 0.32 | 519 |
| Example 13 | Compound 46 | 3.46 | 58.00 | 0.68 | 0.32 | 522 |
| Example 14 | Compound 49 | 3.46 | 59.10 | 0.68 | 0.32 | 507 |
| Example 15 | Compound 53 | 3.45 | 58.30 | 0.68 | 0.32 | 521 |
| Example 16 | Compound 60 | 3.42 | 58.47 | 0.68 | 0.32 | 525 |
| Example 17 | Compound 64 | 3.41 | 58.29 | 0.68 | 0.32 | 520 |
| Example 18 | Compound 69 | 3.43 | 58.31 | 0.68 | 0.32 | 508 |
| Example 19 | Compound 77 | 3.43 | 59.20 | 0.68 | 0.32 | 526 |
| Example 20 | Compound 86 | 3.42 | 58.39 | 0.68 | 0.32 | 514 |
| Example 21 | Compound 95 | 3.44 | 58.60 | 0.68 | 0.32 | 511 |
| Example 22 | Compound 111 | 3.43 | 58.23 | 0.68 | 0.32 | 509 |
| Example 23 | Compound 112 | 3.45 | 58.80 | 0.68 | 0.32 | 512 |
| Example 24 | Compound 118 | 3.42 | 58.70 | 0.68 | 0.32 | 513 |
| Example 25 | Compound 127 | 3.42 | 58.56 | 0.68 | 0.32 | 521 |
| Example 26 | Compound 146 | 3.41 | 58.41 | 0.68 | 0.32 | 524 |
| Example 27 | Compound 211 | 3.43 | 58.34 | 0.68 | 0.32 | 498 |
| Example 28 | Compound 214 | 3.42 | 58.27 | 0.68 | 0.32 | 506 |
| Example 29 | Compound 219 | 3.42 | 58.56 | 0.68 | 0.32 | 512 |
| Example 30 | Compound 221 | 3.42 | 59.98 | 0.68 | 0.32 | 513 |
| Example 31 | Compound 222 | 3.41 | 58.05 | 0.68 | 0.32 | 496 |
| Example 32 | Compound 224 | 3.44 | 62.67 | 0.68 | 0.32 | 474 |
| Example 33 | Compound 232 | 3.45 | 62.56 | 0.68 | 0.32 | 472 |
| Comparative | Compound A | 3.47 | 51.50 | 0.68 | 0.32 | 398 |
| Example 1 | | | | | | |
| Comparative | Compound B | 3.45 | 52.70 | 0.68 | 0.32 | 421 |
| Example 2 | | | | | | |
| Comparative | Compound C | 3.48 | 50.20 | 0.68 | 0.32 | 403 |
| Example 3 | | | | | | |

As can be seen from Table 6 above, compared with Comparative Examples 1-3, when the compounds of the present application were employed as host materials in red organic electroluminescent devices, the luminescence efficiency of the devices was increased by at least 10.0%, and the 195 lifetime was increased by at least 12.1%.

It was found by the present application that the compounds of the present application comprise a phenanthrobenzoxazole/thiazole core structure in their structures, which is connected to a triazine compound, and the compounds respectively serve as electron-transport red-light host materials. Among them, the phenanthrobenzoxazole/thiazole has a large conjugation system, which can enhance the intermolecular interactions and improve the carrier mobility of the compounds after connecting with triazines. When the compounds of the present application are used as the electron transport materials in the mixed host materials, they can improve the carrier balance in the light-emitting layer, broaden the carrier recombination zone, enhance the efficiency of exciton generation and utilization, and improve the luminescence efficiency and lifetime of the device.

Other embodiments of the present application will be readily appreciated by those skilled in the art upon consideration of the specification and practice of the invention disclosed herein. The present application is intended to cover any variations, uses, or adaptations of this application that follow the general principles of this application and include common knowledge or conventional technical means in the art not disclosed herein.

1-13. (canceled)

14. An organic compound, characterized by having a structure shown in Formula 1: 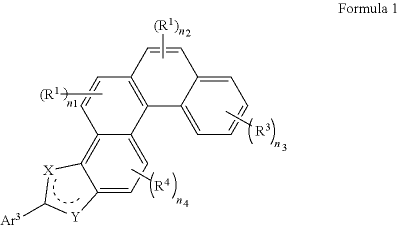
wherein one of X and Y is —O— or —S—, and the other is —N═; 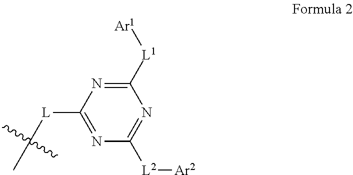
n₁ is the number of R¹ and is selected from the group consisting of 0, 1, and 2; when n₁ is greater than 1, any two R¹s are the same or different;
n₂ is the number of R² and is selected from the group consisting of 0, 1, and 2; when n₂ is greater than 1, any two R²s are the same or different;
n₃ is the number of R³ and is selected from the group consisting of 0, 1, 2, 3, and 4; when n₃ is greater than 1, any two R³s are the same or different;
when n₃ is 1, R³ has the structure shown in Formula 2, and each R¹, R² and R⁴ are each independently selected from the group consisting of deuterium, cyano, halogen, alkyl having 1 to 4 carbon atoms, deuterated alkyl having 1 to 4 carbon atoms, haloalkyl having 1 to 4 carbon atoms, aryl having 6 to 12 carbon atoms, deuterated aryl having 6 to 12 carbon atoms, and heteroaryl having 5 to 12 carbon atoms;
when n₃ is greater than 1, one of R³ has the structure shown in Formula 2, and the rest of R³ and each R¹, R² and R⁴ are each independently selected from the group consisting of deuterium, cyano, halogen, alkyl having 1 to 4 carbon atoms, deuterated alkyl having 1 to 4 carbon atoms, haloalkyl having 1 to 4 carbon atoms, aryl having 6 to 12 carbon atoms, deuterated aryl having 6 to 12 carbon atoms, and heteroaryl having 5 to 12 carbon atoms;
n₄ is the number of R⁴ and is selected from the group consisting of 0, 1, and 2; when n₄ is greater than 1, any two R⁴s are the same or different;
Ar¹ and Ar² are the same or different, and are each independently selected from the group consisting of substituted or unsubstituted phenyl, substituted or unsubstituted biphenyl, substituted or unsubstituted terphenyl, substituted or unsubstituted naphthyl, substituted or unsubstituted phenanthryl, substituted or unsubstituted fluorenyl, substituted or unsubstituted spirobifluorenyl, substituted or unsubstituted triphenylene, substituted or unsubstituted dibenzothienyl, substituted or unsubstituted dibenzofuranyl, and substituted or unsubstituted carbazolyl;
Ar³ is selected from the group consisting of substituted or unsubstituted phenyl, substituted or unsubstituted biphenyl, substituted or unsubstituted naphthyl, substituted or unsubstituted phenanthryl, substituted or unsubstituted dibenzothienyl, and substituted or unsubstituted dibenzofuranyl;
the substituent(s) in Ar¹, Ar², and Ar³ are the same or different, and are each independently selected from the group consisting of deuterium, fluorine, cyano, methyl, ethyl, isopropyl, tert-butyl, phenyl, naphthyl, and pentadeuterophenyl;
L is selected from the group consisting of a single bond, substituted or unsubstituted phenylene, and substituted or unsubstantiated naphthylene;
L¹ and L² are the same or different, and are each independently selected from the group consisting of a single bond, substituted or unsubstituted phenylene, substituted or unsubstantiated naphthylene, and substituted or unsubstantiated biphenylene;
the substituent(s) in L¹ and L² are the same or different, and are each independently selected from the group consisting of deuterium, fluorine, cyano, methyl, ethyl, isopropyl, tert-butyl, and phenyl.

15. The organic compound according to claim 14, characterized in that,
when n₃ is greater than 1, the rest of R³ and each R¹, R² and R⁴ are each independently selected from the group consisting of deuterium, cyano, fluorine, methyl, ethyl, isopropyl, tert-butyl, and phenyl.

16. The organic compound according to claim 14, characterized in that n₃ is 1.

17. The organic compound according to claim 14, characterized in that, Ar¹ and Ar² are the same or different, and are each independently selected from the group consisting of the following groups: 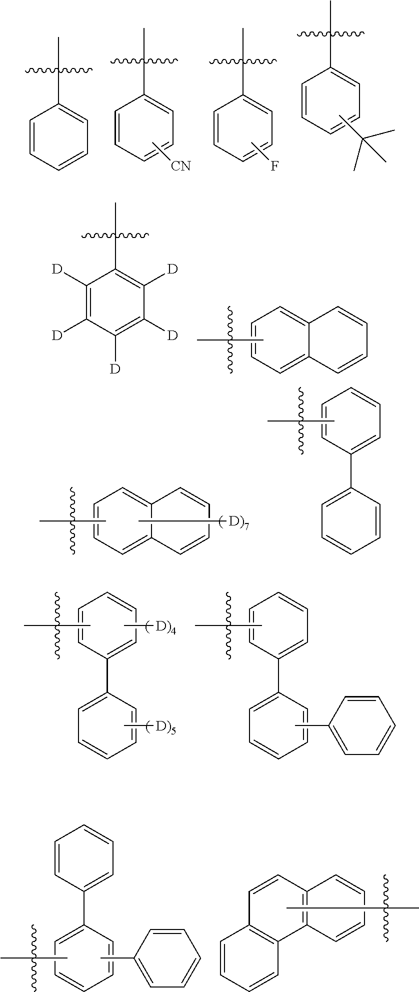 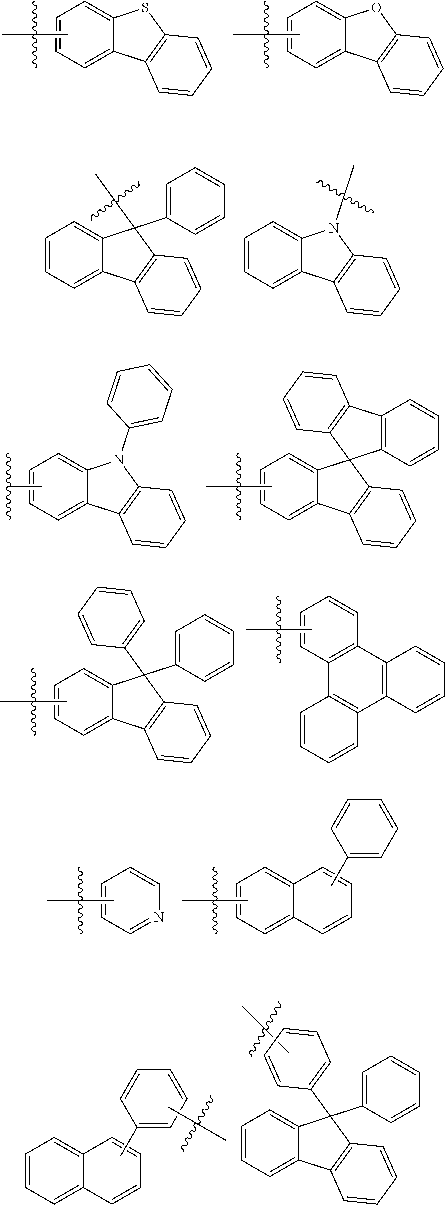 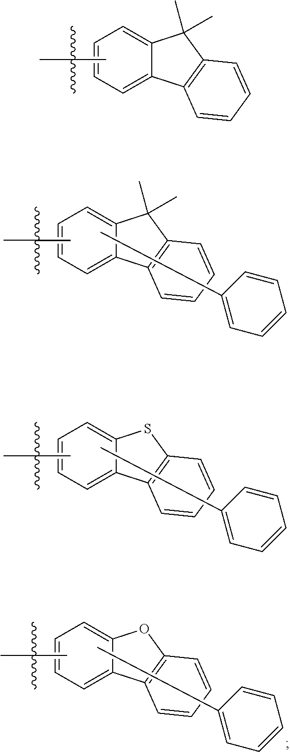
Ar³ is selected from the group consisting of the following groups:

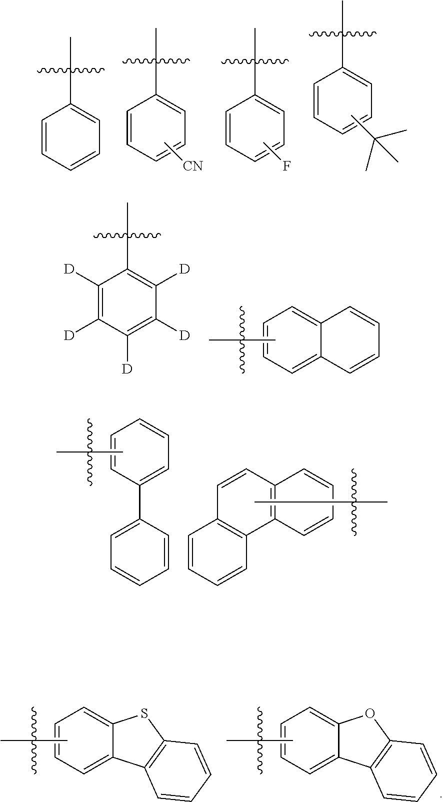

18. The organic compound according to claim 14, characterized in that,
L is selected from the group consisting of a single bond and the following groups: 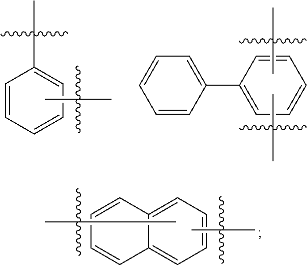
L¹ and L² are the same or different, and are each independently selected from the group consisting of a single bond and the following groups:

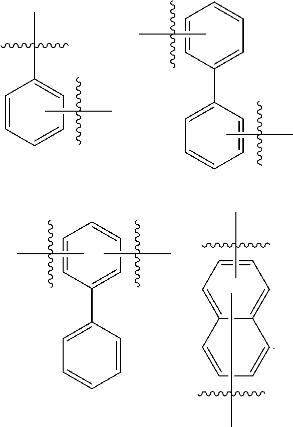

19. The organic compound according to claim 14, characterized in that, 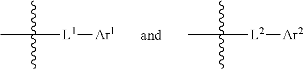
are the same or different, and are each independently selected from the group consisting of the following groups:

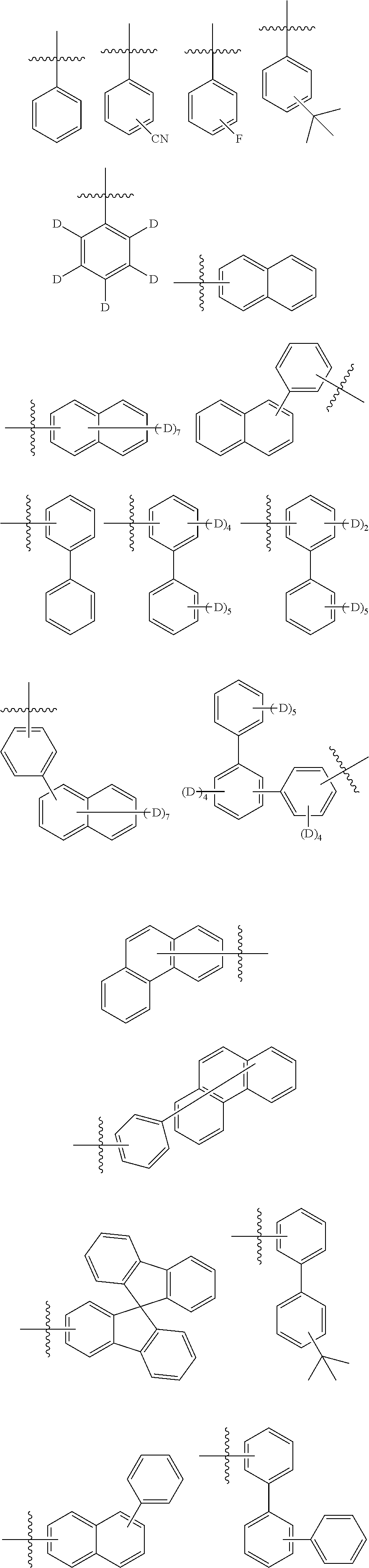

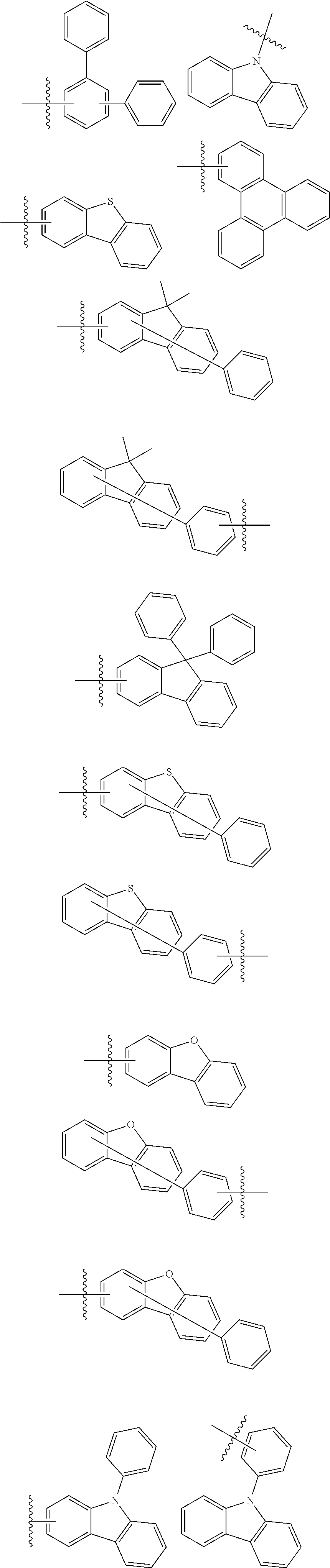

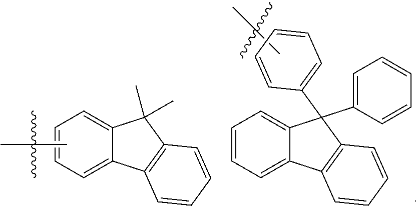

20. The organic compound according to claim 14, characterized in that, the structure 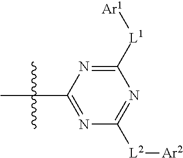
shown in Formula 2 is selected from the group consisting of the following groups:

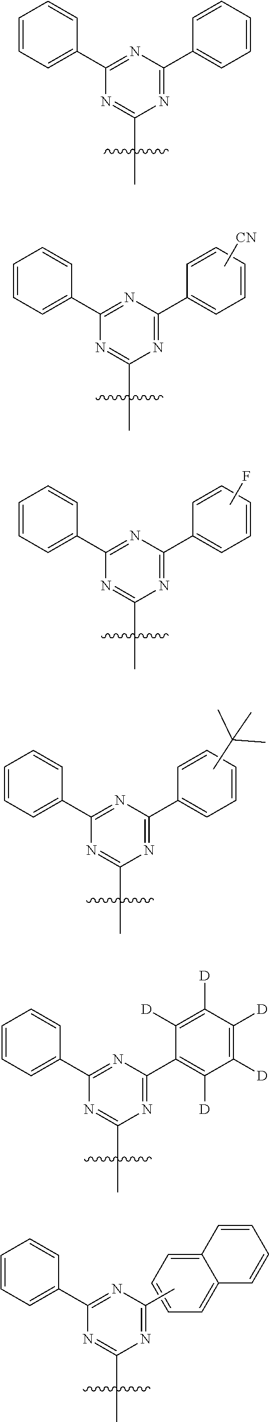

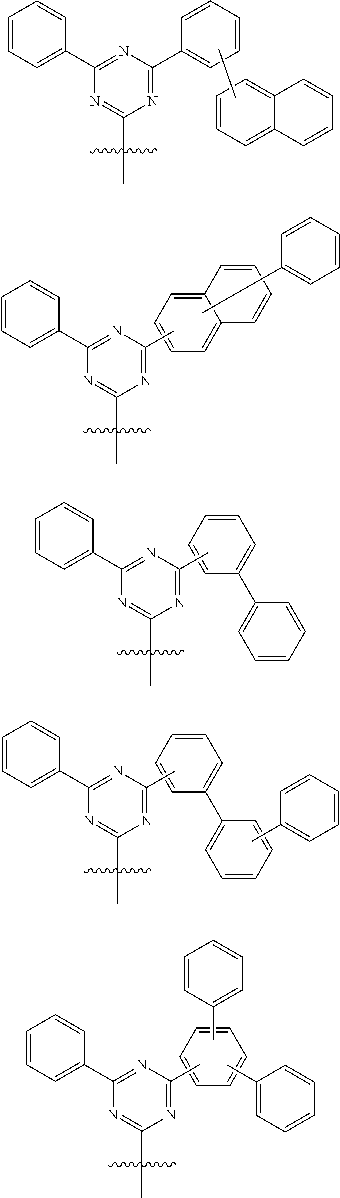

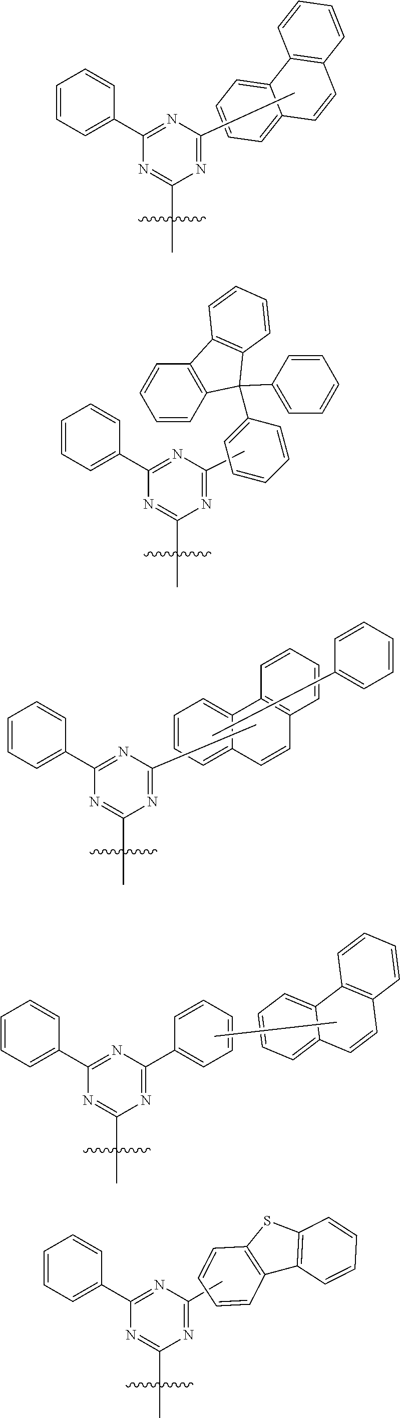

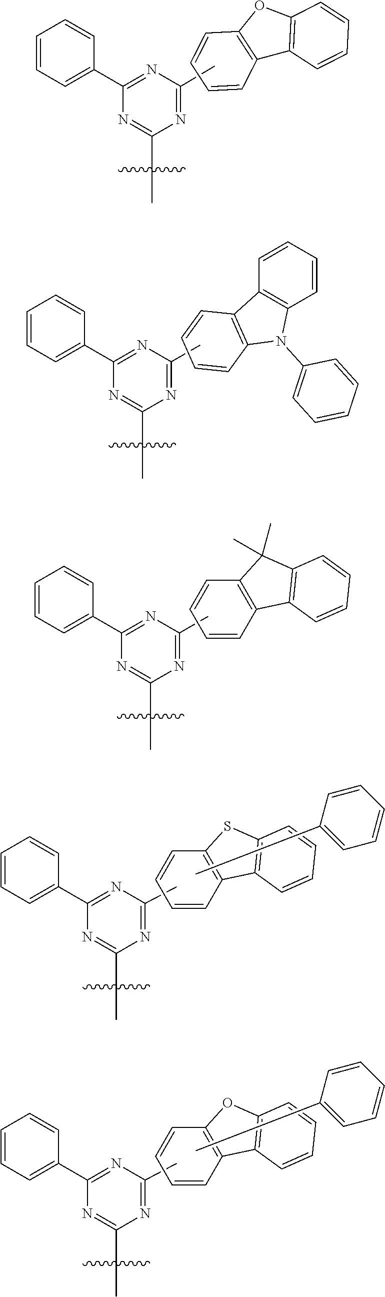

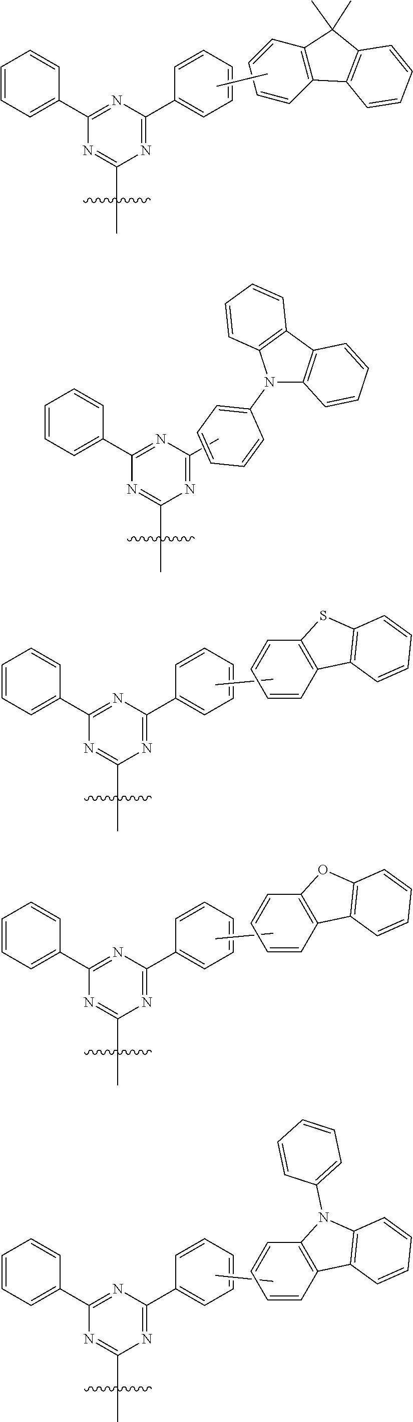

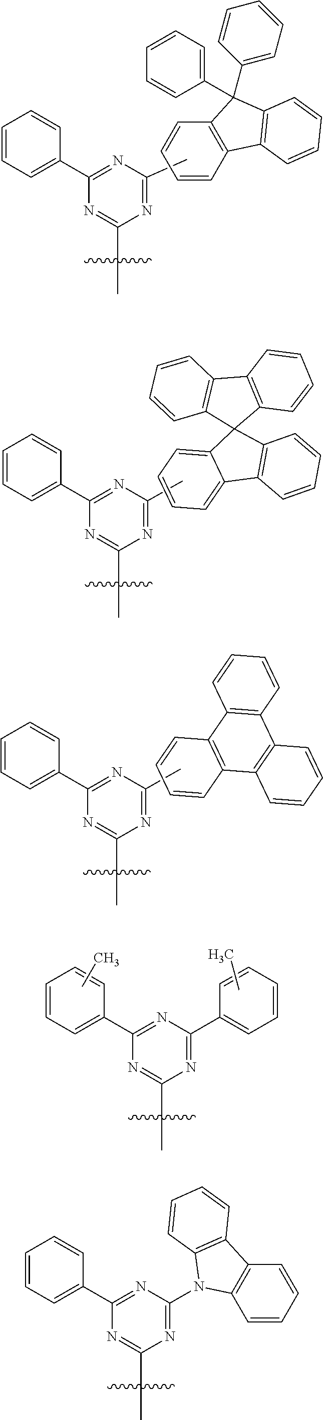

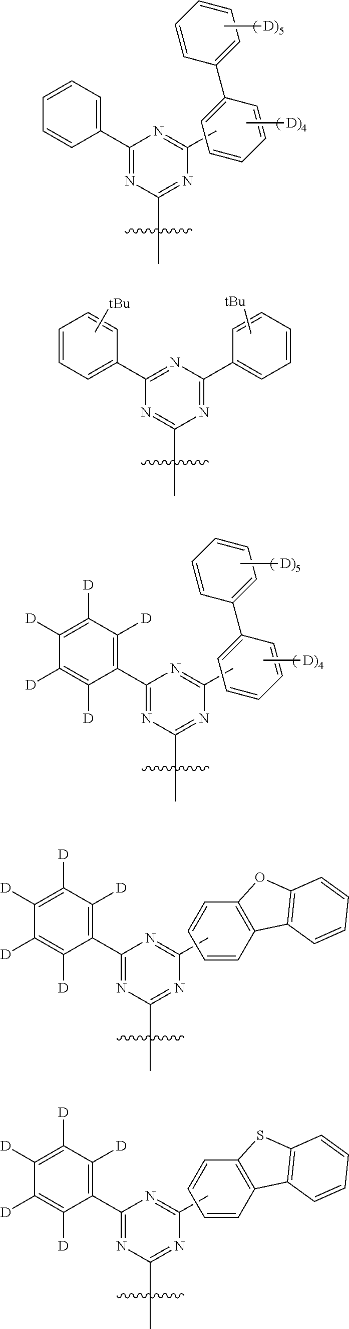

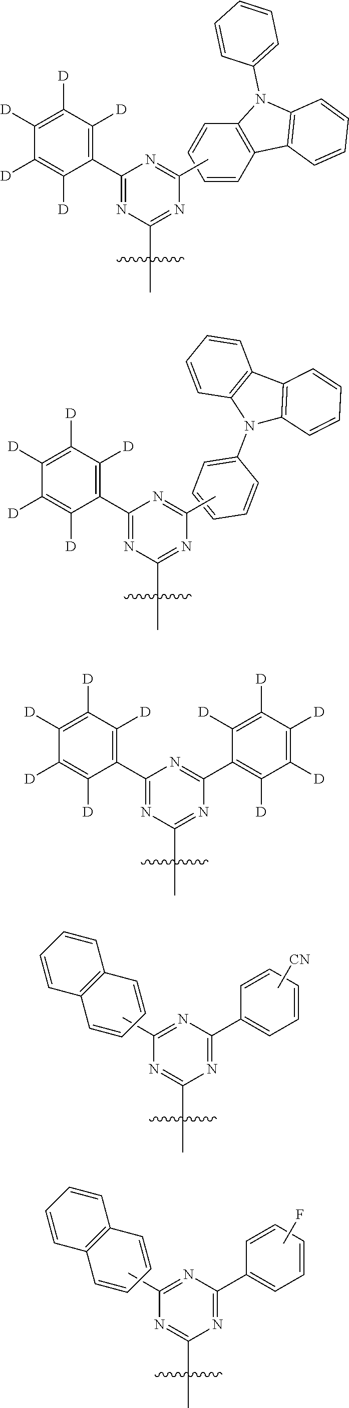

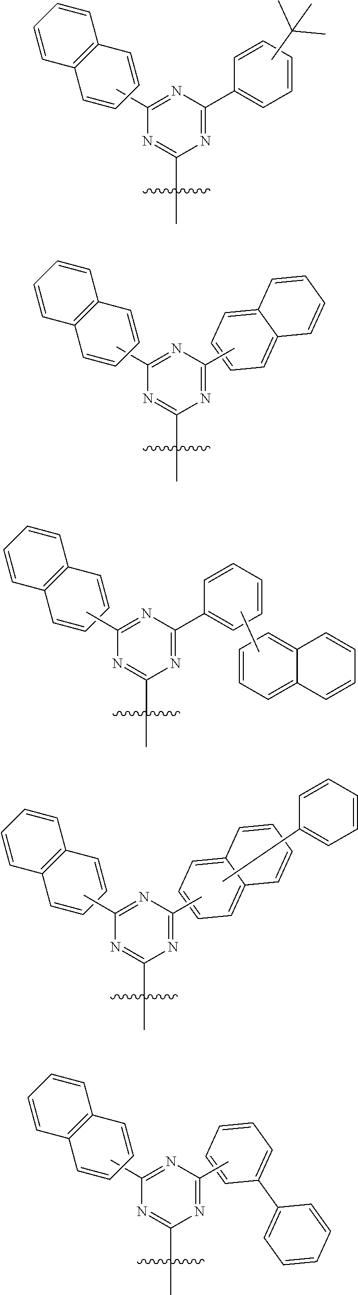

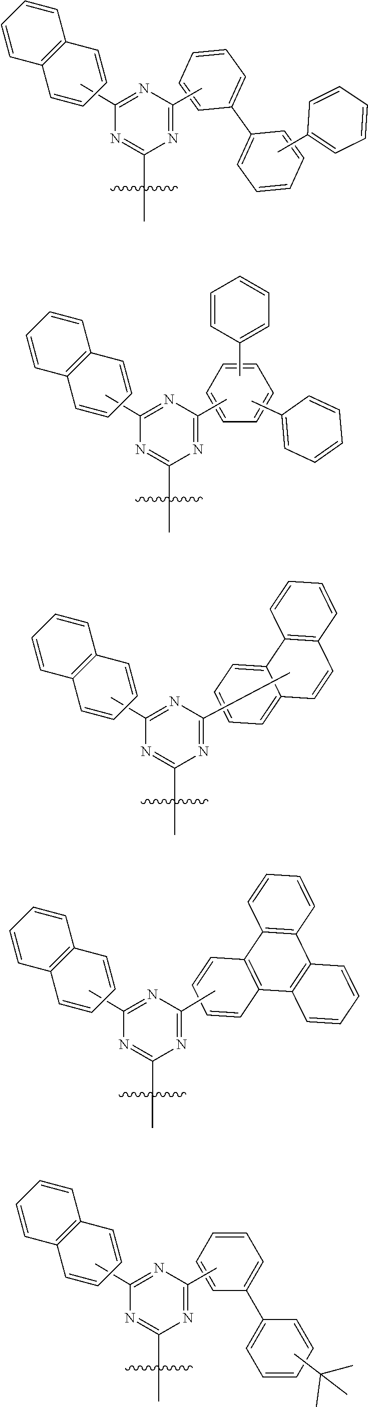

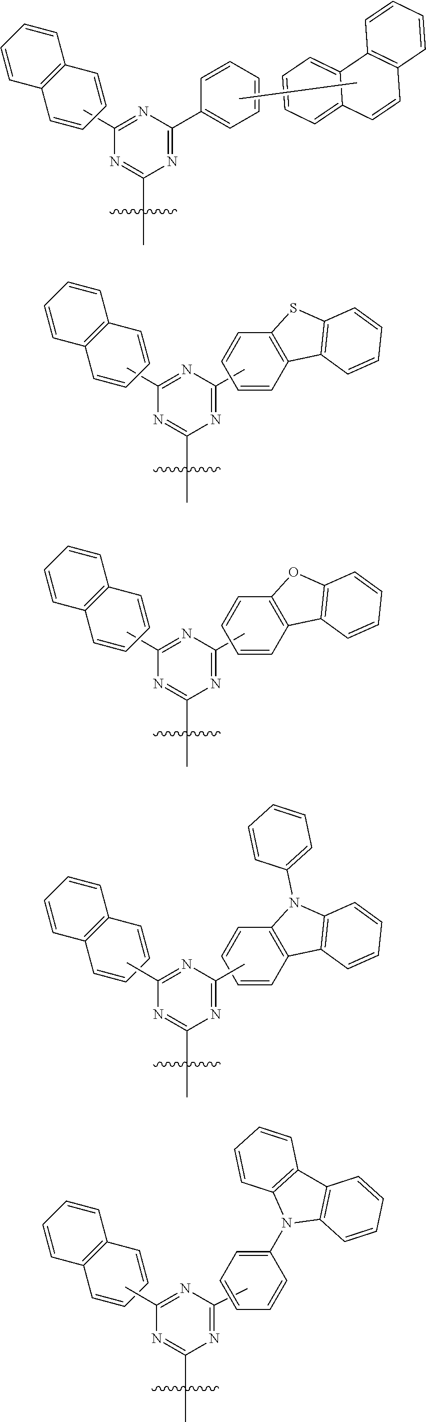

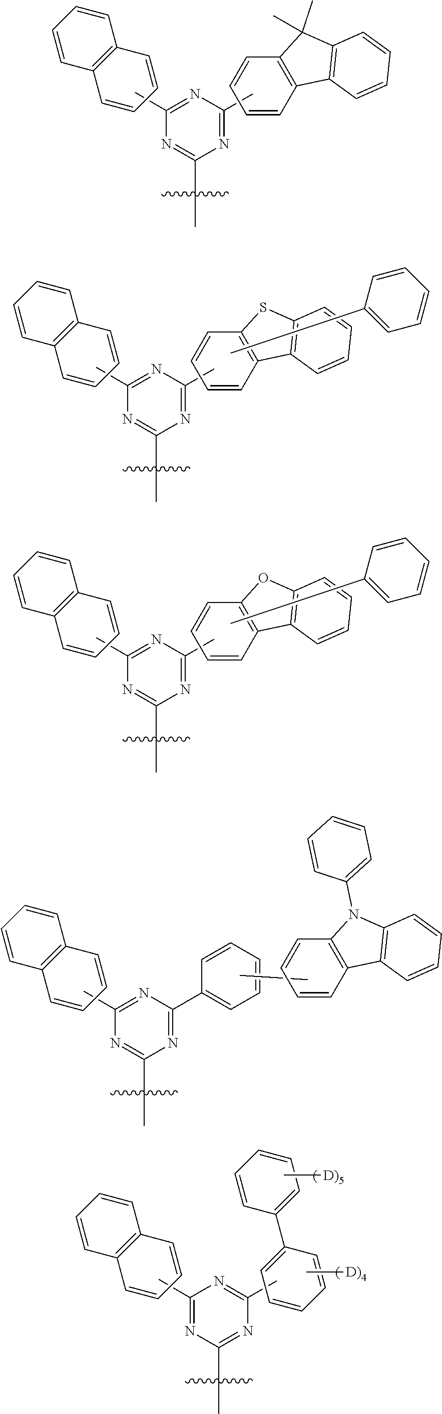

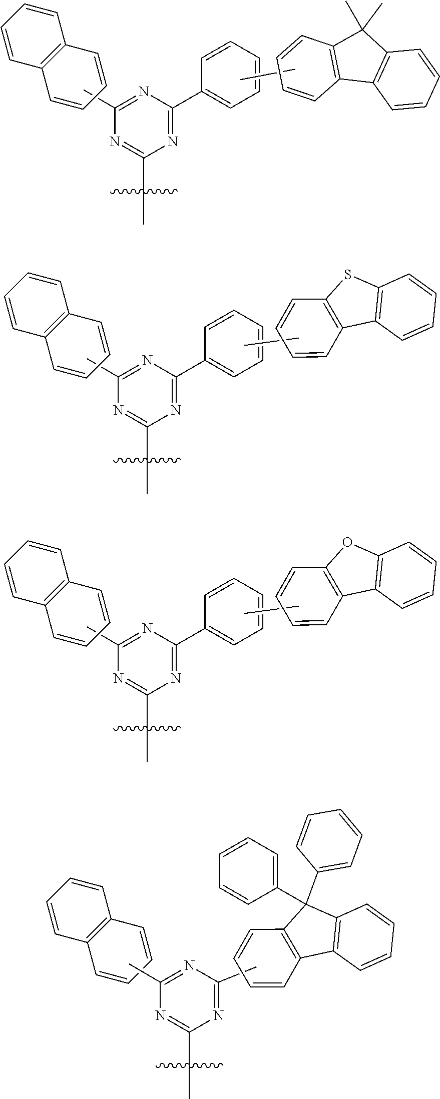

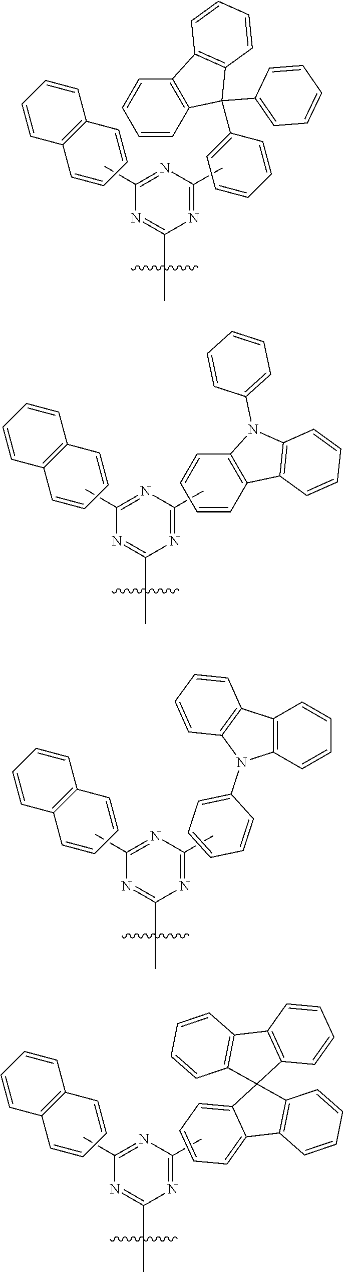

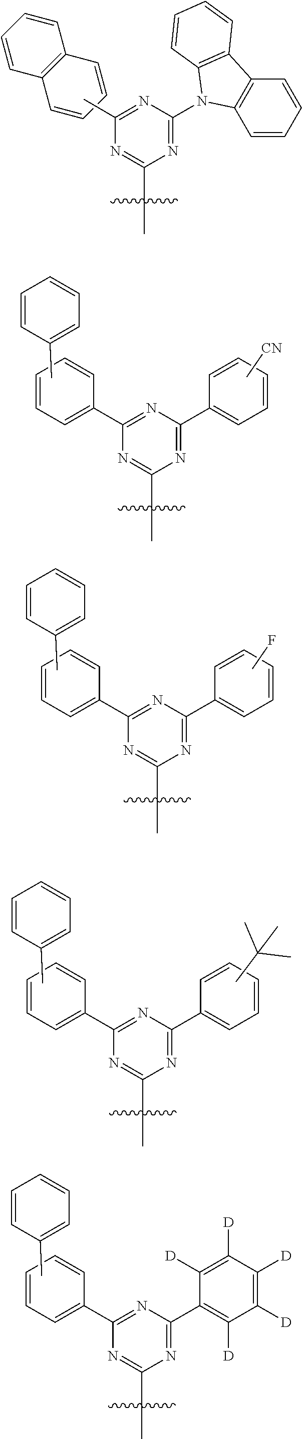

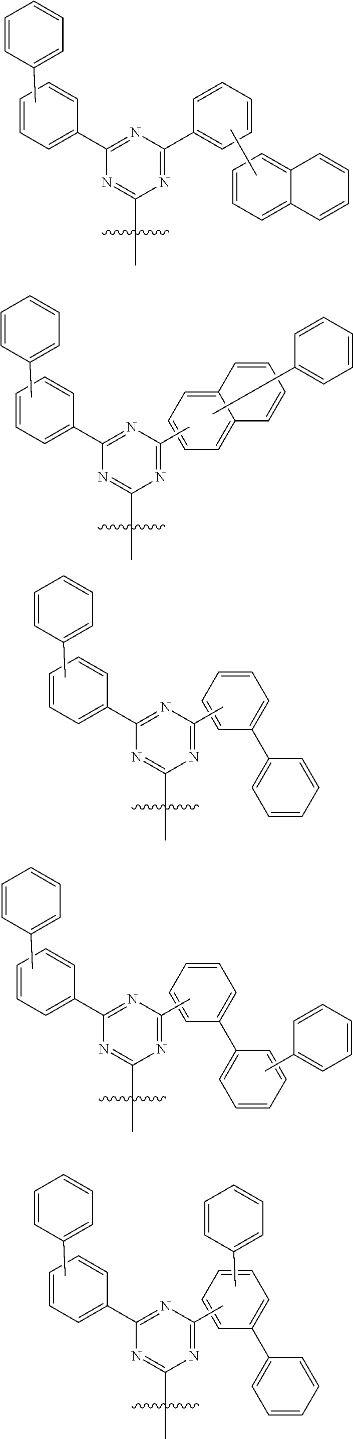

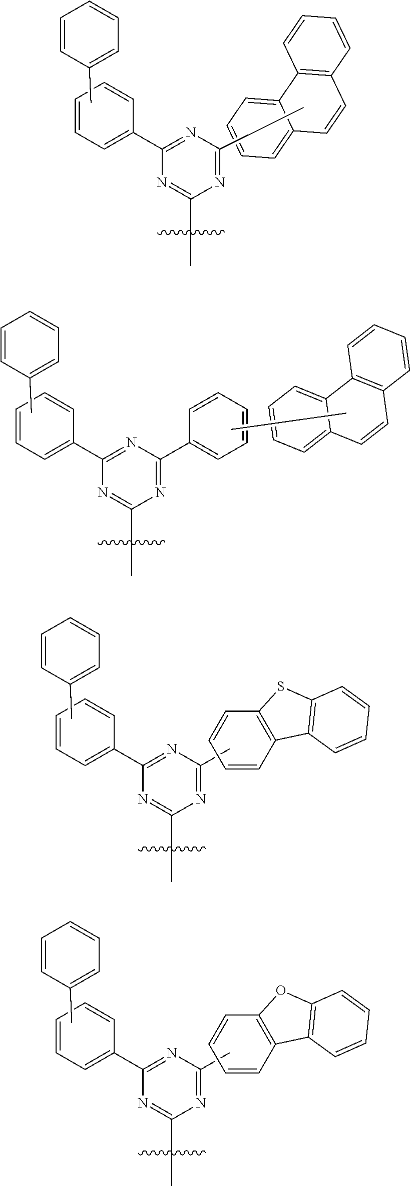

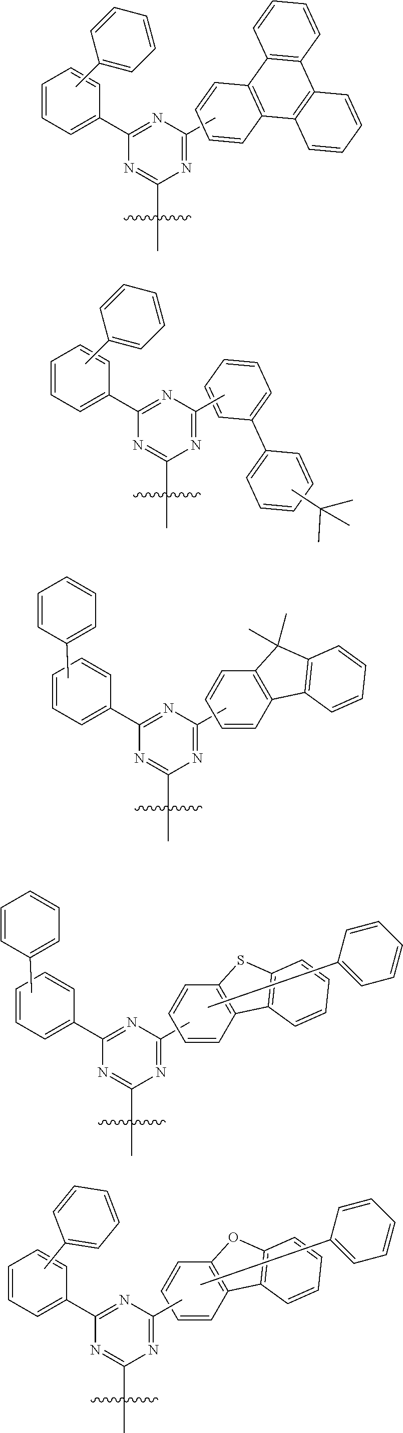

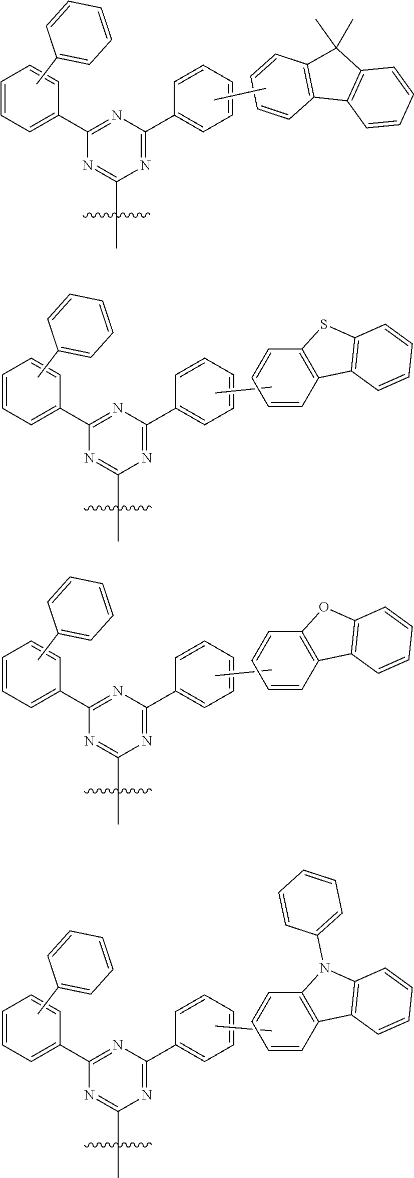

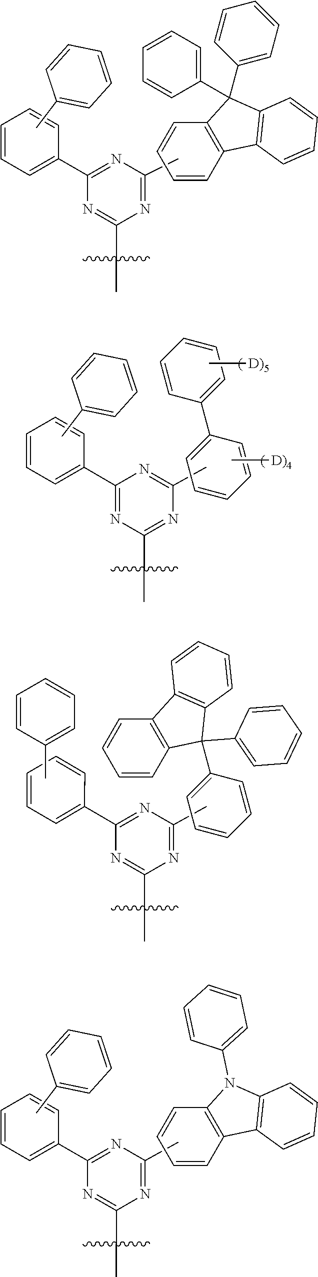

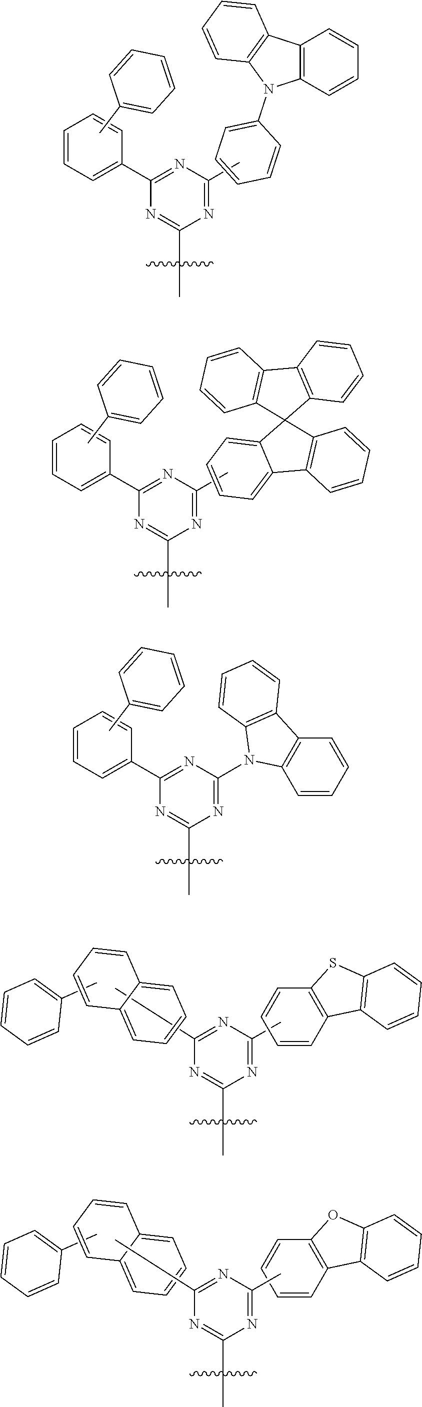

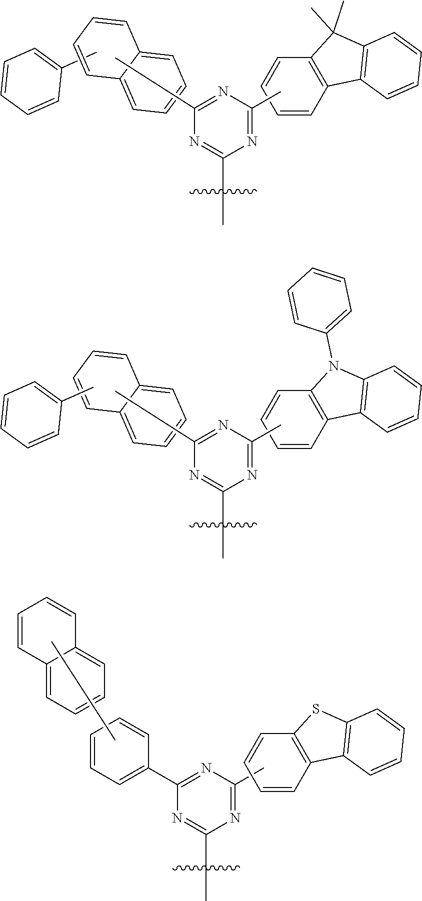

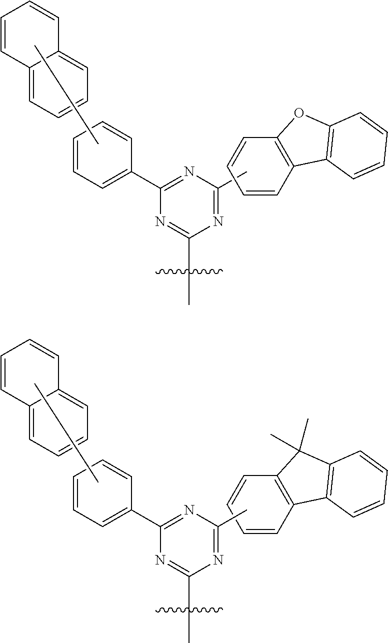

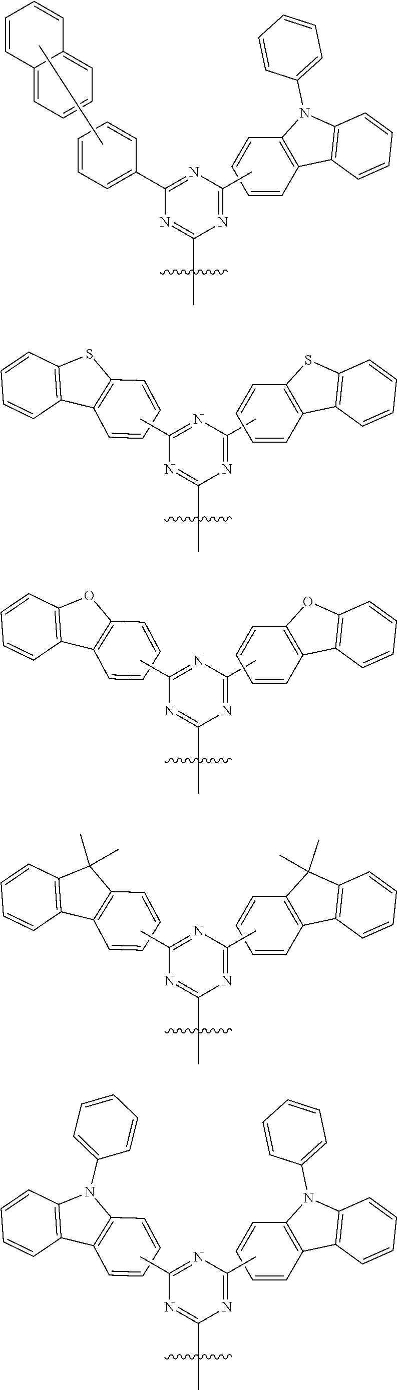

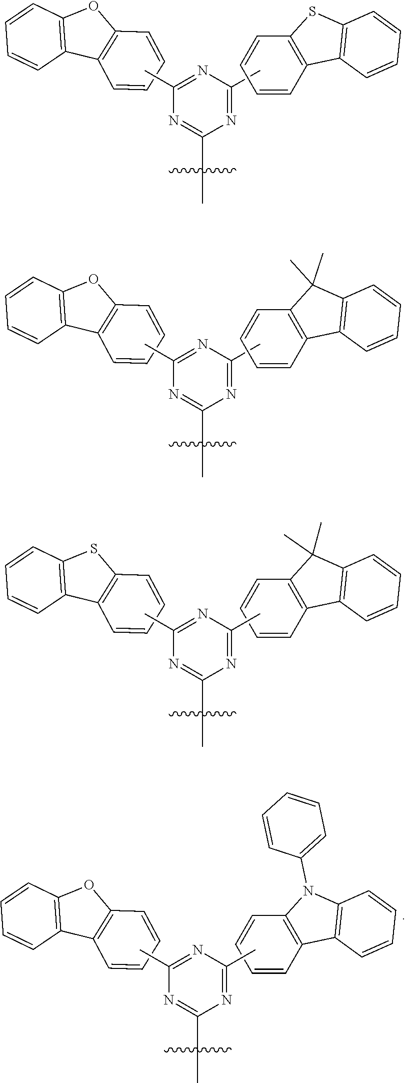

21. The organic compound according to claim 14, characterized in that, the compound is selected from the group consisting of the following compounds:

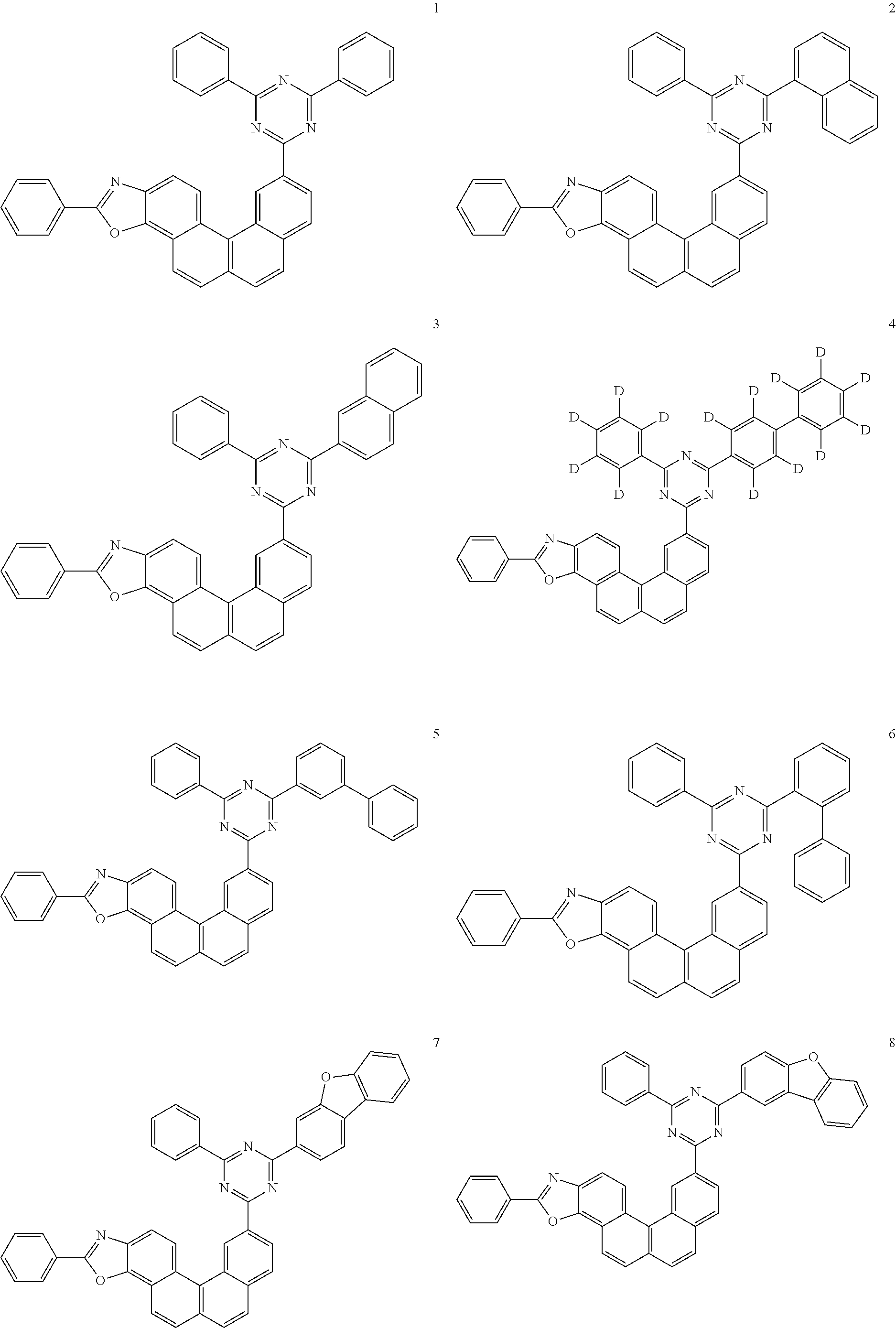

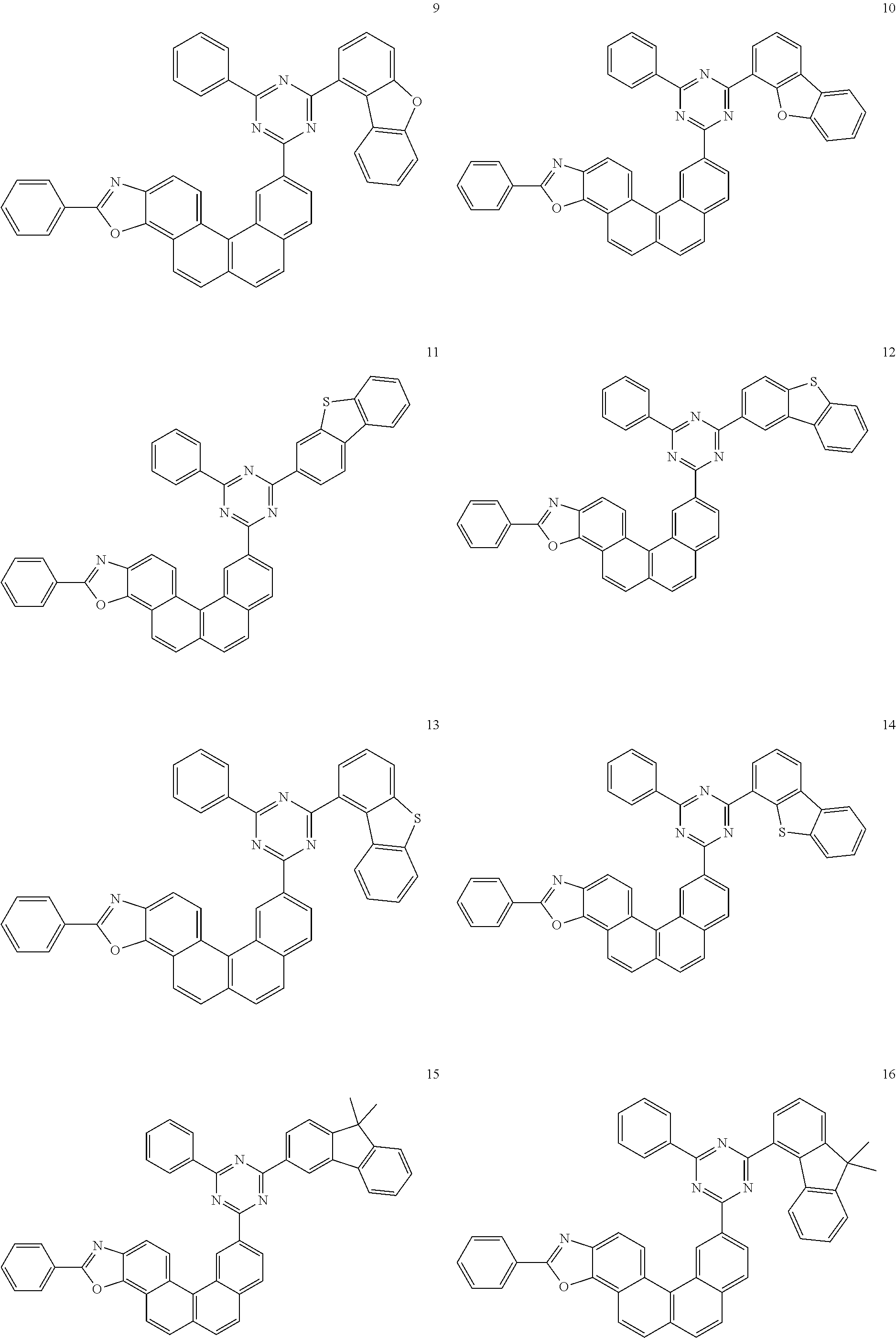

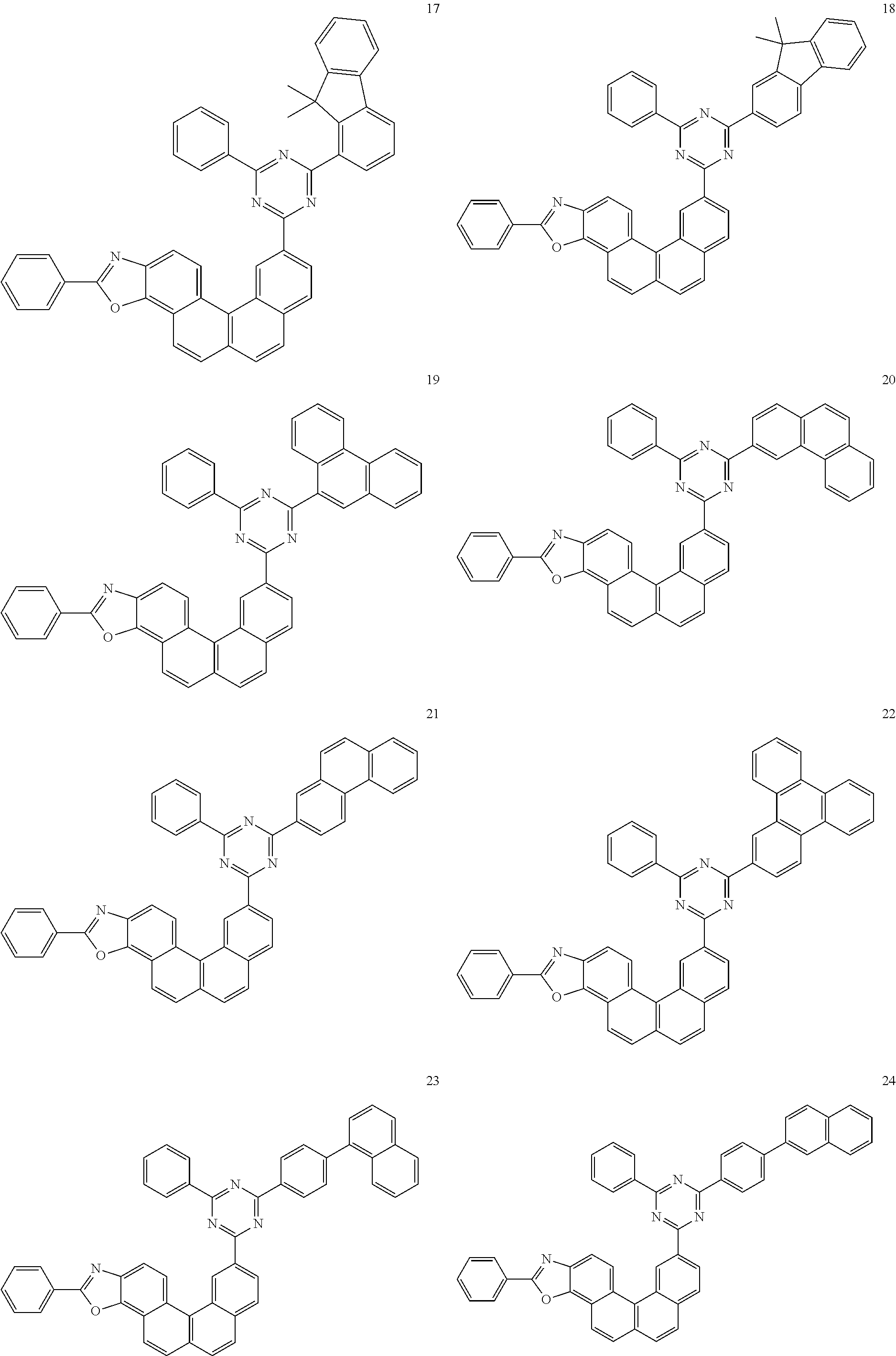

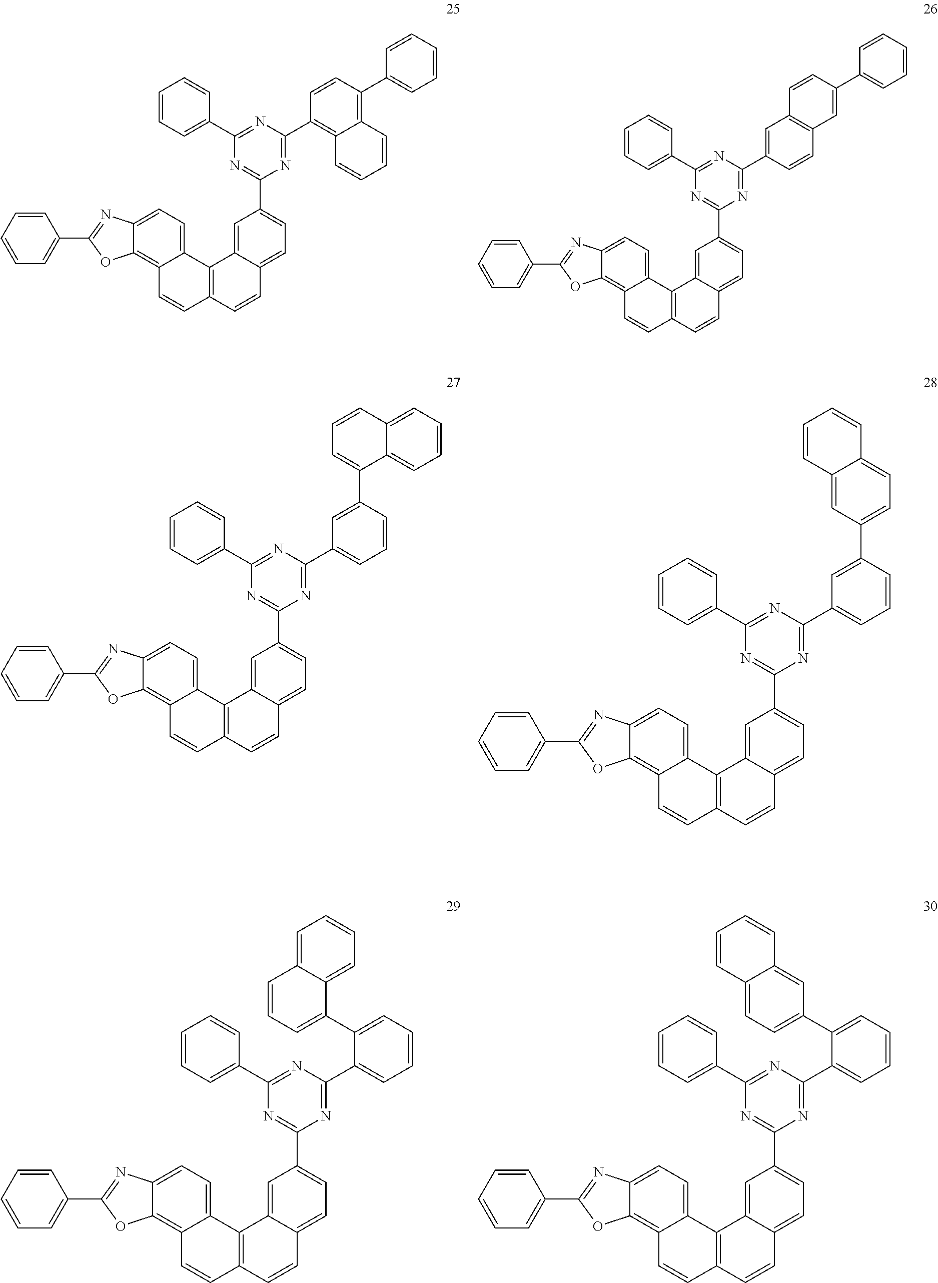

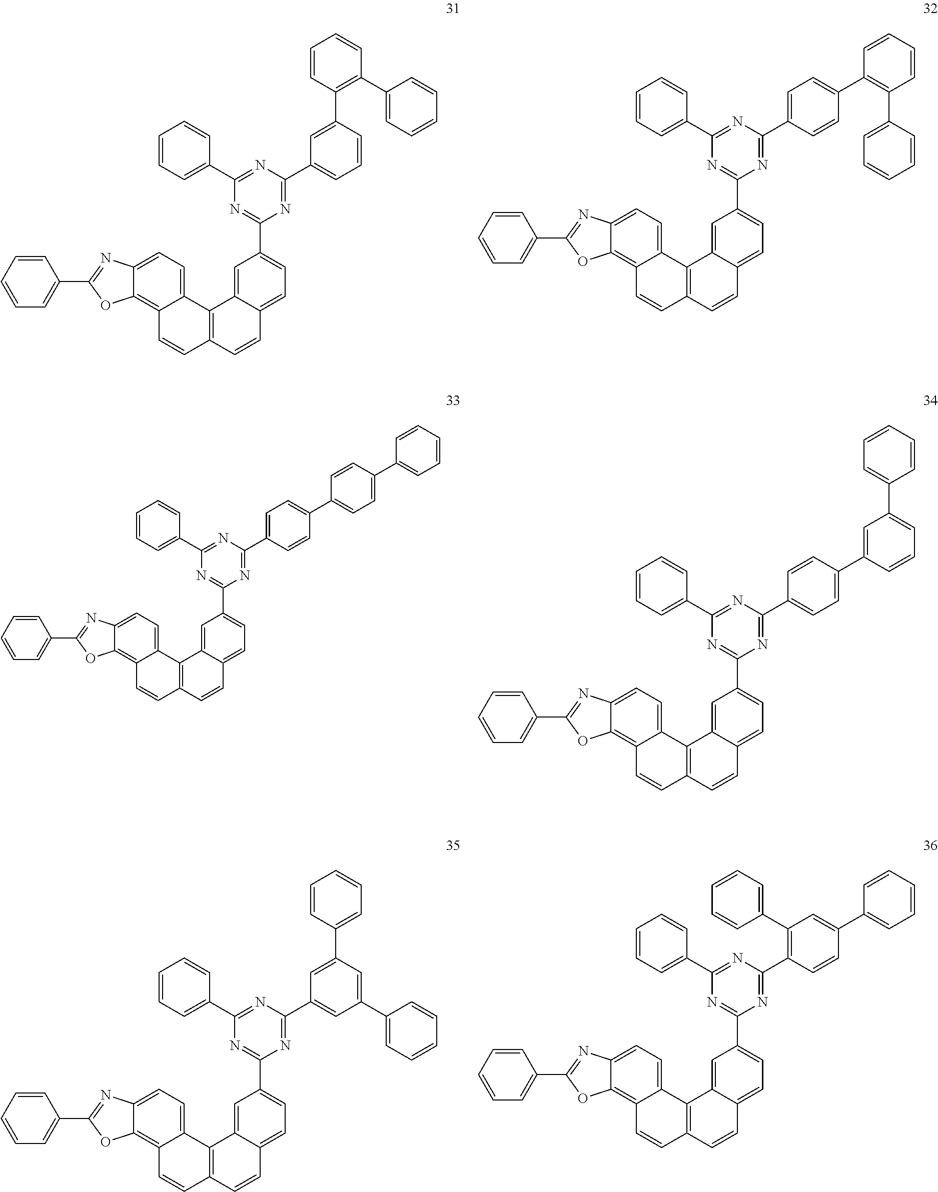

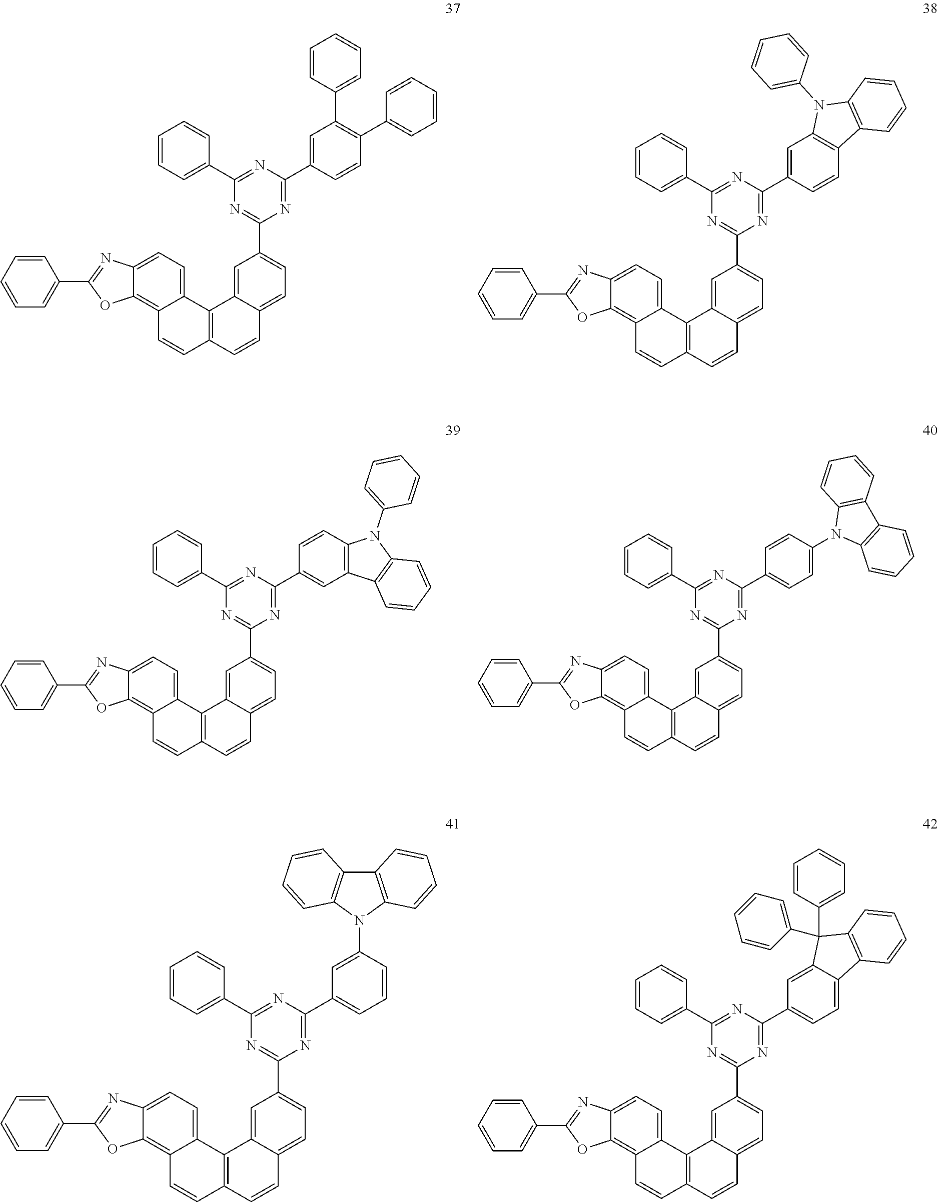

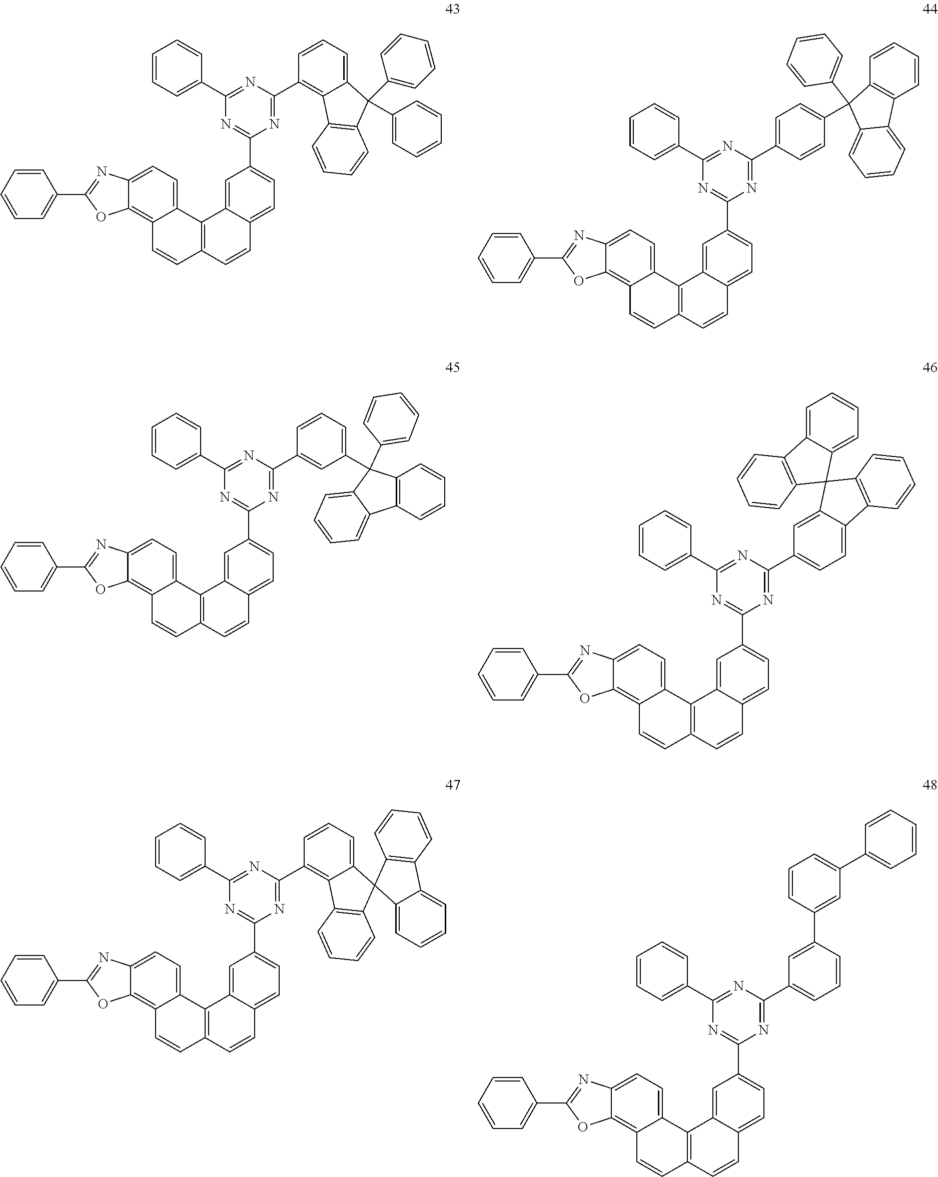

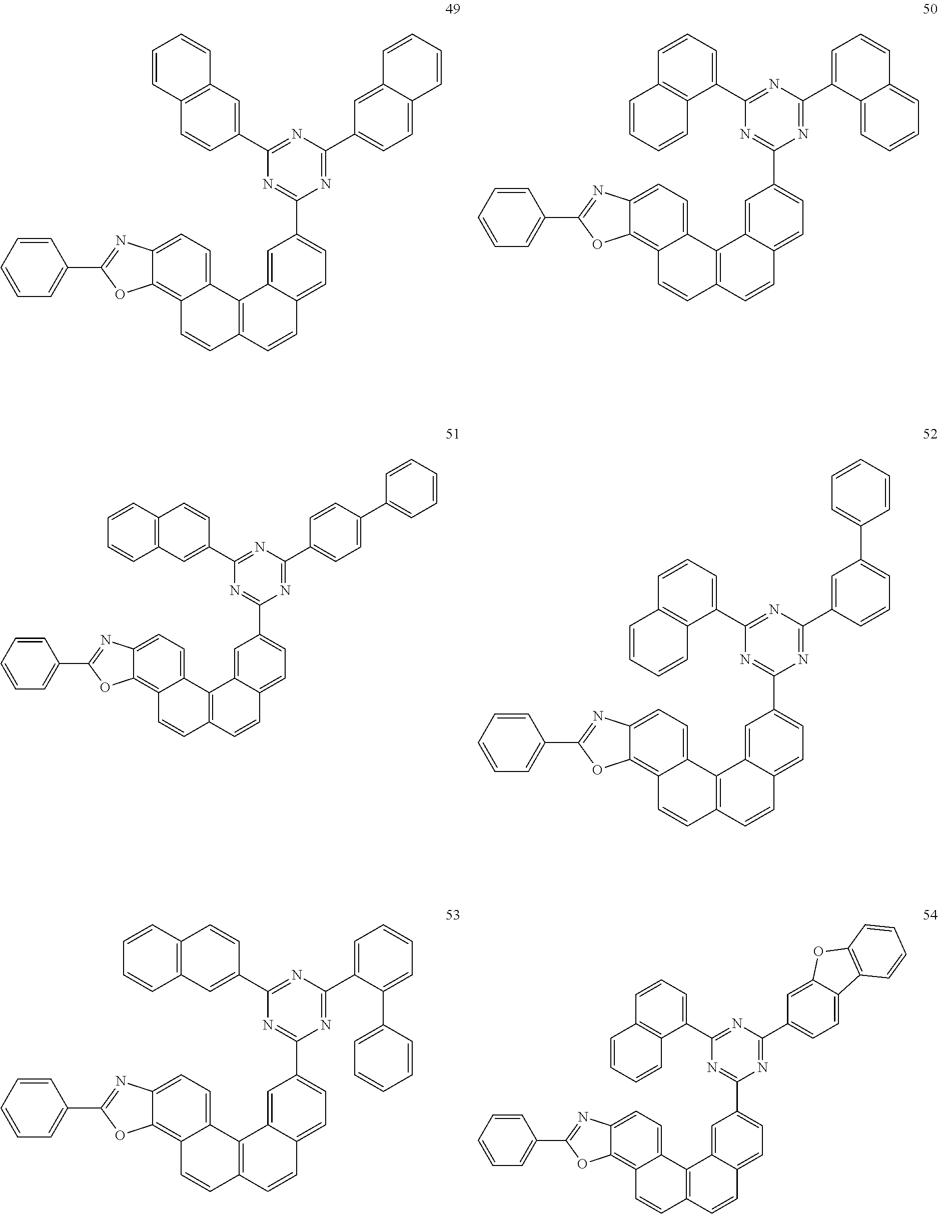

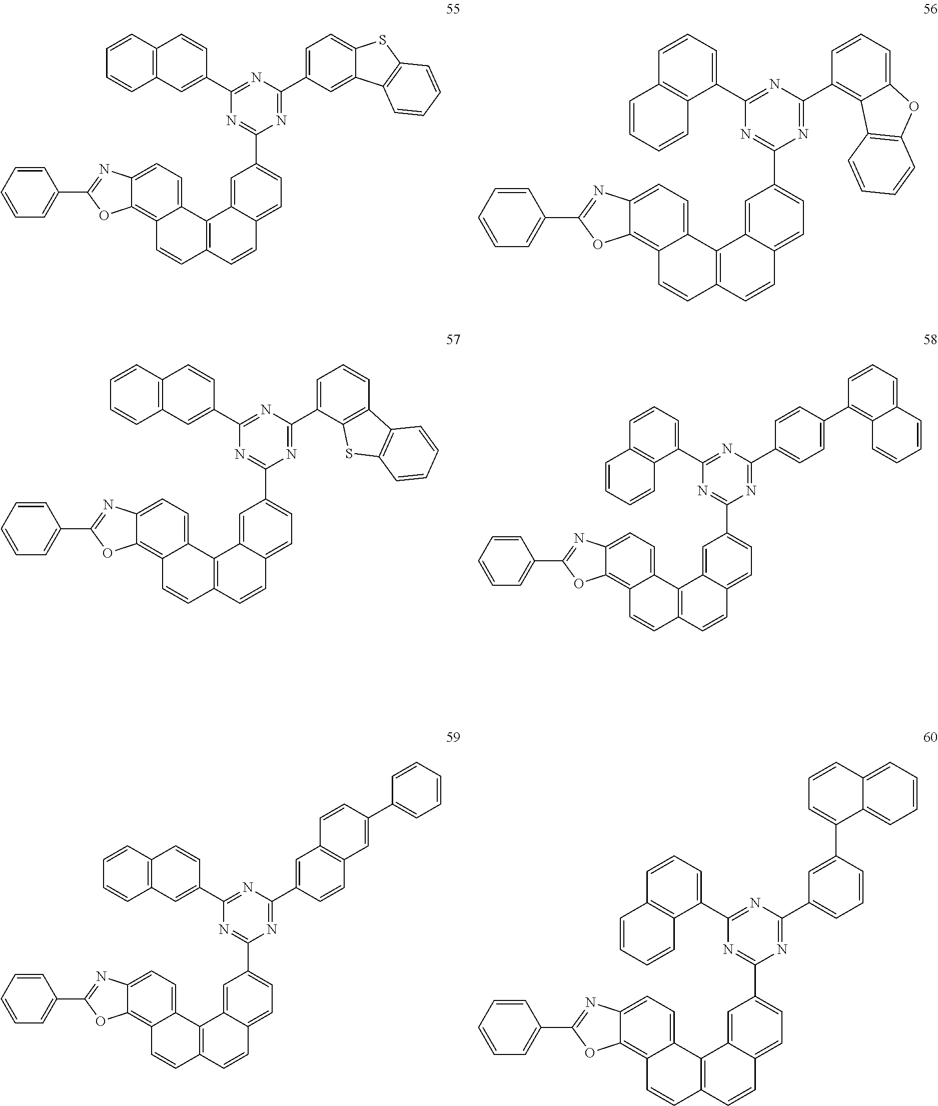

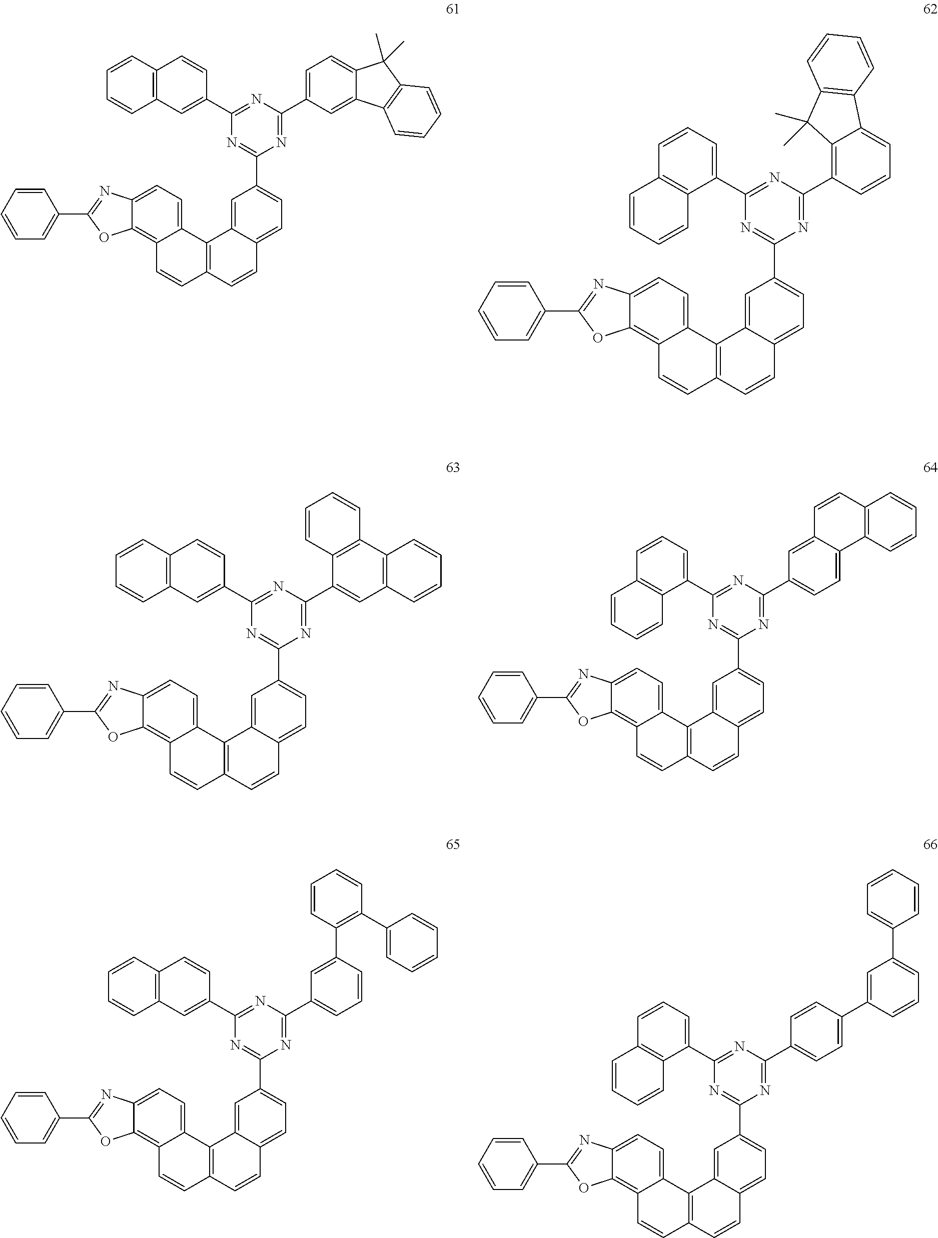

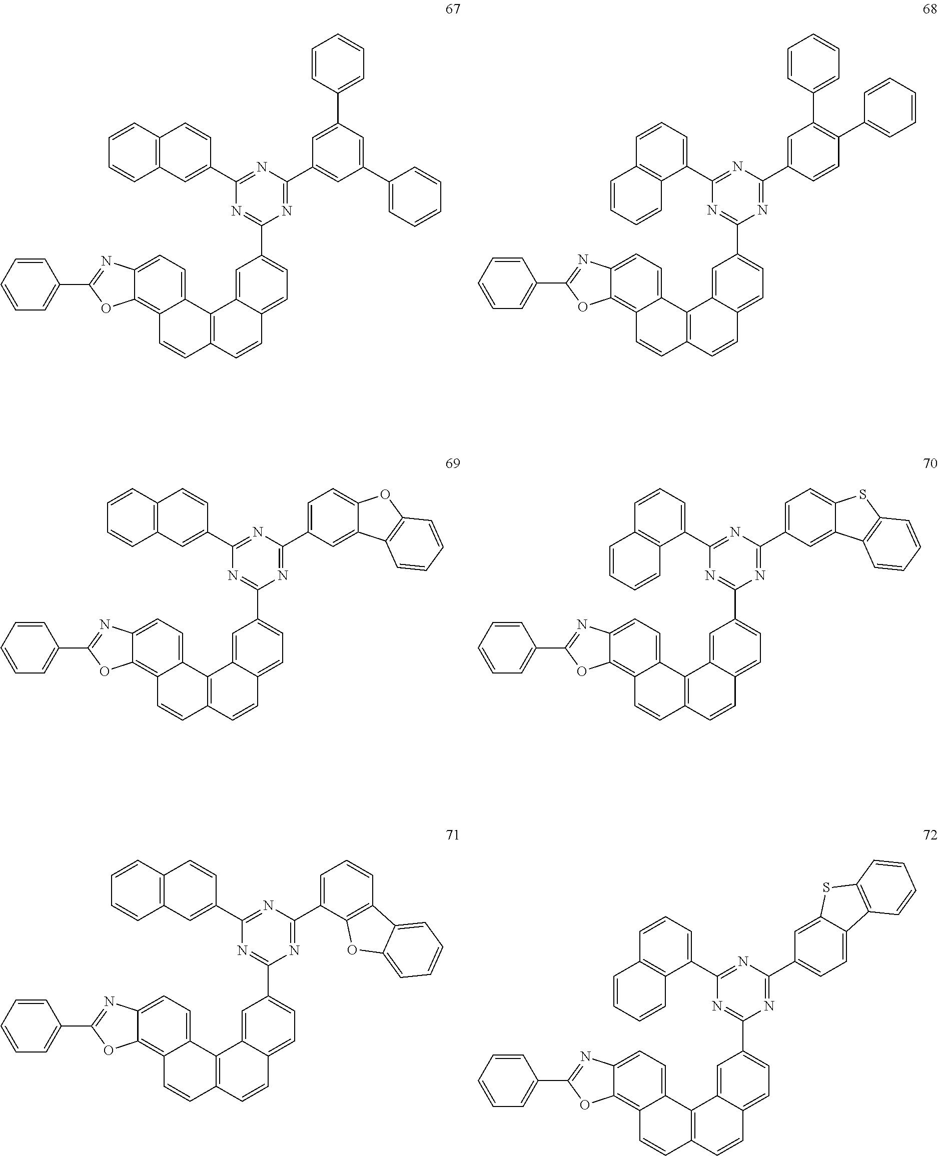

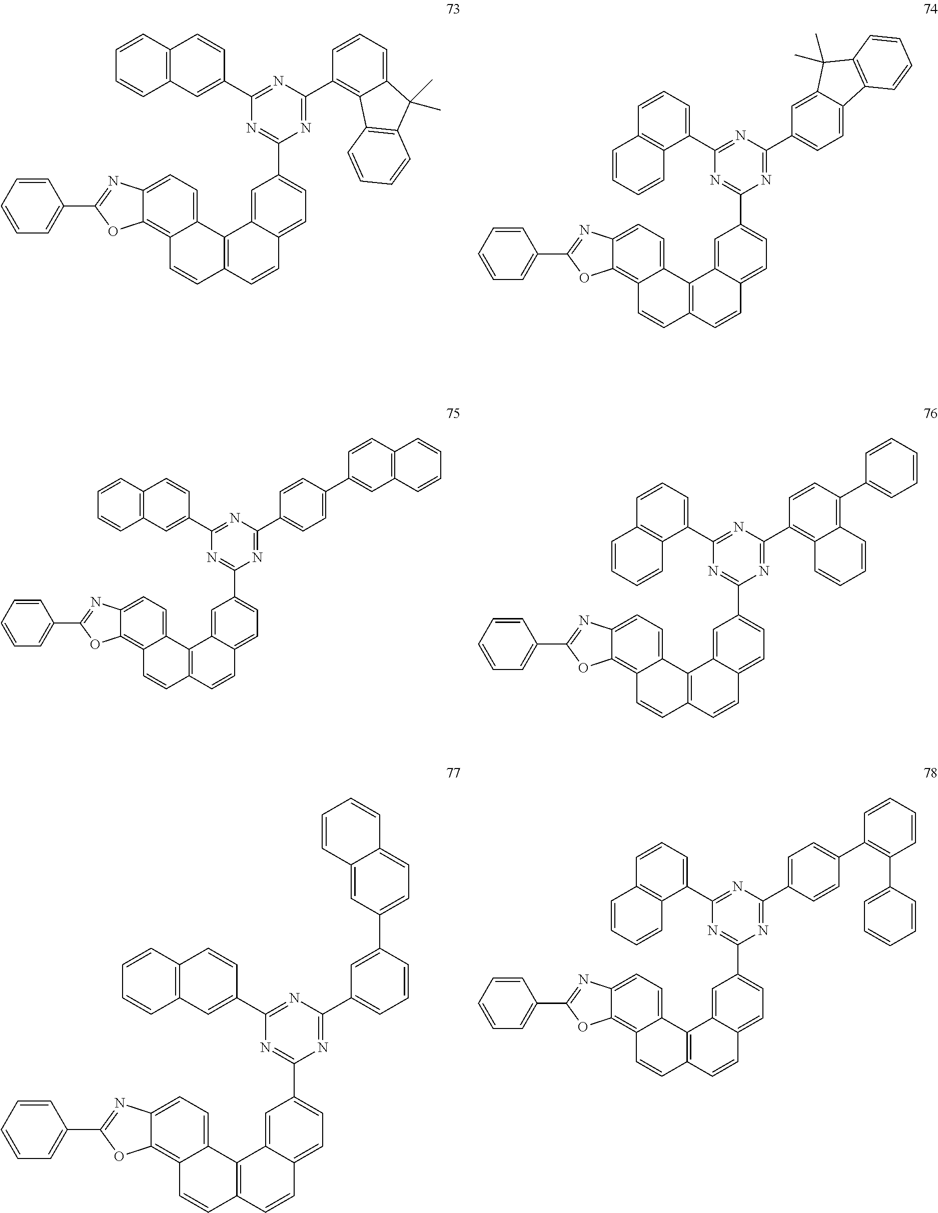

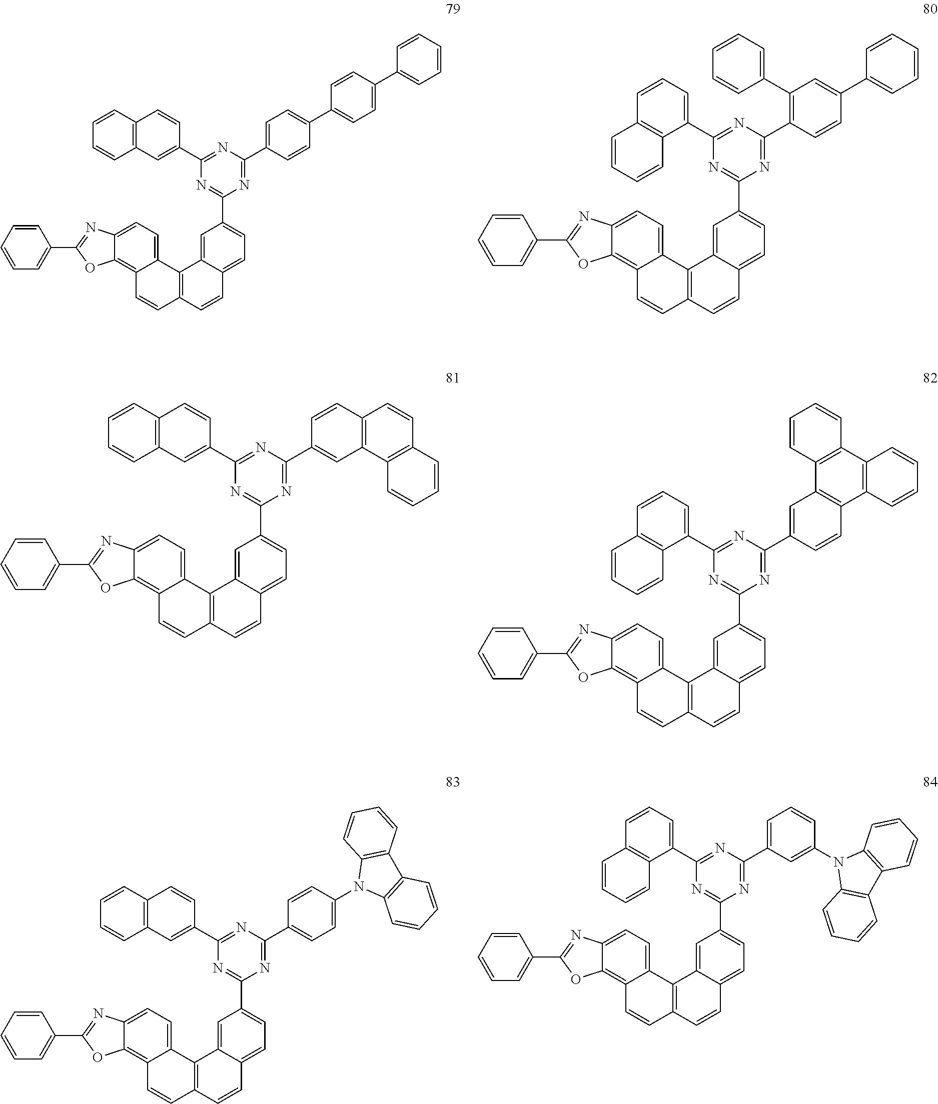

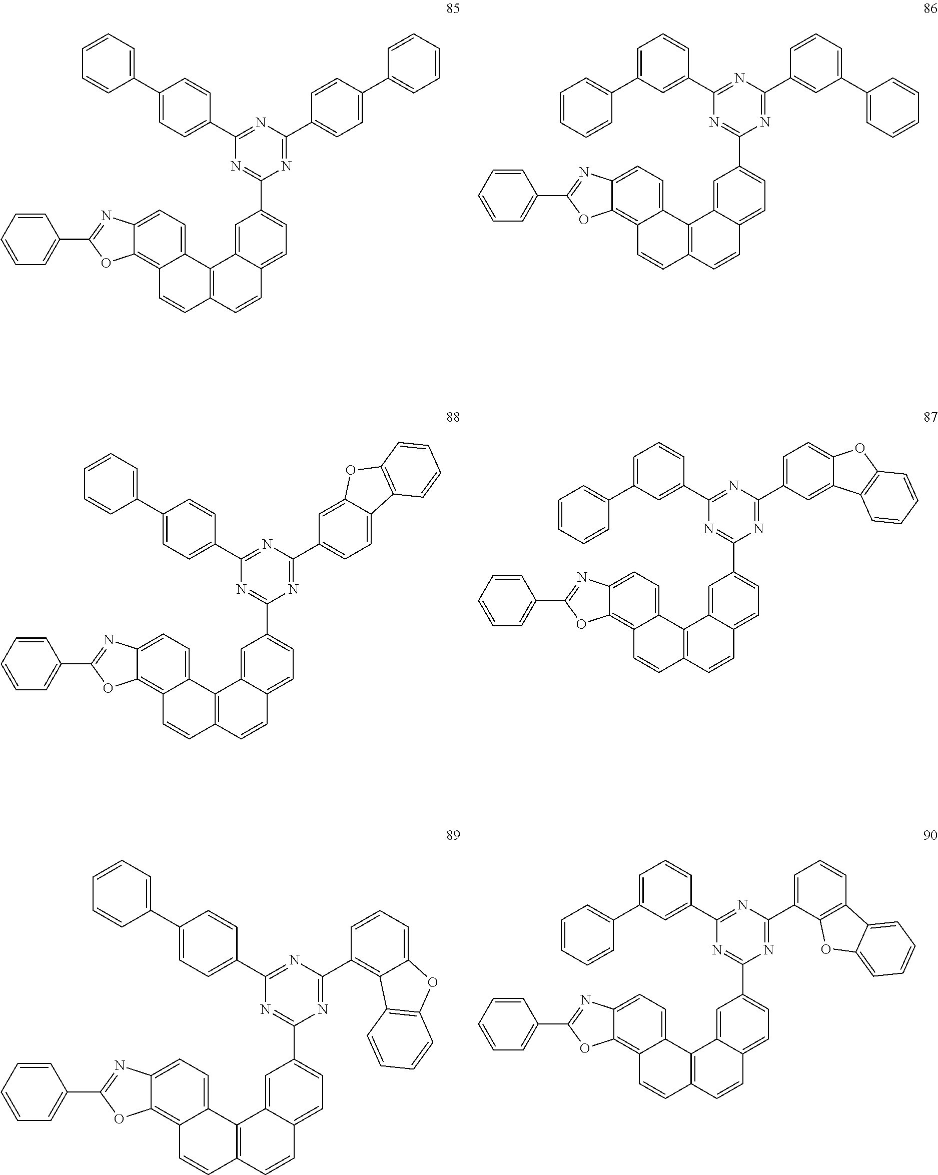

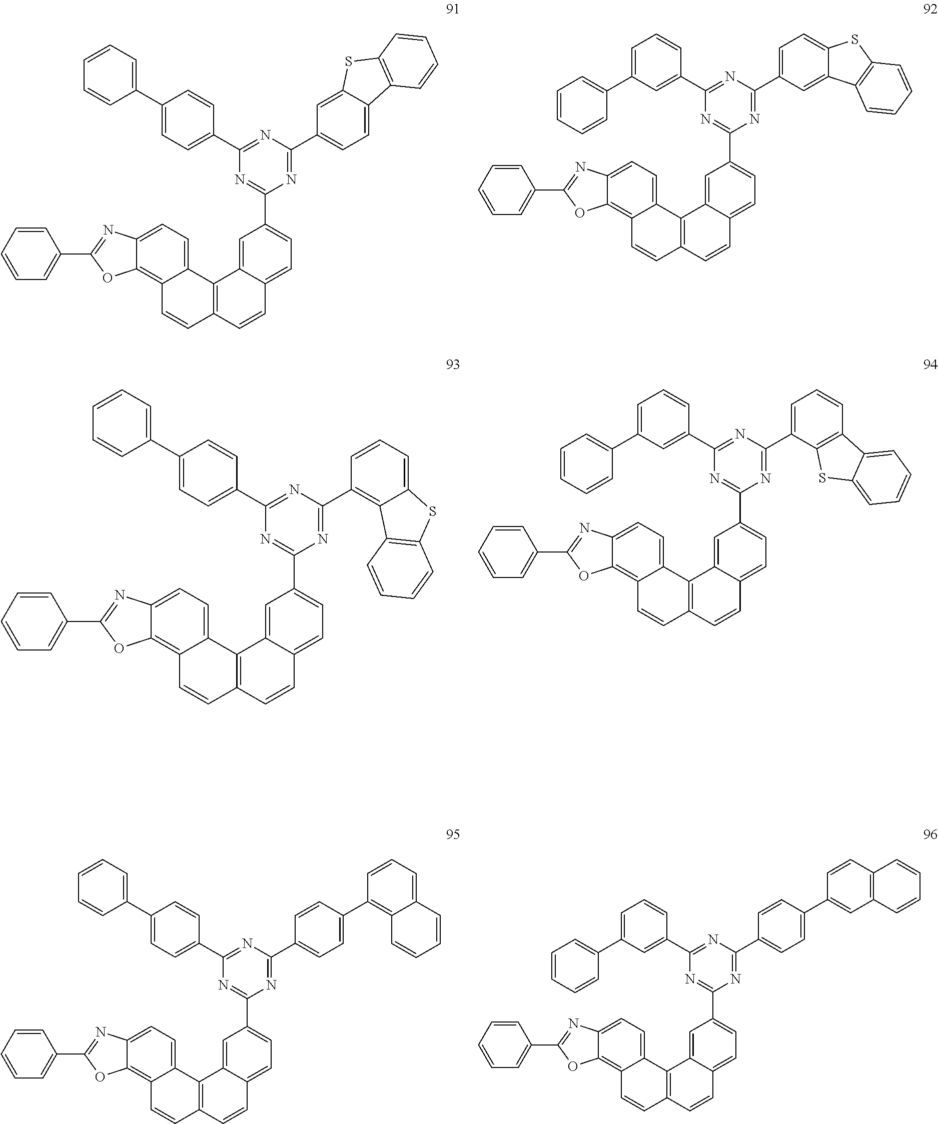

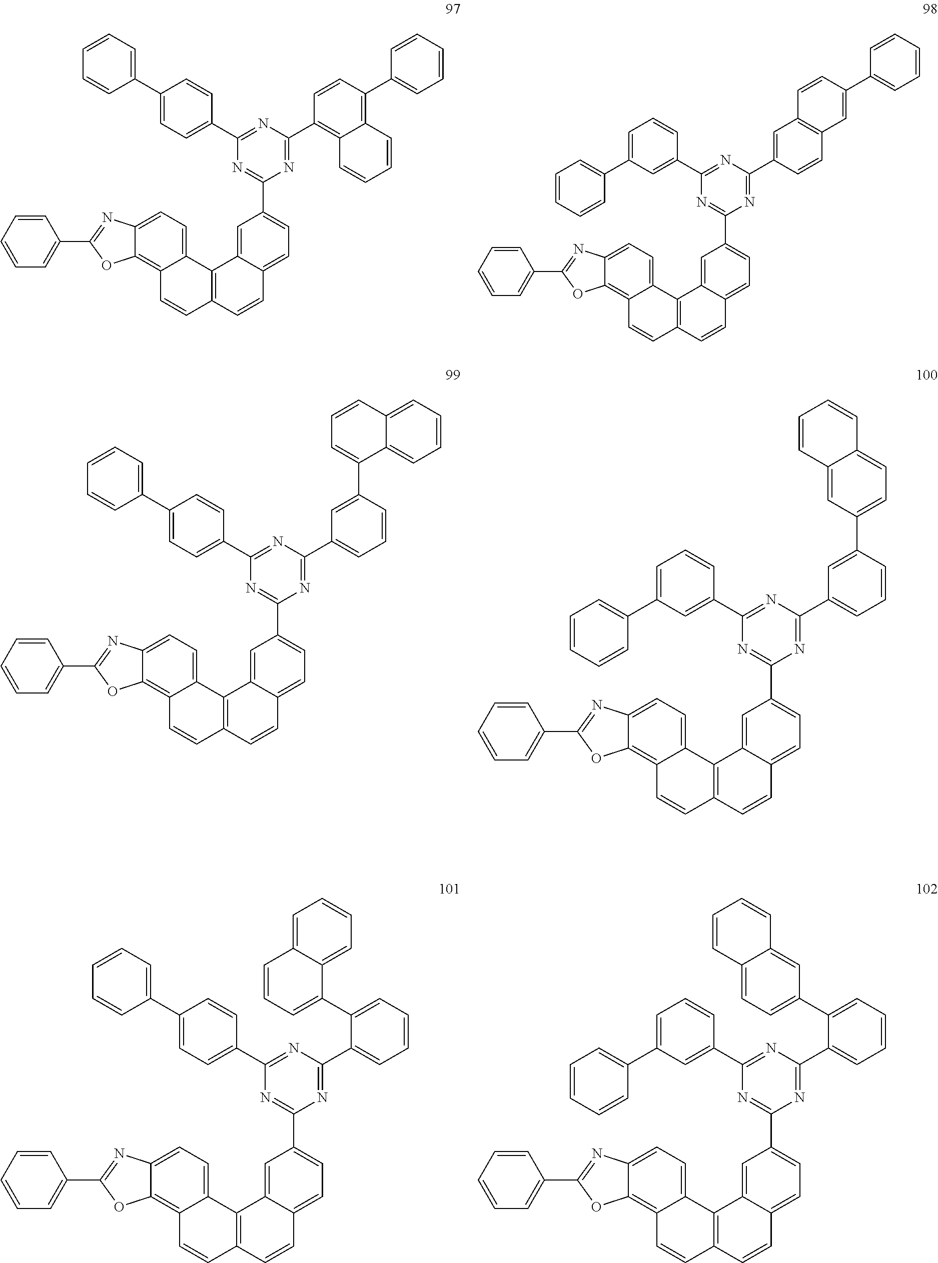

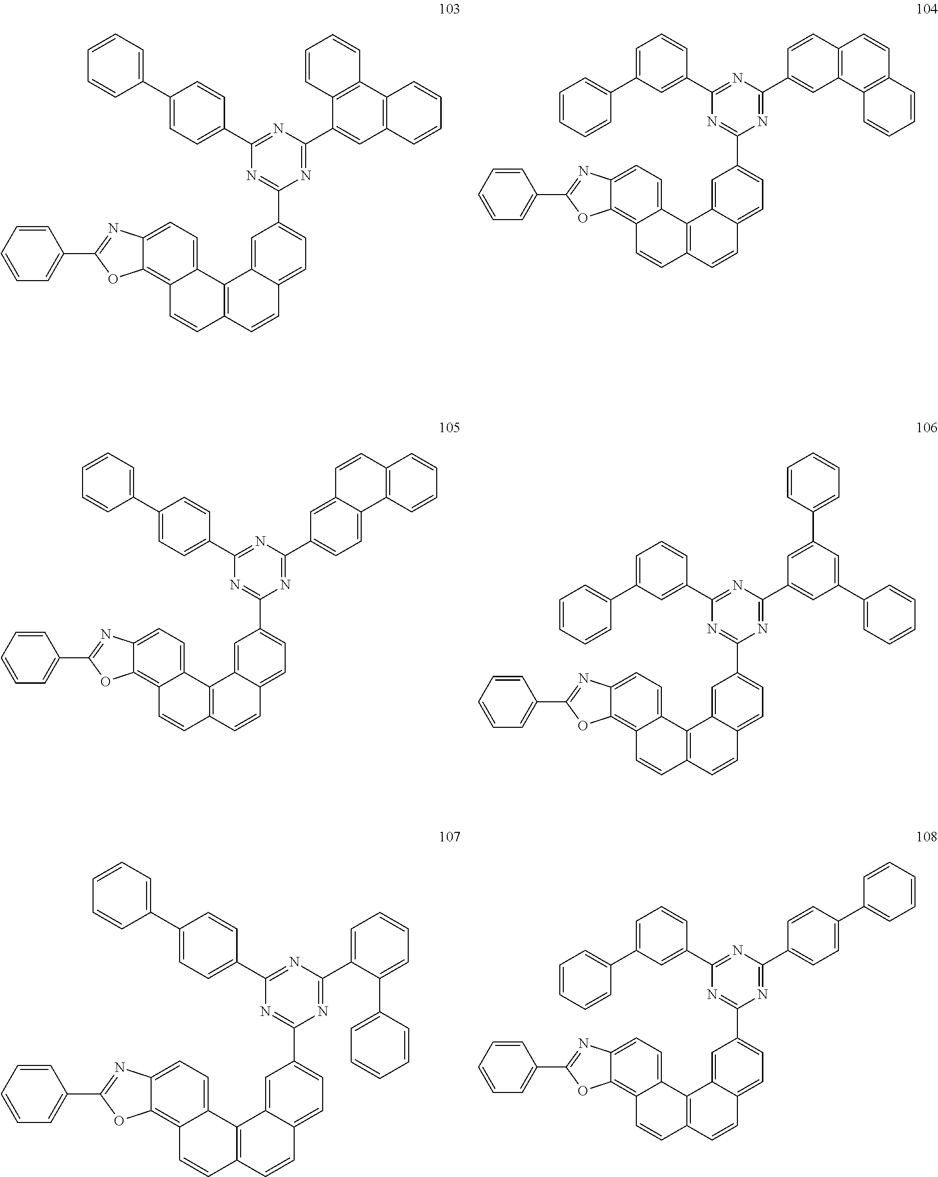

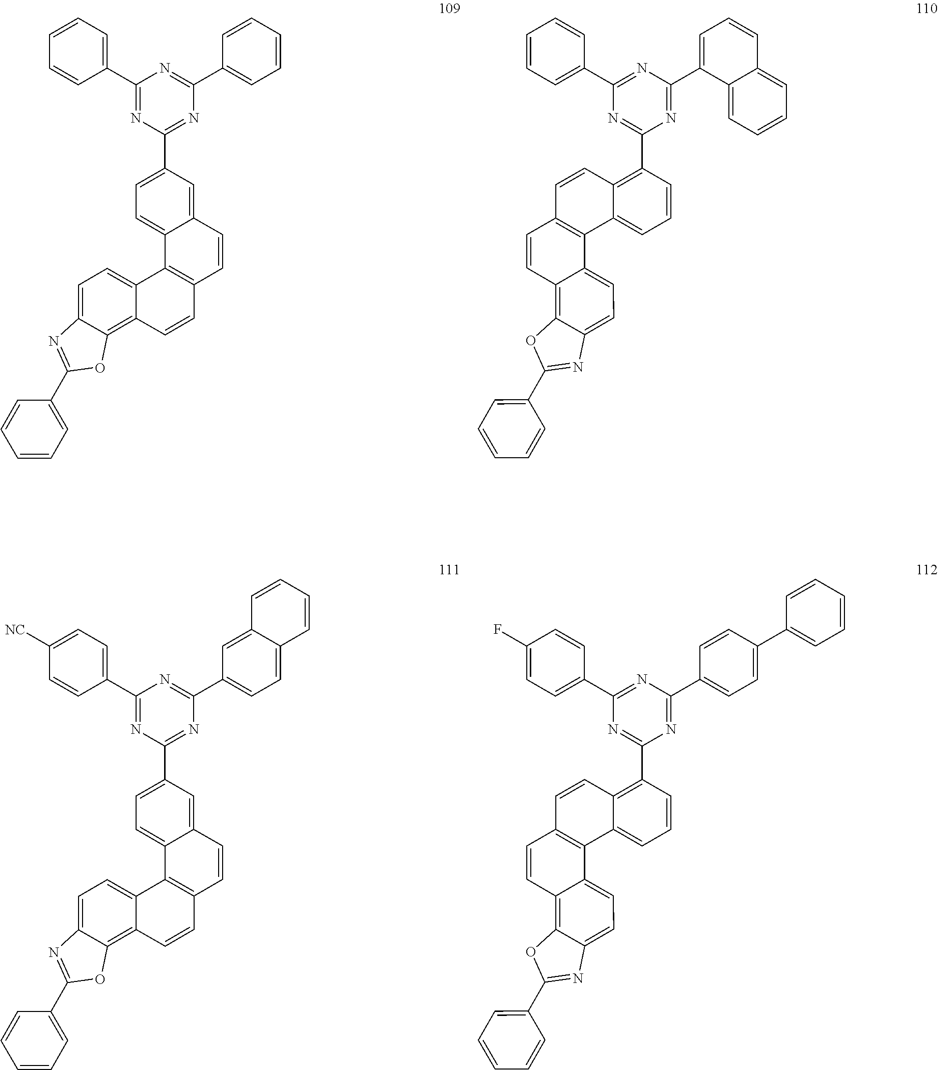 [SMILES: c1ccc(-c2nc(-c3ccccc3)nc(-c3ccc4c(ccc5ccc6c(ccc7nc(-c8ccccc8)oc76)c54)c3)n2)cc1 ; c1ccc(-c2nc(-c3cccc4ccccc34)nc(-c3cccc4c3ccc3ccc5c(ccc6nc(-c7ccccc7)oc65)c34)n2)cc1 ; N#Cc1ccc(-c2nc(-c3ccc4ccccc4c3)nc(-c3ccc4c(ccc5ccc6c(ccc7nc(-c8ccccc8)oc76)c54)c3)n2)cc1 ; Fc1ccc(-c2nc(-c3ccc(-c4ccccc4)cc3)nc(-c3cccc4c3ccc3ccc5c(ccc6nc(-c7ccccc7)oc65)c34)n2)cc1]

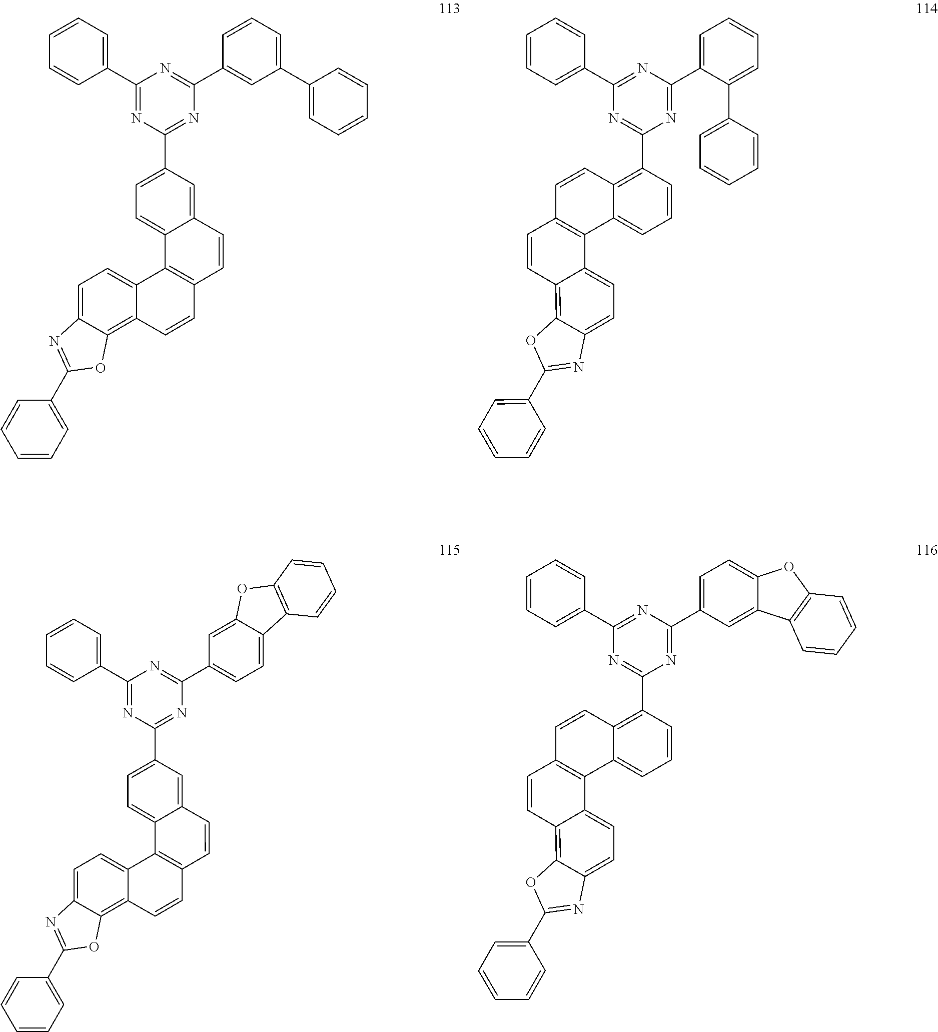 [SMILES: c1ccc(-c2cccc(-c3nc(-c4ccccc4)nc(-c4ccc5c(ccc6ccc7c(ccc8nc(-c9ccccc9)oc87)c65)c4)n3)c2)cc1 ; c1ccc(-c2nc(-c3ccccc3-c3ccccc3)nc(-c3cccc4c3ccc3ccc5c(ccc6nc(-c7ccccc7)oc65)c34)n2)cc1 ; c1ccc(-c2nc(-c3ccc4c(ccc5ccc6c(ccc7nc(-c8ccccc8)oc76)c54)c3)nc(-c3ccc4c(c3)oc3ccccc34)n2)cc1 ; c1ccc(-c2nc(-c3ccc4oc5ccccc5c4c3)nc(-c3cccc4c3ccc3ccc5c(ccc6nc(-c7ccccc7)oc65)c34)n2)cc1]

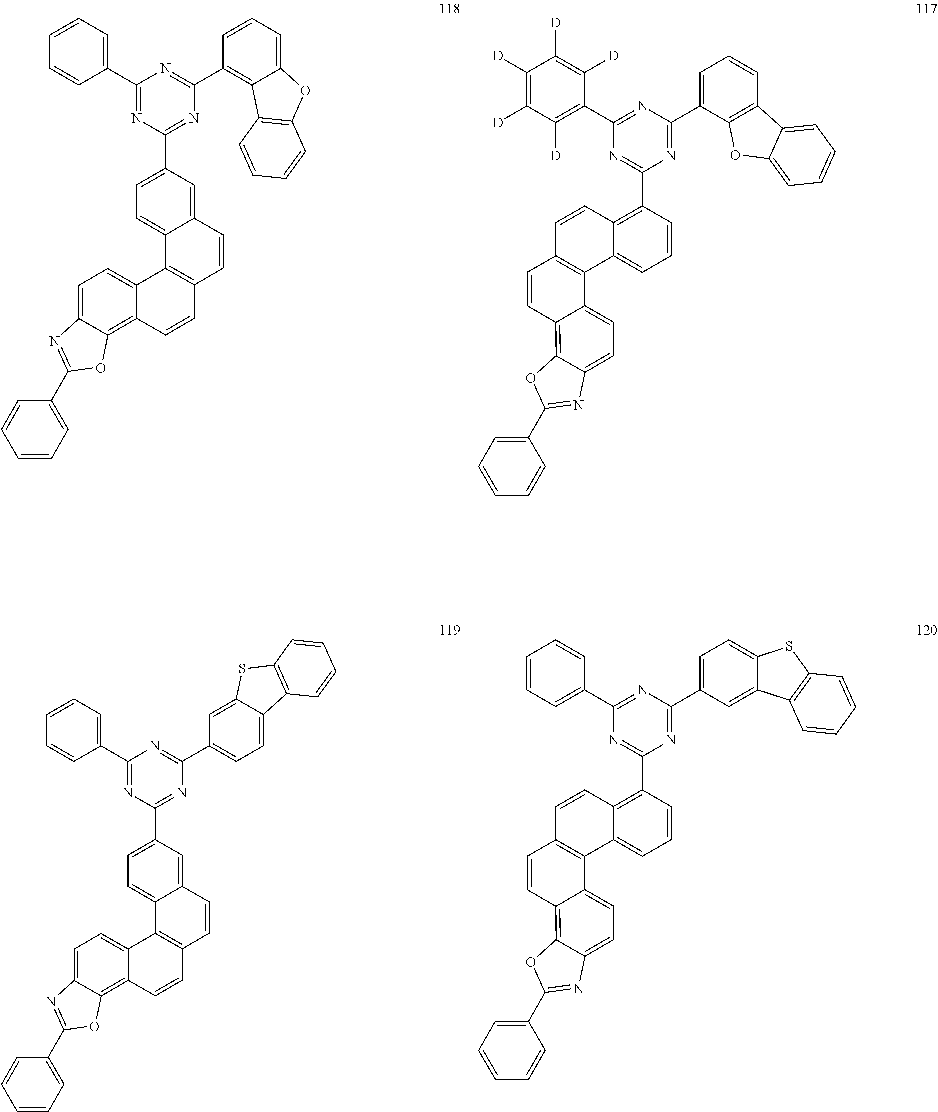 [SMILES: c1ccc(-c2nc(-c3ccc4c(ccc5ccc6c(ccc7nc(-c8ccccc8)oc76)c54)c3)nc(-c3cccc4oc5ccccc5c34)n2)cc1 ; [2H]c1c([2H])c([2H])c(-c2nc(-c3cccc4c3ccc3ccc5c(ccc6nc(-c7ccccc7)oc65)c34)nc(-c3cccc4c3oc3ccccc34)n2)c([2H])c1[2H] ; c1ccc(-c2nc(-c3ccc4c(ccc5ccc6c(ccc7nc(-c8ccccc8)oc76)c54)c3)nc(-c3ccc4c(c3)sc3ccccc34)n2)cc1 ; c1ccc(-c2nc(-c3ccc4sc5ccccc5c4c3)nc(-c3cccc4c3ccc3ccc5c(ccc6nc(-c7ccccc7)oc65)c34)n2)cc1]

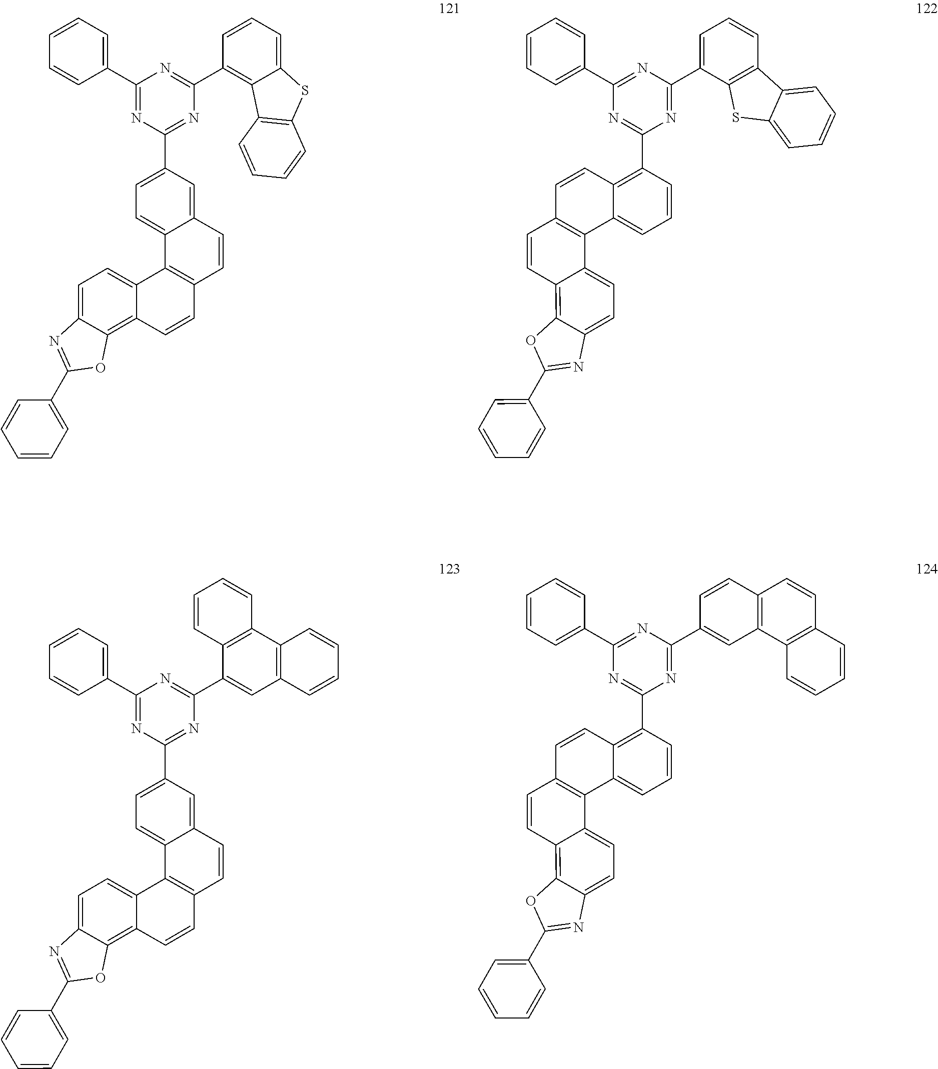 [SMILES: c1ccc(-c2nc(-c3ccc4c(ccc5ccc6c(ccc7nc(-c8ccccc8)oc76)c54)c3)nc(-c3cccc4sc5ccccc5c34)n2)cc1 ; c1ccc(-c2nc(-c3cccc4c3ccc3ccc5c(ccc6nc(-c7ccccc7)oc65)c34)nc(-c3cccc4c3sc3ccccc34)n2)cc1 ; c1ccc(-c2nc(-c3ccc4c(ccc5ccc6c(ccc7nc(-c8ccccc8)oc76)c54)c3)nc(-c3cc4ccccc4c4ccccc34)n2)cc1 ; c1ccc(-c2nc(-c3ccc4ccc5ccccc5c4c3)nc(-c3cccc4c3ccc3ccc5c(ccc6nc(-c7ccccc7)oc65)c34)n2)cc1]

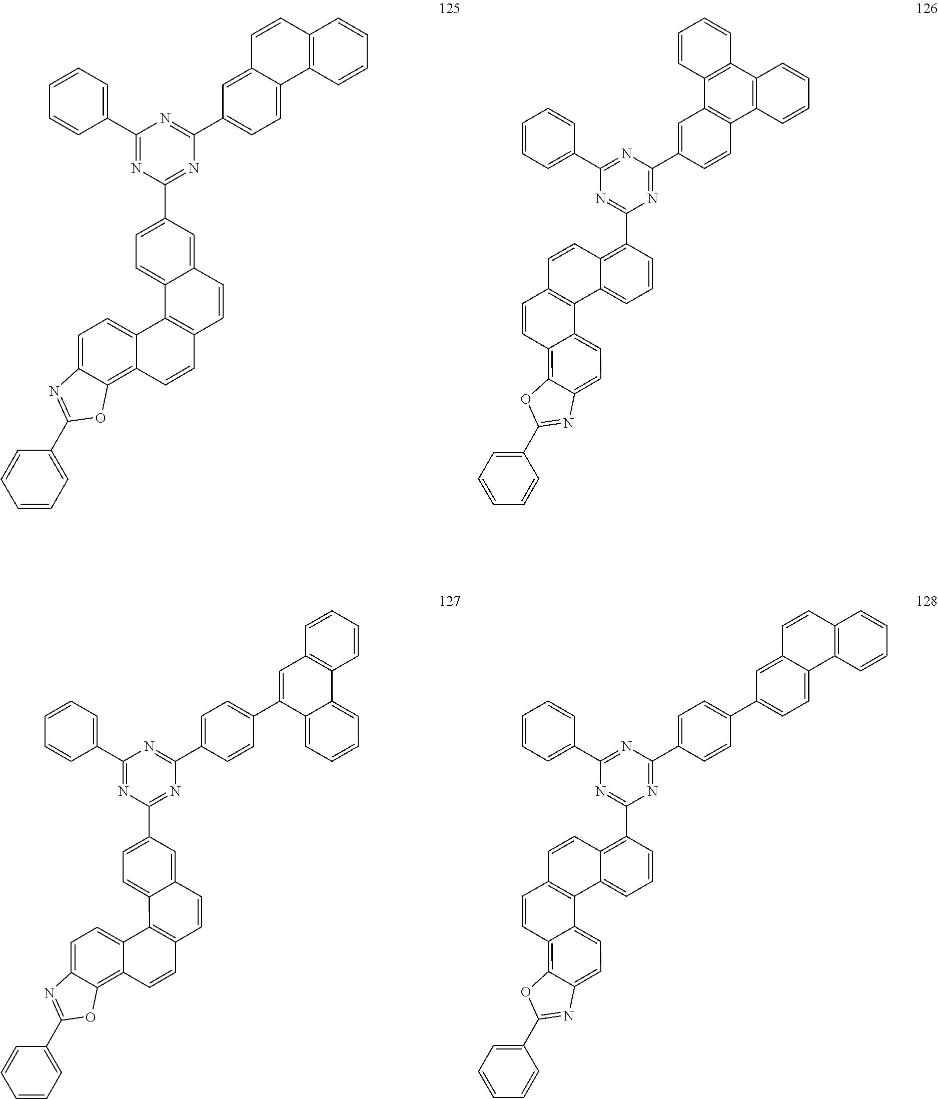 [SMILES: c1ccc(-c2nc(-c3ccc4c(ccc5ccccc54)c3)nc(-c3ccc4c(ccc5ccc6c(ccc7nc(-c8ccccc8)oc76)c54)c3)n2)cc1 ; c1ccc(-c2nc(-c3ccc4c5ccccc5c5ccccc5c4c3)nc(-c3cccc4c3ccc3ccc5c(ccc6nc(-c7ccccc7)oc65)c34)n2)cc1 ; c1ccc(-c2nc(-c3ccc(-c4cc5ccccc5c5ccccc45)cc3)nc(-c3ccc4c(ccc5ccc6c(ccc7nc(-c8ccccc8)oc76)c54)c3)n2)cc1 ; c1ccc(-c2nc(-c3ccc(-c4ccc5c(ccc6ccccc65)c4)cc3)nc(-c3cccc4c3ccc3ccc5c(ccc6nc(-c7ccccc7)oc65)c34)n2)cc1]

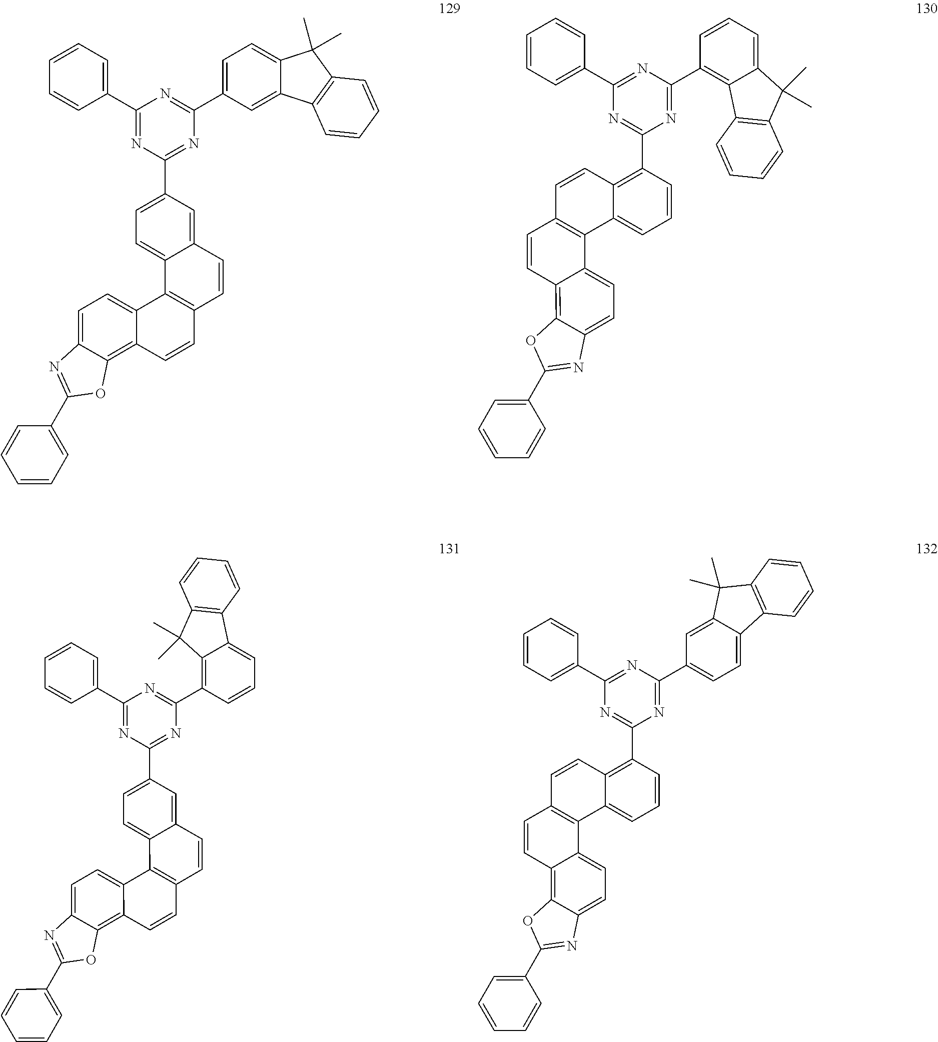 [SMILES: CC1(C)c2ccccc2-c2cc(-c3nc(-c4ccccc4)nc(-c4ccc5c(ccc6ccc7c(ccc8nc(-c9ccccc9)oc87)c65)c4)n3)ccc21 ; CC1(C)c2ccccc2-c2c(-c3nc(-c4ccccc4)nc(-c4cccc5c4ccc4ccc6c(ccc7nc(-c8ccccc8)oc76)c45)n3)cccc21 ; CC1(C)c2ccccc2-c2cccc(-c3nc(-c4ccccc4)nc(-c4ccc5c(ccc6ccc7c(ccc8nc(-c9ccccc9)oc87)c65)c4)n3)c21 ; CC1(C)c2ccccc2-c2ccc(-c3nc(-c4ccccc4)nc(-c4cccc5c4ccc4ccc6c(ccc7nc(-c8ccccc8)oc76)c45)n3)cc21]

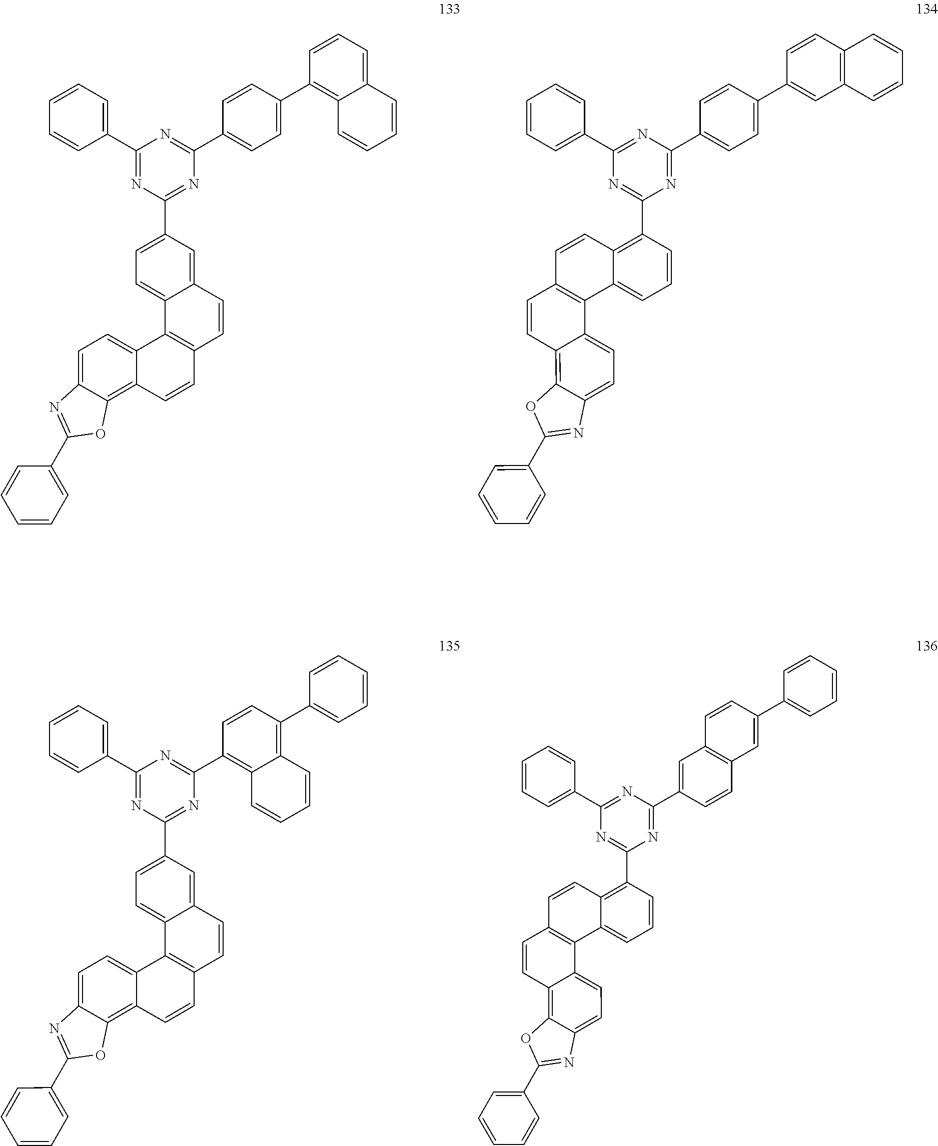 [SMILES: c1ccc(-c2nc(-c3ccc(-c4cccc5ccccc45)cc3)nc(-c3ccc4c(ccc5ccc6c(ccc7nc(-c8ccccc8)oc76)c54)c3)n2)cc1 ; c1ccc(-c2nc(-c3ccc(-c4ccc5ccccc5c4)cc3)nc(-c3cccc4c3ccc3ccc5c(ccc6nc(-c7ccccc7)oc65)c34)n2)cc1 ; c1ccc(-c2nc(-c3ccc4c(ccc5ccc6c(ccc7nc(-c8ccccc8)oc76)c54)c3)nc(-c3ccc(-c4ccccc4)c4ccccc34)n2)cc1 ; c1ccc(-c2ccc3cc(-c4nc(-c5ccccc5)nc(-c5cccc6c5ccc5ccc7c(ccc8nc(-c9ccccc9)oc87)c56)n4)ccc3c2)cc1]

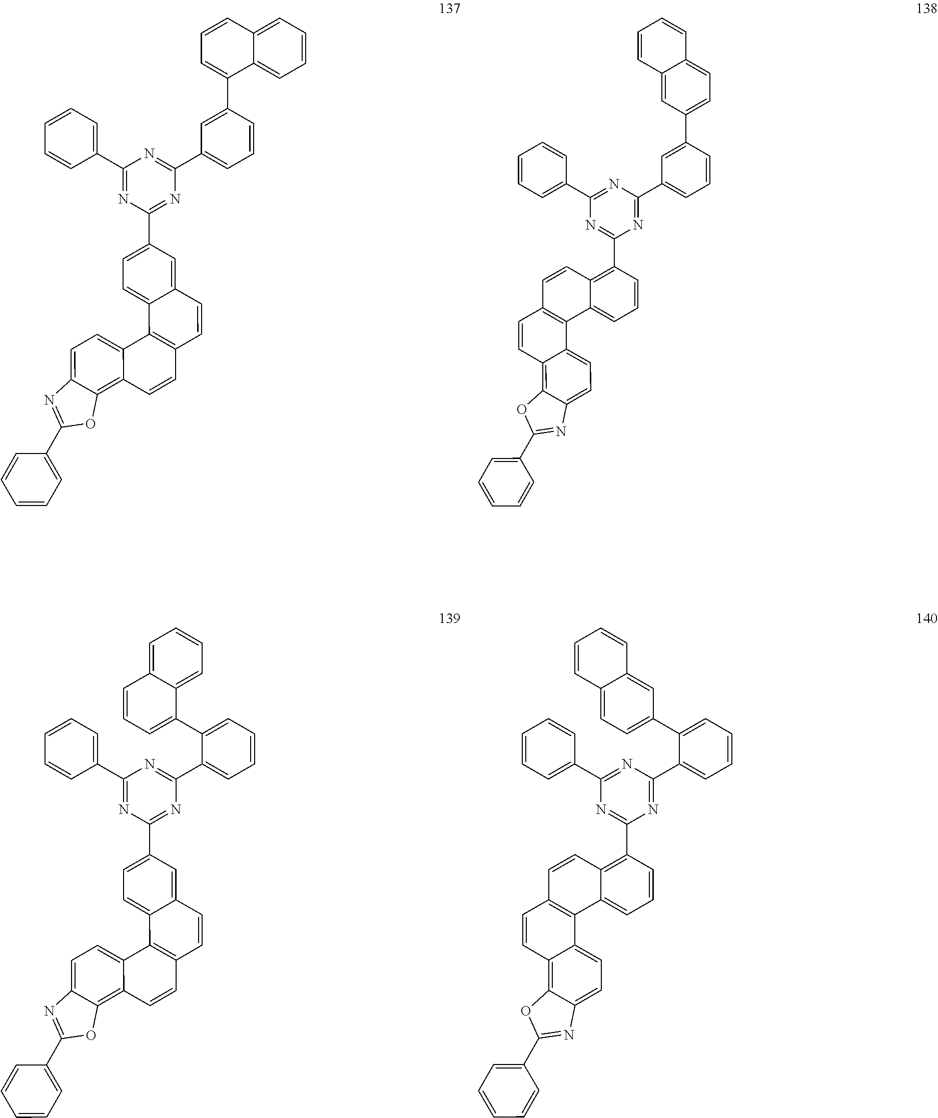 [SMILES: c1ccc(-c2nc(-c3cccc(-c4cccc5ccccc45)c3)nc(-c3ccc4c(ccc5ccc6c(ccc7nc(-c8ccccc8)oc76)c54)c3)n2)cc1 ; c1ccc(-c2nc(-c3cccc(-c4ccc5ccccc5c4)c3)nc(-c3cccc4c3ccc3ccc5c(ccc6nc(-c7ccccc7)oc65)c34)n2)cc1 ; c1ccc(-c2nc(-c3ccc4c(ccc5ccc6c(ccc7nc(-c8ccccc8)oc76)c54)c3)nc(-c3ccccc3-c3cccc4ccccc34)n2)cc1 ; c1ccc(-c2nc(-c3ccccc3-c3ccc4ccccc4c3)nc(-c3cccc4c3ccc3ccc5c(ccc6nc(-c7ccccc7)oc65)c34)n2)cc1]

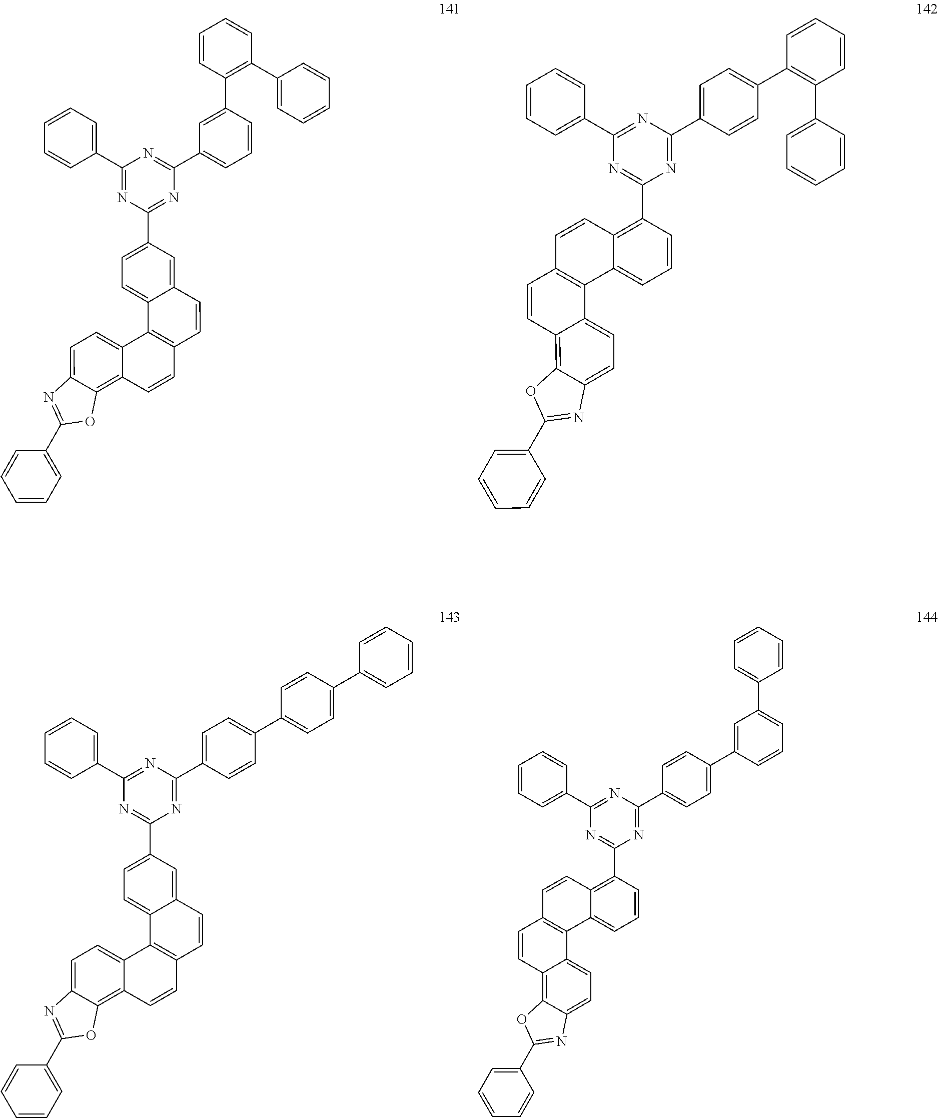 [SMILES: c1ccc(-c2nc(-c3cccc(-c4ccccc4-c4ccccc4)c3)nc(-c3ccc4c(ccc5ccc6c(ccc7nc(-c8ccccc8)oc76)c54)c3)n2)cc1 ; c1ccc(-c2nc(-c3ccc(-c4ccccc4-c4ccccc4)cc3)nc(-c3cccc4c3ccc3ccc5c(ccc6nc(-c7ccccc7)oc65)c34)n2)cc1 ; c1ccc(-c2ccc(-c3ccc(-c4nc(-c5ccccc5)nc(-c5ccc6c(ccc7ccc8c(ccc9nc(-c%10ccccc%10)oc98)c76)c5)n4)cc3)cc2)cc1 ; c1ccc(-c2cccc(-c3ccc(-c4nc(-c5ccccc5)nc(-c5cccc6c5ccc5ccc7c(ccc8nc(-c9ccccc9)oc87)c56)n4)cc3)c2)cc1]

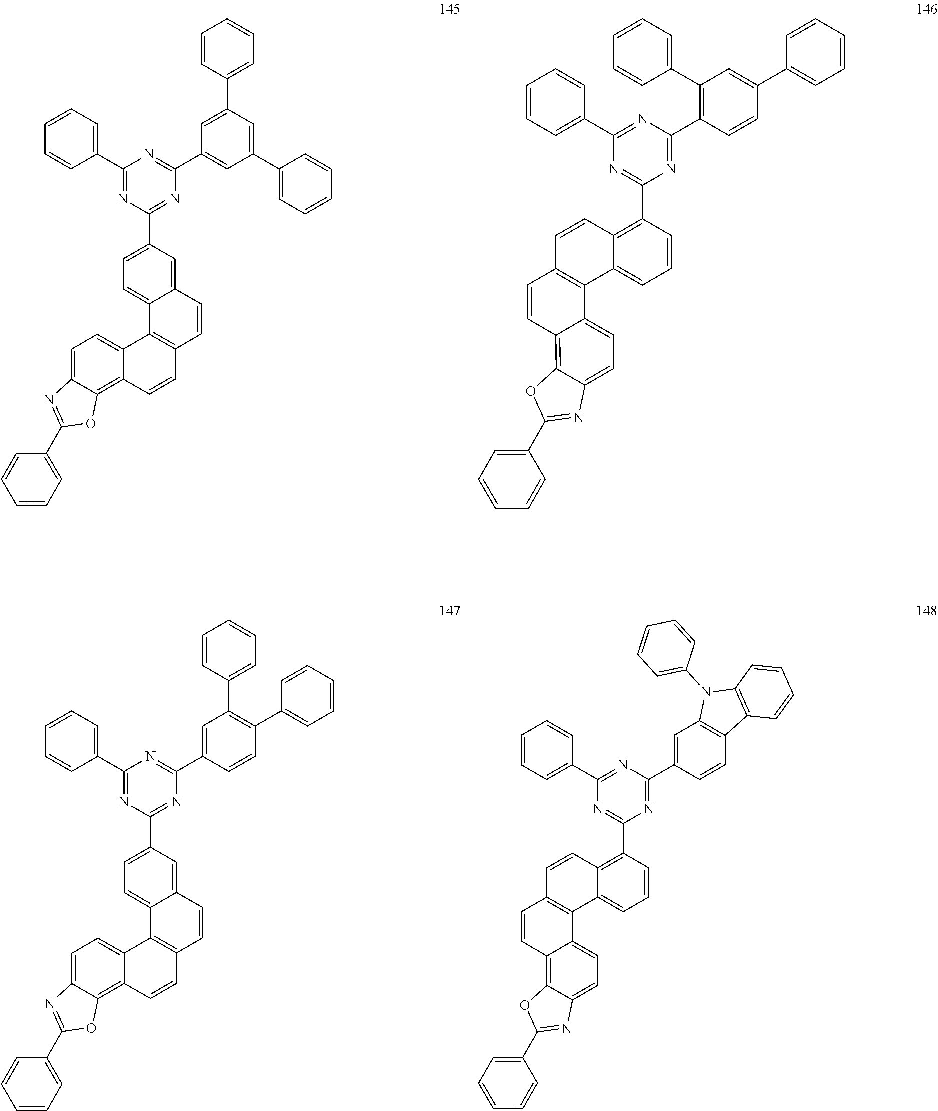 [SMILES: c1ccc(-c2cc(-c3ccccc3)cc(-c3nc(-c4ccccc4)nc(-c4ccc5c(ccc6ccc7c(ccc8nc(-c9ccccc9)oc87)c65)c4)n3)c2)cc1 ; c1ccc(-c2ccc(-c3nc(-c4ccccc4)nc(-c4cccc5c4ccc4ccc6c(ccc7nc(-c8ccccc8)oc76)c45)n3)c(-c3ccccc3)c2)cc1 ; c1ccc(-c2nc(-c3ccc(-c4ccccc4)c(-c4ccccc4)c3)nc(-c3ccc4c(ccc5ccc6c(ccc7nc(-c8ccccc8)oc76)c54)c3)n2)cc1 ; c1ccc(-c2nc(-c3ccc4c5ccccc5n(-c5ccccc5)c4c3)nc(-c3cccc4c3ccc3ccc5c(ccc6nc(-c7ccccc7)oc65)c34)n2)cc1]

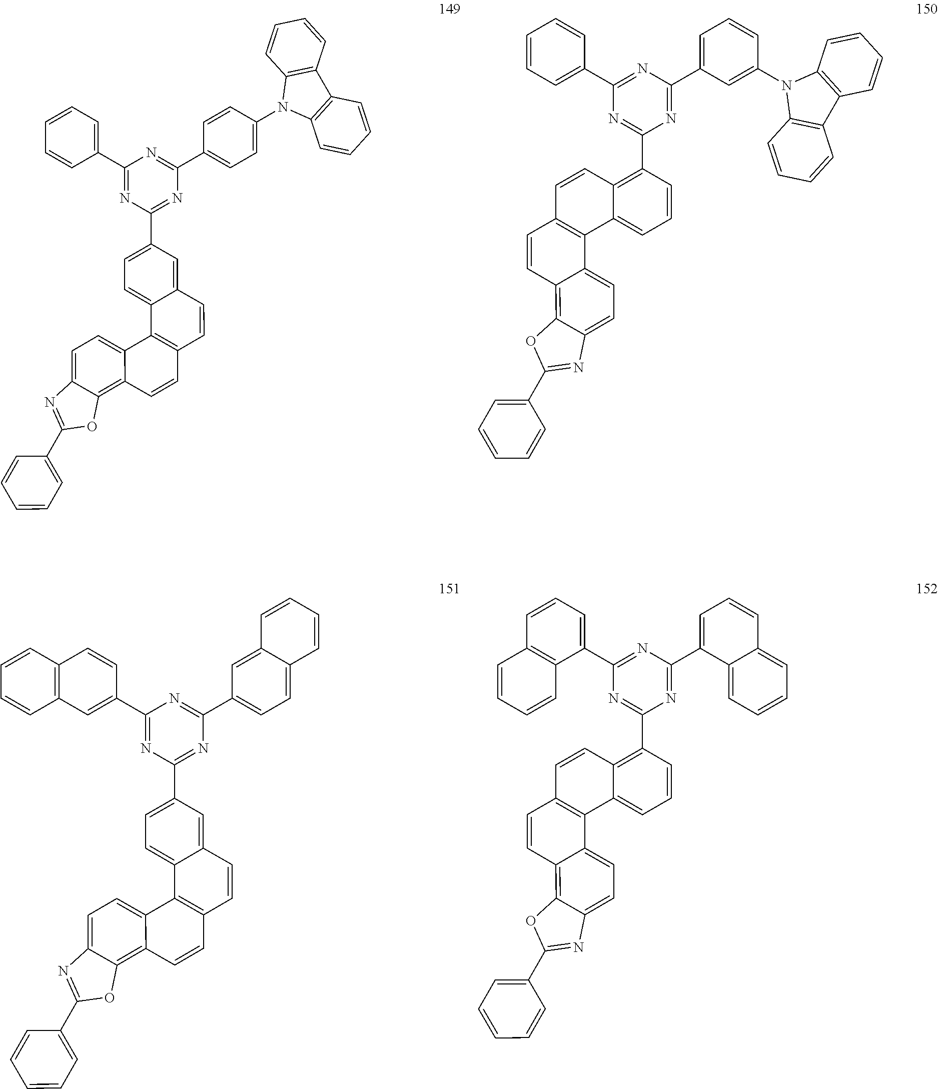 [SMILES: c1ccc(-c2nc(-c3ccc(-n4c5ccccc5c5ccccc54)cc3)nc(-c3ccc4c(ccc5ccc6c(ccc7nc(-c8ccccc8)oc76)c54)c3)n2)cc1 ; c1ccc(-c2nc(-c3cccc(-n4c5ccccc5c5ccccc54)c3)nc(-c3cccc4c3ccc3ccc5c(ccc6nc(-c7ccccc7)oc65)c34)n2)cc1 ; c1ccc(-c2nc3ccc4c(ccc5ccc6cc(-c7nc(-c8ccc9ccccc9c8)nc(-c8ccc9ccccc9c8)n7)ccc6c54)c3o2)cc1 ; c1ccc(-c2nc3ccc4c(ccc5ccc6c(-c7nc(-c8cccc9ccccc89)nc(-c8cccc9ccccc89)n7)cccc6c54)c3o2)cc1]

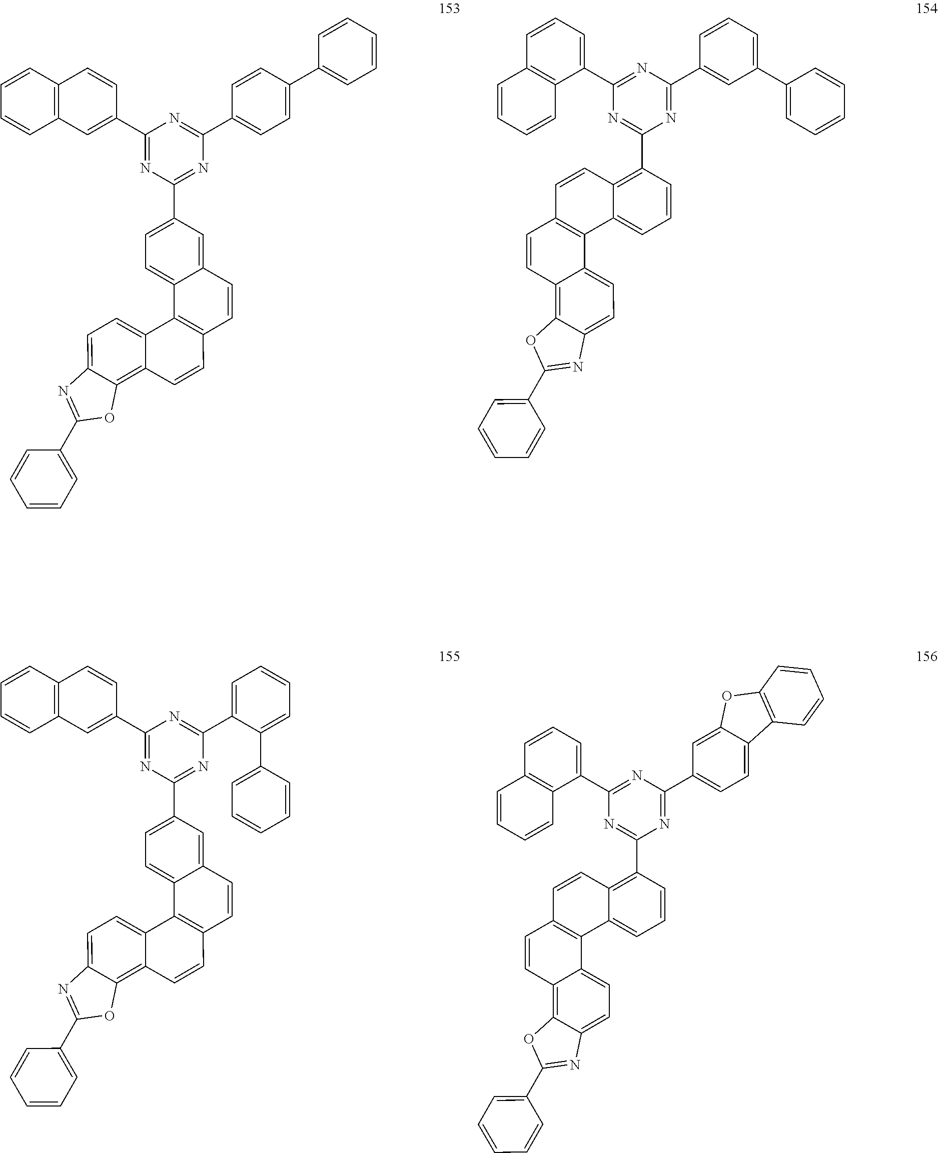 [SMILES: c1ccc(-c2ccc(-c3nc(-c4ccc5ccccc5c4)nc(-c4ccc5c(ccc6ccc7c(ccc8nc(-c9ccccc9)oc87)c65)c4)n3)cc2)cc1 ; c1ccc(-c2cccc(-c3nc(-c4cccc5ccccc45)nc(-c4cccc5c4ccc4ccc6c(ccc7nc(-c8ccccc8)oc76)c45)n3)c2)cc1 ; c1ccc(-c2nc3ccc4c(ccc5ccc6cc(-c7nc(-c8ccc9ccccc9c8)nc(-c8ccccc8-c8ccccc8)n7)ccc6c54)c3o2)cc1 ; c1ccc(-c2nc3ccc4c(ccc5ccc6c(-c7nc(-c8ccc9c(c8)oc8ccccc89)nc(-c8cccc9ccccc89)n7)cccc6c54)c3o2)cc1]

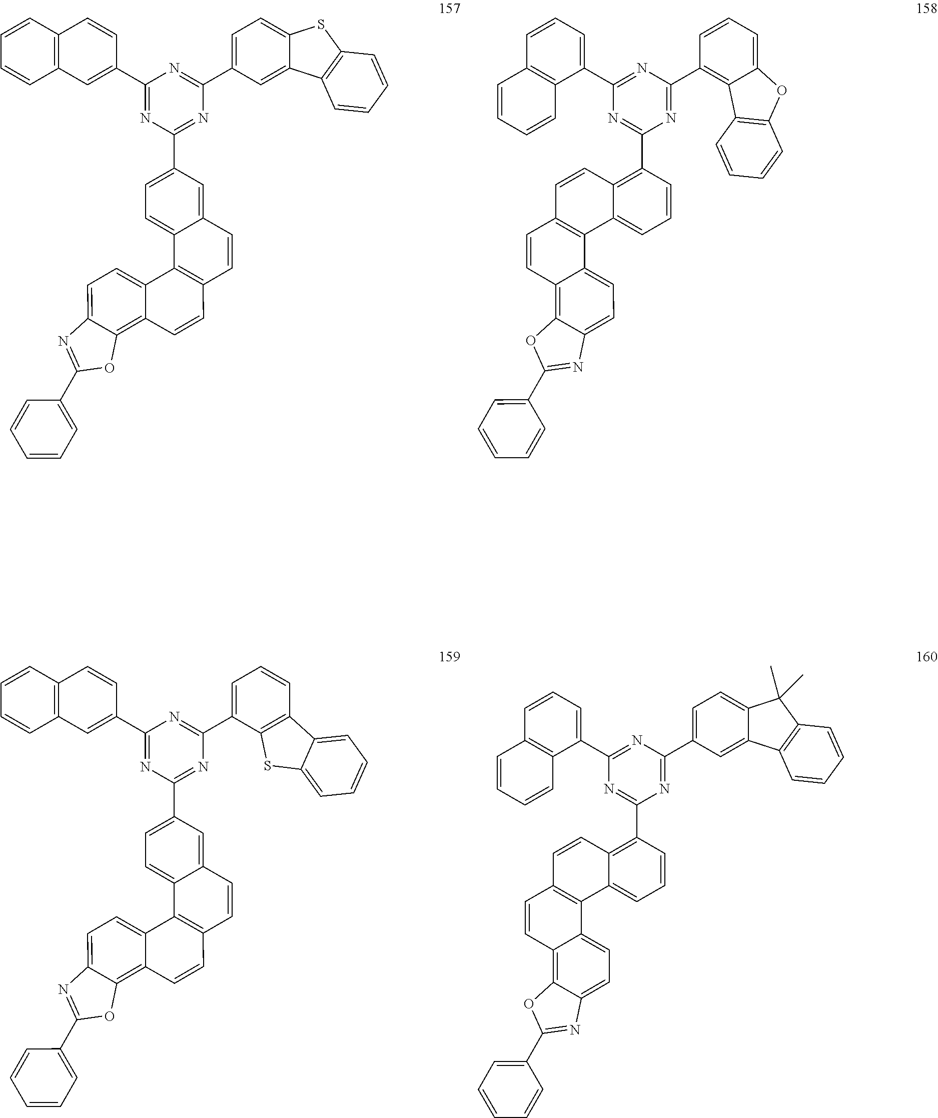 [SMILES: c1ccc(-c2nc3ccc4c(ccc5ccc6cc(-c7nc(-c8ccc9ccccc9c8)nc(-c8ccc9sc%10ccccc%10c9c8)n7)ccc6c54)c3o2)cc1 ; c1ccc(-c2nc3ccc4c(ccc5ccc6c(-c7nc(-c8cccc9ccccc89)nc(-c8cccc9oc%10ccccc%10c89)n7)cccc6c54)c3o2)cc1 ; c1ccc(-c2nc3ccc4c(ccc5ccc6cc(-c7nc(-c8ccc9ccccc9c8)nc(-c8cccc9c8sc8ccccc89)n7)ccc6c54)c3o2)cc1 ; CC1(C)c2ccccc2-c2cc(-c3nc(-c4cccc5ccccc45)nc(-c4cccc5c4ccc4ccc6c(ccc7nc(-c8ccccc8)oc76)c45)n3)ccc21]

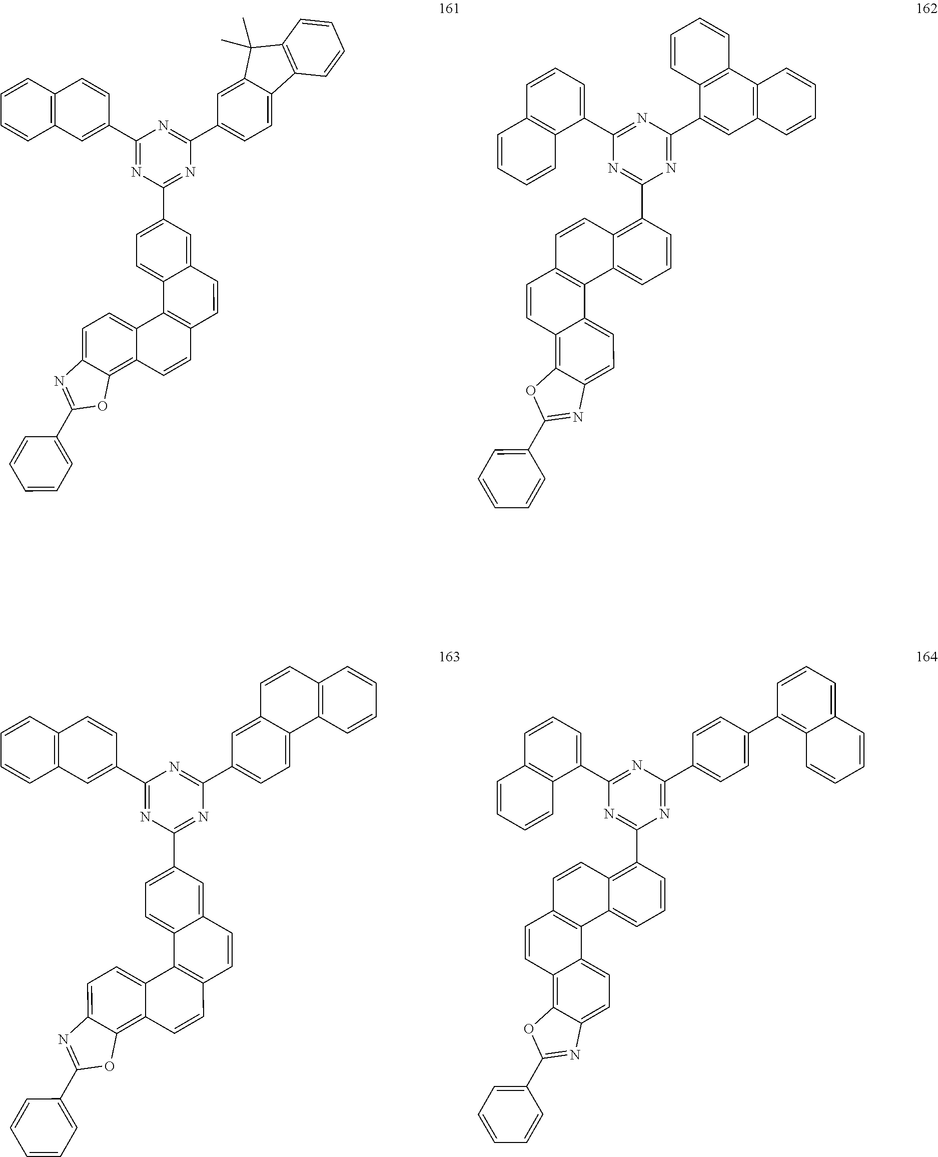 [SMILES: CC1(C)c2ccccc2-c2ccc(-c3nc(-c4ccc5ccccc5c4)nc(-c4ccc5c(ccc6ccc7c(ccc8nc(-c9ccccc9)oc87)c65)c4)n3)cc21 ; c1ccc(-c2nc3ccc4c(ccc5ccc6c(-c7nc(-c8cccc9ccccc89)nc(-c8cc9ccccc9c9ccccc89)n7)cccc6c54)c3o2)cc1 ; c1ccc(-c2nc3ccc4c(ccc5ccc6cc(-c7nc(-c8ccc9ccccc9c8)nc(-c8ccc9c(ccc%10ccccc%109)c8)n7)ccc6c54)c3o2)cc1 ; c1ccc(-c2nc3ccc4c(ccc5ccc6c(-c7nc(-c8ccc(-c9cccc%10ccccc9%10)cc8)nc(-c8cccc9ccccc89)n7)cccc6c54)c3o2)cc1]

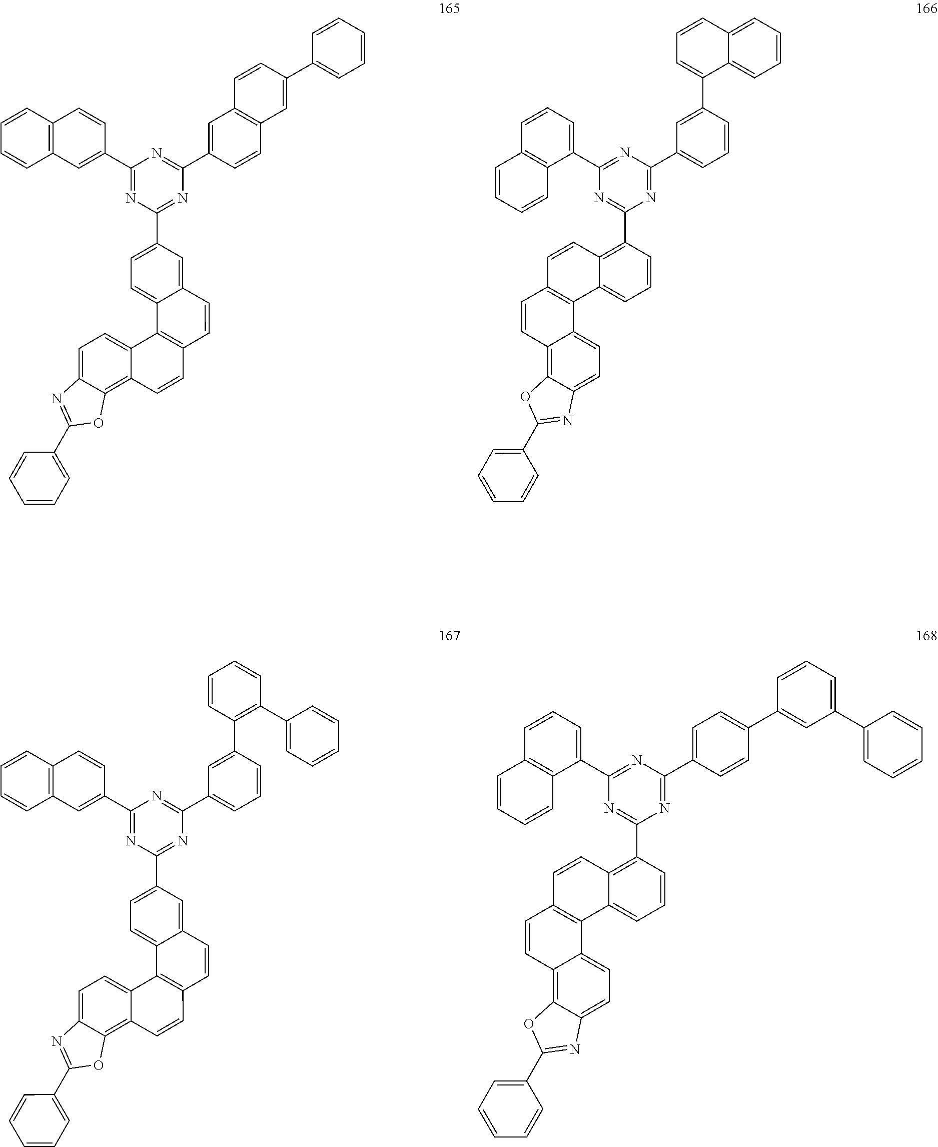 [SMILES: c1ccc(-c2ccc3cc(-c4nc(-c5ccc6ccccc6c5)nc(-c5ccc6c(ccc7ccc8c(ccc9nc(-c%10ccccc%10)oc98)c76)c5)n4)ccc3c2)cc1 ; c1ccc(-c2nc3ccc4c(ccc5ccc6c(-c7nc(-c8cccc(-c9cccc%10ccccc9%10)c8)nc(-c8cccc9ccccc89)n7)cccc6c54)c3o2)cc1 ; c1ccc(-c2nc3ccc4c(ccc5ccc6cc(-c7nc(-c8cccc(-c9ccccc9-c9ccccc9)c8)nc(-c8ccc9ccccc9c8)n7)ccc6c54)c3o2)cc1 ; c1ccc(-c2cccc(-c3ccc(-c4nc(-c5cccc6ccccc56)nc(-c5cccc6c5ccc5ccc7c(ccc8nc(-c9ccccc9)oc87)c56)n4)cc3)c2)cc1]

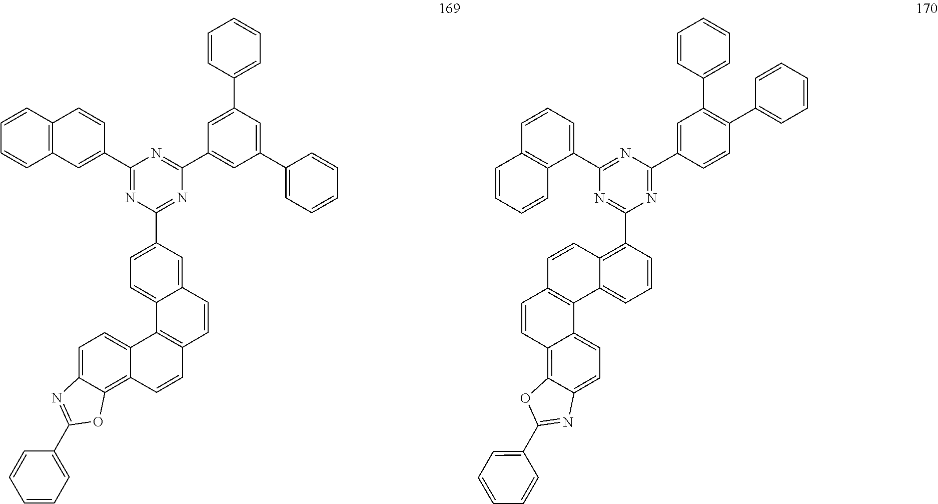 [SMILES: c1ccc(-c2cc(-c3ccccc3)cc(-c3nc(-c4ccc5ccccc5c4)nc(-c4ccc5c(ccc6ccc7c(ccc8nc(-c9ccccc9)oc87)c65)c4)n3)c2)cc1 ; c1ccc(-c2nc3ccc4c(ccc5ccc6c(-c7nc(-c8ccc(-c9ccccc9)c(-c9ccccc9)c8)nc(-c8cccc9ccccc89)n7)cccc6c54)c3o2)cc1]

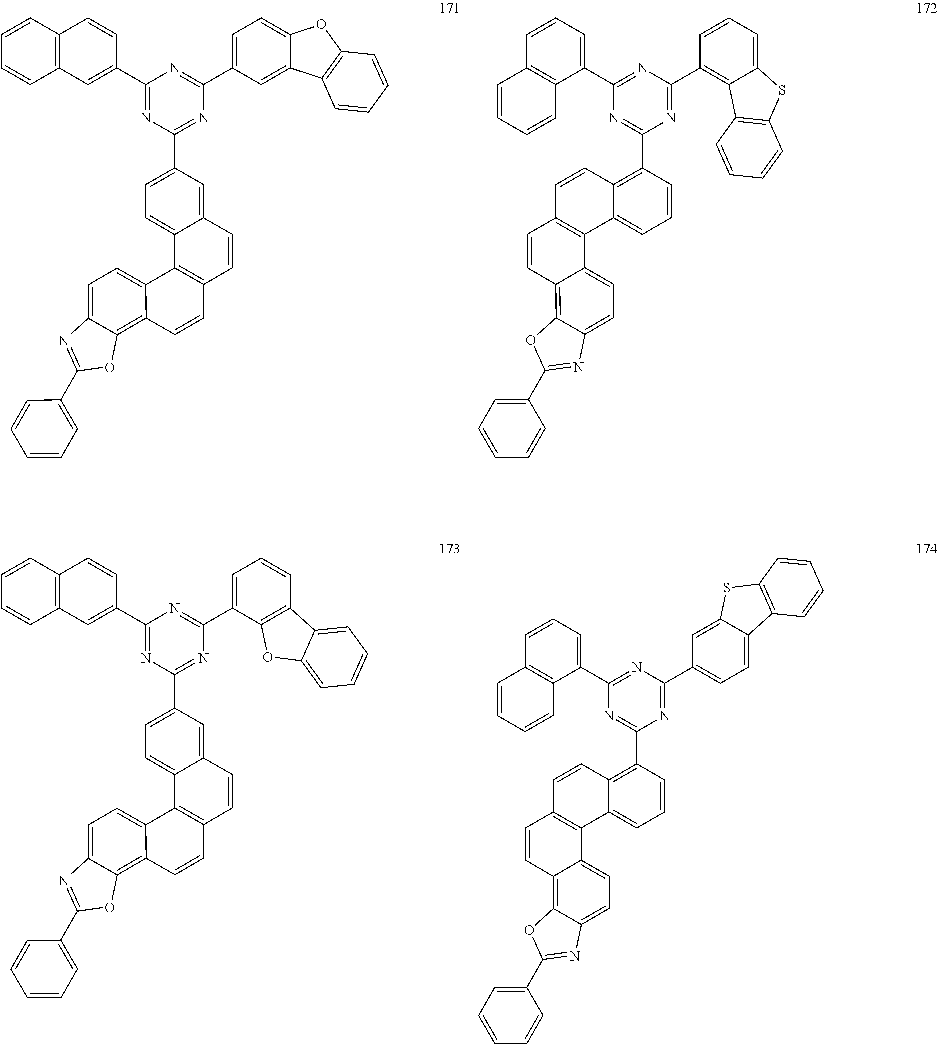 [SMILES: c1ccc(-c2nc3ccc4c(ccc5ccc6cc(-c7nc(-c8ccc9ccccc9c8)nc(-c8ccc9oc%10ccccc%10c9c8)n7)ccc6c54)c3o2)cc1 ; c1ccc(-c2nc3ccc4c(ccc5ccc6c(-c7nc(-c8cccc9ccccc89)nc(-c8cccc9sc%10ccccc%10c89)n7)cccc6c54)c3o2)cc1 ; c1ccc(-c2nc3ccc4c(ccc5ccc6cc(-c7nc(-c8ccc9ccccc9c8)nc(-c8cccc9c8oc8ccccc89)n7)ccc6c54)c3o2)cc1 ; c1ccc(-c2nc3ccc4c(ccc5ccc6c(-c7nc(-c8ccc9c(c8)sc8ccccc89)nc(-c8cccc9ccccc89)n7)cccc6c54)c3o2)cc1]

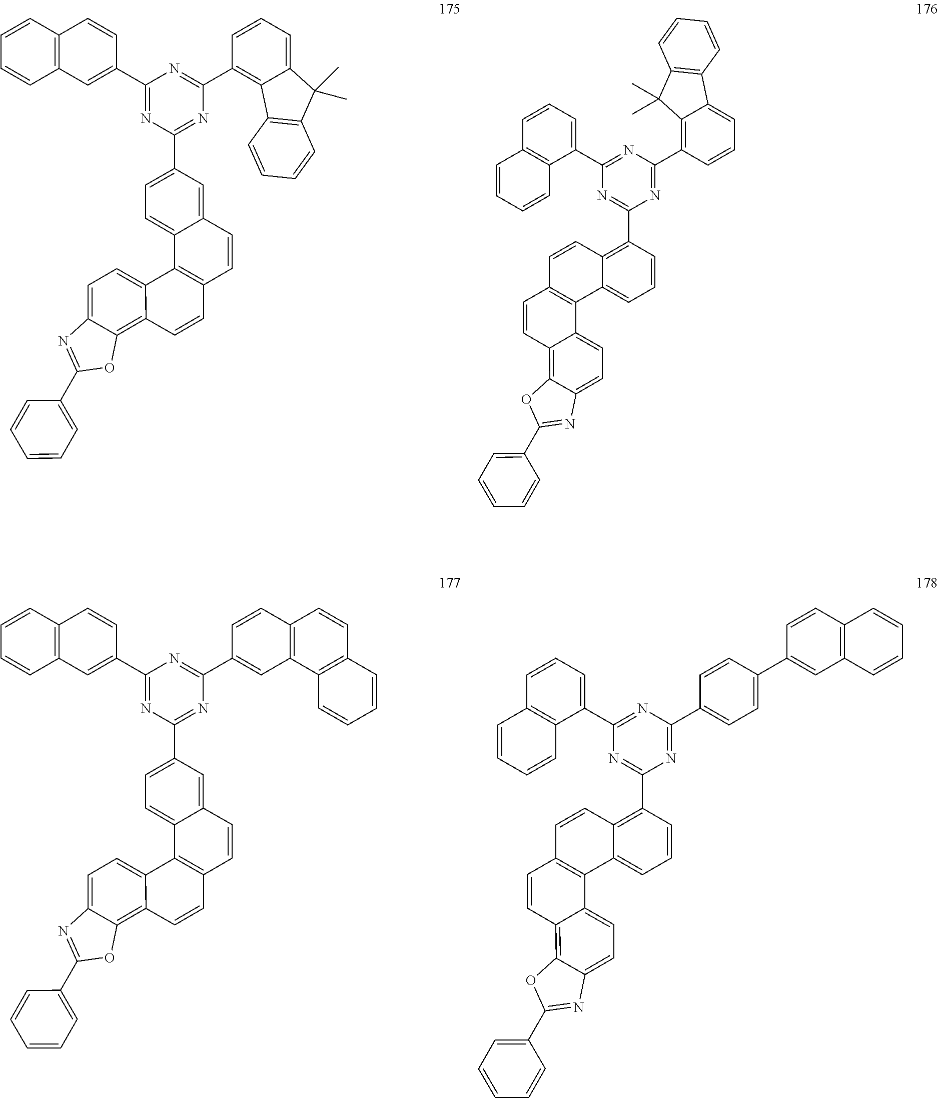 [SMILES: CC1(C)c2ccccc2-c2c(-c3nc(-c4ccc5ccccc5c4)nc(-c4ccc5c(ccc6ccc7c(ccc8nc(-c9ccccc9)oc87)c65)c4)n3)cccc21 ; CC1(C)c2ccccc2-c2cccc(-c3nc(-c4cccc5ccccc45)nc(-c4cccc5c4ccc4ccc6c(ccc7nc(-c8ccccc8)oc76)c45)n3)c21 ; c1ccc(-c2nc3ccc4c(ccc5ccc6cc(-c7nc(-c8ccc9ccccc9c8)nc(-c8ccc9ccc%10ccccc%10c9c8)n7)ccc6c54)c3o2)cc1 ; c1ccc(-c2nc3ccc4c(ccc5ccc6c(-c7nc(-c8ccc(-c9ccc%10ccccc%10c9)cc8)nc(-c8cccc9ccccc89)n7)cccc6c54)c3o2)cc1]

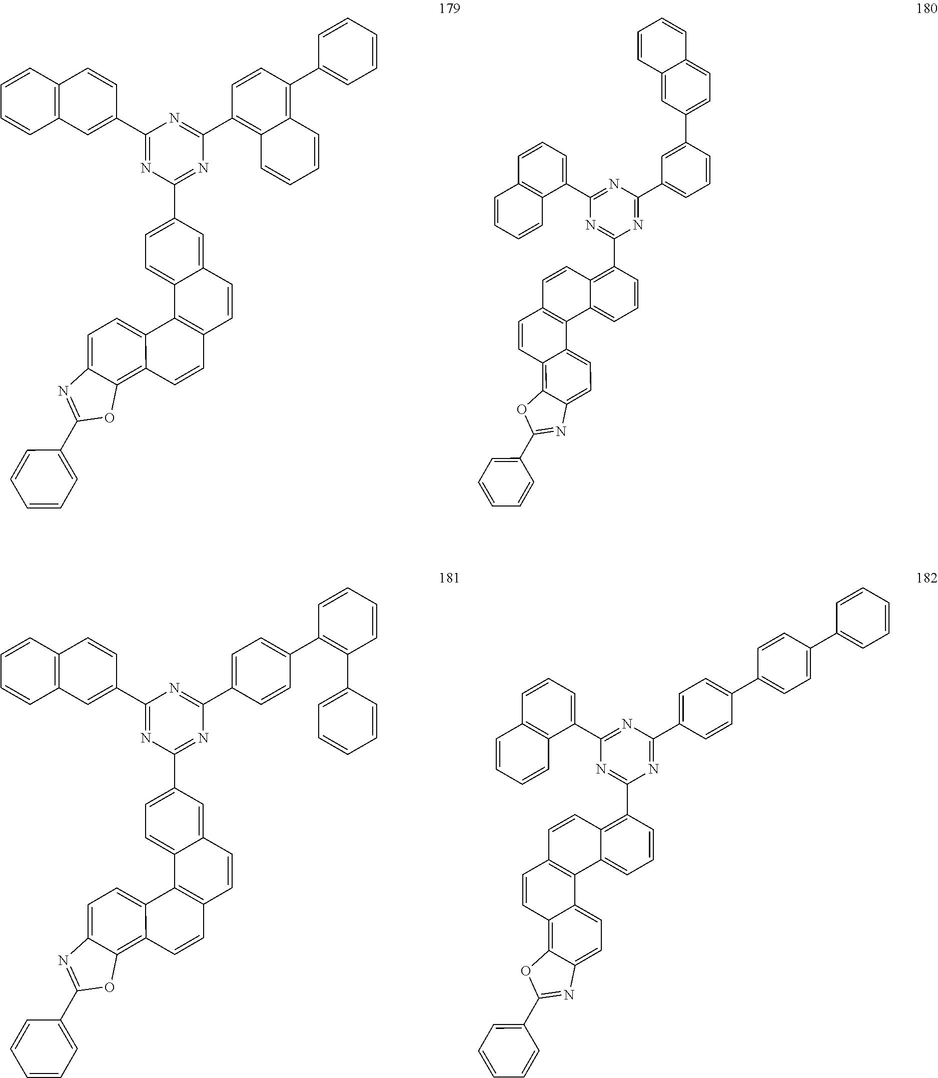 [SMILES: c1ccc(-c2nc3ccc4c(ccc5ccc6cc(-c7nc(-c8ccc9ccccc9c8)nc(-c8ccc(-c9ccccc9)c9ccccc89)n7)ccc6c54)c3o2)cc1 ; c1ccc(-c2nc3ccc4c(ccc5ccc6c(-c7nc(-c8cccc(-c9ccc%10ccccc%10c9)c8)nc(-c8cccc9ccccc89)n7)cccc6c54)c3o2)cc1 ; c1ccc(-c2nc3ccc4c(ccc5ccc6cc(-c7nc(-c8ccc(-c9ccccc9-c9ccccc9)cc8)nc(-c8ccc9ccccc9c8)n7)ccc6c54)c3o2)cc1 ; c1ccc(-c2ccc(-c3ccc(-c4nc(-c5cccc6ccccc56)nc(-c5cccc6c5ccc5ccc7c(ccc8nc(-c9ccccc9)oc87)c56)n4)cc3)cc2)cc1]

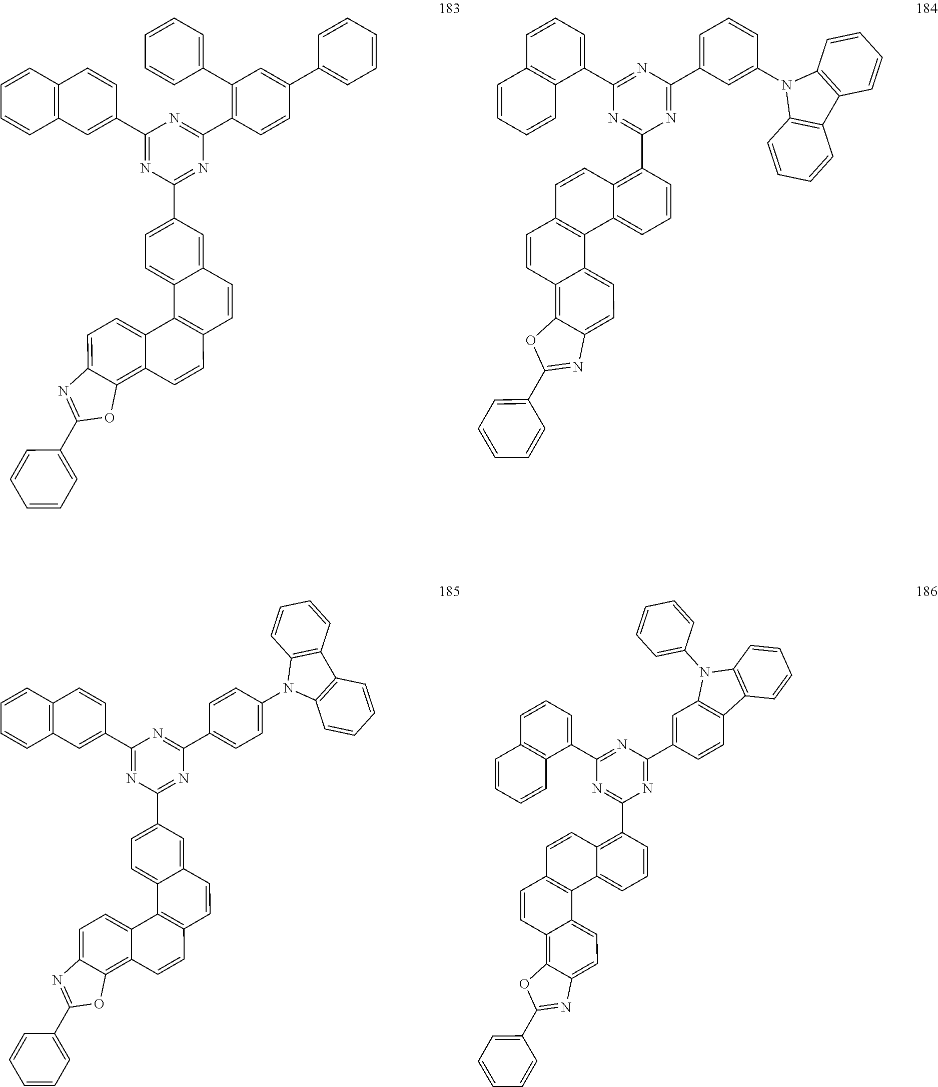 [SMILES: c1ccc(-c2ccc(-c3nc(-c4ccc5ccccc5c4)nc(-c4ccc5c(ccc6ccc7c(ccc8nc(-c9ccccc9)oc87)c65)c4)n3)c(-c3ccccc3)c2)cc1 ; c1ccc(-c2nc3ccc4c(ccc5ccc6c(-c7nc(-c8cccc(-n9c%10ccccc%10c%10ccccc%109)c8)nc(-c8cccc9ccccc89)n7)cccc6c54)c3o2)cc1 ; c1ccc(-c2nc3ccc4c(ccc5ccc6cc(-c7nc(-c8ccc(-n9c%10ccccc%10c%10ccccc%109)cc8)nc(-c8ccc9ccccc9c8)n7)ccc6c54)c3o2)cc1 ; c1ccc(-c2nc3ccc4c(ccc5ccc6c(-c7nc(-c8ccc9c%10ccccc%10n(-c%10ccccc%10)c9c8)nc(-c8cccc9ccccc89)n7)cccc6c54)c3o2)cc1]

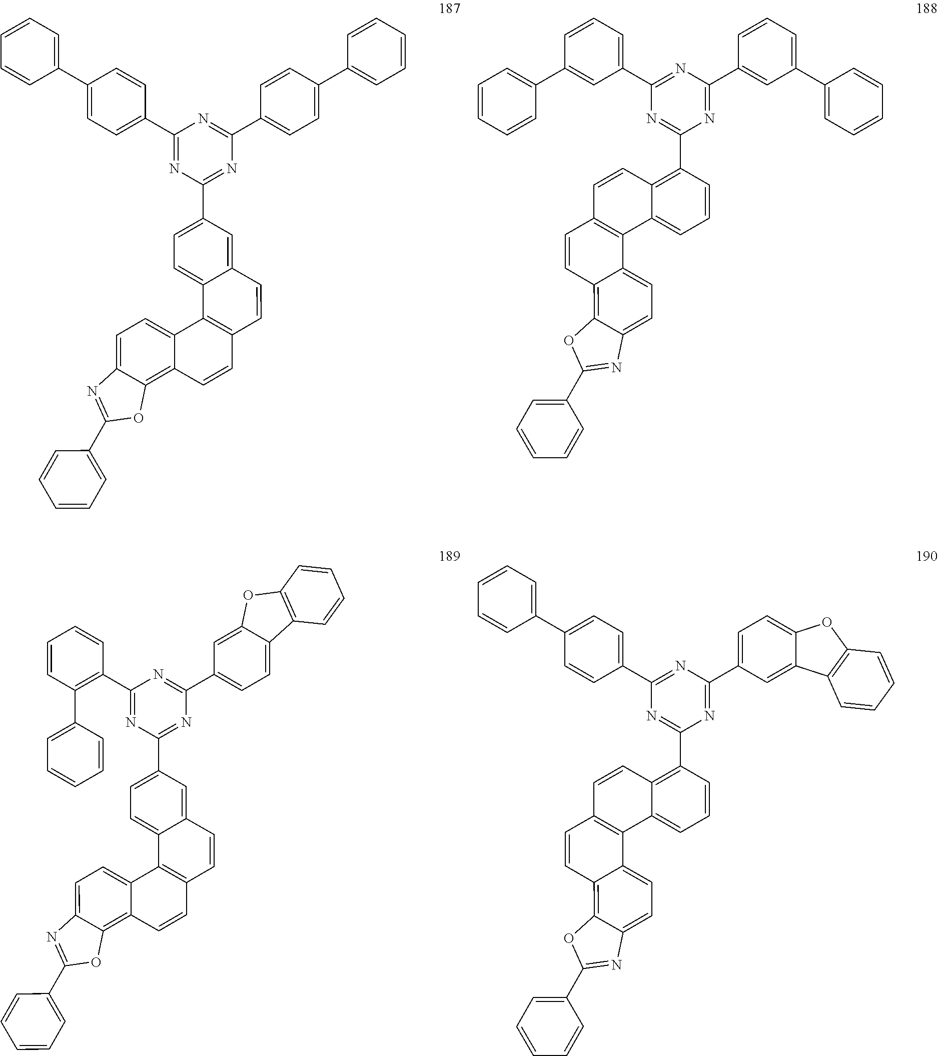 [SMILES: c1ccc(-c2ccc(-c3nc(-c4ccc(-c5ccccc5)cc4)nc(-c4ccc5c(ccc6ccc7c(ccc8nc(-c9ccccc9)oc87)c65)c4)n3)cc2)cc1 ; c1ccc(-c2cccc(-c3nc(-c4cccc(-c5ccccc5)c4)nc(-c4cccc5c4ccc4ccc6c(ccc7nc(-c8ccccc8)oc76)c45)n3)c2)cc1 ; c1ccc(-c2nc3ccc4c(ccc5ccc6cc(-c7nc(-c8ccc9c(c8)oc8ccccc89)nc(-c8ccccc8-c8ccccc8)n7)ccc6c54)c3o2)cc1 ; c1ccc(-c2ccc(-c3nc(-c4ccc5oc6ccccc6c5c4)nc(-c4cccc5c4ccc4ccc6c(ccc7nc(-c8ccccc8)oc76)c45)n3)cc2)cc1]

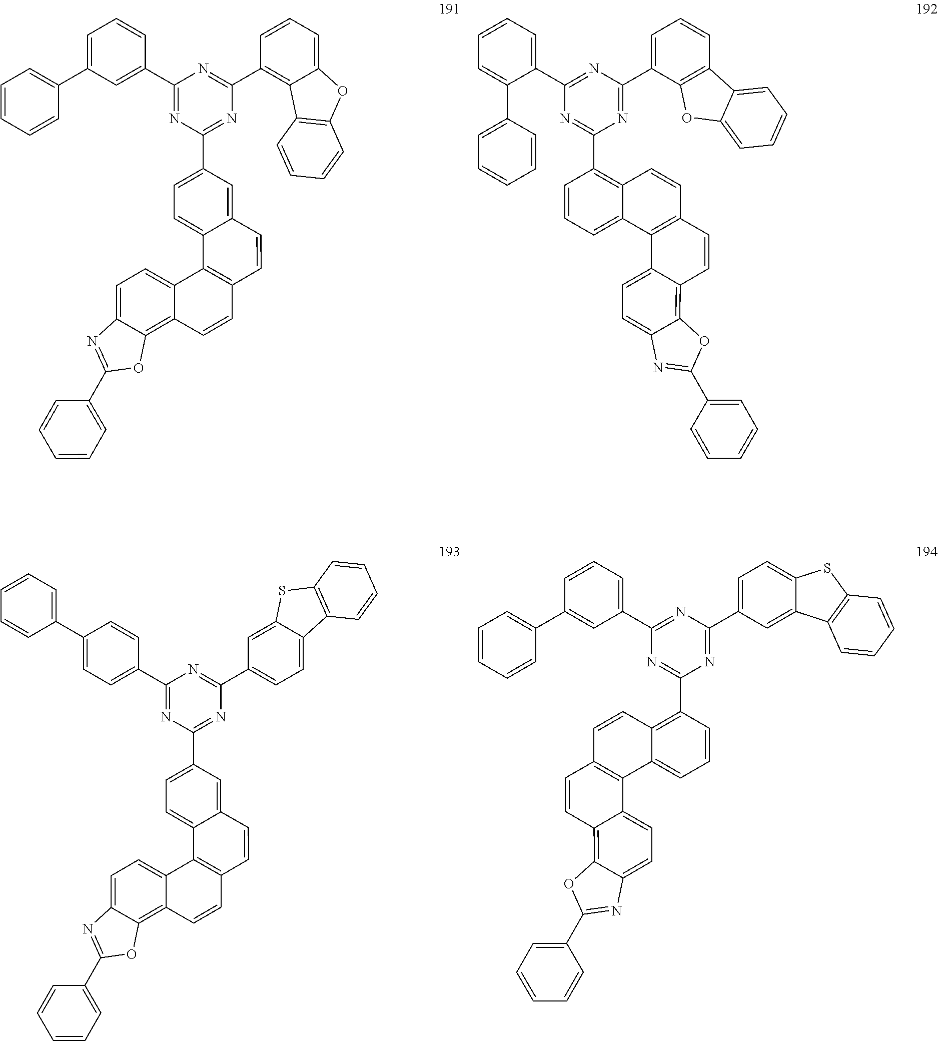 [SMILES: c1ccc(-c2cccc(-c3nc(-c4ccc5c(ccc6ccc7c(ccc8nc(-c9ccccc9)oc87)c65)c4)nc(-c4cccc5oc6ccccc6c45)n3)c2)cc1 ; c1ccc(-c2nc3ccc4c(ccc5ccc6c(-c7nc(-c8ccccc8-c8ccccc8)nc(-c8cccc9c8oc8ccccc89)n7)cccc6c54)c3o2)cc1 ; c1ccc(-c2ccc(-c3nc(-c4ccc5c(ccc6ccc7c(ccc8nc(-c9ccccc9)oc87)c65)c4)nc(-c4ccc5c(c4)sc4ccccc45)n3)cc2)cc1 ; c1ccc(-c2cccc(-c3nc(-c4ccc5sc6ccccc6c5c4)nc(-c4cccc5c4ccc4ccc6c(ccc7nc(-c8ccccc8)oc76)c45)n3)c2)cc1]

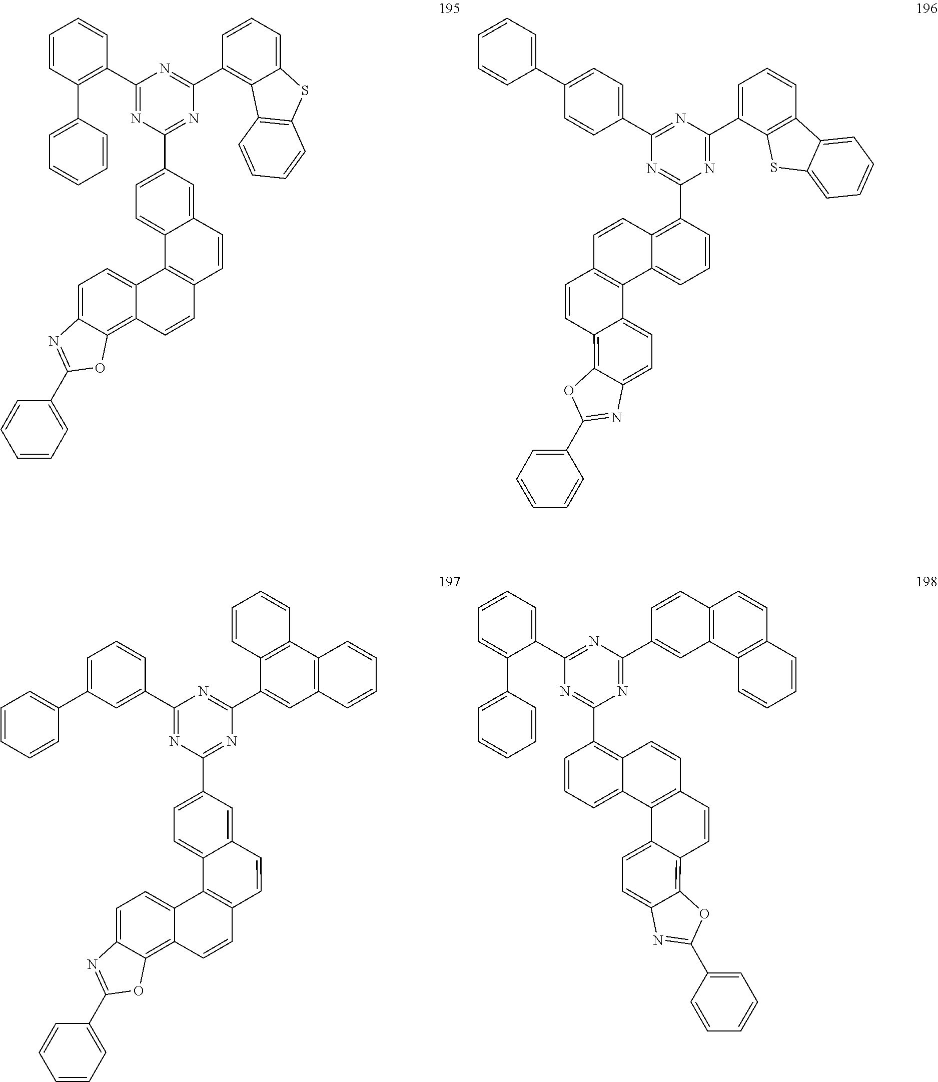 [SMILES: c1ccc(-c2nc3ccc4c(ccc5ccc6cc(-c7nc(-c8ccccc8-c8ccccc8)nc(-c8cccc9sc%10ccccc%10c89)n7)ccc6c54)c3o2)cc1 ; c1ccc(-c2ccc(-c3nc(-c4cccc5c4ccc4ccc6c(ccc7nc(-c8ccccc8)oc76)c45)nc(-c4cccc5c4sc4ccccc45)n3)cc2)cc1 ; c1ccc(-c2cccc(-c3nc(-c4ccc5c(ccc6ccc7c(ccc8nc(-c9ccccc9)oc87)c65)c4)nc(-c4cc5ccccc5c5ccccc45)n3)c2)cc1 ; c1ccc(-c2nc3ccc4c(ccc5ccc6c(-c7nc(-c8ccc9ccc%10ccccc%10c9c8)nc(-c8ccccc8-c8ccccc8)n7)cccc6c54)c3o2)cc1]

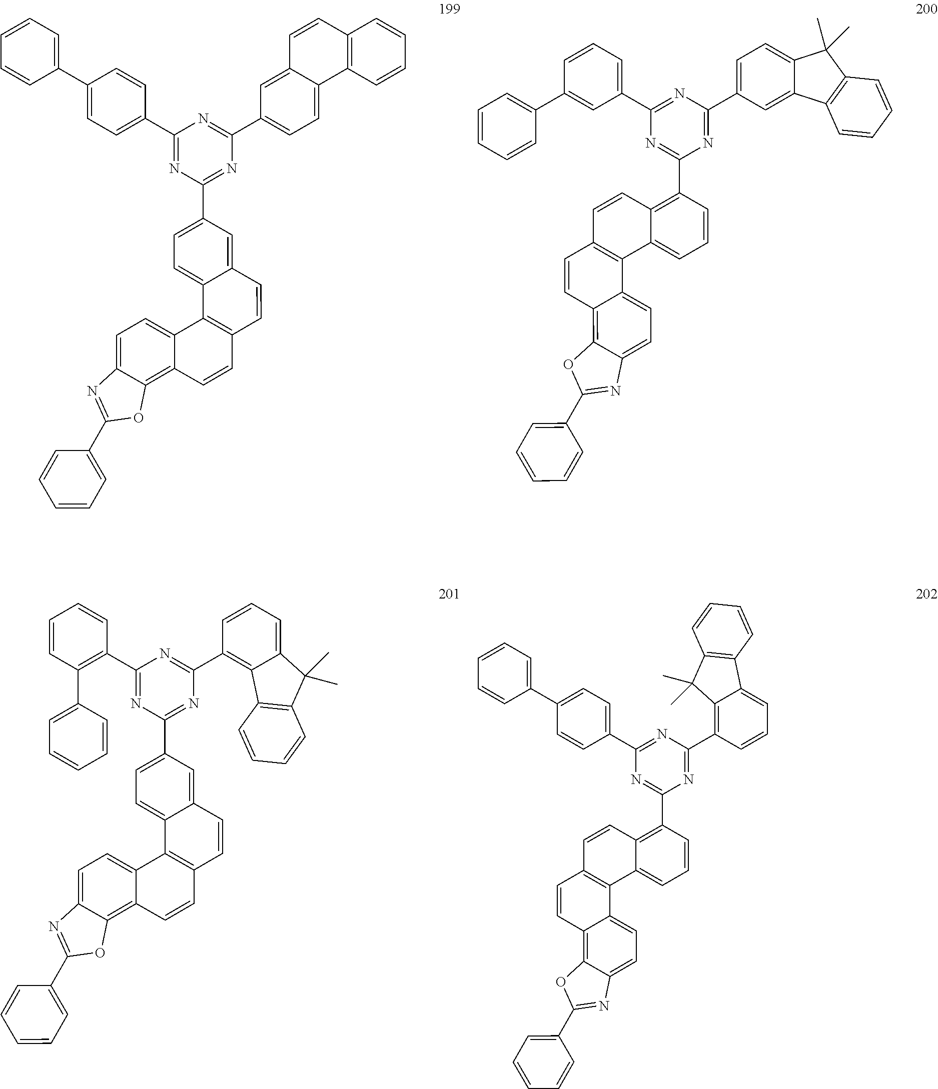 [SMILES: c1ccc(-c2ccc(-c3nc(-c4ccc5c(ccc6ccccc65)c4)nc(-c4ccc5c(ccc6ccc7c(ccc8nc(-c9ccccc9)oc87)c65)c4)n3)cc2)cc1 ; CC1(C)c2ccccc2-c2cc(-c3nc(-c4cccc(-c5ccccc5)c4)nc(-c4cccc5c4ccc4ccc6c(ccc7nc(-c8ccccc8)oc76)c45)n3)ccc21 ; CC1(C)c2ccccc2-c2c(-c3nc(-c4ccc5c(ccc6ccc7c(ccc8nc(-c9ccccc9)oc87)c65)c4)nc(-c4ccccc4-c4ccccc4)n3)cccc21 ; CC1(C)c2ccccc2-c2cccc(-c3nc(-c4ccc(-c5ccccc5)cc4)nc(-c4cccc5c4ccc4ccc6c(ccc7nc(-c8ccccc8)oc76)c45)n3)c21]

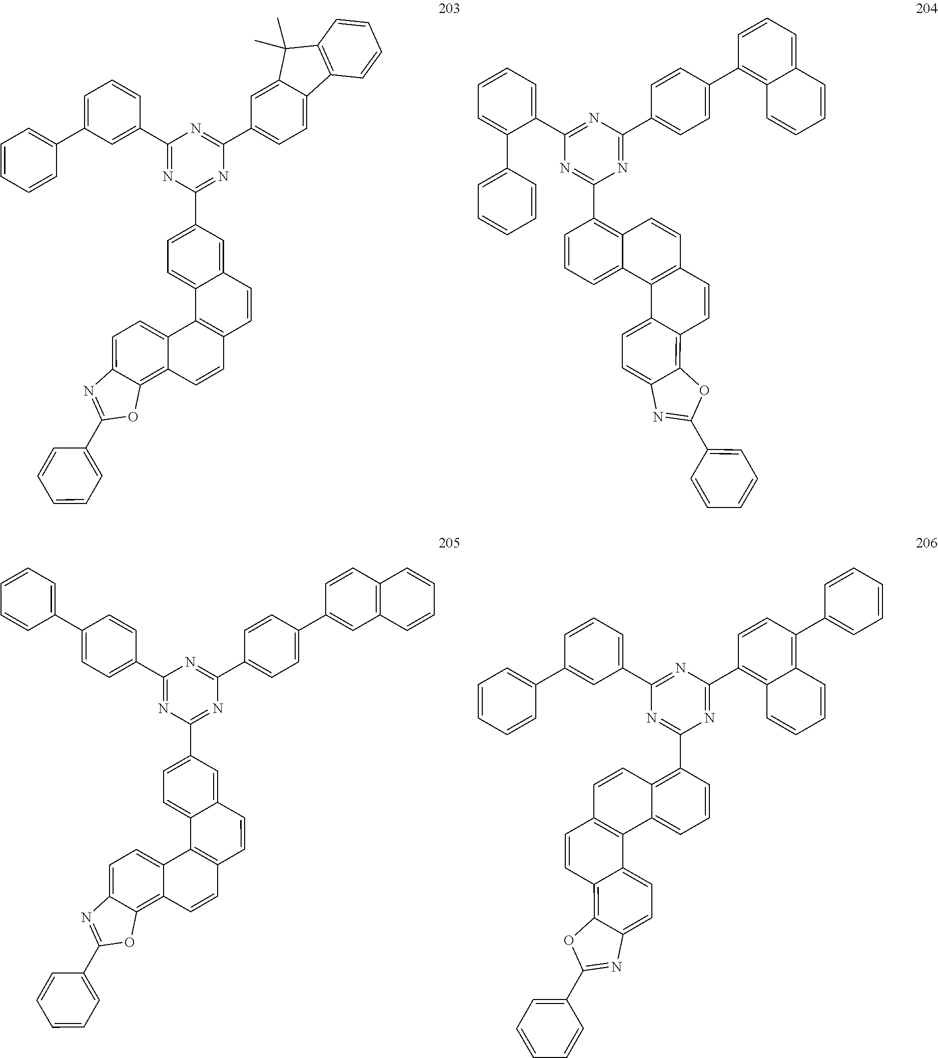 [SMILES: CC1(C)c2ccccc2-c2ccc(-c3nc(-c4cccc(-c5ccccc5)c4)nc(-c4ccc5c(ccc6ccc7c(ccc8nc(-c9ccccc9)oc87)c65)c4)n3)cc21 ; c1ccc(-c2nc3ccc4c(ccc5ccc6c(-c7nc(-c8ccc(-c9cccc%10ccccc9%10)cc8)nc(-c8ccccc8-c8ccccc8)n7)cccc6c54)c3o2)cc1 ; c1ccc(-c2ccc(-c3nc(-c4ccc(-c5ccc6ccccc6c5)cc4)nc(-c4ccc5c(ccc6ccc7c(ccc8nc(-c9ccccc9)oc87)c65)c4)n3)cc2)cc1 ; c1ccc(-c2cccc(-c3nc(-c4ccc(-c5ccccc5)c5ccccc45)nc(-c4cccc5c4ccc4ccc6c(ccc7nc(-c8ccccc8)oc76)c45)n3)c2)cc1]

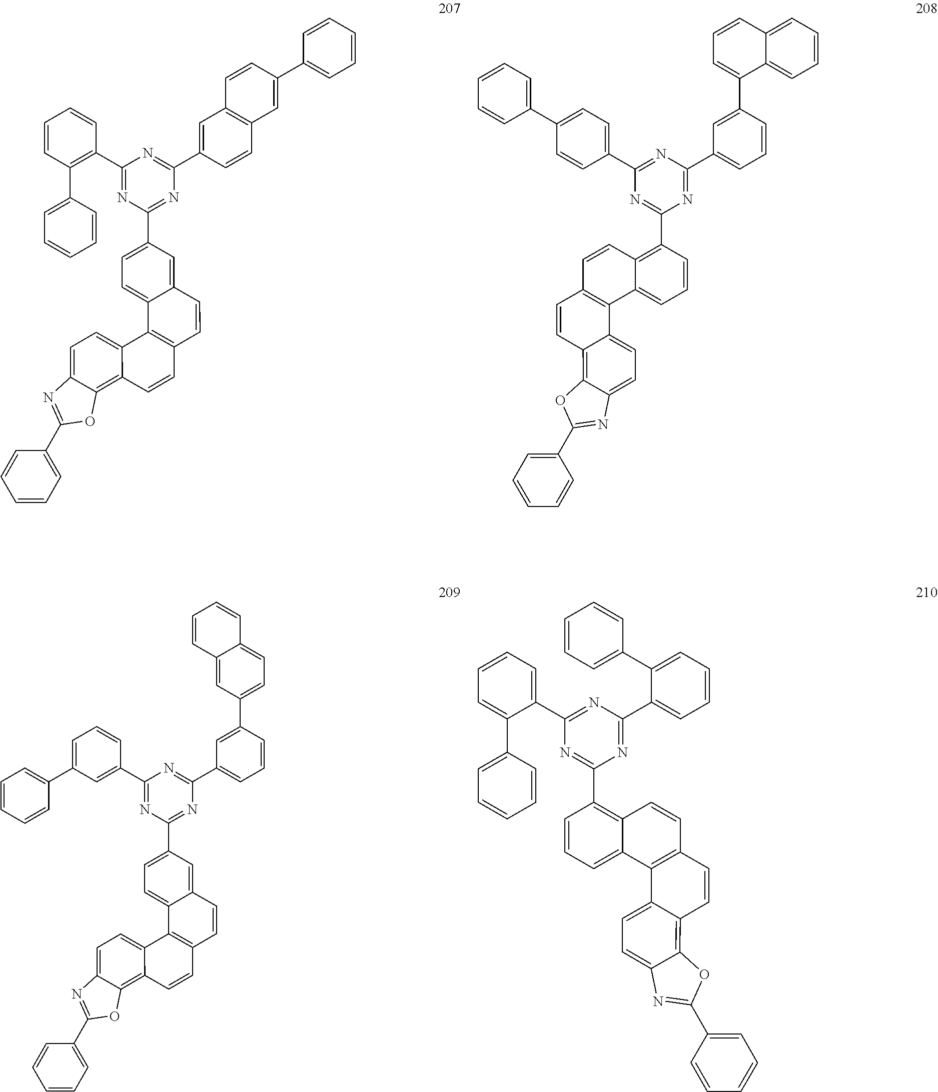 [SMILES: c1ccc(-c2ccc3cc(-c4nc(-c5ccc6c(ccc7ccc8c(ccc9nc(-c%10ccccc%10)oc98)c76)c5)nc(-c5ccccc5-c5ccccc5)n4)ccc3c2)cc1 ; c1ccc(-c2ccc(-c3nc(-c4cccc(-c5cccc6ccccc56)c4)nc(-c4cccc5c4ccc4ccc6c(ccc7nc(-c8ccccc8)oc76)c45)n3)cc2)cc1 ; c1ccc(-c2cccc(-c3nc(-c4cccc(-c5ccc6ccccc6c5)c4)nc(-c4ccc5c(ccc6ccc7c(ccc8nc(-c9ccccc9)oc87)c65)c4)n3)c2)cc1 ; c1ccc(-c2nc3ccc4c(ccc5ccc6c(-c7nc(-c8ccccc8-c8ccccc8)nc(-c8ccccc8-c8ccccc8)n7)cccc6c54)c3o2)cc1]

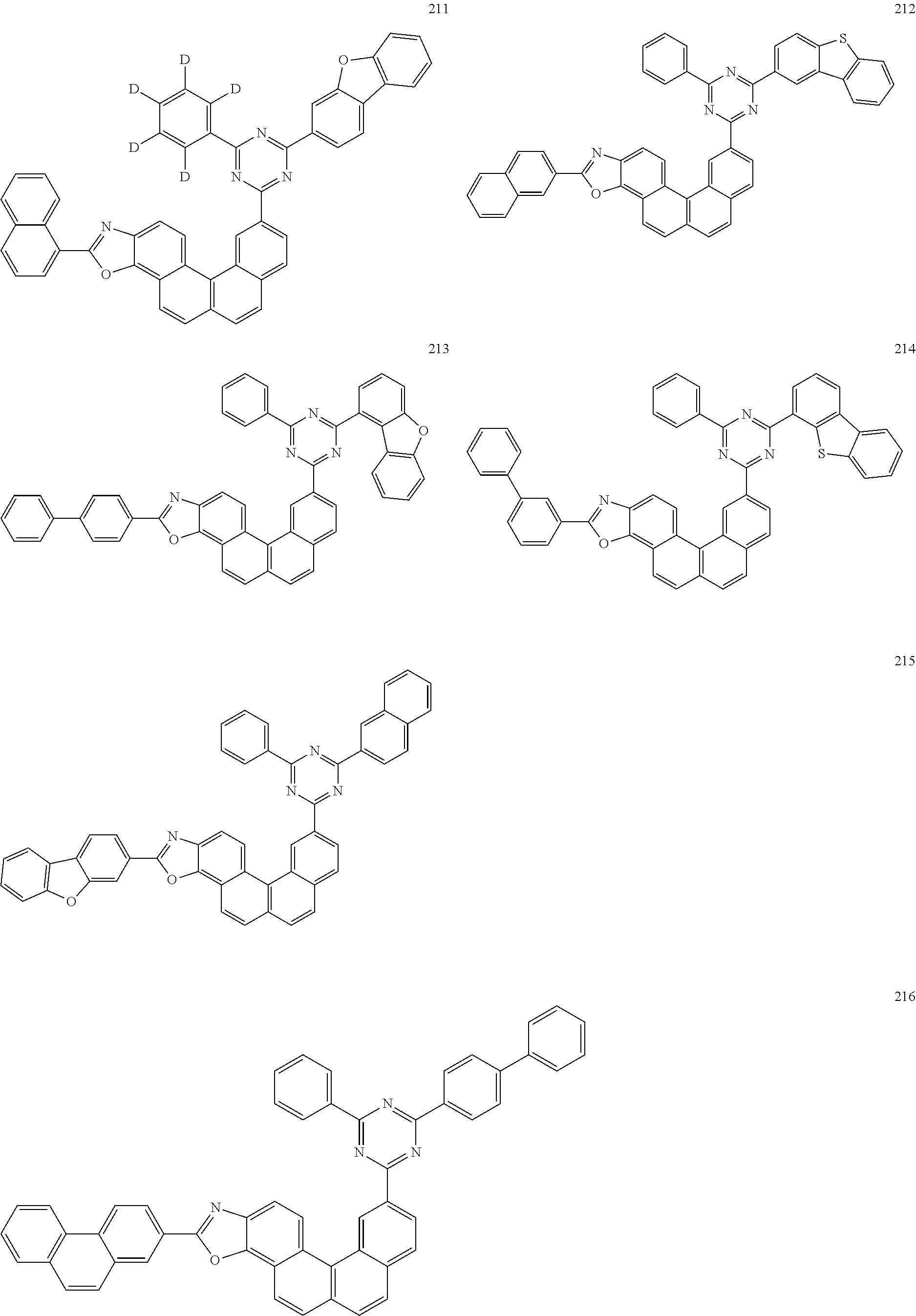

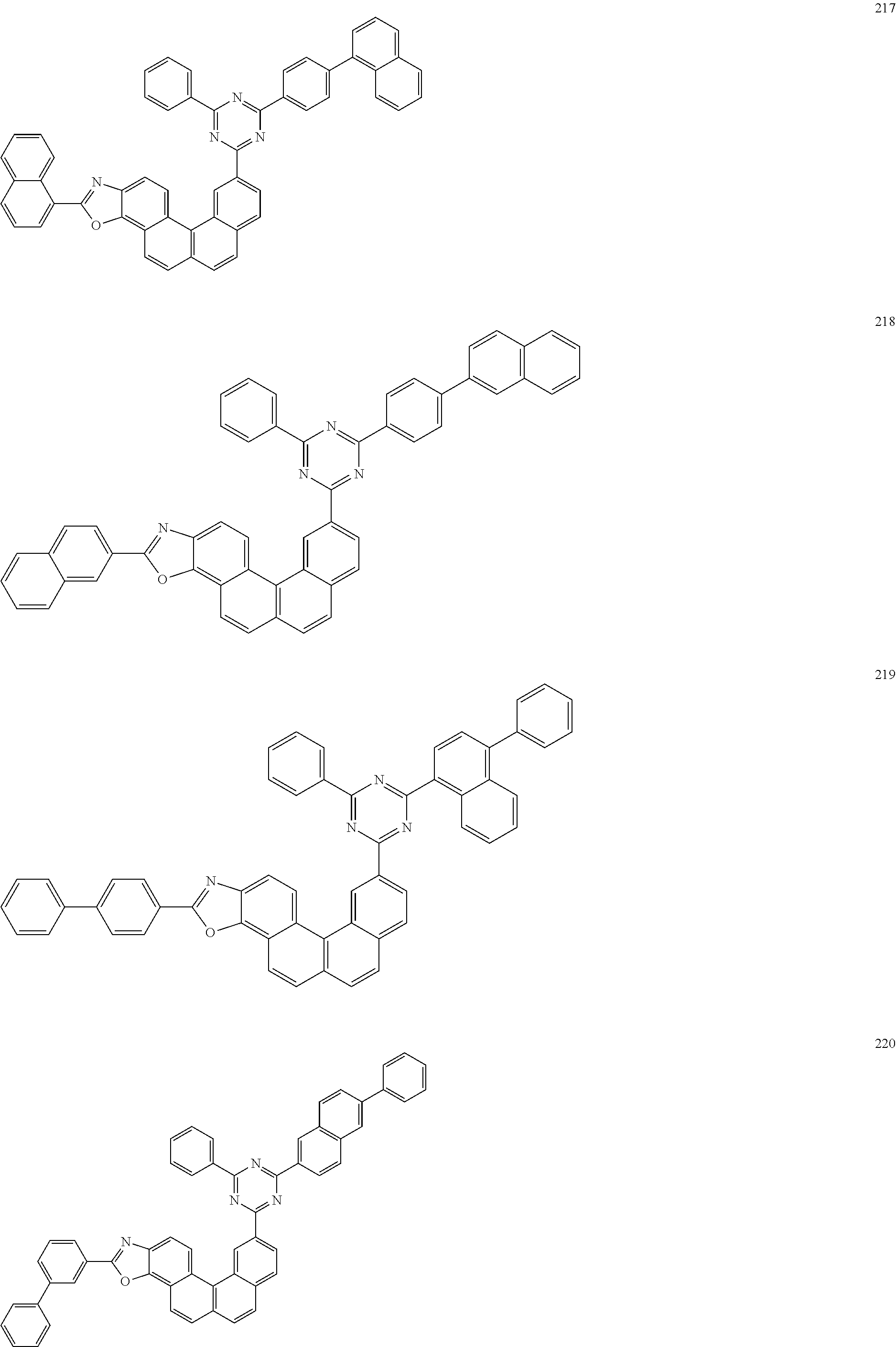 [SMILES: c1ccc(-c2nc(-c3ccc(-c4cccc5ccccc45)cc3)nc(-c3ccc4ccc5ccc6c(ccc7nc(-c8cccc9ccccc89)oc76)c5c4c3)n2)cc1 ; c1ccc(-c2nc(-c3ccc(-c4ccc5ccccc5c4)cc3)nc(-c3ccc4ccc5ccc6c(ccc7nc(-c8ccc9ccccc9c8)oc76)c5c4c3)n2)cc1 ; c1ccc(-c2ccc(-c3nc4ccc5c(ccc6ccc7ccc(-c8nc(-c9ccccc9)nc(-c9ccc(-c%10ccccc%10)c%10ccccc9%10)n8)cc7c65)c4o3)cc2)cc1 ; c1ccc(-c2cccc(-c3nc4ccc5c(ccc6ccc7ccc(-c8nc(-c9ccccc9)nc(-c9ccc%10cc(-c%11ccccc%11)ccc%10c9)n8)cc7c65)c4o3)c2)cc1]

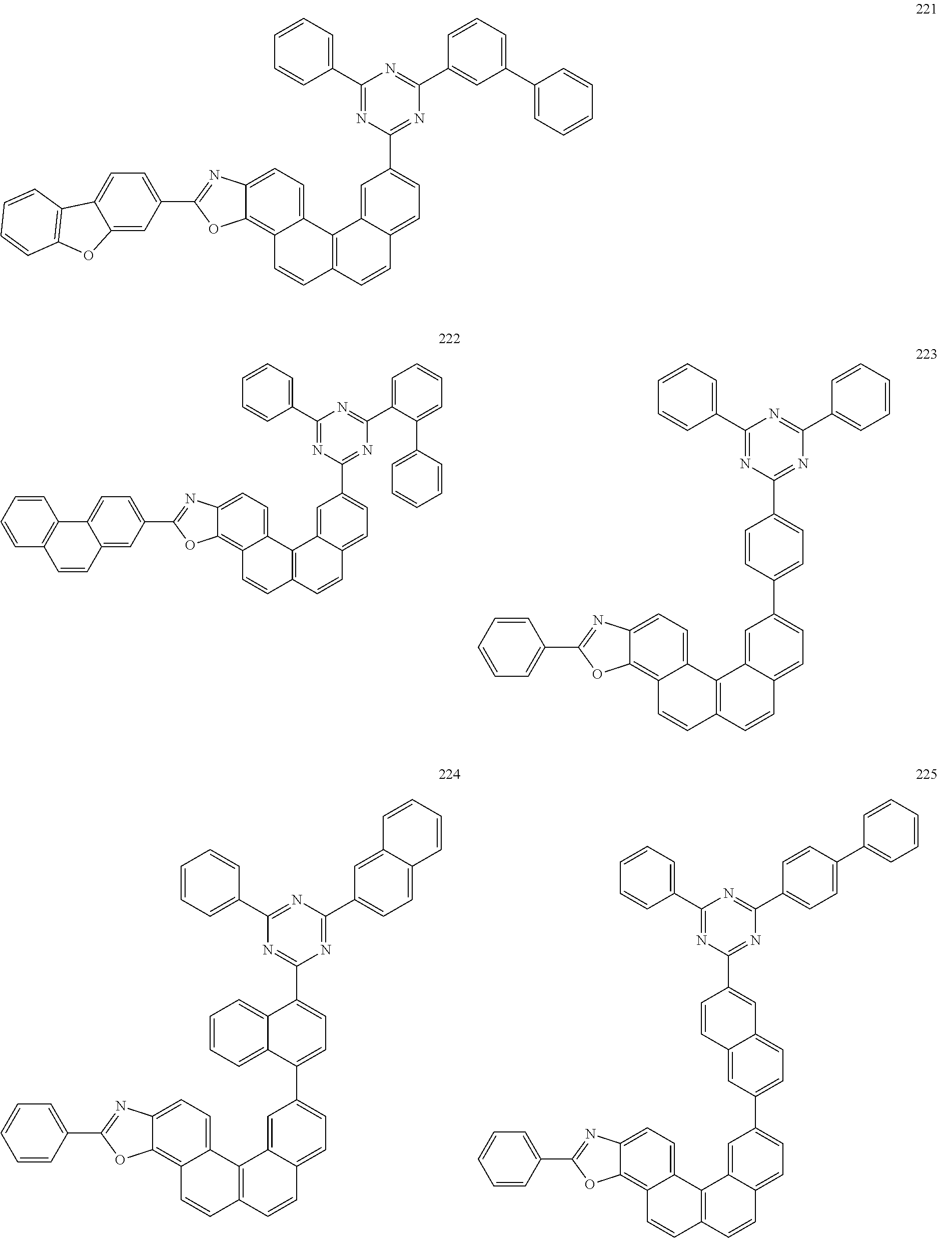

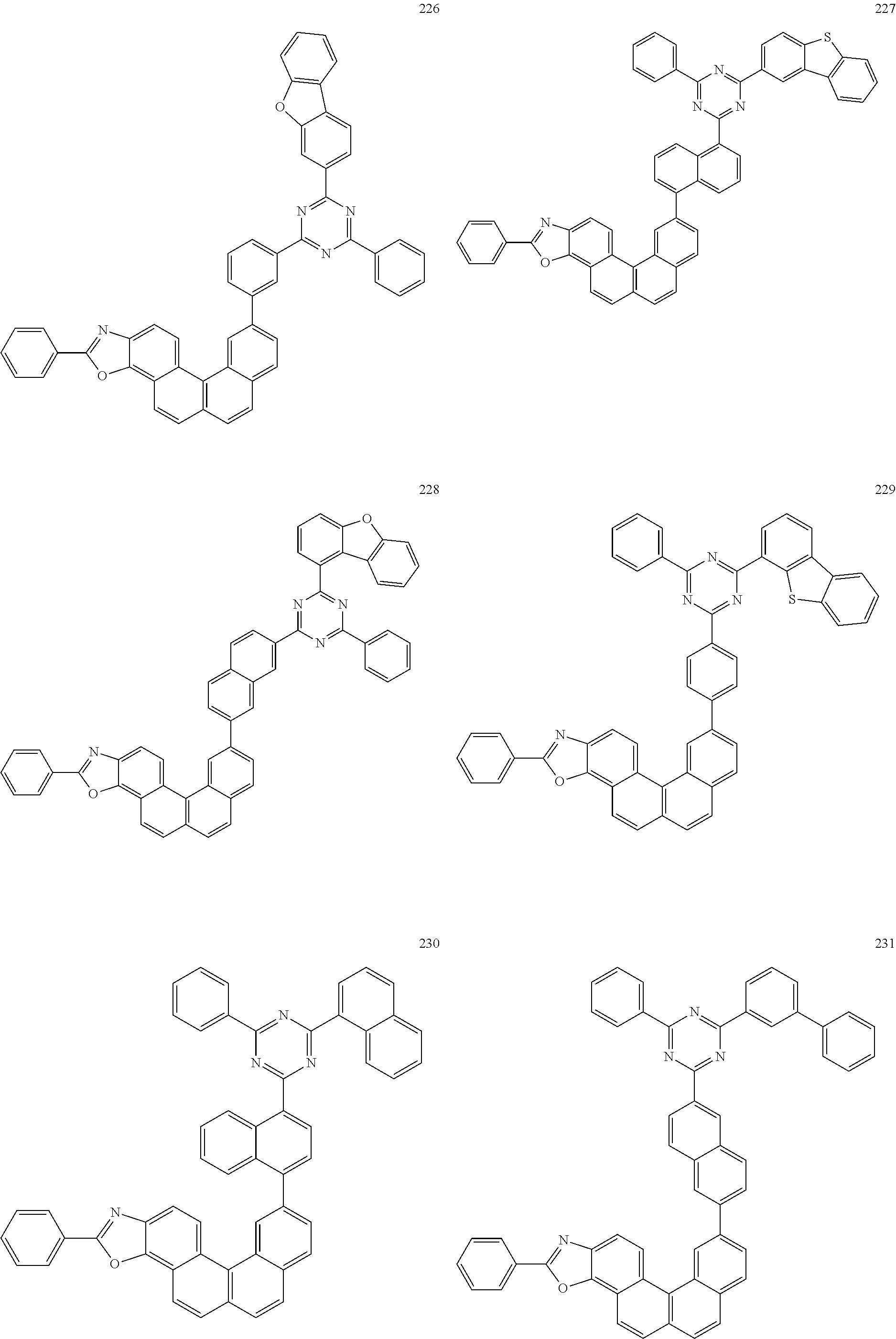

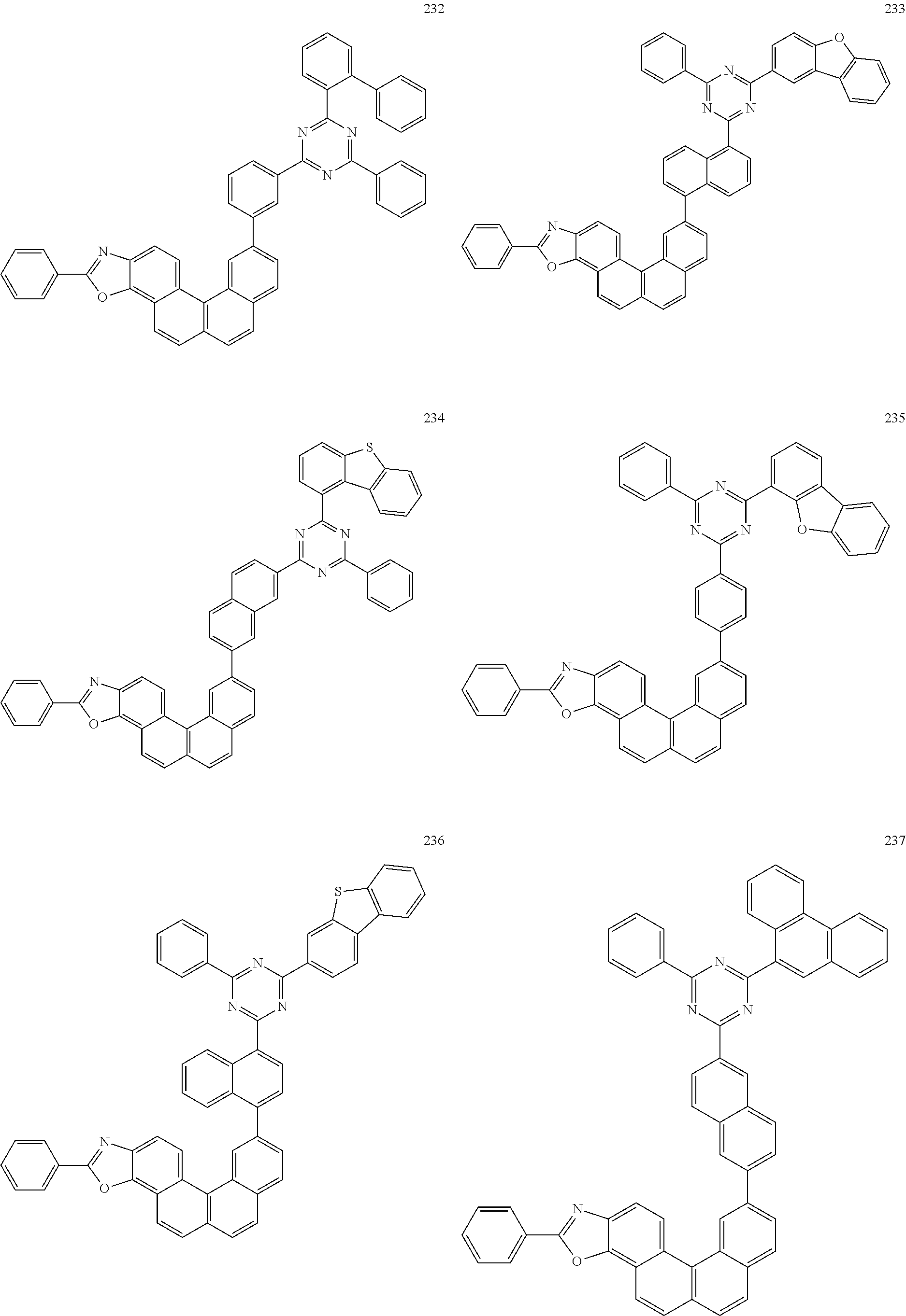

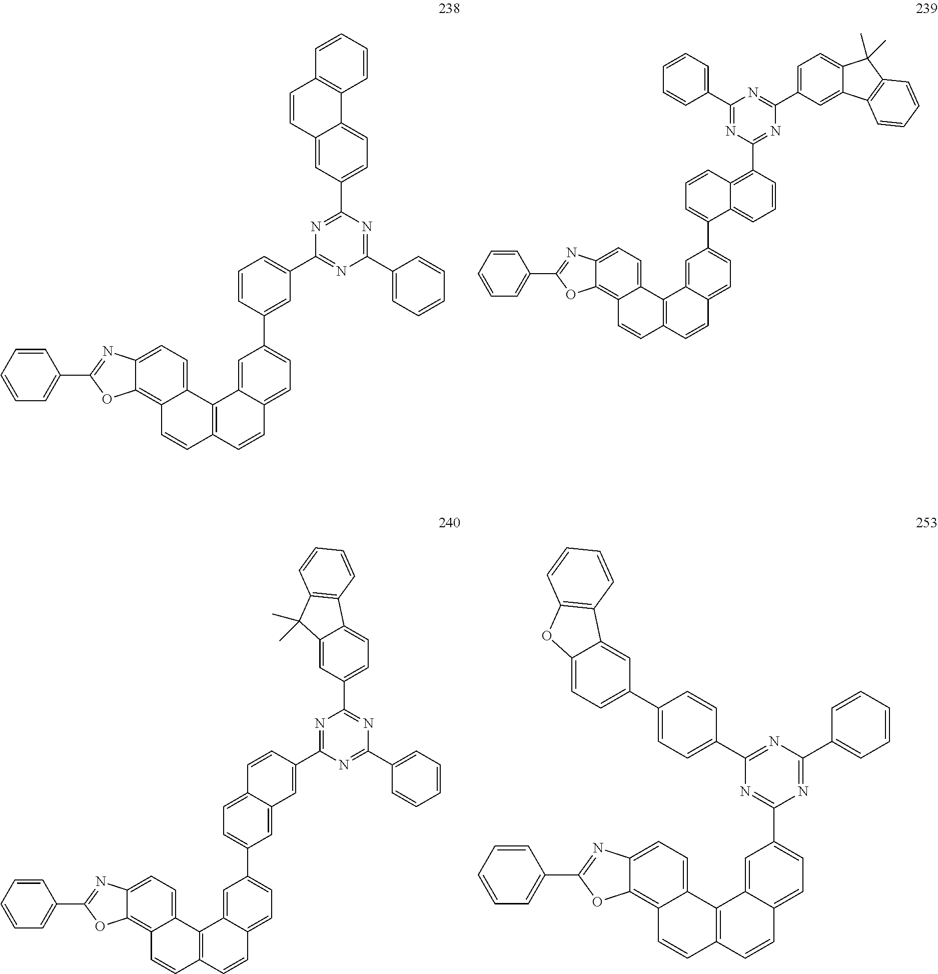

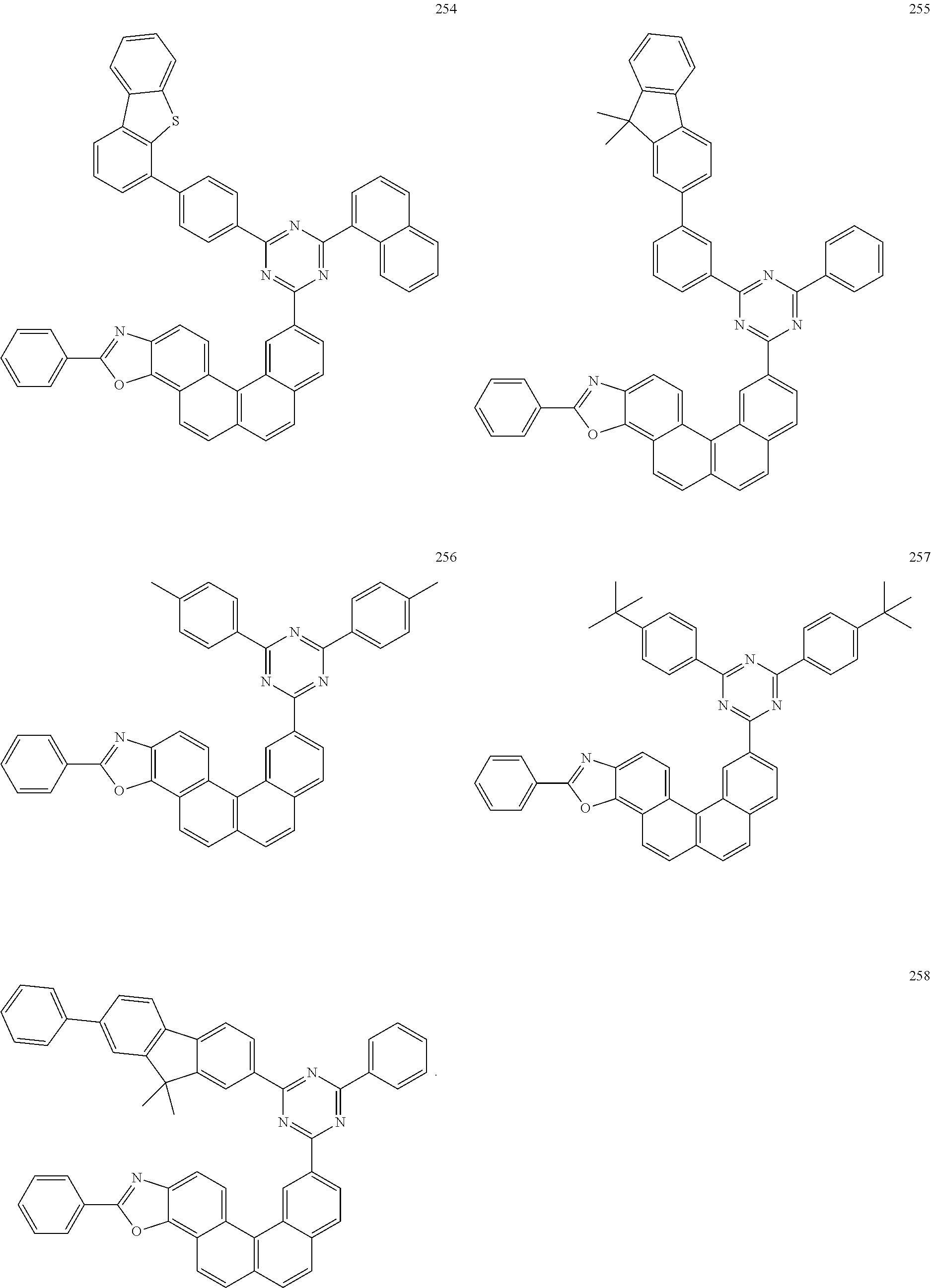

22. An organic electroluminescent device, comprising an anode and a cathode disposed opposite each other, and a functional layer disposed between the anode and the cathode; characterize in that the functional layer comprises an organic light-emitting layer, the organic light-emitting layer comprising the organic compound according to claim 14.

23. An electronic apparatus comprising the organic electroluminescent device according to claim 22.
